SEQUENCE LISTING (55 sequences)

SEQ ID NO: 1 - protein, 352 aa, synthetic construct
MDSYLLMWGLLTFIMVPGCQADVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDVAVAGCVFLLISVLLLSGLTWQRRQRKSRRTI

SEQ ID NO: 2 - protein, 636 aa, synthetic construct
MAAPALSWRLPLLILLLPLATSWASADVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 3 - protein, 428 aa, synthetic construct
MLKPSLPFTSLLFLQLPLLGVGDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 4 - protein, 539 aa, synthetic construct
MTILGTTFGMVFSLLQVVSGDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 5 - protein, 391 aa, synthetic construct
MAPRRARGCRTLGLPALLLLLLLRPPATRGDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDVAISTSTVLLCGLSAVSLLACYLKSRQTPPLASVEMEAMEALPVTWGTSSRDEDLENCSHHL

SEQ ID NO: 6 - protein, 624 aa, synthetic construct
MPRGWAAPLLLLLLQGGWGDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 7 - protein, 1444 aa, synthetic construct
MAAPALSWRLPLLILLLPLATSWASADVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVTGSGATNFSLLKQAGDVEENPGPAMLKPSLPFTSLLFLQLPLLGVGDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGEGRGSLLTCGDVEENPGPWEMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 8 - DNA, 1062 bp, synthetic construct
  source 1..1062: mol_type = other DNA
atggacagctacctgctgatgtggggcctgctgaccttcatcatggtgcctggctgtcaggccgacgtggtcatgacacagacacctctgagcctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggacttcgcctgtgatgtggccgtggccggatgtgtgtttctgctgatctctgtgctgctgctgagcggcctgacttggcaaagacggcagagaaagagccggcggaccatctgataa

SEQ ID NO: 9 - DNA, 1914 bp, synthetic construct
  source 1..1914: mol_type = other DNA
atggctgctccagctctgtcttggagactgcccctgctgattctgctgctgcctctggctacatcttgggcctctgccgacgtggtcatgacacagacaccactgagcctgcctgtgtctctgggagatcaggccagcatcagctgcagatccagccagtctctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggatttcgcctgcgacatcccctggctgggacatctgcttgttggactgtctggcgccttcggcttcatcatcctggtgtatctgctgatcaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagcccaggtggactggcccctgagattagccctctggaagtgctggaacgggacaaagtgacccagctgctcctccagcaggataaggtgccagaacctgccagcctgtccagcaatcacagcctgaccagctgctttaccaaccagggctacttcttcttccatctgcctgacgctctggaaatcgaggcctgccaggtgtacttcacctacgatccctacagcgaagaggaccccgatgaaggtgttgctggcgcccctacaggatcttctccacagcctctgcaacctctgagcggcgaggatgatgcctactgcacctttccaagcagggacgacctgctcctgttcagcccatctctgctcggaggaccatctcctccatctacagctccaggcggatctggcgctggcgaggaaagaatgccacctagcctgcaagagcgggtgcccagagattgggatcctcaacctctcggccctccaacacctggcgtgccagatctcgtggactttcagcctcctccagagctggtgctgagagaagctggcgaagaagtgccagacgctggccctagagagggcgttagctttccttggagcagacctcctggacagggcgagtttagggccctgaatgcaagactgcctctgaacaccgacgcctacctgtctctgcaagaactgcagggacaagaccccacacacctggtgtaatga

SEQ ID NO: 10 - DNA, 1290 bp, synthetic construct
  source 1..1290: mol_type = other DNA
atgctgaagcccagcctgccttttaccagcctgctgttcctgcagctgcctctgcttggcgtgggagatgtggtcatgacacagaccccactgagcctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggacttcgcctgcgacgtggtcatctctgtgggctctatgggcctgatcatctccctgctgtgtgtgtacttttggctggaacggaccatgcctcggatccccacactgaagaacctggaagatctggtcaccgagtaccacggcaacttcagtgcttggagcggcgtgtcaaaaggactggccgaaagcctgcagcctgactactccgagagactgtgcctggtgtctgagatccctcctaaaggcggcgctctcggagaaggacctggtgcctctccatgcaatcagcacagcccttattgggcccctccttgctacaccctgaaacctgagacatgatga

SEQ ID NO: 11 - DNA, 3174 bp, synthetic construct
  source 1..3174: mol_type = other DNA
atgacaatcctgggcaccaccttcggcatggtgttcagtctgctgcaggtcgtgtctggcgacgtggtcatgacacagaccccactgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcacctttggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagcctctgtctctgaggcccgaagcttgtagacctgctgccggcggagctgtgcatacaagaggcctggatttcgcctgcgatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctggaagagagcgagaagcagagactcggaggcgacgtgcagagccctaattgcccttctgaggacgtcgtgatcacccctgagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgagcctgggaaccaccaatagcacactgcctccaccattcagcctgcagagcggcatcctgacactgaaccctgttgctcagggccagccaatcctgaccagcctgggcagcaatcaagaagaggcctacgtcaccatgagcagcttctaccagaaccagtgatgatgggaatggaacctaccagacctgggtggccagctcactgagccatgcgctatgtccgggaatggaacctaccagacctgggtggccaccaggatttgccaaggagaggagcagaggttcacctgctacatggaacacagcgggaatcacagcactcaccctgtgccctctgggaaagtgctggtgcttcagagtcattggcagacattccatgtttcttaggctcgaggtcgacggtatcgataagcttgatatccgcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacgcgtaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtga

SEQ ID NO: 12 - DNA, 1179 bp, synthetic construct
  source 1..1179: mol_type = other DNA
atggctcctcggagagccagaggctgtagaacacttggactgcccgctctgctgctgctcctgcttcttagacctcctgccacaagaggcgacgtggtcatgacacagacccctctgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggacttcgcctgtgacgtggccatcagcacaagcaccgttctgctgtgtggcctgtcagccgttagcctgctggcttgctacctgaagtccagacagacacctcctctggccagcgtggaaatggaagccatggaagctctgccagtgacctggggcacctccagcagagatgaggatctggaaaactgcagccaccacctgtgatga

SEQ ID NO: 13 - DNA, 1878 bp, synthetic construct
  source 1..1878: mol_type = other DNA
atgccaagaggatgggccgctcctcttctcctgttgctgcttcaaggcggctggggcgacgttgtgatgacacagacaccactgagcctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggactttgcctgcgacggctggaatcctcatctgttgttgttgctcctcctggtcatcgtgttcatccccgccttttggagcctgaaaacacaccctctgtggcggctgtggaagaaaatctgggccgtgccatctcctgagcggttcttcatgcctctgtacaagggctgcagcggcgacttcaagaaatgggtcggagccccttttaccggcagctctctggaacttggaccttggagccctgaagtgcccagcacactggaagtgtacagctgtcaccctcctagaagccccgccaagagactgcagctcacagagctgcaagagcctgccgagctggtggaatctgatggcgtgcccaagcctagcttctggcccaccgctcaaaattctggcggcagcgcctacagcgaggaaagagatagaccttacggcctggtgtccatcgacaccgtgacagtgctggatgccgagggaccttgtacctggccttgtagctgcgaggacgatggctaccctgctctggatctggacgcaggactggaaccttctccaggcctcgaagatcctctgctggacgccggaacaacagtgctgtcttgtggctgtgtgtccgccggatctcctggacttggaggacctctgggaagcctgctggacagactgaaacctcctctggccgatggcgaagattgggctggtggacttccttggggcggaagatctccaggcggagtgtctgaatctgaggccggttctccactggccggcctggatatggataccttcgatagcggcttcgtgggcagcgattgcagcagccctgtggaatgcgacttcacatctcctggcgacgagggcccacctagaagctatctcagacagtgggtcgtgatccctccacctctgtctagtcctggaccacaggccagctgatga

SEQ ID NO: 14 - DNA, 4338 bp, synthetic construct
  source 1..4338: mol_type = other DNA
atggctgctccagctctgtcttggagactgcccctgctgattctgctgctgcctctggctacatcttgggcctctgccgacgtggtcatgacacagacaccactgagcctgcctgtgtctctgggagatcaggccagcatcagctgcagatccagccagtctctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggatttcgcctgcgacatcccctggctgggacatctgcttgttggactgtctggcgccttcggcttcatcatcctggtgtatctgctgatcaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagcccaggtggactggcccctgagattagccctctggaagtgctggaacgggacaaagtgacccagctgctcctccagcaggataaggtgccagaacctgccagcctgtccagcaatcacagcctgaccagctgctttaccaaccagggctacttcttcttccatctgcctgacgctctggaaatcgaggcctgccaggtgtacttcacctacgatccctacagcgaagaggaccccgatgaaggtgttgctggcgcccctacaggatcttctccacagcctctgcaacctctgagcggcgaggatgatgcctactgcacctttccaagcagggacgacctgctcctgttcagcccatctctgctcggaggaccatctcctccatctacagctccaggcggatctggcgctggcgaggaaagaatgccacctagcctgcaagagcgggtgcccagagattgggatcctcaacctctcggccctccaacacctggcgtgccagatctcgtggactttcagcctcctccagagctggtgctgagagaagctggcgaagaagtgccagacgctggccctagagagggcgttagctttccttggagcagacctcctggacagggcgagtttagggccctgaatgcaagactgcctctgaacaccgacgcctacctgtctctgcaagaactgcagggacaagaccccacacacctggtgactggatctggagcaacaaacttctcactactcaaacaagcaggtgacgtggaggagaatcccgggcctgccatgctgaagcccagcctgccttttaccagcctgctgttcctgcagctgcctctgcttggcgtgggagatgtggtcatgacacagaccccactgagcctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggacttcgcctgcgacgtggtcatctctgtgggctctatgggcctgatcatctccctgctgtgtgtgtacttttggctggaacggaccatgcctcggatccccacactgaagaacctggaagatctggtcaccgagtaccacggcaacttcagtgcttggagcggcgtgtcaaaaggactggccgaaagcctgcagcctgactactccgagagactgtgcctggtgtctgagatccctcctaaaggcggcgctctcggagaaggacctggtgcctctccatgcaatcagcacagcccttattgggcccctccttgctacaccctgaaacctgagacaggctccggcgagggcaggggaagtcttctaacatgcggggacgtggaggaaaatcccggcccatgggaaatgcctcctcctcggctgctgttcttcctgctgtttctgacccctatggaagtgcggcccgaggaacctctggtggtcaaagttgaagagggcgacaacgccgtgctgcagtgtctgaagggcacatctgatggccccacacagcagctgacctggtctagagagagccctctgaagcccttcctgaagctgtctctgggactgcctggactgggcatccatatgaggcctctggccatctggctgttcatcttcaacgtgtcccagcagatgggcggcttctacctgtgtcaacctggacctccaagcgagaaggcttggcagcctggctggaccgtgaatgtggaaggatccggcgagctgttccggtggaatgtgtctgatctcggcggcctcggatgcggcctgaagaatagatctagcgagggccctagcagccccagcggaaaactgatgagccccaagctgtacgtgtgggccaaagacagacccgagatttgggagggcgagcctccttgtctgcctcctagagacagcctgaaccagagcctgagccaggacctgacaatggcccctggatctacactgtggctgagctgtggcgtgccacctgacagtgtgtctagaggccctctgtcttggacccacgtgcaccctaagggccctaagtctctgctgagcctggaactgaaggacgacaggcccgccagagatatgtgggtcatggaaacaggcctgctgctgcctagagccacagcacaggatgccggcaagtactactgccacagaggcaacctgaccatgagcttccacctggaaatcaccgccagacctgtcctgtggcactggctgcttagaaccggcggctggaaagtgtctgccgtgactctggcctacctgatcttctgcctgtgtagcctcgtgggcatcctgcatctgcagagagcactggtcctgcggcggaagcggaagagaatgaccgatcctaccagacggttctgatga

SEQ ID NO: 15 - protein, 21 aa, synthetic construct
MDSYLLMWGLLTFIMVPGCQA

SEQ ID NO: 16 - protein, 26 aa, synthetic construct
MAAPALSWRLPLLILLLPLATSWASA

SEQ ID NO: 17 - protein, 22 aa, synthetic construct
MLKPSLPFTSLLFLQLPLLGVG

SEQ ID NO: 18 - protein, 20 aa, synthetic construct
MTILGTTFGMVFSLLQVVSG

SEQ ID NO: 19 - protein, 30 aa, synthetic construct
MAPRRARGCRTLGLPALLLLLLLRPPATRG

SEQ ID NO: 20 - protein, 19 aa, synthetic construct
MPRGWAAPLLLLLLQGGWG

SEQ ID NO: 21 - protein, 254 aa, synthetic construct
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVS

SEQ ID NO: 22 - protein, 45 aa, synthetic construct
TTTPAPRPPTPAPTIASQPLSLRPEACRPAAGGAVHTRGLDFACD

SEQ ID NO: 23 - protein, 19 aa, synthetic construct
VAVAGCVFLLISVLLLSGL

SEQ ID NO: 24 - protein, 25 aa, synthetic construct
IPWLGHLLVGLSGAFGFIILVYLLI

SEQ ID NO: 25 - protein, 21 aa, synthetic construct
VVISVGSMGLIISLLCVYFWL

SEQ ID NO: 26 - protein, 25 aa, synthetic construct
PILLTISILSFFSVALLVILACVLW

SEQ ID NO: 27 - protein, 23 aa, synthetic construct
VAISTSTVLLCGLSAVSLLACYL

SEQ ID NO: 28 - protein, 21 aa, synthetic construct
GWNPHLLLLLLLVIVFIPAFW

SEQ ID NO: 29 - protein, 13 aa, synthetic construct
TWQRRQRKSRRTI

SEQ ID NO: 30 - protein, 286 aa, synthetic construct
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 31 - protein, 86 aa, synthetic construct
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 32 - protein, 195 aa, synthetic construct
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 33 - protein, 39 aa, synthetic construct
KSRQTPPLASVEMEAMEALPVTWGTSSRDEDLENCSHHL

SEQ ID NO: 34 - protein, 285 aa, synthetic construct
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 35 - protein, 22 aa, synthetic construct
GSGATNFSLLKQAGDVEENPGP

SEQ ID NO: 36 - protein, 21 aa, synthetic construct
GSGEGRGSLLTCGDVEENPGP

SEQ ID NO: 37 - protein, 23 aa, synthetic construct
GSGQCTNYALLKLAGDVESNPGP

SEQ ID NO: 38 - protein, 25 aa, synthetic construct
GSGVKQTLNFDLLKLAGDVESNPGP

SEQ ID NO: 39 - protein, 333 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 40 - DNA, 2550 bp, synthetic construct
  source 1..2550: mol_type = other DNA
atggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatctggcgccacaaacttctcactgctgaaacaggccggcgacgtcgaagagaatcctgggcctatgcctcctcctcggctgctgttcttcctgctgtttctgacccctatggaagtgcggcccgaggaacctctggtcgtgaaagttgaagagggcgacaacgccgtgctgcagtgtctgaagggcacatctgatggccccacacagcagctgacctggtctagagagagccctctgaagcccttcctgaagctcagtctgggactgcctggactgggcatccatatgaggccactggccatctggctgttcatctttaacgtgtcccagcagatgggaggcttctacctgtgtcagcctggacctcctagcgagaaagcttggcagcctggctggaccgtgaatgtggaaggatccggcgagctgttccggtggaatgtctctgatctcggcggcctcggatgcggcctgaagaatagatctagcgagggccctagcagcccctccggaaaactgatgagccccaagctgtacgtgtgggccaaagacagacccgagatttgggagggcgagcctccttgtctgccacctagagacagcctgaatcagagcctgagccaggacctgacaatggcccctggatctacactgtggctgagctgcggagtgcctcctgacagtgtgtctagaggcccactgagctggacacacgtgcaccctaagggccctaagagcctgctgagtctggaactgaaggacgacaggcccgccagagatatgtgggtcatggaaaccggactgctgctccctagagccactgctcaagatgccggcaagtactattgccaccggggcaacctgaccatgagcttccacctggaaattaccgccagaccagtgctgtggcattggctgcttagaaccggcggatggaaggtgtcagccgtgactctggcctacctgatcttttgtctgtgcagcctcgtgggcatcctgcatctgcaaagagccctggtcctgcggcggaagcggaagagaatgaccgatcctaccagacggttctgatga

SEQ ID NO: 41 - DNA, 1500 bp, synthetic construct
  source 1..1500: mol_type = other DNA
atggctctgcctgtcaccgctctgctgctgcctctggctctgctgctgcacgctgctcgccctcaggtcaaactggaagaaagtgggggaggcctggtgcaggcaggacggagcctgcgcctgagctgcgcagcatccgagcacaccttcagctcccacgtgatgggctggtttcggcaggccccaggcaaggagagagagtccgtggccgtgatcggctggagggacatctccacatcttacgccgattctgtgaagggccggttcaccatcagcagagacaacgccaagaagacactgtatctgcagatgaatagcctgaagcccgaggacaccgccgtgtactattgcgcagcaaggagaatcgacgcagcagactttgattcctggggccagggcacccaggtgacagtgtctagcggaggaggaggatctggaggaggaggaagcggaggaggaggatccgaggtgcagctggtggagtctggaggcggcctggtgcaggccggaggctctctgaggctgagctgtgcagcatccggaagaaccttcacaatgggctggtttaggcaggcaccaggaaaggagagggagttcgtggcagcaatcagcctgtcccctaccctggcctactatgccgagtccgtgaagggcaggtttaccatctctcgcgataacgccaagaatacagtggtgctgcagatgaacagcctgaaacctgaggacacagccctgtactattgtgccgccgatcggaagagcgtgatgagcattagacccgattattggggacagggcacacaggtgacagtgagtagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatga

SEQ ID NO: 42 - DNA, 1473 bp, synthetic construct
  source 1..1473: mol_type = other DNA
atggccctgccagtaactgctcttctcctgccactcgctcttctgctgcacgccgcacggcctcaaattcaacttgtgcagagtggccccgagctgaaaaagcccggcgaaacagtaaagatctcttgtaaggcgtctggatatatctttaccaattacggtatgaactgggtcaaacaggcacccggtaaaagctttaaatggatgggctggataaatacttataccggagaaagcacgtatagcgccgacttcaagggtagatttgcgttcagcctggaaacgtccgccagcactgcatacttgcacatcaatgacttgaaaaatgaggataccgcgacctacttttgtgctagaagtggtggatacgatcccatggattactggggacaggggacctctgtcacagtcagttctgggggcggcggttccggaggtggcggctcaggtggaggtgggtcagatatcgtgttgacacaaagccctgcttccctggccgtcagcttgggacagagagctactatctcctgtagagcaagtgagtctgtcgacaattacggcaacacctttatgcactggtaccagcagaagccaggccagccaccgaagctgttgatttaccgggcctcaaacctcgaaagcgggattccagctagattttctggcagcgggagtcgaaccgacttcacactgactattaacccagtggaagcagatgacgtagccacatattactgtcagcagagcaacgaagaccccccgacgtttggtgctggaaccaaactggagctgaaaacgacaacccccgctccacgacctcctactcccgctcccacaatcgcatctcagccactctccttgaggcctgaggcctgccgaccagctgcaggcggcgcagtacacaccagaggcctcgactttgcttgcgatatctacatatgggcgcccctggccgggacgtgtggggtattgctcctgagccttgtgatcactctttactgcaaaagaggtcggaaaaagcttctttacatctttaaacagccgttcatgcgcccggtgcaaacaacacaggaggaggatggatgcagctgcaggtttccggaggaggaggaaggcgggtgtgagctccgggtaaaattctccagaagcgcagacgcgccagcctatcagcaggggcagaatcagctctataacgaactcaatcttgggcgacgggaagaatacgatgtacttgataaacgccgaggtcgcgaccccgaaatgggaggcaagcctcagagacgcaagaatccccaagaaggcctgtataatgaactccagaaggataaaatggcggaggcctacagcgaaattggtatgaagggagaaagacgacgcggtaaaggccacgacggcctttaccagggcctgtcaacagctaccaaagatacgtatgatgcacttcacatgcaagccctgccgccccggtgatga

SEQ ID NO: 43 - DNA, 1473 bp, synthetic construct
  source 1..1473: mol_type = other DNA
atggctctgcctgtcaccgctctgctgctgcctctggctctgctgctgcacgctgctcgccctcaggtgcagctgcaggagtctggcccaggcctggtgaagcccagccagaccctgagcatcacctgcaccgtgagcggcttcagcctggccagctacaacatccactgggtgcggcagcccccaggcaagggcctggagtggctgggcgtgatctgggctggcggcagcaccaactacaacagcgccctgatgagccggctgaccatcagcaaggacaacagcaagaaccaggtgttcctgaagatgagcagcctgacagccgccgacaccgccgtgtactactgcgccaagcggagcgacgactacagctggttcgcctactggggccagggcaccctggtgaccgtgagctctggcggaggcggctctggcggaggcggctctggcggaggcggcagcgagaaccagatgacccagagccccagcagcttgagcgccagcgtgggcgaccgggtgaccatgacctgcagagccagcagcagcgtgagcagcagctacctgcactggtaccagcagaagagcggcaaggccccaaaggtgtggatctacagcaccagcaacctggccagcggcgtgaccagccggttcagcggcagcggcagcggcaccgactacaccctgaccatcagcagcctgcagcccgaggacttcgccacctactactgccagcagtacagcggctaccccatcaccttcggccagggcaccaaggtggagatcaagcggtcggatcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatga

SEQ ID NO: 44 - DNA, 1503 bp, synthetic construct
  source 1..1503: mol_type = other DNA
atggctctgccagtgacagctctgctgcttcctctggctctcctgctgcatgccgctagacctgagcagaaactcatctctgaagaggatctgcacgtgaaactgcaagagtctggccccggactggtgcagccttctcagtctctctctctgacctgtaccgtgtccggcttcagcctgaccgattatggcgtgcactgggttcgacaatccccaggcaaaggactcgagtggctgggagtgatttggagcggcggaggcacagcctataacacagccctgatcagcagactgaacatctaccgggacaactccaagaaccaggttttcctggaaatgaactccctgcaggcagaggacaccgccatgtactactgcgccagaagaggcagctacccctacaattacttcgacgcctggggctgtggcacaaccgtgacagtttctagtggcggaggcggatctggtggtggtggtagcggtggcggaggatctcaggccgtggttattcaagaaagcgccctgacaacccctcctggcgagacagtgacactgacatgtggcagctctacaggcgccgtgaccgccagcaattacgccaattgggtgcaagagaagcccgaccactgcttcacaggcctgatcggcggccacaacaatagacctccaggcgtgccagctagattcagcggatccctgatcggagacaaggccgctctgacaatcgccggcacacagacagaggacgaggccatctacttttgcgccctgtggtacagcgaccactgggttatcggcggaggaaccagactgacagtgctgggcacaacaacacccgcacctagaccaccaactccagcaccaacaatcgcctctcaacccctgagtctgaggccagaggcatgcagaccagccgctggcggtgcagttcacactagaggactggactttgcctgtgacatctacatctgggcccctctggctggaacatgtggtgtcctgctgctgtccctggtcatcaccctgtactgcaagcggggcagaaagaaactgctgtacatcttcaagcagcccttcatgcggcccgtgcagaccacacaagaggaagatggctgctcctgcagattccccgaggaagaagaaggcggctgcgagctgagagtgaagttctccagatctgccgacgctcccgcctatcagcagggacagaaccagctgtacaacgagctgaacctggggagaagagaagagtacgacgtgctggacaagcggagaggcagagatcctgagatgggcggaaagccccagcggagaaagaatcctcaagagggcctgtataatgagctgcaaaaggacaagatggccgaggcctacagcgagatcggaatgaagggcgagcgcagaagaggaaagggacacgacggactgtaccagggcctgagcacagccaccaaggatacctatgacgccctgcacatgcaggccctgcctccaagatgatga

SEQ ID NO: 45 - DNA, 1512 bp, synthetic construct
  source 1..1512: mol_type = other DNA
atggctctgcctgtgacagctctgctgctgcctctggctctgcttctgcatgccgccagacctgacgtggtcatgacacagacacctctgagcctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggatttcgcctgcgacttctgggtgctcgtggttgttggcggagtgctggcttgttactccctgctggttaccgtggccttcatcatcttttgggtcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggaaagccccagcggagaaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgccacctagatgatga

SEQ ID NO: 46 - DNA, 4866 bp, synthetic construct
  source 1..4866: mol_type = other DNA
atggctgctcctgctctgtcttggagactgcccctgctgattctgctgctgcctctggctacatcttgggcctctgccgattacaaggatgacgacgataaggacgtggtcatgacacagacaccactgagcctgcctgtgtctctgggagatcaggccagcatcagctgcagatccagccagtctctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgcaaagaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggatttcgcctgcgacatcccctggctgggacatctgcttgttggactgtctggcgccttcggcttcatcatcctggtgtatctgctgatcaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagcccaggtggactggcccctgagattagccctctggaagtgctggaacgggacaaagtgacccagctgctcctccagcaggataaggtgccagaacctgccagcctgtccagcaatcacagcctgaccagctgctttaccaaccagggctacttcttcttccatctgcctgacgctctggaaatcgaggcctgccaggtgtacttcacctacgatccctacagcgaagaggaccccgatgaaggtgttgctggcgcccctacaggatcttctccacagcctctgcaacctctgagcggcgaggatgatgcctactgcacctttccaagcagggacgacctgctcctgttcagcccatctctgctcggaggaccatctcctccatctacagctccaggcggatctggcgctggcgaggaaagaatgccacctagcctgcaagagcgggtgcccagagattgggatcctcaacctctcggccctccaacacctggcgtgccagatctcgtggactttcagcctcctccagagctggtgctgagagaagctggcgaagaagtgccagacgctggccctagagagggcgttagctttccttggagcagacctcctggacagggcgagtttagggccctgaatgctagactgcctctgaacaccgacgcctacctgtctctgcaagaactgcagggacaagaccccacacacctcgttggaagcggagtgaagcagaccctgaacttcgacctgctgaaactggccggcgacgtggaaagcaaccctggacctatgctgaagcccagcctgccttttaccagcctgctgttcctgcagctcccactgcttggcgtgggatacccatacgatgttccagattacgctgatgtcgtgatgacccagactccactctctctgcctgtcagcctgggcgatcaagcctccatctcctgtagaagcagccagagcctggtccactccaatggcaatacctatctcagatggtatctccaaaagcctgggcagagcccaaaagtcctcatctacaaagtctctaatcgcgtcagcggagtgcccgacaggtttagcggttctggaagcggcacagatttcacgctcaagattaaccgcgtcgaggccgaagatctgggagtctatttctgcagtcagagcacacatgtgccctggacattcggcggaggcaccaagctcgagatcaagtctagcgccgatgatgctaagaaagatgcagctaaaaaggatgatgccaagaaggacgacgccaagaaagatggcggagtcaaactcgatgagactggcggaggcctggtgcaacccggtggtgctatgaagctgtcttgcgtgacctccggctttacatttgggcattattggatgaattgggttcgccaatctccagagaagggcctcgagtgggttgcacagtttcggaacaaaccgtacaattacgagacatattactccgactccgtgaaaggccggttcacaatctcccgcgacgactccaagtcctctgtctatcttcaaatgaacaatctgagagtcgaggacacggggatctactattgcacaggcgcctcttatggaatggaataccttggacagggaacctctgtgaccgtcagcaccacaacacccgctcctagacctccaactcctgctccaacaatcgcctctcaacccctcagcctcagacctgaggcatgtagaccagcagctggcggtgcagttcacaccagaggcctggactttgcctgtgacgtcgtgatcagcgtgggcagcatgggcctgatcatctccctgctgtgtgtgtacttttggctcgagcggaccatgcctcggatccccacactgaagaacctcgaggatctggtcaccgagtaccacggcaacttctctgcttggagcggcgtgtcaaaaggactggccgaaagcctgcagcctgactactccgagagactgtgcctggtgtctgagatccctcctaaaggcggcgctctcggagaaggacctggtgcctctccatgcaatcagcacagcccttattgggcccctccttgctacaccctgaaacctgagacaggcagcggcgaaggcagaggctctcttcttacatgtggcgacgtcgaagagaatcccggaccaatggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctgatga

SEQ ID NO: 47 - DNA, 4254 bp, synthetic construct
  source 1..4254: mol_type = other DNA
atgaccatcctgggcaccaccttcggcatggtgtttagcctgctgcaggtcgtgtctggcgattacaaggatgacgacgataaggacgtggtcatgacacagaccccactgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgcaaagaaagacgatgccaaaaaggatggcggcgtgaagctggacgaaacaggcggaggacttgttcagcctggcggagccatgaagctgagctgtgtgaccagcggcttcacctttggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagcctctgtctctgaggcccgaagcttgtagacctgctgccggcggagctgtgcatacaagaggcctggatttcgcctgcgatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctggaagagagcgagaagcagagactcggaggcgacgtgcagagccctaattgcccttctgaggacgtcgtgatcacccctgagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgagcctgggaaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaaggctccggcgaaggcagaggctctctgcttacatgcggagatgtggaagagaaccccggacctatggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatctggcgccacaaacttctcactgctgaaacaggccggcgacgtcgaagagaatcctgggcctatgcctcctcctcggctgctgttcttcctgctgtttctgacccctatggaagtgcggcccgaggaacctctggtcgtgaaagttgaagagggcgacaacgccgtgctgcagtgtctgaagggcacatctgatggccccacacagcagctgacctggtctagagagagccctctgaagcccttcctgaagctcagtctgggactgcctggactgggcatccatatgaggccactggccatctggctgttcatctttaacgtgtcccagcagatgggaggcttctacctgtgtcagcctggacctcctagcgagaaagcttggcagcctggctggaccgtgaatgtggaaggatccggcgagctgttccggtggaatgtctctgatctcggcggcctcggatgcggcctgaagaatagatctagcgagggccctagcagcccctccggaaaactgatgagccccaagctgtacgtgtgggccaaagacagacccgagatttgggagggcgagcctccttgtctgccacctagagacagcctgaatcagagcctgagccaggacctgacaatggcccctggatctacactgtggctgagctgcggagtgcctcctgacagtgtgtctagaggcccactgagctggacacacgtgcaccctaagggccctaagagcctgctgagtctggaactgaaggacgacaggcccgccagagatatgtgggtcatggaaaccggactgctgctccctagagccactgctcaagatgccggcaagtactattgccaccggggcaacctgaccatgagcttccacctggaaattaccgccagaccagtgctgtggcattggctgcttagaaccggcggatggaaggtgtcagccgtgactctggcctacctgatcttttgtctgtgcagcctcgtgggcatcctgcatctgcaaagagccctggtcctgcggcggaagcggaagagaatgaccgatcctaccagacggttctgatga

SEQ ID NO: 48 - DNA, 3204 bp, synthetic construct
  source 1..3204: mol_type = other DNA
atgaccatcctgggcaccaccttcggcatggtgtttagcctgctgcaggtcgtgtctggcgattacaaggatgacgacgataaggacgtggtcatgacacagaccccactgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgcaaagaaagacgatgccaaaaaggatggcggcgtgaagctggacgaaacaggcggaggacttgttcagcctggcggagccatgaagctgagctgtgtgaccagcggcttcacctttggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagcctctgtctctgaggcccgaagcttgtagacctgctgccggcggagctgtgcatacaagaggcctggatttcgcctgcgatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctggaagagagcgagaagcagagactcggaggcgacgtgcagagccctaattgcccttctgaggacgtcgtgatcacccctgagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgagcctgggaaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaaggctccggcgaaggcagaggctctctgcttacatgcggagatgtggaagagaaccccggacctatggctctgcctgtcaccgctctgctgctgcctctggctctgctgctgcacgctgctcgccctcaggtcaaactggaagaaagtgggggaggcctggtgcaggcaggacggagcctgcgcctgagctgcgcagcatccgagcacaccttcagctcccacgtgatgggctggtttcggcaggccccaggcaaggagagagagtccgtggccgtgatcggctggagggacatctccacatcttacgccgattctgtgaagggccggttcaccatcagcagagacaacgccaagaagacactgtatctgcagatgaatagcctgaagcccgaggacaccgccgtgtactattgcgcagcaaggagaatcgacgcagcagactttgattcctggggccagggcacccaggtgacagtgtctagcggaggaggaggatctggaggaggaggaagcggaggaggaggatccgaggtgcagctggtggagtctggaggcggcctggtgcaggccggaggctctctgaggctgagctgtgcagcatccggaagaaccttcacaatgggctggtttaggcaggcaccaggaaaggagagggagttcgtggcagcaatcagcctgtcccctaccctggcctactatgccgagtccgtgaagggcaggtttaccatctctcgcgataacgccaagaatacagtggtgctgcagatgaacagcctgaaacctgaggacacagccctgtactattgtgccgccgatcggaagagcgtgatgagcattagacccgattattggggacagggcacacaggtgacagtgagtagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatga

SEQ ID NO: 49 - DNA, 3177 bp, synthetic construct
  source 1..3177: mol_type = other DNA
atgaccatcctgggcaccaccttcggcatggtgtttagcctgctgcaggtcgtgtctggcgattacaaggatgacgacgataaggacgtggtcatgacacagaccccactgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgcaaagaaagacgatgccaaaaaggatggcggcgtgaagctggacgaaacaggcggaggacttgttcagcctggcggagccatgaagctgagctgtgtgaccagcggcttcacctttggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagcctctgtctctgaggcccgaagcttgtagacctgctgccggcggagctgtgcatacaagaggcctggatttcgcctgcgatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctggaagagagcgagaagcagagactcggaggcgacgtgcagagccctaattgcccttctgaggacgtcgtgatcacccctgagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgagcctgggaaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaaggctccggcgaaggcagaggctctctgcttacatgcggagatgtggaagagaaccccggacctatggccctgccagtaactgctcttctcctgccactcgctcttctgctgcacgccgcacggcctcaaattcaacttgtgcagagtggccccgagctgaaaaagcccggcgaaacagtaaagatctcttgtaaggcgtctggatatatctttaccaattacggtatgaactgggtcaaacaggcacccggtaaaagctttaaatggatgggctggataaatacttataccggagaaagcacgtatagcgccgacttcaagggtagatttgcgttcagcctggaaacgtccgccagcactgcatacttgcacatcaatgacttgaaaaatgaggataccgcgacctacttttgtgctagaagtggtggatacgatcccatggattactggggacaggggacctctgtcacagtcagttctgggggcggcggttccggaggtggcggctcaggtggaggtgggtcagatatcgtgttgacacaaagccctgcttccctggccgtcagcttgggacagagagctactatctcctgtagagcaagtgagtctgtcgacaattacggcaacacctttatgcactggtaccagcagaagccaggccagccaccgaagctgttgatttaccgggcctcaaacctcgaaagcgggattccagctagattttctggcagcgggagtcgaaccgacttcacactgactattaacccagtggaagcagatgacgtagccacatattactgtcagcagagcaacgaagaccccccgacgtttggtgctggaaccaaactggagctgaaaacgacaacccccgctccacgacctcctactcccgctcccacaatcgcatctcagccactctccttgaggcctgaggcctgccgaccagctgcaggcggcgcagtacacaccagaggcctcgactttgcttgcgatatctacatatgggcgcccctggccgggacgtgtggggtattgctcctgagccttgtgatcactctttactgcaaaagaggtcggaaaaagcttctttacatctttaaacagccgttcatgcgcccggtgcaaacaacacaggaggaggatggatgcagctgcaggtttccggaggaggaggaaggcgggtgtgagctccgggtaaaattctccagaagcgcagacgcgccagcctatcagcaggggcagaatcagctctataacgaactcaatcttgggcgacgggaagaatacgatgtacttgataaacgccgaggtcgcgaccccgaaatgggaggcaagcctcagagacgcaagaatccccaagaaggcctgtataatgaactccagaaggataaaatggcggaggcctacagcgaaattggtatgaagggagaaagacgacgcggtaaaggccacgacggcctttaccagggcctgtcaacagctaccaaagatacgtatgatgcacttcacatgcaagccctgccgccccggtgatga

SEQ ID NO: 50 - DNA, 3177 bp, synthetic construct
  source 1..3177: mol_type = other DNA
atgaccatcctgggcaccaccttcggcatggtgtttagcctgctgcaggtcgtgtctggcgattacaaggatgacgacgataaggacgtggtcatgacacagaccccactgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgcaaagaaagacgatgccaaaaaggatggcggcgtgaagctggacgaaacaggcggaggacttgttcagcctggcggagccatgaagctgagctgtgtgaccagcggcttcacctttggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagcctctgtctctgaggcccgaagcttgtagacctgctgccggcggagctgtgcatacaagaggcctggatttcgcctgcgatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctggaagagagcgagaagcagagactcggaggcgacgtgcagagccctaattgcccttctgaggacgtcgtgatcacccctgagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgagcctgggaaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaaggctccggcgaaggcagaggctctctgcttacatgcggagatgtggaagagaaccccggacctatggctctgcctgtcaccgctctgctgctgcctctggctctgctgctgcacgctgctcgccctcaggtgcagctgcaggagtctggcccaggcctggtgaagcccagccagaccctgagcatcacctgcaccgtgagcggcttcagcctggccagctacaacatccactgggtgcggcagcccccaggcaagggcctggagtggctgggcgtgatctgggctggcggcagcaccaactacaacagcgccctgatgagccggctgaccatcagcaaggacaacagcaagaaccaggtgttcctgaagatgagcagcctgacagccgccgacaccgccgtgtactactgcgccaagcggagcgacgactacagctggttcgcctactggggccagggcaccctggtgaccgtgagctctggcggaggcggctctggcggaggcggctctggcggaggcggcagcgagaaccagatgacccagagccccagcagcttgagcgccagcgtgggcgaccgggtgaccatgacctgcagagccagcagcagcgtgagcagcagctacctgcactggtaccagcagaagagcggcaaggccccaaaggtgtggatctacagcaccagcaacctggccagcggcgtgaccagccggttcagcggcagcggcagcggcaccgactacaccctgaccatcagcagcctgcagcccgaggacttcgccacctactactgccagcagtacagcggctaccccatcaccttcggccagggcaccaaggtggagatcaagcggtcggatcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatga

SEQ ID NO: 51 - DNA, 3207 bp, synthetic construct
  source 1..3207: mol_type = other DNA
atgaccatcctgggcaccaccttcggcatggtgtttagcctgctgcaggtcgtgtctggcgattacaaggatgacgacgataaggacgtggtcatgacacagaccccactgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgcaaagaaagacgatgccaaaaaggatggcggcgtgaagctggacgaaacaggcggaggacttgttcagcctggcggagccatgaagctgagctgtgtgaccagcggcttcacctttggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagcctctgtctctgaggcccgaagcttgtagacctgctgccggcggagctgtgcatacaagaggcctggatttcgcctgcgatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctggaagagagcgagaagcagagactcggaggcgacgtgcagagccctaattgcccttctgaggacgtcgtgatcacccctgagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgagcctgggaaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaaggctccggcgaaggcagaggctctctgcttacatgcggagatgtggaagagaaccccggacctatggctctgccagtgacagctctgctgcttcctctggctctcctgctgcatgccgctagacctgagcagaaactcatctctgaagaggatctgcacgtgaaactgcaagagtctggccccggactggtgcagccttctcagtctctctctctgacctgtaccgtgtccggcttcagcctgaccgattatggcgtgcactgggttcgacaatccccaggcaaaggactcgagtggctgggagtgatttggagcggcggaggcacagcctataacacagccctgatcagcagactgaacatctaccgggacaactccaagaaccaggttttcctggaaatgaactccctgcaggcagaggacaccgccatgtactactgcgccagaagaggcagctacccctacaattacttcgacgcctggggctgtggcacaaccgtgacagtttctagtggcggaggcggatctggtggtggtggtagcggtggcggaggatctcaggccgtggttattcaagaaagcgccctgacaacccctcctggcgagacagtgacactgacatgtggcagctctacaggcgccgtgaccgccagcaattacgccaattgggtgcaagagaagcccgaccactgcttcacaggcctgatcggcggccacaacaatagacctccaggcgtgccagctagattcagcggatccctgatcggagacaaggccgctctgacaatcgccggcacacagacagaggacgaggccatctacttttgcgccctgtggtacagcgaccactgggttatcggcggaggaaccagactgacagtgctgggcacaacaacacccgcacctagaccaccaactccagcaccaacaatcgcctctcaacccctgagtctgaggccagaggcatgcagaccagccgctggcggtgcagttcacactagaggactggactttgcctgtgacatctacatctgggcccctctggctggaacatgtggtgtcctgctgctgtccctggtcatcaccctgtactgcaagcggggcagaaagaaactgctgtacatcttcaagcagcccttcatgcggcccgtgcagaccacacaagaggaagatggctgctcctgcagattccccgaggaagaagaaggcggctgcgagctgagagtgaagttctccagatctgccgacgctcccgcctatcagcagggacagaaccagctgtacaacgagctgaacctggggagaagagaagagtacgacgtgctggacaagcggagaggcagagatcctgagatgggcggaaagccccagcggagaaagaatcctcaagagggcctgtataatgagctgcaaaaggacaagatggccgaggcctacagcgagatcggaatgaagggcgagcgcagaagaggaaagggacacgacggactgtaccagggcctgagcacagccaccaaggatacctatgacgccctgcacatgcaggccctgcctccaagatgatga

SEQ ID NO: 52 - DNA, 3186 bp, synthetic construct
  source 1..3186: mol_type = other DNA
atgaccatcctgggcaccaccttcggcatggtgtttagcctgctgcaggtcgtgtctggcgattacaaggatgacgacgataagcacgtgaaactgcaagagtctggccccggactggtgcagccttctcagtctctctctctgacctgtaccgtgtccggcttcagcctgaccgattatggcgtgcactgggttcgacaatccccaggcaaaggactcgagtggctgggagtgatttggagcggcggaggcacagcctataacacagccctgatcagcagactgaacatctaccgggacaactccaagaaccaggttttcctggaaatgaactccctgcaggcagaggacaccgccatgtactactgcgccagaagaggcagctacccctacaattacttcgacgcctggggctgtggcacaaccgtgacagtttctagtggcggaggcggatctggtggtggtggtagcggtggcggaggatctcaggccgtggttattcaagaaagcgccctgacaacccctcctggcgagacagtgacactgacatgtggcagctctacaggcgccgtgaccgccagcaattacgccaattgggtgcaagagaagcccgaccactgcttcacaggcctgatcggcggccacaacaatagacctccaggcgtgccagctagattcagcggatccctgatcggagacaaggccgctctgacaatcgccggcacacagacagaggacgaggccatctacttttgcgccctgtggtacagcgaccactgggttatcggcggaggaaccagactgacagtgctgggcacaacaacccctgctcctcggcctcctacaccagctcctacaattgccagccagcctctgtctctgaggcccgaagcttgtagacctgctgccggcggagctgtgcatacaagaggcctggatttcgcctgcgatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctggaagagagcgagaagcagagactcggaggcgacgtgcagagccctaattgcccttctgaggacgtcgtgatcacccctgagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgagcctgggaaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaaggctccggcgaaggcagaggctctctgcttacatgcggagatgtggaagagaaccccggacctatggctctgcctgtgacagctctgctgctgcctctggctctgcttctgcatgccgccagacctgacgtggtcatgacacagacacctctgagcctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggatttcgcctgcgacttctgggtgctcgtggttgttggcggagtgctggcttgttactccctgctggttaccgtggccttcatcatcttttgggtcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggaaagccccagcggagaaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgccacctagatgatga

SEQ ID NO: 53 - DNA, 2640 bp, synthetic construct
  source 1..2640: mol_type = other DNA
atgaccatcctgggcaccaccttcggcatggtgtttagcctgctgcaggtcgtgtctggcgattacaaggatgacgacgataaggacgtggtcatgacacagaccccactgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgcaaagaaagacgatgccaaaaaggatggcggcgtgaagctggacgaaacaggcggaggacttgttcagcctggcggagccatgaagctgagctgtgtgaccagcggcttcacctttggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagcctctgtctctgaggcccgaagcttgtagacctgctgccggcggagctgtgcatacaagaggcctggatttcgcctgcgatcccatcctgctgacaatcagcatcctgagctttttcagcgtggccctgctggtcatcctggcctgtgtgctgtggaagaagcggatcaagcccatcggctccggcgaaggcagaggctctctgcttacatgcggagatgtggaagagaaccccggacctatggctctgcctgtcaccgctctgctgctgcctctggctctgctgctgcacgctgctcgccctcaggtcaaactggaagaaagtgggggaggcctggtgcaggcaggacggagcctgcgcctgagctgcgcagcatccgagcacaccttcagctcccacgtgatgggctggtttcggcaggccccaggcaaggagagagagtccgtggccgtgatcggctggagggacatctccacatcttacgccgattctgtgaagggccggttcaccatcagcagagacaacgccaagaagacactgtatctgcagatgaatagcctgaagcccgaggacaccgccgtgtactattgcgcagcaaggagaatcgacgcagcagactttgattcctggggccagggcacccaggtgacagtgtctagcggaggaggaggatctggaggaggaggaagcggaggaggaggatccgaggtgcagctggtggagtctggaggcggcctggtgcaggccggaggctctctgaggctgagctgtgcagcatccggaagaaccttcacaatgggctggtttaggcaggcaccaggaaaggagagggagttcgtggcagcaatcagcctgtcccctaccctggcctactatgccgagtccgtgaagggcaggtttaccatctctcgcgataacgccaagaatacagtggtgctgcagatgaacagcctgaaacctgaggacacagccctgtactattgtgccgccgatcggaagagcgtgatgagcattagacccgattattggggacagggcacacaggtgacagtgagtagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatga

SEQ ID NO: 54 - DNA, 2775 bp, synthetic construct
  source 1..2775: mol_type = other DNA
atgaccatcctgggcaccaccttcggcatggtgtttagcctgctgcaggtcgtgtctggcgattacaaggatgacgacgataaggacgtggtcatgacacagaccccactgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcacaacaacccctgctcctcggcctcctacaccagctcctacaattgccagccagcctctgtctctgaggcccgaagcttgtagacctgctgccggcggagctgtgcatacaagaggcctggatttcgcctgcgatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctggaagagagcgagaagcagagactcggaggcgacgtgcagagccctaattgcccttctgaggacgtcgtgatcacccctgagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgagcctgggaaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaaggctccggcgaaggcagaggctctctgcttacatgcggagatgtggaagagaaccccggacctatggctctgcctgtcaccgctctgctgctgcctctggctctgctgctgcacgctgctcgccctcaggtcaaactggaagaaagtgggggaggcctggtgcaggcaggacggagcctgcgcctgagctgcgcagcatccgagcacaccttcagctcccacgtgatgggctggtttcggcaggccccaggcaaggagagagagtccgtggccgtgatcggctggagggacatctccacatcttacgccgattctgtgaagggccggttcaccatcagcagagacaacgccaagaagacactgtatctgcagatgaatagcctgaagcccgaggacaccgccgtgtactattgcgcagcaaggagaatcgacgcagcagactttgattcctggggccagggcacccaggtgacagtgtctagcggaggaggaggatctggaggaggaggaagcggaggaggaggatccgaggtgcagctggtggagtctggaggcggcctggtgcaggccggaggctctctgaggctgagctgtgcagcatccggaagaaccttcacaatgggctggtttaggcaggcaccaggaaaggagagggagttcgtggcagcaatcagcctgtcccctaccctggcctactatgccgagtccgtgaagggcaggtttaccatctctcgcgataacgccaagaatacagtggtgctgcagatgaacagcctgaaacctgaggacacagccctgtactattgtgccgccgatcggaagagcgtgatgagcattagacccgattattggggacagggcacacaggtgacagtgagtagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatga

SEQ ID NO: 55 - DNA, 3078 bp, synthetic construct
  source 1..3078: mol_type = other DNA
atgaccatcctgggcaccaccttcggcatggtgtttagcctgctgcaggtcgtgtctggcgattacaaggatgacgacgataaggacgtggtcatgacacagaccccactgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgcaaagaaagacgatgccaaaaaggatggcggcgtgaagctggacgaaacaggcggaggacttgttcagcctggcggagccatgaagctgagctgtgtgaccagcggcttcacctttggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctggatctggccccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctggaagagagcgagaagcagagactcggaggcgacgtgcagagccctaattgcccttctgaggacgtcgtgatcacccctgagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgagcctgggaaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaaggctccggcgaaggcagaggctctctgcttacatgcggagatgtggaagagaaccccggacctatggctctgcctgtcaccgctctgctgctgcctctggctctgctgctgcacgctgctcgccctcaggtcaaactggaagaaagtgggggaggcctggtgcaggcaggacggagcctgcgcctgagctgcgcagcatccgagcacaccttcagctcccacgtgatgggctggtttcggcaggccccaggcaaggagagagagtccgtggccgtgatcggctggagggacatctccacatcttacgccgattctgtgaagggccggttcaccatcagcagagacaacgccaagaagacactgtatctgcagatgaatagcctgaagcccgaggacaccgccgtgtactattgcgcagcaaggagaatcgacgcagcagactttgattcctggggccagggcacccaggtgacagtgtctagcggaggaggaggatctggaggaggaggaagcggaggaggaggatccgaggtgcagctggtggagtctggaggcggcctggtgcaggccggaggctctctgaggctgagctgtgcagcatccggaagaaccttcacaatgggctggtttaggcaggcaccaggaaaggagagggagttcgtggcagcaatcagcctgtcccctaccctggcctactatgccgagtccgtgaagggcaggtttaccatctctcgcgataacgccaagaatacagtggtgctgcagatgaacagcctgaaacctgaggacacagccctgtactattgtgccgccgatcggaagagcgtgatgagcattagacccgattattggggacagggcacacaggtgacagtgagtagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatga

(19) United States
(12) Patent Application Publication
(10) Pub. No.: US 2025/0297022 A1
(43) Pub. Date: Sep. 25, 2025

(54) ENGINEERED SWITCHES FOR IMMUNE CELL ACTIVITY AND METHODS OF USE THEREOF

(71) Applicant: Dynamic Cell Therapies, Inc., Watertown, MA (US)

(72) Inventors: Jun Ren, Brookline, MA (US); Alberto Nobili, Boston, MA (US); Carl D. Novina, Newton, MA (US); Craig Stewart McKay, Watertown, MA (US); John R. Newcomb, Woburn, MA (US)

(21) Appl. No.: 19/229,806

(22) Filed: Jun. 5, 2025

Related U.S. Application Data
(63) Continuation of application No. PCT/US2023/082603, filed on Dec. 5, 2023.
(60) Provisional application No. 63/386,296, filed on Dec. 6, 2022.
(60) Provisional application No. 63/386,293, filed on Dec. 6, 2022.
(60) Provisional application No. 63/386,450, filed on Dec. 7, 2022.

(51) Int. Cl. C07K 16/28; A61K 35/17; A61K 40/31; A61P 35/00; C07K 14/705; C07K 14/715; C12N 5/0783

(52) U.S. Cl. CPC C07K 16/2878; A61K 35/17; A61K 40/31; A61P 35/00; C07K 14/70517; C07K 14/70596; C07K 14/7155; C12N 5/0636; A61K 2239/17; A61K 2239/21; A61K 2239/22; C07K 2317/32; C07K 2317/53; C07K 2317/569; C07K 2317/622; C07K 2319/02; C07K 2319/03; C07K 2319/70

(57) ABSTRACT

Described herein are engineered cytokine receptor switches that can include a signal peptide, an extracellular activator binding domain, a hinge, a transmembrane domain, and/or an intracellular signaling domain. Binding of an activator to the activator binding domain can activate cytokine signaling through the intracellular signaling domain. These cytokine receptor switches can be expressed in immune cells, sometimes in combination with a chimeric antigen receptor (CAR), to increase immune cell persistence by promoting adoption of memory-like phenotypes. Also described herein are methods of using engineered cytokine receptors in immune cell therapies, such as CAR T-cell therapy, to improve patient outcomes and prevent disease relapse.

### CROSS-REFERENCE TO RELATED APPLICATION(S)

The present application is a continuation of International Application No. PCT/US2023/082603, filed Dec. 5, 2023, which claims the benefit of priority to U.S. Provisional Application No. 63/386,293, filed Dec. 6, 2022, U.S. Provisional Application No. 63/386,296, filed Dec. 6, 2022, and U.S. Provisional Application No. 63/386,450, filed Dec. 7, 2022, each of which is incorporated by reference herein in its entirety.

### INCORPORATION BY REFERENCE OF SEQUENCE LISTING

The present application contains an electronic Sequence Listing in XML file format named “DCT_001WO_SL,” created on Nov. 27, 2023, and having a size of 110 kilobytes, the contents of which are incorporated by reference herein in their entirety.

### TECHNICAL FIELD

The present technology relates to immunotherapy and, in particular, to engineered switches for immune cell activity and methods of use thereof.

### BACKGROUND

Since its development, chimeric antigen receptor (CAR) T-cell therapy has shown promise for treating cancers, particularly blood cancers, that may not be effectively treated using more conventional cancer therapies, such as chemoradiation therapy. However, cancer relapse following CAR T-cell therapy continues to be a concern. One mechanism of relapse following CAR T-cell therapy is due to poor persistence of T-cells in the patient over time. As such, there remains a need for methods to improve T-cell persistence in CAR T-cell therapies to prevent cancer relapse and improve patient outcomes.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A schematically illustrates an example of a domain layout of an engineered cytokine receptor switch. From N-terminus to C-terminus, an engineered cytokine receptor switch may contain a signal peptide (“SP”), an activator binding domain (“ABD”), a hinge, a transmembrane domain, and an intracellular domain.

FIG. 1B schematically illustrates an example of a domain layout of an engineered dual-chain cytokine receptor switch.

FIG. 2A schematically illustrates examples of engineered single-chain cytokine receptor switches with intracellular domains derived from different wild-type cytokine receptor switches. A single-chain cytokine receptor switch activates intracellular signaling pathways through forming a homodimer with the same single-chain cytokine receptor switch or through forming a heterodimer with an endogenous cytokine receptor chain. The intracellular domains of the illustrated single-chain cytokine receptor switches are derived from wild-type interleukin 2 receptor subunit α (IL2Rα), interleukin 2 receptor subunit β (IL2Rβ), interleukin 2 receptor subunit γ (IL2Rγ), interleukin 7 receptor subunit α (IL7Rα), interleukin 15 receptor subunit α (IL15Rα), and interleukin 21 receptor subunit α (IL21Rα), respectively.

FIG. 2B schematically illustrates examples of engineered dual-chain cytokine receptor switches with intracellular domains derived from different cytokine receptor switches. A dual-chain cytokine receptor switch includes two different cytokine receptor chains that activate intracellular signaling pathways by heterodimerizing with each other. The intracellular domains of the illustrated dual-chain cytokine receptor switches are derived from interleukin 2 receptor subunit β (IL2Rβ) and interleukin 2 receptor subunit γ (IL2Rγ) (left), interleukin 7 receptor subunit α (IL7Rα) and interleukin 2 receptor subunit γ (IL2Rγ) (center), or interleukin 21 receptor subunit α (IL21Rα) and interleukin 2 receptor subunit γ (IL2Rγ) (right), which heterodimerize to activate intracellular cytokine signaling.

FIG. 2C schematically illustrates examples of engineered single-chain cytokine receptor switches with chimeric, tandem, or mutant intracellular domains derived from cytokine receptor chains. A single-chain cytokine receptor switch activates intracellular signaling pathways through forming a homodimer with the same single-chain cytokine receptor switch or through forming a heterodimer with an endogenous cytokine receptor chain. The intracellular domains of the illustrated single-chain cytokine receptor switches are derived from chimeric, tandem, or mutant interleukin 2 receptor subunit β (IL2Rβ), interleukin 2 receptor subunit γ (IL2Rγ), interleukin 7 receptor subunit α (IL7Rα), interleukin 15 receptor subunit α (IL15Rα), or interleukin 21 receptor subunit α (IL21Rα).

FIG. 3A schematically illustrates an example of activation of an engineered single-chain cytokine receptor switch. When an antibody-activator conjugate (“antibody-small molecule conjugate”) is not bound to the engineered single-chain cytokine receptor switch (B), the single-chain cytokine receptor switch is undimerized with an endogenous cytokine receptor (A) (top) and the single-chain receptor switch is in an inactive “off” state. Upon heterodimerization with the endogenous cytokine receptor and binding of the antibody-small molecule conjugate to the single-chain cytokine receptor switch (bottom), the single-chain cytokine receptor switch is turned “on,” activating intracellular signaling through the intracellular cytokine receptor domains.

FIG. 3B schematically illustrates another example of activation of an engineered single-chain cytokine receptor switch. In the “off” state (top), when an antibody-activator conjugate (“antibody-small molecule conjugate”) is not bound to either single-chain cytokine receptor switch (C), the single-chain cytokine receptor switches remain undimerized and are inactive. Upon binding of the antibody-small molecular conjugate to both single-chain cytokine receptor switches (bottom), the single-chain receptor switches homodimerize with each other and are turned “on,” activating intracellular signaling through the intracellular cytokine receptor domains.

FIG. 3C schematically illustrates an example of activation of an engineered dual-chain cytokine receptor switch. In the “off” state (top), when an antibody-activator conjugate (“antibody-small molecule conjugate”) is not bound to either a first cytokine receptor chain (A) or a second cytokine receptor chain (B) of the dual-chain cytokine receptor switch, the chains remain undimerized and are inactive. Upon binding of the antibody-small molecular conjugate to the first and second cytokine receptor chains (bottom), the first and second cytokine receptor chains heterodimerize with each other and are turned “on,” activating intracellular signaling through the intracellular cytokine receptor domains.

FIG. 4A illustrates the cell surface markers used to differentiate various T-cell memory phenotypes assayed in FIG. 4B and FIG. 4C. T-cells having a stem-cell memory (T_{scm}) phenotype are positive for CCR7, CD45RA, and CD95. T-cells having a central memory (T_{cm}) phenotype are positive for CCR7 but negative for CD45RA. T-cells having an effector memory (T_{cm}) phenotype are negative for both CCR7 and CD45RA. T-cells having an effector memory re-expressing CD45RA (Tₑₘᵣₐ) phenotype are positive for CD45RA but negative for CCR7.

FIG. 4B is a bar graph showing the percentage of T-cells expressing an engineered cytokine receptor switch derived from IL7Rα (“IL7Rα SMAR”) having a particular T-cell phenotype with (right) or without (left) stimulation with a surface coated with 100 μg/mL of FITC-dextran as a small molecule activator. Both the stimulated and unstimulated treatment groups were grown in the presence of 10 U/mL of IL2. The bars for each treatment group represent, from left to right, a stem-cell memory (T_{scm}) phenotype, a central memory (T_{cm}) phenotype, an effector memory (Tₑₘ) phenotype, and an effector memory re-expressing CD45RA (Tₑₘᵣₐ) phenotype.

FIG. 4C is a bar graph showing the number of T-cells expressing the IL7Rα SMAR having a particular T-cell phenotype with (right) or without (left) stimulation with a surface coated with 100 μg/mL of FITC-dextran as a small molecule activator. Both the stimulated and unstimulated treatment groups were grown in the presence of 10 U/mL of IL2. The bars for each treatment group represent, from left to right, a stem-cell memory (T_{scm}) phenotype, a central memory (T_{cm}) phenotype, an effector memory (Tₑₘ) phenotype, and an effector memory re-expressing CD45RA (Tₑₘᵣₐ) phenotype.

FIG. 5 schematically illustrates examples of chimeric antigen receptor T-cells (CAR T-cells), with or without an engineered cytokine receptor switch, interacting with a tumor cell expressing a tumor cell antigen. In a first CAR T-cell (i), the T-cell expresses a traditional chimeric antigen receptor (CAR, e.g., bb2121) that binds to the tumor cell antigen. In a second CAR T-cell (ii), the T-cell expresses a first subunit (A) and a second subunit (B) of an engineered dual-chain cytokine receptor switch and a traditional CAR (e.g., bb2121) that binds to the tumor cell antigen. The first and second cytokine receptor chains of the engineered dual-chain cytokine receptor switch are activated with a small molecule activator during CAR T-cell manufacturing. In a third CAR T-cell (iii), the T-cell expresses an engineered single-chain cytokine receptor switch (C) and a traditional CAR (e.g., bb2121) that binds to the tumor cell antigen. The engineered cytokine receptor switch is activated with a small molecule activator during CAR T-cell manufacturing.

FIG. 6 schematically illustrates a timeline for a tumor rechallenge assay used in FIGS. 7A-7C and FIGS. 8A-8C. On day 0, the same number of CAR T-cells expressing a BCMA-targeting CAR (“bb2121 CAR”) with or without an engineered cytokine receptor switch, are co-cultured with MM.1S tumor cells in an effector to target ratio (E/T) of 1/4 or 1/8. On Day 2, half of the initial co-culture is taken for analysis and the first rechallenge is performed by adding either 4×10⁵ (for effector to target ratio (E/T) of 1/4) or 8×10⁵ (for E/T of 1/8) MM.1S tumor cells to the culture. On Day 4, half of the first rechallenge co-culture is taken for analysis and the second rechallenge is performed by adding either 4×10⁵ or 8×10⁵ MM.1S tumor cells to the culture. On Day 6, half of the second rechallenge co-culture is taken for analysis. No exogenous cytokine and no beads are included in the culture during the whole tumor challenge process.

FIG. 7A is a bar graph showing the number of viable tumor cells following the initial killing (E/T of 1/8, collected on Day 2, as illustrated in FIG. 6) for cells expressing, from left to right, a bb2121 CAR and no engineered cytokine receptor switch (“bb2121”), a bb2121 CAR and an engineered dual-chain cytokine receptor switch with a first chain derived from IL2Rβ and a second chain derived from IL2Rγ (IL2Rβ/γ SMAR) (“IL-2Rβ/γ SMAR bb2121”), or a bb2121 CAR and an IL7Rα SMAR (“IL-7Rα SMAR bb2121”). Error bars represent ±standard error of the mean (SEM), and p values were determined using a one-way ANOVA with Tukey post hoc test.

FIG. 7B is a bar graph showing the number of viable tumor cells following the first rechallenge (E/T of 1/8, collected on Day 4, as illustrated in FIG. 6) for cells expressing, from left to right, a bb2121 CAR and no engineered cytokine receptor switch (“bb2121”), a bb2121 CAR and the IL2Rβ/γ SMAR (“IL-2Rβ/γ SMAR bb2121”), or a bb2121 CAR and the IL7Rα SMAR (“IL-7Rα SMAR bb2121”). Error bars represent ±standard error of the mean (SEM), and p values were determined using a one-way ANOVA with Tukey post hoc test.

FIG. 7C is a bar graph showing the number of viable tumor cells following the second rechallenge (E/T of 1/8, collected on Day 6, as illustrated in FIG. 6) for cells expressing, from left to right, a bb2121 CAR and no engineered cytokine receptor switch (“bb2121”), a bb2121 CAR and the IL2Rβ/γ SMAR (“IL-2Rβ/γ SMAR bb2121”), or a bb2121 CAR and the IL7Rα SMAR (“IL-7Rα SMAR bb2121”). Error bars represent ±standard error of the mean (SEM), and p values were determined using a one-way ANOVA with Tukey post hoc test.

FIG. 8A is a bar graph showing the number of viable CAR-positive (CAR+) T-cells following the initial killing (E/T of 1/8, collected on Day 2, as illustrated in FIG. 6) for cells expressing, from left to right, a bb2121 CAR and no engineered cytokine receptor switch (“bb2121”), a bb2121 CAR and the IL2Rβ/γ SMAR (“IL-2Rβ/γ SMAR bb2121”), or a bb2121 CAR and the IL7Rα SMAR (“IL-7Rα SMAR bb2121”). The broken line denotes the number of viable CAR-positive T-cells (1×10⁵) present in the initial culture. Error bars represent ±standard error of the mean (SEM), and p values were determined using a one-way ANOVA with Tukey post hoc test.

FIG. 8B is a bar graph showing the number of viable CAR-positive (CAR+) T-cells following the first rechallenge (E/T of 1/8, collected on Day 4, as illustrated in FIG. 6) for cells expressing, from left to right, a bb2121 CAR and no engineered cytokine receptor switch (“bb2121”), a bb2121 CAR and the IL2Rβ/γ SMAR (“IL-2Rβ/γ SMAR bb2121”), or a bb2121 CAR and the IL7Rα SMAR (“IL-7Rα SMAR bb2121”). Error bars represent ±standard error of the mean (SEM), and p values were determined using a one-way ANOVA with Tukey post hoc test.

FIG. 8C is a bar graph showing the number of viable CAR-positive (CAR⁺) T-cells following the second rechallenge (E/T of 1/8, collected on Day 6, as illustrated in FIG. 6) for cells expressing, from left to right, a bb2121 CAR and no engineered cytokine receptor switch (“bb2121”), a bb2121 CAR and the IL2Rβ/γ SMAR (“IL-2Rβ/γ SMAR bb2121”), or a bb2121 CAR and the IL7Rα SMAR (“IL-7Rα SMAR bb2121”). Error bars represent ±standard error of the mean (SEM), and p values were determined using a one-way ANOVA with Tukey post hoc test.

FIG. 9A schematically illustrates an example of a timeline for evaluating tumor killing function in mice of T-cells expressing a CAR with or without an engineered cytokine receptor switch.

FIG. 9B schematically illustrates another example of a timeline for evaluating tumor killing function in mice of T-cells expressing a CAR with or without an engineered cytokine receptor switch.

FIG. 10A provides an image of a container with an unfunctionalized well, and which contains immune cells engineered to express an engineered cytokine receptor switch configured to bind fluorescein.

FIG. 10B provides an image of a container with a fluorescein-functionalized well, and which contains immune cells engineered to express an engineered cytokine receptor switch configured to bind fluorescein.

FIG. 11A schematically illustrates an engineered immune cell expressing a CAR and an engineered cytokine receptor switch comprising an activator binding domain. Also illustrated is a bispecific agent comprising a lymphoid targeting protein linked to an activator that binds to the activator binding domain of the engineered cytokine receptor switch.

FIG. 11B schematically illustrates recruitment of an engineered immune cell to a lymphatic organ and targeting of a cancer cell. Panel 1 illustrates a bispecific agent and an engineered immune cell expressing a CAR and an engineered cytokine receptor switch. Panel 2 illustrates recruitment of the engineered immune cell to the lymphoid organ by binding the engineered cytokine receptor switch to the bispecific agent and binding the bispecific agent to a surface marker on a lymphoid organ cell. Panel 3 illustrates targeting a cancer cell by binding the CAR to a surface marker on the cancer cell.

FIG. 12A is a schematic illustration of a domain layout of a construct for expression of a SMAR.

FIG. 12B is a schematic illustration of a domain layout of an example construct for expression of an IL7Rα SMAR.

FIG. 12C is a schematic illustration of a domain layout of an example construct for expression of an IL2Rβ/γ SMAR.

FIG. 12D is a schematic illustration of a domain layout of an example construct for expression of an anti-BCMA CAR (bb2121).

FIG. 12E is a schematic illustration of a domain layout of an example construct for expression of an anti-BCMA CAR (Carvykti).

FIG. 12F is a schematic illustration of a domain layout of an example construct for expression of an anti-CD123 CAR.

FIG. 12G is a schematic illustration of a domain layout of an example construct for expression of an anti-GD2 CAR.

FIG. 12H is a schematic illustration of a domain layout of an example construct for expression of an anti-DOTA indirect CAR.

FIG. 12I is a schematic illustration of a domain layout of an example construct for expression of an anti-FITC indirect CAR.

FIG. 12J is a schematic illustration of a domain layout of an example construct for expression of an IL7Rα SMAR and an anti-BCMA CAR (bb2121).

FIG. 12K is a schematic illustration of a domain layout of an example construct for expression of an IL7Rα SMAR and an anti-BCMA CAR (Carvykti).

FIG. 12L is a schematic illustration of a domain layout of an example construct for expression of an IL7Rα SMAR and an anti-CD123 CAR.

FIG. 12M is a schematic illustration of a domain layout of an example construct for expression of an IL7Rα SMAR and an anti-GD2 CAR.

FIG. 12N is a schematic illustration of a domain layout of an example construct for expression of an IL7Rα SMAR and an anti-DOTA indirect CAR.

FIG. 12O is a schematic illustration of a domain layout of an example construct for expression of an IL7Rα SMAR and an anti-FITC indirect CAR.

FIG. 12P is a schematic illustration of a domain layout of an example construct for expression of an IL2Rβ/γ SMAR and an anti-BCMA CAR (bb2121).

FIG. 12Q is a schematic illustration of a domain layout of an example construct for expression of an IL2Rβ/γ SMAR and an anti-BCMA CAR (Carvykti).

FIG. 12R is a schematic illustration of a domain layout of an example construct for expression of an IL2Rβ/γ SMAR and an anti-CD123 CAR.

FIG. 12S is a schematic illustration of a domain layout of an example construct for expression of an IL2Rβ/γ SMAR and an anti-GD2 CAR.

FIG. 12T is a schematic illustration of a domain layout of an example construct for expression of an IL2Rβ/γ SMAR and an anti-DOTA indirect CAR.

FIG. 12U is a schematic illustration of a domain layout of an example construct for expression of an IL2Rβ/γ SMAR and an anti-FITC indirect CAR.

FIG. 13 illustrates activator-independent activity in immune cells expressing IL7Rα SMAR constructs and an anti-BCMA CAR (Carvykti).

FIGS. 14A and 14B are flow cytometry plots (FIG. 14A) and a graph of the flow cytometry data (FIG. 14B) showing that cytokine receptor SMARs increased the persistence of CAR+CD8+ cells during an in vitro tumor re-challenge.

FIG. 15A provides a timeline for an in vitro tumor re-challenge assay.

FIGS. 15B and 15C are graphs showing that CAR T-cells including an IL7Rα SMAR demonstrated superior tumor cell killing (FIG. 15C) and T-cell expansion (FIG. 15B) than CAR T-cells expressing the bb2121 CAR alone during a tumor re-challenge assay. Data presented are mean±SEM. Statistical analysis was conducted using a one-way ANOVA with Dunnett's multiple comparison test (asterisks indicate statistical significance relative to the bb2121 CAR alone).

FIG. 16 is a series of graphs showing the effect of an IL7Rα SMAR on cytokine expression in bb2121 CAR T-cells.

FIG. 17A is a graph showing that IL7Rα SMAR activation in Carvykti promoted differentiation to the central memory (Tₑₘ) phenotype.

FIG. 17B is a series of graphs showing a comparison of T-cell phenotypes between populations with CD3/CD28 re-stimulation (right) and without re-stimulation (left).

FIG. 17C is a series of flow cytometry plots showing a comparison of T-cell phenotypes between populations with CD3/CD28 re-stimulation (right) and without re-stimulation (left), and with and without FITC activation.

FIG. 18A is a graph illustrating tumor cell killing by T-cell populations grown on non-coated plates in the absence of CD3/CD28 restimulation.

FIG. 18B is a graph illustrating tumor cell killing by T-cell populations grown on FITC-coated plates with CD3/CD28 re-stimulation.

FIG. 19A is a series of graphs illustrating cytokine expression in cells without CD3/CD28 re-stimulation and without FITC activation.

FIG. 19B is a series of graphs illustrating cytokine expression in cells with CD3/CD28 re-stimulation and FITC activation.

FIG. 20A is a graph illustrating tumor cell killing through Day 18 in a MM.1S mouse model treated with varying dosages of T-cells expressing an anti-BMCA CAR (bb2121) alone (“bb2121”) and with an IL7Rα SMAR (“IL7Rα SMAR bb2121”).

FIG. 20B is a graph illustrating tumor cell killing through Day 60 in a MM.1S mouse model treated with varying dosages of T-cells expressing an anti-BMCA CAR (bb2121) alone (“bb2121”) and with an IL7Rα SMAR (“IL7Rα SMAR bb2121”).

FIGS. 21A-21C are graphs illustrating survival (top) and tumor cell killing (bottom) in mice treated with 0.5×10⁶ (FIG. 21A), 2×10⁶ (FIG. 21B), and 5×10⁶ (FIG. 21C) bb2121 or IL7Rα-bb2121 cells.

FIGS. 22A-22C are graphs illustrating survival (FIG. 22A) and tumor cell killing (FIGS. 22B and 22C) in mice treated with varying dosages of bb2121 or IL7Rα-bb2121 cells.

FIGS. 23A-23C are graphs illustrating percentages of CD8+/CD4+ cells (FIG. 23A), CAR+ cell counts (FIG. 23B), and CAR+CD8+ cell counts (FIG. 23C) in mice treated with bb2121 or IL7Rα-bb2121 cells.

FIGS. 24A-24C are graphs illustrating tumor cell killing in a MM.1S model treated with T-cells expressing an anti-BMCA CAR (bb2121) alone (“bb2121”) and with an IL7Rα SMAR (“IL7Rα SMAR bb2121”). FIG. 24A illustrates tumor cell killing across the various study groups (arrow indicates the MM.1S rechallenge on Day 55), FIG. 24B illustrates tumor cell killing in individual mice in the bb2121 group, and FIG. 24C illustrates tumor cell killing in individual mice in the IL7Rα SMAR bb2121 group.

FIG. 25A is a series of graphs illustrating cell counts in the blood, lymph nodes, and spleen at Day 8 (top) and Day 22 (bottom) in mice treated with bb2121 or IL7Rα SMAR bb2121.

FIG. 25B is a series of graphs illustrating cell phenotypes in the blood (left), lymph nodes (center), and spleen (right) at Day 8 in mice treated with bb2121 or IL7Rα SMAR bb2121.

FIG. 25C is a series of graphs illustrating cell phenotypes in the blood (left), lymph nodes (center), and spleen (right) at Day 22 in mice treated with bb2121 or IL7Rα SMAR bb2121.

FIG. 25D is a series of graphs illustrating cell counts in the blood, lymph nodes, and spleen at Day 66 (11 days after rechallenge) in mice treated with bb2121 or IL7Rα SMAR bb2121.

FIG. 25E is a series of graphs illustrating cell phenotypes in the blood (left), lymph nodes (center), and spleen (right) at Day 66 (11 days after rechallenge) in mice treated with bb2121 or IL7Rα SMAR bb2121.

FIG. 26A is a graph illustrating tumor cell killing in a MM.1S model treated with T-cells expressing an anti-BMCA CAR (Carvykti) alone (“Carvykti”) and with an IL7Rα SMAR (“SMAR-Carvykti”).

FIGS. 26B-26D are graphs illustrating tumor cell killing in mice treated with doses of 0.5×10⁶ (FIG. 26B), 2×10⁶ (FIG. 26C), and 5×10⁶ (FIG. 26D) Carvykti or SMAR-Carvykti cells.

FIG. 27 is a series of graphs illustrating CAR+ cell counts in the bone marrow (0.5×10⁶ group only, leftmost graph) and blood (0.5×10⁶ group, second to left graph; 2×10⁶, second to right graph; 5×10⁶, rightmost graph) of mice treated with Carvykti or SMAR-Carvykti cells.

FIG. 28A is a series of graphs illustrating percentages of CD8+/CD4+ cells in the bone marrow (“BM,” left) and blood (right) of mice treated with Carvykti or SMAR-Carvykti cells.

FIG. 28B is a series of graphs illustrating CAR+CD8+ cell counts in the bone marrow and blood of mice treated with Carvykti or SMAR-Carvykti cells.

FIGS. 29A and 29B are a series of graphs illustrating cell phenotypes by percentage (FIG. 29A) and by cell counts (FIG. 29B) in the bone marrow and blood of mice treated with Carvykti or SMAR-Carvykti cells.

### DETAILED DESCRIPTION

Described herein are cytokine receptor switches, and compositions and uses thereof, that are engineered to control an intracellular signaling domain, e.g., through binding of an exogenous activator (e.g., a small molecule) to an extracellular domain (e.g., an activator binding domain). Such engineered cytokine receptor switches may be expressed in a cell, such as an immune cell, to activate cytokine signaling pathways through binding of the activator. When expressed and activated in an immune cell, such as a T-cell, an engineered cytokine receptor switch of the present disclosure may promote adoption of memory-like phenotypes which enable the immune cell to persist long-term in a subject. Memory immune cells may rapidly convert to effector immune cells upon encountering a target antigen, thereby promoting long-term immunity against the target.

Also described herein are compositions and methods to direct engineered immune cells to the lymphoid organs. A composition of the present disclosure may comprise an engineered immune cell expressing an exogenous receptor (e.g., an engineered cytokine receptor switch) comprising an extracellular domain (e.g., an activator binding domain) that binds an activator (e.g., a small molecule). In some embodiments, the composition may comprise a bispecific agent comprising the activator conjugated to a lymphoid-targeting protein that binds to a lymphoid surface marker. The bispecific agent may bind to both the exogenous receptor on the engineered immune cell via the activator and to the lymphoid surface marker on a cell in a lymphoid organ via the lymphoid-targeting protein, thereby recruiting the engineered immune cell to the lymphoid organ. In some embodiments, the engineered cytokine receptor switch may comprise an extracellular domain that binds directly to a lymphoid surface marker.

One implementation of the engineered cytokine receptor switches described herein is for chimeric antigen receptor (CAR) T-cell therapy. The compositions and methods described herein may be used to increase the efficacy of a CAR T-cell therapy by increasing the persistence of CAR T-cells in the subject. In traditional CAR T-cell therapy, T-cells are collected from a patient with cancer or from another donor, and the T-cells are engineered to express a CAR that binds a tumor cell antigen associated with the cancer. The engineered T-cells are amplified and returned to the patient. In the patient, an engineered T-cell binds to a tumor cell via the CAR and activates a cytotoxic response against the tumor cell, killing it. However, cancer relapse is common in patients receiving traditional CAR T-cell therapy. One mechanism of relapse following traditional CAR T-cell therapy is poor persistence of the CAR T-cells, resulting in a loss of CAR T-cells over time. To prevent relapse due to poor CAR T-cell persistence, an engineered cytokine receptor switch may be co-expressed with the CAR in T-cells. In contrast to traditional CAR T-cells expressing only a CAR, T-cells co-expressing a CAR and an engineered cytokine receptor switch may persist longer in patients due to formation of memory T-cell phenotypes triggered by activation of the engineered cytokine receptor switch and/or recruitment to the lymphoid organs. The memory T-cells may proliferate in the patient and may rapidly convert to effector T-cells upon exposure to the tumor cell antigen, thereby preventing cancer relapse. The T-cells expressing the engineered cytokine receptor switch may also be dynamically controlled, e.g., by administering an activator to the patient to control the activation of engineered cytokine receptor switch in vivo and/or by administering a bispecific agent to the patient that binds to both the engineered T-cell and another target (e.g., a lymphoid surface marker).

The engineered cytokine receptor switches may be expressed in an immune cell, such as a T-cell, to promote formation of memory phenotypes that are able to persist in a subject to facilitate long-term immune response. In some embodiments, an engineered cytokine receptor switch may be co-expressed with a CAR in an immune cell to enhance a function of the immune cell (e.g., by promoting persistence of the immune cell over time).

### I. ENGINEERED CYTOKINE RECEPTOR SWITCHES

As described herein, a cytokine receptor switch, also referred to as a small molecule activated receptor (SMAR) switch, may be engineered to activate an intracellular response (e.g., a cytokine pathway) upon binding of an activator to an extracellular domain. In some embodiments, an engineered cytokine receptor switch may comprise an activator binding domain, a transmembrane domain, and an intracellular signaling domain. The activator binding domain may bind an activator (e.g., a small molecule, a peptide, an oligonucleotide, or a protein) to activate the intracellular signaling domain. In some embodiments, the activator binding domain is a small molecule binding domain that binds a small molecule (e.g., fluorescein or a fluorescein derivative (e.g., fluorescein isothiocyanate (FITC)), tetraxetan (DOTA), biotin or linker-specific biotin, or 4-[(6-methylpyrazin-2-yl) oxy]benzoate (MPOB)). The activation signal may be communicated through the transmembrane domain to convert an extracellular stimulus (e.g., binding of the activator) to an intracellular effect (e.g., activation of a cytokine signaling pathway).

In some embodiments, the engineered cytokine receptor switch may further comprise a hinge connecting the activator binding domain to the transmembrane domain. A hinge may increase flexibility of the engineered cytokine receptor switch, which may reduce spatial constraints between the activator binding domain and the activator (e.g., a small molecule activator adhered to a surface). The engineered cytokine receptor switch may further comprise a signal peptide to direct expression of the engineered cytokine receptor switch to the endoplasmic reticulum (ER). In some embodiments, a signal peptide present at the N-terminus of the protein may direct the protein to be synthesized in the ER membrane and subsequently trafficked to the plasma membrane as a transmembrane protein.

An engineered cytokine receptor switch of the present disclosure may comprise a domain (e.g., an intracellular signaling domain, a transmembrane domain, a hinge, a signal peptide, or combinations thereof) derived from an endogenous cytokine receptor. In some embodiments, an engineered cytokine receptor switch may comprise a domain derived from an interleukin 2 receptor subunit α (IL2Rα), an interleukin 2 receptor subunit β (IL2Rβ), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (IL1R), a CD123, a CD124, an interleukin 5 receptor subunit α (IL5Rα), an interleukin 5 receptor subunit β (IL5Rβ), a CD126, a CD132, a CD129, an interleukin 11 receptor subunit α (IL11Rα), an interleukin 12 receptor subunit β1 (IL12Rβ1), an interleukin 12 receptor subunit β2 (IL12Rβ2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, a CD8, a CD3, a CD4, a CD28, a 4-1BB, a CD28, an OX40, an inducible T cell costimulatory (ICOS), a CD27, or combinations thereof.

Examples of engineered cytokine receptor switches and their associated polynucleotide sequences are provided in Table 1.

TABLE 1
Representative Examples of Engineered Cytokine Receptor Switches

| | SEQ ID | |
|---|---|---|
| SMAR | NO | Sequence |
| IL2Rα | SEQ ID | MDSYLLMWGLLTFIMVPGCQADVVMTQTPLSLPVSLGDQASISCRSSQSLVHSNG |
| SMAR | NO: 1 | NTYLRWYLQKPGQSPKVLIYKVSNRVSGVPDRFSGSGSGTDFTLKINRVEAEDLG |
| | | VYFCSQSTHVPWTFGGGTKLEIKSSADDAKKDAAKKDDAKKDDAKKDGGVKLD |
| | | ETGGGLVQPGGAMKLSCVTSGFTFGHYWMNWVRQSPEKGLEWVAQFRNKPYN |
| | | YETYYSDSVKGRFTISRDDSKSSVYLQMNNLRVEDTGIYYCTGASYGMEYLGQG |
| | | TSVTVSTTTPAPRPPTPAPTIASQPLSLRPEACRPAAGGAVHTRGLDFACD |
| | | VAVAGCVFLLISVLLLSGL TWQRRQRKSRRTI |
| IL2Rβ | SEQ ID | MAAPALSWRLPLLILLLPLATSWASADVVMTQTPLSLPVSLGDQASISCRSSQSLV |
| SMAR | NO: 2 | HSNGNTYLRWYLQKPGQSPKVLIYKVSNRVSGVPDRFSGSGSGTDFTLKINRVEA |
| | | EDLGVYFCSQSTHVPWTFGGGTKLEIKSSADDAKKDAAKKDDAKKDDAKKDGG |
| | | VKLDETGGGLVQPGGAMKLSCVTSGFTFGHYWMNWVRQSPEKGLEWVAQFRN |
| | | KPYNYETYYSDSVKGRFTISRDDSKSSVYLQMNNLRVEDTGIYYCTGASYGMEYL |
| | | GQGTSVTVSTTTPAPRPPTPAPTIASQPLSLRPEACRPAAGGAVHTRGLDFACDIPW |
| | | LGHLLVGLSGAFGFIILVYLLINCRNTGPWLKKVLKCNTPDPSKFFSQLSSEHGGD |
| | | VQKWLSSPFPSSSFSPGGLAPEISPLEVLERDKVTQLLLQQDKVPEPASLSSNHSLT |
| | | SCFTNQGYFFFHLPDALEIEACQVYFTYDPYSEEDPDEGVAGAPTGSSPQPLQPLS |
| | | GEDDAYCTFPSRDDLLLFSPSLLGGPSPPSTAPGGSGAGEERMPPSLQERVPRDWD |
| | | PQPLGPPTPGVPDLVDFQPPPELVLREAGEEVPDAGPREGVSFPWSRPPGQGEFRA |
| | | LNARLPLNTDAYLSLQELQGQDPTHLV |
| IL2Rγ | SEQ ID | MLKPSLPFTSLLFLQLPLLGVGDVVMTQTPLSLPVSLGDQASISCRSSQSLVHSNG |
| SMAR | NO: 3 | NTYLRWYLQKPGQSPKVLIYKVSNRVSGVPDRFSGSGSGTDFTLKINRVEAEDLG |
| | | VYFCSQSTHVPWTFGGGTKLEIKSSADDAKKDAAKKDDAKKDDAKKDGGVKLD |
| | | ETGGGLVQPGGAMKLSCVTSGFTFGHYWMNWVRQSPEKGLEWVAQFRNKPYN |
| | | YETYYSDSVKGRFTISRDDSKSSVYLQMNNLRVEDTGIYYCTGASYGMEYLGQG |
| | | TSVTVSTTTPAPRPPTPAPTIASQPLSLRPEACRPAAGGAVHTRGLDFACDVVISVG |
| | | SMGLIISLLCVYFWLERTMPRIPTLKNLEDLVTEYHGNFSAWSGVSKGLAESLQPD |
| | | YSERLCLVSEIPPKGGALGEGPGASPCNQHSPYWAPPCYTLKPET |
| IL7Rα | SEQ ID | MTILGTTFGMVFSLLQVVSGDVVMTQTPLSLPVSLGDQASISCRSSQSLVHSNGNT |
| SMAR | NO: 4 | YLRWYLQKPGQSPKVLIYKVSNRVSGVPDRFSGSGSGTDFTLKINRVEAEDLGVY |
| | | FCSQSTHVPWTFGGGTKLEIKSSADDAKKDAAKKDDAKKDDAKKDGGVKLDET |
| | | GGGLVQPGGAMKLSCVTSGFTFGHYWMNWVRQSPEKGLEWVAQFRNKPYNYE |
| | | TYYSDSVKGRFTISRDDSKSSVYLQMNNLRVEDTGIYYCTGASYGMEYLGQGTSV |
| | | TVSTTTPAPRPPTPAPTIASQPLSLRPEACRPAAGGAVHTRGLDFACDPILLTISILSF |
| | | FSVALLVILACVLWKKRIKPIVWPSLPDHKKTLEHLCKKPRKNLNVSFNPESFLDC |
| | | QIHRVDDIQARDEVEGFLQDTFPQQLEESEKQRLGGDVQSPNCPSEDVVITPESFG |
| | | RDSSLTCLAGNVSACDAPILSSSRSLDCRESGKNGPHVYQDLLLSLGTTNSTLPPPF |
| | | SLQSGILTLNPVAQGQPILTSLGSNQEEAYVTMSSFYQNQ |
| IL15Rα | SEQ ID | MAPRRARGCRTLGLPALLLLLLLRPPATRGDVVMTQTPLSLPVSLGDQASISCRSS |
| SMAR | NO: 5 | QSLVHSNGNTYLRWYLQKPGQSPKVLIYKVSNRVSGVPDRFSGSGSGTDFTLKIN |
| | | RVEAEDLGVYFCSQSTHVPWTFGGGTKLEIKSSADDAKKDAAKKDDAKKDDAK |
| | | KDGGVKLDETGGGLVQPGGAMKLSCVTSGFTFGHYWMNWVRQSPEKGLEWVA |
| | | QFRNKPYNYETYYSDSVKGRFTISRDDSKSSVYLQMNNLRVEDTGIYYCTGASYG |
| | | MEYLGQGTSVTVSTTTPAPRPPTPAPTIASQPLSLRPEACRPAAGGAVHTRGLDFA |
| | | CDVAISTSTVLLCGLSAVSLLACYLKSRQTPPLASVEMEAMEALPVTWGTSSRDE |
| | | DLENCSHHL |
| IL21Rα | SEQ ID | MPRGWAAPLLLLLLQGGWGDVVMTQTPLSLPVSLGDQASISCRSSQSLVHSNGN |
| SMAR | NO: 6 | TYLRWYLQKPGQSPKVLIYKVSNRVSGVPDRFSGSGSGTDFTLKINRVEAEDLGV |
| | | YFCSQSTHVPWTFGGGTKLEIKSSADDAKKDAAKKDDAKKDDAKKDGGVKLDE |
| | | TGGGLVQPGGAMKLSCVTSGFTFGHYWMNWVRQSPEKGLEWVAQFRNKPYNY |
| | | ETYYSDSVKGRFTISRDDSKSSVYLQMNNLRVEDTGIYYCTGASYGMEYLGQGTS |
| | | VTVSTTTPAPRPPTPAPTIASQPLSLRPEACRPAAGGAVHTRGLDFACDGWNPHLL |
| | | LLLLLVIVFIPAFWSLKTHPLWRLWKKIWAVPSPERFFMPLYKGCSGDFKKWVGA |
| | | PFTGSSLELGPWSPEVPSTLEVYSCHPPRSPAKRLQLTELQEPAELVESDGVPKPSF |
| | | WPTAQNSGGSAYSEERDRPYGLVSIDTVTVLDAEGPCTWPCSCEDDGYPALDLD |
| | | AGLEPSPGLEDPLLDAGTTVLSCGCVSAGSPGLGGPLGSLLDRLKPPLADGEDWA |
| | | GGLPWGGRSPGGVSESEAGSPLAGLDMDTFDSGFVGSDCSSPVECDFTSPGDEGP |
| | | PRSYLRQWVVIPPPLSSPGPQAS |
| IL2Rβ/γ | SEQ ID | MAAPALSWRLPLLILLLPLATSWASADVVMTQTPLSLPVSLGDQASISCRSSQSLV |
| SMAR | NO: 7 | HSNGNTYLRWYLQKPGQSPKVLIYKVSNRVSGVPDRFSGSGSGTDFTLKINRVEA |
| | | EDLGVYFCSQSTHVPWTFGGGTKLEIKSSADDAKKDAAKKDDAKKDDAKKDGG |
| | | VKLDETGGGLVQPGGAMKLSCVTSGFTFGHYWMNWVRQSPEKGLEWVAQFRN |
| | | KPYNYETYYSDSVKGRFTISRDDSKSSVYLQMNNLRVEDTGIYYCTGASYGMEYL |
| | | GQGTSVTVSTTTPAPRPPTPAPTIASQPLSLRPEACRPAAGGAVHTRGLDFACDIPW |
| | | LGHLLVGLSGAFGFIILVYLLINCRNTGPWLKKVLKCNTPDPSKFFSQLSSEHGGD |
| | | VQKWLSSPFPSSSFSPGGLAPEISPLEVLERDKVTQLLLQQDKVPEPASLSSNHSLT |
| | | SCFTNQGYFFFHLPDALEIEACQVYFTYDPYSEEDPDEGVAGAPTGSSPQPLQPLS |
| | | GEDDAYCTFPSRDDLLLFSPSLLGGPSPPSTAPGGSGAGEERMPPSLQERVPRDWD |
| | | PQPLGPPTPGVPDLVDFQPPPELVLREAGEEVPDAGPREGVSFPWSRPPGQGEFRA |
| | | LNARLPLNTDAYLSLQELQGQDPTHLVTGSGATNFSLLKQAGDVEENPGPAMLKP |
| | | SLPFTSLLFLQLPLLGVGDVVMTQTPLSLPVSLGDQASISCRSSQSLVHSNGNTYLR |
| | | WYLQKPGQSPKVLIYKVSNRVSGVPDRFSGSGSGTDFTLKINRVEAEDLGVYFCS |
| | | QSTHVPWTFGGGTKLEIKSSADDAKKDAAKKDDAKKDDAKKDGGVKLDETGGG |
| | | LVQPGGAMKLSCVTSGFTFGHYWMNWVRQSPEKGLEWVAQFRNKPYNYETYYS |
| | | DSVKGRFTISRDDSKSSVYLQMNNLRVEDTGIYYCTGASYGMEYLGQGTSVTVST |
| | | TTPAPRPPTPAPTIASQPLSLRPEACRPAAGGAVHTRGLDFACDVVISVGSMGLIISL |
| | | LCVYFWLERTMPRIPTLKNLEDLVTEYHGNFSAWSGVSKGLAESLQPDYSERLCL |
| | | VSEIPPKGGALGEGPGASPCNQHSPYWAPPCYTLKPETGSGEGRGSLLTCGDVEEN |
| | | PGPWEMPPPRLLFFLLFLTPMEVRPEEPLVVKVEEGDNAVLQCLKGTSDGPTQQL |
| | | TWSRESPLKPFLKLSLGLPGLGIHMRPLAIWLFIFNVSQQMGGFYLCQPGPPSEKA |
| | | WQPGWTVNVEGSGELFRWNVSDLGGLGCGLKNRSSEGPSSPSGKLMSPKLYVW |
| | | AKDRPEIWEGEPPCLPPRDSLNQSLSQDLTMAPGSTLWLSCGVPPDSVSRGPLSWT |
| | | HVHPKGPKSLLSLELKDDRPARDMWVMETGLLLPRATAQDAGKYYCHRGNLTM |
| | | SFHLEITARPVLWHWLLRTGGWKVSAVTLAYLIFCLCSLVGILHLQRALVLRRKR |
| | | KRMTDPTRRF |
| IL2Rα | SEQ ID | ATGGACAGCTACCTGCTGATGTGGGGCCTGCTGACCTTCATCATGGTGCCTGG |
| SMAR | NO: 8 | CTGTCAGGCCGACGTGGTCATGACACAGACACCTCTGAGCCTGCCTGTGTCTC |
| | | TGGGAGATCAGGCCAGCATCAGCTGCAGATCTAGCCAGAGCCTGGTGCACAG |
| | | CAACGGCAACACCTACCTGCGGTGGTATCTGCAGAAGCCCGGCCAGTCTCCTA |
| | | AGGTGCTGATCTACAAGGTGTCCAACAGAGTGTCCGGCGTGCCCGATAGATTT |
| | | TCTGGCAGCGGCTCTGGCACCGACTTCACCCTGAAGATCAATAGAGTGGAAGC |
| | | CGAGGACCTGGGCGTGTACTTCTGTAGCCAGTCTACCCACGTGCCATGGACCT |
| | | TTGGCGGCGGAACAAAGCTGGAAATCAAGAGCAGCGCCGACGACGCCAAGAA |
| | | GGACGCCGCTAAGAAGGATGACGCCAAAAAAGACGATGCCAAAAAGGATGG |
| | | CGGCGTGAAGCTGGACGAAACAGGCGGAGGACTTGTTCAGCCTGGCGGAGCC |
| | | ATGAAGCTGAGCTGTGTGACCAGCGGCTTCACCTTCGGCCACTACTGGATGAA |
| | | CTGGGTCCGACAGAGCCCTGAGAAAGGCCTGGAATGGGTCGCCCAGTTCAGA |
| | | AACAAGCCCTACAACTACGAAACCTACTACAGCGACAGCGTGAAGGGCAGAT |
| | | TCACCATCAGCCGGGACGACAGCAAGTCCAGCGTGTACCTGCAGATGAACAA |
| | | CCTGCGCGTGGAAGATACCGGCATCTACTACTGTACCGGCGCCAGCTACGGCA |
| | | TGGAATATCTCGGCCAGGGCACCAGCGTGACCGTGTCTACAACAACCCCTGCT |
| | | CCTCGGCCTCCTACACCAGCTCCTACAATTGCCAGCCAGCCACTGTCTCTGAG |
| | | GCCCGAAGCTTGTAGACCTGCTGCAGGCGGAGCCGTGCATACAAGAGGACTG |
| | | GACTTCGCCTGTGATGTGGCCGTGGCCGGATGTGTGTTTCTGCTGATCTCTGTG |
| | | CTGCTGCTGAGCGGCCTGACTTGGCAAAGACGGCAGAGAAAGAGCCGGCGGA |
| | | CCATCTGATAA |
| IL2Rβ | SEQ ID | ATGGCTGCTCCAGCTCTGTCTTGGAGACTGCCCCTGCTGATTCTGCTGCTGCCT |
| SMAR | NO: 9 | CTGGCTACATCTTGGGCCTCTGCCGACGTGGTCATGACACAGACACCACTGAG |
| | | CCTGCCTGTGTCTCTGGGAGATCAGGCCAGCATCAGCTGCAGATCCAGCCAGT |
| | | CTCTGGTGCACAGCAACGGCAACACCTACCTGCGGTGGTATCTGCAGAAGCCC |
| | | GGCCAGTCTCCTAAGGTGCTGATCTACAAGGTGTCCAACAGAGTGTCCGGCGT |
| | | GCCCGATAGATTTTCTGGCAGCGGCTCTGGCACCGACTTCACCCTGAAGATCA |
| | | ATAGAGTGGAAGCCGAGGACCTGGGCGTGTACTTCTGTAGCCAGTCTACCCAC |
| | | GTGCCATGGACCTTTGGCGGCGGAACAAAGCTGGAAATCAAGAGCAGCGCCG |
| | | ACGACGCCAAGAAGGACGCCGCTAAGAAGGATGACGCCAAAAAAGACGATG |
| | | CCAAAAAGGATGGCGGCGTGAAGCTGGACGAAACAGGCGGAGGACTTGTTCA |
| | | GCCTGGCGGAGCCATGAAGCTGAGCTGTGTGACCAGCGGCTTCACCTTCGGCC |
| | | ACTACTGGATGAACTGGGTCCGACAGAGCCCTGAGAAAGGCCTGGAATGGGT |
| | | CGCCCAGTTCAGAAACAAGCCCTACAACTACGAAACCTACTACAGCGACAGC |
| | | GTGAAGGGCAGATTCACCATCAGCCGGGACGACAGCAAGTCCAGCGTGTACC |
| | | TGCAGATGAACAACCTGCGCGTGGAAGATACCGGCATCTACTACTGTACCGGC |
| | | GCCAGCTACGGCATGGAATATCTCGGCCAGGGCACCAGCGTGACCGTGTCTAC |
| | | AACAACCCCTGCTCCTCGGCCTCCTACACCAGCTCCTACAATTGCCAGCCAGC |
| | | CACTGTCTCTGAGGCCCGAAGCTTGTAGACCTGCTGCAGGCGGAGCCGTGCAT |
| | | ACAAGAGGACTGGATTTCGCCTGCGACATCCCCTGGCTGGGACATCTGCTTGT |
| | | TGGACTGTCTGGCGCCTTCGGCTTCATCATCCTGGTGTATCTGCTGATCAACTG |
| | | CCGGAACACAGGCCCTTGGCTGAAGAAAGTGCTGAAGTGCAACACCCCTGAT |
| | | CCGAGCAAGTTCTTTAGCCAGCTGAGCAGCGAGCATGGCGGCGACGTTCAGA |
| | | AATGGCTGTCTAGCCCATTTCCTAGCAGCAGCTTCAGCCCAGGTGGACTGGCC |
| | | CCTGAGATTAGCCCTCTGGAAGTGCTGGAACGGGACAAAGTGACCCAGCTGCT |
| | | CCTCCAGCAGGATAAGGTGCCAGAACCTGCCAGCCTGTCCAGCAATCACAGCC |
| | | TGACCAGCTGCTTTACCAACCAGGGCTACTTCTTCTTCCATCTGCCTGACGCTC |
| | | TGGAAATCGAGGCCTGCCAGGTGTACTTCACCTACGATCCCTACAGCGAAGAG |
| | | GACCCCGATGAAGGTGTTGCTGGCGCCCCTACAGGATCTTCTCCACAGCCTCT |
| | | GCAACCTCTGAGCGGCGAGGATGATGCCTACTGCACCTTTCCAAGCAGGGACG |
| | | ACCTGCTCCTGTTCAGCCCATCTCTGCTCGGAGGACCATCTCCTCCATCTACAG |
| | | CTCCAGGCGGATCTGGCGCTGGCGAGGAAAGAATGCCACCTAGCCTGCAAGA |
| | | GCGGGTGCCCAGAGATTGGGATCCTCAACCTCTCGGCCCTCCAACACCTGGCG |
| | | TGCCAGATCTCGTGGACTTTCAGCCTCCTCCAGAGCTGGTGCTGAGAGAAGCT |
| | | GGCGAAGAAGTGCCAGACGCTGGCCCTAGAGAGGGCGTTAGCTTTCCTTGGA |
| | | GCAGACCTCCTGGACAGGGCGAGTTTAGGGCCCTGAATGCAAGACTGCCTCTG |
| | | AACACCGACGCCTACCTGTCTCTGCAAGAACTGCAGGGACAAGACCCCACAC |
| | | ACCTGGTGTAATGA |
| IL2Rγ | SEQ ID | ATGCTGAAGCCCAGCCTGCCTTTTACCAGCCTGCTGTTCCTGCAGCTGCCTCTG |
| SMAR | NO: 10 | CTTGGCGTGGGAGATGTGGTCATGACACAGACCCCACTGAGCCTGCCTGTGTC |
| | | TCTGGGAGATCAGGCCAGCATCAGCTGCAGATCTAGCCAGAGCCTGGTGCACA |
| | | GCAACGGCAACACCTACCTGCGGTGGTATCTGCAGAAGCCCGGCCAGTCTCCT |
| | | AAGGTGCTGATCTACAAGGTGTCCAACAGAGTGTCCGGCGTGCCCGATAGATT |
| | | TTCTGGCAGCGGCTCTGGCACCGACTTCACCCTGAAGATCAATAGAGTGGAAG |
| | | CCGAGGACCTGGGCGTGTACTTCTGTAGCCAGTCTACCCACGTGCCATGGACC |
| | | TTTGGCGGCGGAACAAAGCTGGAAATCAAGAGCAGCGCCGACGACGCCAAGA |
| | | AGGACGCCGCTAAGAAGGATGACGCCAAAAAAGACGATGCCAAAAAGGATG |
| | | GCGGCGTGAAGCTGGACGAAACAGGCGGAGGACTTGTTCAGCCTGGCGGAGC |
| | | CATGAAGCTGAGCTGTGTGACCAGCGGCTTCACCTTCGGCCACTACTGGATGA |
| | | ACTGGGTCCGACAGAGCCCTGAGAAAGGCCTGGAATGGGTCGCCCAGTTCAG |
| | | AAACAAGCCCTACAACTACGAAACCTACTACAGCGACAGCGTGAAGGGCAGA |
| | | TTCACCATCAGCCGGGACGACAGCAAGTCCAGCGTGTACCTGCAGATGAACA |
| | | ACCTGCGCGTGGAAGATACCGGCATCTACTACTGTACCGGCGCCAGCTACGGC |
| | | ATGGAATATCTCGGCCAGGGCACCAGCGTGACCGTGTCTACAACAACCCCTGC |
| | | TCCTCGGCCTCCTACACCAGCTCCTACAATTGCCAGCCAGCCACTGTCTCTGAG |
| | | GCCCGAAGCTTGTAGACCTGCTGCAGGCGGAGCCGTGCATACAAGAGGACTG |
| | | GACTTCGCCTGCGACGTGGTCATCTCTGTGGGCTCTATGGGCCTGATCATCTCC |
| | | CTGCTGTGTGTGTACTTTTGGCTGGAACGGACCATGCCTCGGATCCCCACACTG |
| | | AAGAACCTGGAAGATCTGGTCACCGAGTACCACGGCAACTTCAGTGCTTGGAG |
| | | CGGCGTGTCAAAAGGACTGGCCGAAAGCCTGCAGCCTGACTACTCCGAGAGA |
| | | CTGTGCCTGGTGTCTGAGATCCCTCCTAAAGGCGGCGCTCTCGGAGAAGGACC |
| | | TGGTGCCTCTCCATGCAATCAGCACAGCCCTTATTGGGCCCCTCCTTGCTACAC |
| | | CCTGAAACCTGAGACATGATGA |
| IL7Rα | SEQ ID | ATGACAATCCTGGGCACCACCTTCGGCATGGTGTTCAGTCTGCTGCAGGTCGT |
| SMAR | NO: 11 | GTCTGGCGACGTGGTCATGACACAGACCCCACTGTCTCTGCCTGTGTCTCTGG |
| | | GAGATCAGGCCAGCATCAGCTGCAGATCTAGCCAGAGCCTGGTGCACAGCAA |
| | | CGGCAACACCTACCTGCGGTGGTATCTGCAGAAGCCCGGCCAGTCTCCTAAGG |
| | | TGCTGATCTACAAGGTGTCCAACAGAGTGTCCGGCGTGCCCGATAGATTTTCT |
| | | GGCAGCGGCTCTGGCACCGACTTCACCCTGAAGATCAATAGAGTGGAAGCCG |
| | | AGGACCTGGGCGTGTACTTCTGTAGCCAGTCTACCCACGTGCCATGGACCTTT |
| | | GGCGGCGGAACAAAGCTGGAAATCAAGAGCAGCGCCGACGACGCCAAGAAG |
| | | GACGCCGCTAAGAAGGATGACGCCAAAAAAGACGATGCCAAAAAGGATGGC |
| | | GGCGTGAAGCTGGACGAAACAGGCGGAGGACTTGTTCAGCCTGGCGGAGCCA |
| | | TGAAGCTGAGCTGTGTGACCAGCGGCTTCACCTTTGGCCACTACTGGATGAAC |
| | | TGGGTCCGACAGAGCCCTGAGAAAGGCCTGGAATGGGTCGCCCAGTTCAGAA |
| | | ACAAGCCCTACAACTACGAAACCTACTACAGCGACAGCGTGAAGGGCAGATT |
| | | CACCATCAGCCGGGACGACAGCAAGTCCAGCGTGTACCTGCAGATGAACAAC |
| | | CTGCGCGTGGAAGATACCGGCATCTACTACTGTACCGGCGCCAGCTACGGCAT |
| | | GGAATATCTCGGCCAGGGCACCAGCGTGACCGTGTCTACAACAACCCCTGCTC |
| | | CTCGGCCTCCTACACCAGCTCCTACAATTGCCAGCCAGCCTCTGTCTCTGAGGC |
| | | CCGAAGCTTGTAGACCTGCTGCCGGCGGAGCTGTGCATACAAGAGGCCTGGAT |
| | | TTCGCCTGCGATCCCATCCTGCTGACAATCAGCATCCTGAGCTTTTTCAGCGTG |
| | | GCCCTGCTGGTCATCCTGGCCTGTGTGCTGTGGAAGAAGCGGATCAAGCCCAT |
| | | CGTGTGGCCCAGCCTGCCTGACCACAAGAAAACCCTGGAACACCTGTGCAAG |
| | | AAGCCCCGGAAGAACCTGAACGTGTCCTTCAATCCCGAGAGCTTCCTGGACTG |
| | | CCAGATCCACAGAGTGGACGACATCCAGGCCAGGGACGAAGTGGAAGGCTTT |
| | | CTGCAGGACACATTCCCTCAGCAGCTGGAAGAGAGCGAGAAGCAGAGACTCG |
| | | GAGGCGACGTGCAGAGCCCTAATTGCCCTTCTGAGGACGTCGTGATCACCCCT |
| | | GAGAGCTTCGGCAGAGATAGCAGCCTGACATGTCTGGCCGGCAATGTGTCCGC |
| | | CTGTGATGCCCCTATCCTGAGCAGCAGCAGAAGCCTGGATTGCAGAGAGAGC |
| | | GGCAAGAACGGCCCTCACGTGTACCAGGATCTGCTCCTGAGCCTGGGAACCAC |
| | | CAATAGCACACTGCCTCCACCATTCAGCCTGCAGAGCGGCATCCTGACACTGA |
| | | ACCCTGTTGCTCAGGGCCAGCCAATCCTGACCAGCCTGGGCAGCAATCAAGAA |
| | | GAGGCCTACGTCACCATGAGCAGCTTCTACCAGAACCAGTGATGATGGGAATG |
| | | GAACCTACCAGACCTGGGTGGCCAGCTCACTGAGCCATGCGCTATGTCCGGGA |
| | | ATGGAACCTACCAGACCTGGGTGGCCACCAGGATTTGCCAAGGAGAGGAGCA |
| | | GAGGTTCACCTGCTACATGGAACACAGCGGGAATCACAGCACTCACCCTGTGC |
| | | CCTCTGGGAAAGTGCTGGTGCTTCAGAGTCATTGGCAGACATTCCATGTTTCTT |
| | | AGGCTCGAGGTCGACGGTATCGATAAGCTTGATATCCGCCCCCCCCCCTAACG |
| | | TTACTGGCCGAAGCCGCTTGGAATAAGGCCGGTGTGCGTTTGTCTATATGTTAT |
| | | TTTCCACCATATTGCCGTCTTTTGGCAATGTGAGGGCCCGGAAACCTGGCCCTG |
| | | TCTTCTTGACGAGCATTCCTAGGGGTCTTTCCCCTCTCGCCAAAGGAATGCAAG |
| | | GTCTGTTGAATGTCGTGAAGGAAGCAGTTCCTCTGGAAGCTTCTTGAAGACAA |
| | | ACAACGTCTGTAGCGACCCTTTGCAGGCAGCGGAACCCCCCACCTGGCGACAG |
| | | GTGCCTCTGCGGCCAAAAGCCACGTGTATAAGATACACCTGCAAAGGCGGCA |
| | | CAACCCCAGTGCCACGTTGTGAGTTGGATAGTTGTGGAAAGAGTCAAATGGCT |
| | | CTCCTCAAGCGTATTCAACAAGGGGCTGAAGGATGCCCAGAAGGTACCCCATT |
| | | GTATGGGATCTGATCTGGGGCCTCGGTGCACATGCTTTACATGTGTTTAGTCGA |
| | | GGTTAAAAAAACGTCTAGGCCCCCCGAACCACGGGGACGTGGTTTTCCTTTGA |
| | | AAAACACGATGATAATATGGCCACAACGCGTACCATGGTGAGCAAGGGCGAG |
| | | GAGGATAACATGGCCATCATCAAGGAGTTCATGCGCTTCAAGGTGCACATGGA |
| | | GGGCTCCGTGAACGGCCACGAGTTCGAGATCGAGGGCGAGGGCGAGGGCCGC |
| | | CCCTACGAGGGCACCCAGACCGCCAAGCTGAAGGTGACCAAGGGTGGCCCCC |
| | | TGCCCTTCGCCTGGGACATCCTGTCCCCTCAGTTCATGTACGGCTCCAAGGCCT |
| | | ACGTGAAGCACCCCGCCGACATCCCCGACTACTTGAAGCTGTCCTTCCCCGAG |
| | | GGCTTCAAGTGGGAGCGCGTGATGAACTTCGAGGACGGCGGCGTGGTGACCG |
| | | TGACCCAGGACTCCTCCCTGCAGGACGGCGAGTTCATCTACAAGGTGAAGCTG |
| | | CGCGGCACCAACTTCCCCTCCGACGGCCCCGTAATGCAGAAGAAGACCATGG |
| | | GCTGGGAGGCCTCCTCCGAGCGGATGTACCCCGAGGACGGCGCCCTGAAGGG |
| | | CGAGATCAAGCAGAGGCTGAAGCTGAAGGACGGCGGCCACTACGACGCTGAG |
| | | GTCAAGACCACCTACAAGGCCAAGAAGCCCGTGCAGCTGCCCGGCGCCTACA |
| | | ACGTCAACATCAAGTTGGACATCACCTCCCACAACGAGGACTACACCATCGTG |
| | | GAACAGTACGAACGCGCCGAGGGCCGCCACTCCACCGGCGGCATGGACGAGC |
| | | TGTACAAGTGA |
| IL15Rα | SEQ ID | ATGGCTCCTCGGAGAGCCAGAGGCTGTAGAACACTTGGACTGCCCGCTCTGCT |
| SMAR | NO: 12 | GCTGCTCCTGCTTCTTAGACCTCCTGCCACAAGAGGCGACGTGGTCATGACAC |
| | | AGACCCCTCTGTCTCTGCCTGTGTCTCTGGGAGATCAGGCCAGCATCAGCTGC |
| | | AGATCTAGCCAGAGCCTGGTGCACAGCAACGGCAACACCTACCTGCGGTGGT |
| | | ATCTGCAGAAGCCCGGCCAGTCTCCTAAGGTGCTGATCTACAAGGTGTCCAAC |
| | | AGAGTGTCCGGCGTGCCCGATAGATTTTCTGGCAGCGGCTCTGGCACCGACTT |
| | | CACCCTGAAGATCAATAGAGTGGAAGCCGAGGACCTGGGCGTGTACTTCTGTA |
| | | GCCAGTCTACCCACGTGCCATGGACCTTTGGCGGCGGAACAAAGCTGGAAATC |
| | | AAGAGCAGCGCCGACGACGCCAAGAAGGACGCCGCTAAGAAGGATGACGCC |
| | | AAAAAAGACGATGCCAAAAAGGATGGCGGCGTGAAGCTGGACGAAACAGGC |
| | | GGAGGACTTGTTCAGCCTGGCGGAGCCATGAAGCTGAGCTGTGTGACCAGCG |
| | | GCTTCACCTTCGGCCACTACTGGATGAACTGGGTCCGACAGAGCCCTGAGAAA |
| | | GGCCTGGAATGGGTCGCCCAGTTCAGAAACAAGCCCTACAACTACGAAACCT |
| | | ACTACAGCGACAGCGTGAAGGGCAGATTCACCATCAGCCGGGACGACAGCAA |
| | | GTCCAGCGTGTACCTGCAGATGAACAACCTGCGCGTGGAAGATACCGGCATCT |
| | | ACTACTGTACCGGCGCCAGCTACGGCATGGAATATCTCGGCCAGGGCACCAGC |
| | | GTGACCGTGTCTACAACAACCCCTGCTCCTCGGCCTCCTACACCAGCTCCTACA |
| | | ATTGCCAGCCAGCCACTGTCTCTGAGGCCCGAAGCTTGTAGACCTGCTGCAGG |
| | | CGGAGCCGTGCATACAAGAGGACTGGACTTCGCCTGTGACGTGGCCATCAGCA |
| | | CAAGCACCGTTCTGCTGTGTGGCCTGTCAGCCGTTAGCCTGCTGGCTTGCTACC |
| | | TGAAGTCCAGACAGACACCTCCTCTGGCCAGCGTGGAAATGGAAGCCATGGA |
| | | AGCTCTGCCAGTGACCTGGGGCACCTCCAGCAGAGATGAGGATCTGGAAAAC |
| | | TGCAGCCACCACCTGTGATGA |
| IL21Rα | SEQ ID | ATGCCAAGAGGATGGGCCGCTCCTCTTCTCCTGTTGCTGCTTCAAGGCGGCTG |
| SMAR | NO: 13 | GGGCGACGTTGTGATGACACAGACACCACTGAGCCTGCCTGTGTCTCTGGGAG |
| | | ATCAGGCCAGCATCAGCTGCAGATCTAGCCAGAGCCTGGTGCACAGCAACGG |
| | | CAACACCTACCTGCGGTGGTATCTGCAGAAGCCCGGCCAGTCTCCTAAGGTGC |
| | | TGATCTACAAGGTGTCCAACAGAGTGTCCGGCGTGCCCGATAGATTTTCTGGC |
| | | AGCGGCTCTGGCACCGACTTCACCCTGAAGATCAATAGAGTGGAAGCCGAGG |
| | | ACCTGGGCGTGTACTTCTGTAGCCAGTCTACCCACGTGCCATGGACCTTTGGC |
| | | GGCGGAACAAAGCTGGAAATCAAGAGCAGCGCCGACGACGCCAAGAAGGAC |
| | | GCCGCTAAGAAGGATGACGCCAAAAAAGACGATGCCAAAAAGGATGGCGGC |
| | | GTGAAGCTGGACGAAACAGGCGGAGGACTTGTTCAGCCTGGCGGAGCCATGA |
| | | AGCTGAGCTGTGTGACCAGCGGCTTCACCTTCGGCCACTACTGGATGAACTGG |
| | | GTCCGACAGAGCCCTGAGAAAGGCCTGGAATGGGTCGCCCAGTTCAGAAACA |
| | | AGCCCTACAACTACGAAACCTACTACAGCGACAGCGTGAAGGGCAGATTCAC |
| | | CATCAGCCGGGACGACAGCAAGTCCAGCGTGTACCTGCAGATGAACAACCTG |
| | | CGCGTGGAAGATACCGGCATCTACTACTGTACCGGCGCCAGCTACGGCATGGA |
| | | ATATCTCGGCCAGGGCACCAGCGTGACCGTGTCTACAACAACCCCTGCTCCTC |
| | | GGCCTCCTACACCAGCTCCTACAATTGCCAGCCAGCCACTGTCTCTGAGGCCC |
| | | GAAGCTTGTAGACCTGCTGCAGGCGGAGCCGTGCATACAAGAGGACTGGACT |
| | | TTGCCTGCGACGGCTGGAATCCTCATCTGTTGTTGTTGCTCCTCCTGGTCATCG |
| | | TGTTCATCCCCGCCTTTTGGAGCCTGAAAACACACCCTCTGTGGCGGCTGTGG |
| | | AAGAAAATCTGGGCCGTGCCATCTCCTGAGCGGTTCTTCATGCCTCTGTACAA |
| | | GGGCTGCAGCGGCGACTTCAAGAAATGGGTCGGAGCCCCTTTTACCGGCAGCT |
| | | CTCTGGAACTTGGACCTTGGAGCCCTGAAGTGCCCAGCACACTGGAAGTGTAC |
| | | AGCTGTCACCCTCCTAGAAGCCCCGCCAAGAGACTGCAGCTCACAGAGCTGCA |
| | | AGAGCCTGCCGAGCTGGTGGAATCTGATGGCGTGCCCAAGCCTAGCTTCTGGC |
| | | CCACCGCTCAAAATTCTGGCGGCAGCGCCTACAGCGAGGAAAGAGATAGACC |
| | | TTACGGCCTGGTGTCCATCGACACCGTGACAGTGCTGGATGCCGAGGGACCTT |
| | | GTACCTGGCCTTGTAGCTGCGAGGACGATGGCTACCCTGCTCTGGATCTGGAC |
| | | GCAGGACTGGAACCTTCTCCAGGCCTCGAAGATCCTCTGCTGGACGCCGGAAC |
| | | AACAGTGCTGTCTTGTGGCTGTGTGTCCGCCGGATCTCCTGGACTTGGAGGAC |
| | | CTCTGGGAAGCCTGCTGGACAGACTGAAACCTCCTCTGGCCGATGGCGAAGAT |
| | | TGGGCTGGTGGACTTCCTTGGGGCGGAAGATCTCCAGGCGGAGTGTCTGAATC |
| | | TGAGGCCGGTTCTCCACTGGCCGGCCTGGATATGGATACCTTCGATAGCGGCT |
| | | TCGTGGGCAGCGATTGCAGCAGCCCTGTGGAATGCGACTTCACATCTCCTGGC |
| | | GACGAGGGCCCACCTAGAAGCTATCTCAGACAGTGGGTCGTGATCCCTCCACC |
| | | TCTGTCTAGTCCTGGACCACAGGCCAGCTGATGA |
| IL2Rβ/γ | SEQ ID | ATGGCTGCTCCAGCTCTGTCTTGGAGACTGCCCCTGCTGATTCTGCTGCTGCCT |
| SMAR | NO: 14 | CTGGCTACATCTTGGGCCTCTGCCGACGTGGTCATGACACAGACACCACTGAG |
| | | CCTGCCTGTGTCTCTGGGAGATCAGGCCAGCATCAGCTGCAGATCCAGCCAGT |
| | | CTCTGGTGCACAGCAACGGCAACACCTACCTGCGGTGGTATCTGCAGAAGCCC |
| | | GGCCAGTCTCCTAAGGTGCTGATCTACAAGGTGTCCAACAGAGTGTCCGGCGT |
| | | GCCCGATAGATTTTCTGGCAGCGGCTCTGGCACCGACTTCACCCTGAAGATCA |
| | | ATAGAGTGGAAGCCGAGGACCTGGGCGTGTACTTCTGTAGCCAGTCTACCCAC |
| | | GTGCCATGGACCTTTGGCGGCGGAACAAAGCTGGAAATCAAGAGCAGCGCCG |
| | | ACGACGCCAAGAAGGACGCCGCTAAGAAGGATGACGCCAAAAAAGACGATG |
| | | CCAAAAAGGATGGCGGCGTGAAGCTGGACGAAACAGGCGGAGGACTTGTTCA |
| | | GCCTGGCGGAGCCATGAAGCTGAGCTGTGTGACCAGCGGCTTCACCTTCGGCC |
| | | ACTACTGGATGAACTGGGTCCGACAGAGCCCTGAGAAAGGCCTGGAATGGGT |
| | | CGCCCAGTTCAGAAACAAGCCCTACAACTACGAAACCTACTACAGCGACAGC |
| | | GTGAAGGGCAGATTCACCATCAGCCGGGACGACAGCAAGTCCAGCGTGTACC |
| | | TGCAGATGAACAACCTGCGCGTGGAAGATACCGGCATCTACTACTGTACCGGC |
| | | GCCAGCTACGGCATGGAATATCTCGGCCAGGGCACCAGCGTGACCGTGTCTAC |
| | | AACAACCCCTGCTCCTCGGCCTCCTACACCAGCTCCTACAATTGCCAGCCAGC |
| | | CACTGTCTCTGAGGCCCGAAGCTTGTAGACCTGCTGCAGGCGGAGCCGTGCAT |
| | | ACAAGAGGACTGGATTTCGCCTGCGACATCCCCTGGCTGGGACATCTGCTTGT |
| | | TGGACTGTCTGGCGCCTTCGGCTTCATCATCCTGGTGTATCTGCTGATCAACTG |
| | | CCGGAACACAGGCCCTTGGCTGAAGAAAGTGCTGAAGTGCAACACCCCTGAT |
| | | CCGAGCAAGTTCTTTAGCCAGCTGAGCAGCGAGCATGGCGGCGACGTTCAGA |
| | | AATGGCTGTCTAGCCCATTTCCTAGCAGCAGCTTCAGCCCAGGTGGACTGGCC |
| | | CCTGAGATTAGCCCTCTGGAAGTGCTGGAACGGGACAAAGTGACCCAGCTGCT |
| | | CCTCCAGCAGGATAAGGTGCCAGAACCTGCCAGCCTGTCCAGCAATCACAGCC |
| | | TGACCAGCTGCTTTACCAACCAGGGCTACTTCTTCTTCCATCTGCCTGACGCTC |
| | | TGGAAATCGAGGCCTGCCAGGTGTACTTCACCTACGATCCCTACAGCGAAGAG |
| | | GACCCCGATGAAGGTGTTGCTGGCGCCCCTACAGGATCTTCTCCACAGCCTCT |
| | | GCAACCTCTGAGCGGCGAGGATGATGCCTACTGCACCTTTCCAAGCAGGGACG |
| | | ACCTGCTCCTGTTCAGCCCATCTCTGCTCGGAGGACCATCTCCTCCATCTACAG |
| | | CTCCAGGCGGATCTGGCGCTGGCGAGGAAAGAATGCCACCTAGCCTGCAAGA |
| | | GCGGGTGCCCAGAGATTGGGATCCTCAACCTCTCGGCCCTCCAACACCTGGCG |
| | | TGCCAGATCTCGTGGACTTTCAGCCTCCTCCAGAGCTGGTGCTGAGAGAAGCT |
| | | GGCGAAGAAGTGCCAGACGCTGGCCCTAGAGAGGGCGTTAGCTTTCCTTGGA |
| | | GCAGACCTCCTGGACAGGGCGAGTTTAGGGCCCTGAATGCAAGACTGCCTCTG |
| | | AACACCGACGCCTACCTGTCTCTGCAAGAACTGCAGGGACAAGACCCCACAC |
| | | ACCTGGTGACTGGATCTGGAGCAACAAACTTCTCACTACTCAAACAAGCAGGT |
| | | GACGTGGAGGAGAATCCCGGGCCTGCCATGCTGAAGCCCAGCCTGCCTTTTAC |
| | | CAGCCTGCTGTTCCTGCAGCTGCCTCTGCTTGGCGTGGGAGATGTGGTCATGA |
| | | CACAGACCCCACTGAGCCTGCCTGTGTCTCTGGGAGATCAGGCCAGCATCAGC |
| | | TGCAGATCTAGCCAGAGCCTGGTGCACAGCAACGGCAACACCTACCTGCGGTG |
| | | GTATCTGCAGAAGCCCGGCCAGTCTCCTAAGGTGCTGATCTACAAGGTGTCCA |
| | | ACAGAGTGTCCGGCGTGCCCGATAGATTTTCTGGCAGCGGCTCTGGCACCGAC |
| | | TTCACCCTGAAGATCAATAGAGTGGAAGCCGAGGACCTGGGCGTGTACTTCTG |
| | | TAGCCAGTCTACCCACGTGCCATGGACCTTTGGCGGCGGAACAAAGCTGGAAA |
| | | TCAAGAGCAGCGCCGACGACGCCAAGAAGGACGCCGCTAAGAAGGATGACGC |
| | | CAAAAAAGACGATGCCAAAAAGGATGGCGGCGTGAAGCTGGACGAAACAGG |
| | | CGGAGGACTTGTTCAGCCTGGCGGAGCCATGAAGCTGAGCTGTGTGACCAGCG |
| | | GCTTCACCTTCGGCCACTACTGGATGAACTGGGTCCGACAGAGCCCTGAGAAA |
| | | GGCCTGGAATGGGTCGCCCAGTTCAGAAACAAGCCCTACAACTACGAAACCT |
| | | ACTACAGCGACAGCGTGAAGGGCAGATTCACCATCAGCCGGGACGACAGCAA |
| | | GTCCAGCGTGTACCTGCAGATGAACAACCTGCGCGTGGAAGATACCGGCATCT |
| | | ACTACTGTACCGGCGCCAGCTACGGCATGGAATATCTCGGCCAGGGCACCAGC |
| | | GTGACCGTGTCTACAACAACCCCTGCTCCTCGGCCTCCTACACCAGCTCCTACA |
| | | ATTGCCAGCCAGCCACTGTCTCTGAGGCCCGAAGCTTGTAGACCTGCTGCAGG |
| | | CGGAGCCGTGCATACAAGAGGACTGGACTTCGCCTGCGACGTGGTCATCTCTG |
| | | TGGGCTCTATGGGCCTGATCATCTCCCTGCTGTGTGTGTACTTTTGGCTGGAAC |
| | | GGACCATGCCTCGGATCCCCACACTGAAGAACCTGGAAGATCTGGTCACCGAG |
| | | TACCACGGCAACTTCAGTGCTTGGAGCGGCGTGTCAAAAGGACTGGCCGAAA |
| | | GCCTGCAGCCTGACTACTCCGAGAGACTGTGCCTGGTGTCTGAGATCCCTCCT |
| | | AAAGGCGGCGCTCTCGGAGAAGGACCTGGTGCCTCTCCATGCAATCAGCACA |
| | | GCCCTTATTGGGCCCCTCCTTGCTACACCCTGAAACCTGAGACAGGCTCCGGC |
| | | GAGGGCAGGGGAAGTCTTCTAACATGCGGGGACGTGGAGGAAAATCCCGGCC |
| | | CATGGGAAATGCCTCCTCCTCGGCTGCTGTTCTTCCTGCTGTTTCTGACCCCTA |
| | | TGGAAGTGCGGCCCGAGGAACCTCTGGTGGTCAAAGTTGAAGAGGGCGACAA |
| | | CGCCGTGCTGCAGTGTCTGAAGGGCACATCTGATGGCCCCACACAGCAGCTGA |
| | | CCTGGTCTAGAGAGAGCCCTCTGAAGCCCTTCCTGAAGCTGTCTCTGGGACTG |
| | | CCTGGACTGGGCATCCATATGAGGCCTCTGGCCATCTGGCTGTTCATCTTCAAC |
| | | GTGTCCCAGCAGATGGGCGGCTTCTACCTGTGTCAACCTGGACCTCCAAGCGA |
| | | GAAGGCTTGGCAGCCTGGCTGGACCGTGAATGTGGAAGGATCCGGCGAGCTG |
| | | TTCCGGTGGAATGTGTCTGATCTCGGCGGCCTCGGATGCGGCCTGAAGAATAG |
| | | ATCTAGCGAGGGCCCTAGCAGCCCCAGCGGAAAACTGATGAGCCCCAAGCTG |
| | | TACGTGTGGGCCAAAGACAGACCCGAGATTTGGGAGGGCGAGCCTCCTTGTCT |
| | | GCCTCCTAGAGACAGCCTGAACCAGAGCCTGAGCCAGGACCTGACAATGGCC |
| | | CCTGGATCTACACTGTGGCTGAGCTGTGGCGTGCCACCTGACAGTGTGTCTAG |
| | | AGGCCCTCTGTCTTGGACCCACGTGCACCCTAAGGGCCCTAAGTCTCTGCTGA |
| | | GCCTGGAACTGAAGGACGACAGGCCCGCCAGAGATATGTGGGTCATGGAAAC |
| | | AGGCCTGCTGCTGCCTAGAGCCACAGCACAGGATGCCGGCAAGTACTACTGCC |
| | | ACAGAGGCAACCTGACCATGAGCTTCCACCTGGAAATCACCGCCAGACCTGTC |
| | | CTGTGGCACTGGCTGCTTAGAACCGGCGGCTGGAAAGTGTCTGCCGTGACTCT |
| | | GGCCTACCTGATCTTCTGCCTGTGTAGCCTCGTGGGCATCCTGCATCTGCAGAG |
| | | AGCACTGGTCCTGCGGCGGAAGCGGAAGAGAATGACCGATCCTACCAGACGG |
| | | TTCTGATGA |

An engineered cytokine receptor switch may comprise a signal peptide, an activator binding domain, a hinge, a transmembrane domain, and an intracellular signaling domain. In some embodiments, an engineered cytokine receptor switch may comprise a signal peptide of any one of SEQ ID NO: 15-SEQ ID NO: 20, an activator binding domain of SEQ ID NO: 21, a transmembrane domain of any one of SEQ ID NO: 23-SEQ ID NO: 28, and an intracellular signaling domain of any one of SEQ ID NO: 29-SEQ ID NO: 34. In some embodiments, an engineered cytokine receptor switch may further comprise a hinge (e.g., SEQ ID NO: 22), a cleavage sequence (e.g., any one of SEQ ID NO: 35-SEQ ID NO: 38), a marker (e.g., SEQ ID NO: 39), or combinations thereof.

In some embodiments, an engineered cytokine receptor switch may comprise a sequence having at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to any one of SEQ ID NO: 1-SEQ ID NO: 7. In some embodiments, an engineered cytokine receptor switch is encoded by a sequence having at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to any one of SEQ ID NO: 8-SEQ ID NO: 14. In some embodiments, the engineered cytokine receptor switch may comprise a sequence of any one of SEQ ID NO: 1-SEQ ID NO: 7. In some embodiments, the engineered cytokine receptor switch is encoded by a sequence of any one of SEQ ID NO: 8-SEQ ID NO: 14.

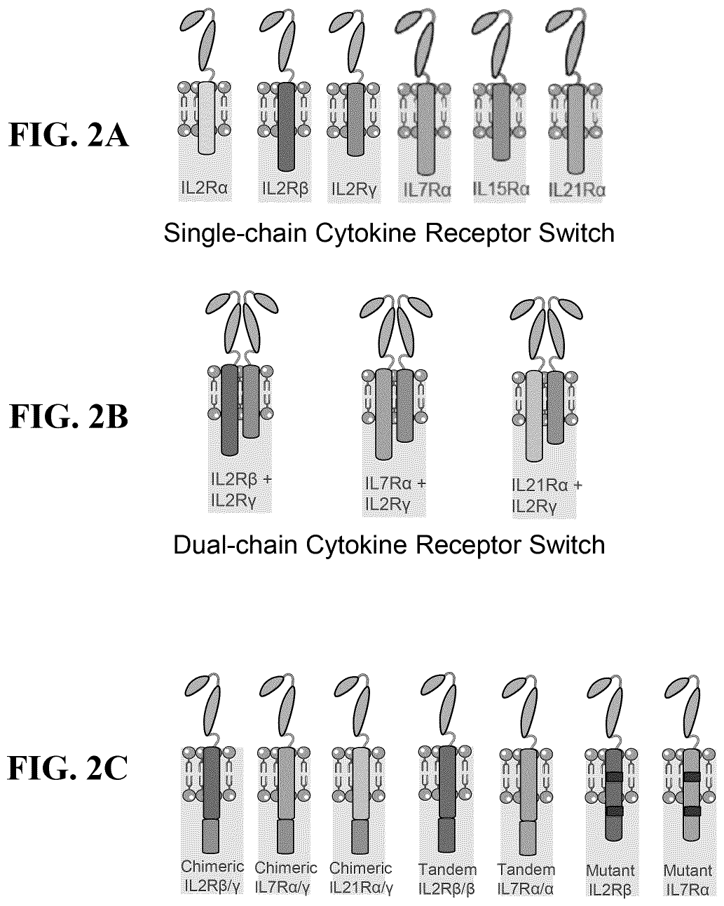

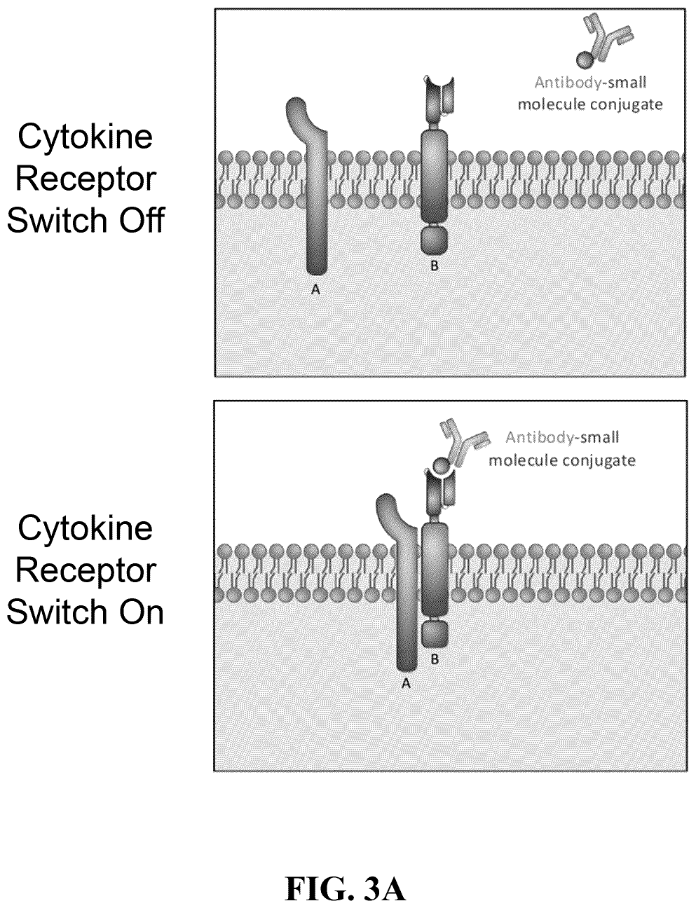

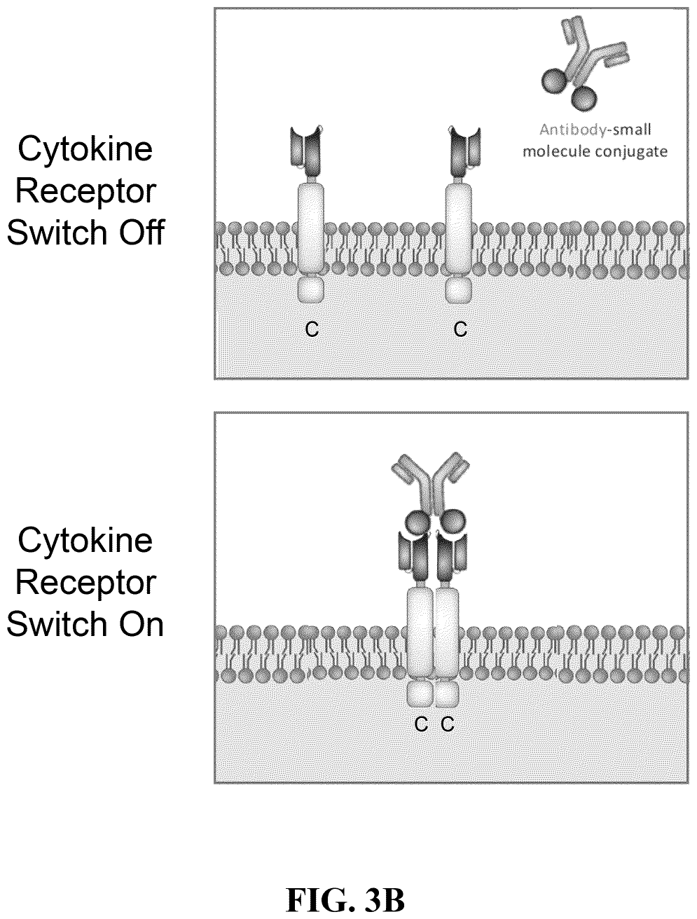

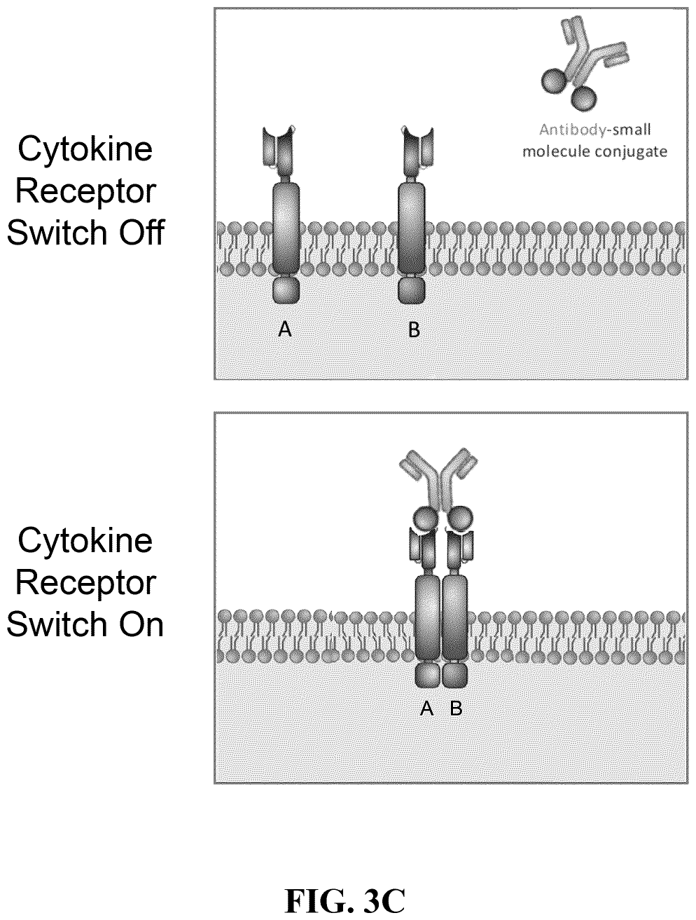

In some embodiments, an engineered cytokine receptor switch may be a single-chain cytokine receptor switch. Examples of single-chain cytokine receptor switches are illustrated in FIG. 2A, and may include any one of SEQ ID NO: 1-SEQ ID NO: 6 (based on IL2Rα, IL2Rβ, IL2Rγ, IL7Rα, IL15Rα, and IL21Rα, respectively). A single-chain cytokine receptor switch can be derived from a single cytokine receptor chain (e.g., an a, 3, or γ chain). The cytokine receptor chain may be a wild-type cytokine receptor chain, or may be a chimeric or mutant cytokine receptor chain. In some embodiments, the single cytokine receptor chain is capable of initiating signaling via dimerization with an endogenous cytokine receptor chain. For example, IL7 signaling occurs through the IL7R receptor, which is composed of the IL7Rα and IL2Rγ chains. The IL2Rγ chain (also known as the common gamma chain (yc)) is shared by other members of the in the common gamma chain receptor family. Accordingly, a single-chain cytokine receptor switch (e.g., derived from IL7Rα) may heterodimerize with an endogenous cytokine receptor (e.g., IL2Rγ) and bind to an activator to initiate intracellular signaling, e.g., as shown in FIG. 3A. Some cytokine receptor chains are capable of initiating signaling via homodimerization (e.g., IL7Rα can form homodimers and initiate IL7 signaling without IL2Rγ). Thus, in some embodiments, a pair of single-chain cytokine receptor switches (e.g., derived from IL7Rα) may bind to respective activators and homodimerize with each other to initiate intracellular signaling, e.g., as shown in FIG. 3B. Single-chain receptor switches may be used to initiate novel signaling pathways by dimerization with endogenous cytokine receptor chains, depending on how the dimerization occurs and which receptor chains are dimerized. Additionally, in some embodiments, production of viral vectors and engineered immune cells may be easier for single-chain cytokine receptor switches.

In some embodiments, an engineered cytokine receptor switch may be a dual-chain cytokine receptor switch. Representative examples of dual-chain cytokine receptor switches are illustrated in FIG. 2B. For example, a dual-chain cytokine receptor switch may include a first cytokine receptor chain of SEQ ID NO: 2 (based on IL2Rβ) and a second cytokine receptor chain of SEQ ID NO: 3 (based on IL2Rγ) (FIG. 2B, left). As another example, a dual-chain cytokine receptor switch may include a first cytokine receptor chain of SEQ ID NO: 4 (based on IL7Rα) and a second cytokine receptor chain of SEQ ID NO: 3 (based on IL2Rγ) (FIG. 2B, center). In a further example, a dual-chain cytokine receptor switch may include a first cytokine receptor chain of SEQ ID NO: 6 (based on IL21Rα) and a second cytokine receptor chain of SEQ ID NO:3 (based on IL2Rγ) (FIG. 2B, right). A dual-chain cytokine receptor switch can be derived from two cytokine receptor chains (e.g., a combination of α, β, or γ chains), each of which can be independently selected from any of the cytokine receptor chains described herein. For example, a dual-chain cytokine receptor switch including a first cytokine receptor chain derived from IL2Rβ and a second cytokine receptor chain derived from IL2Rγ can mimic the IL2-IL2R signaling pathway. In some embodiments, each chain of a dual-chain cytokine receptor switch may bind to a respective activator and heterodimerize with each other to activate intracellular signaling, e.g., as shown in FIG. 3C. Optionally, a dual-chain cytokine receptor switch can be expressed as a single protein including both cytokine receptor chains. The single protein can be subsequently cleaved (e.g., via the inclusion of a 2A peptide or other cleavage sequence) to produce the two separate cytokine receptor chains. SEQ ID NO: 7 provides an example of a dual-chain cytokine receptor switch that is initially expressed as a single protein.

In some embodiments, an engineered cytokine receptor switch includes one or more cytokine receptor chains with one or more chimeric, tandem, and/or mutant intracellular domains. Representative examples of single-chain cytokine receptor switches with chimeric, tandem, and/or mutant intracellular domains are shown in FIG. 2C. For example, a cytokine receptor switch can include a chimeric cytokine receptor chain including a first intracellular domain derived from IL2Rβ and a second intracellular domain derived from IL2Rγ (“chimeric IL2Rβ/γ” in FIG. 2C). As another example, a cytokine receptor switch can include a chimeric cytokine receptor chain including a first intracellular domain derived from IL7Rα and a second intracellular domain derived from IL2Rγ (“chimeric IL7Rα/γ” in FIG. 2C). In another example, a cytokine receptor switch can include a chimeric cytokine receptor chain including a first intracellular domain derived from IL21Rα and a second intracellular domain derived from IL2Rγ (“chimeric IL21Rα/γ” in FIG. 2C). In a further example, a cytokine receptor switch can include a tandem cytokine receptor chain including first and second intracellular domains derived from IL2Rβ (“tandem IL21Rβ/β” in FIG. 2C). As yet another example, a cytokine receptor switch can include a tandem cytokine receptor chain including first and second intracellular domains derived from IL7Rα (“tandem IL7Rα/α” in FIG. 2C). As another example, a cytokine receptor switch can include a mutant cytokine receptor chain including a mutant intracellular domain derived from IL2Rβ (“mutant IL21Rβ” in FIG. 2C). In another example, a cytokine receptor switch can include a mutant cytokine receptor chain including a mutant intracellular domain derived from IL7Rα (“mutant IL7Rα” in FIG. 2C). A mutant intracellular domain can include one or more mutations relative to the wild-type intracellular domain, such as point mutations, truncations, etc.

Although FIGS. 3A-3C illustrate the activator (“small molecule”) as being part of a soluble complex (“antibody-small molecule conjugate”), this is not intended to be limiting, and in other embodiments, the activator can be attached to a surface or other substrate, as described below.

Optionally, the engineered cytokine receptor switch may exhibit activity (e.g., a cytokine signaling activity) without binding of an activator to the activator binding domain, referred to herein as “activator-independent activity” or “ligand-independent activity.” Without wishing to be bound by theory, it is hypothesized that activator-independent activity may be due to dimerization of a cytokine receptor chain of an engineered cytokine receptor switch with another cytokine receptor chain (e.g., of the engineered cytokine receptor switch or an endogenous cytokine receptor) that occurs even in the absence of the activator. Dimerization may occur between the extracellular and/or transmembrane domains of the cytokine receptor chains. Activator-independent activity may also occur due to interactions of the engineered cytokine receptor switch with other co-expressed receptors, such as a CAR. Such interactions may comprise physical interactions (e.g., dimerization) as well as interactions in downstream signaling pathways. The strength of activator-independent activity can be increased or decreased by changing the extracellular domain of the engineered cytokine receptor switch, and/or by increasing or decreasing the length of the hinge between domains of the cytokine receptor switch.

In some embodiments, activator-independent activity provides similar effects as activation of the engineered cytokine receptor switch (e.g., enhancement of memory phenotypes and/or lymphoid homing), but with reduced magnitude and/or shorter duration. In some embodiments, activator-independent activity primes the immune cell for subsequent activation, e.g., the magnitude and/or duration of the effects following administration of the activator is greater if the immune cell has previously exhibited activator-independent activity, versus an immune cell that does not exhibit activator-independent activity.

In some embodiments, the level of activator-independent activity exhibited by an engineered cytokine receptor switch depends at least partially on the structure of the engineered cytokine receptor switch. For instance, a shorter hinge may be associated with higher levels of activator-independent activity, e.g., due to enhanced dimerization facilitated by the reduced flexibility of the extracellular and/or transmembrane domains of the engineered cytokine receptor switch. The shorter hinge can be no more than 30 amino acids, 25 amino acids, 20 amino acids, 15 amino acids, 10 amino acids, 9 amino acids, 8 amino acids, 7 amino acids, 6 amino acids, 5 amino acids, 4 amino acids, 3 amino acids, 2 amino acids, or 1 amino acid in length. Conversely, a longer hinge may be associated with lower levels of activator-independent activity, e.g., due to reduced dimerization attributable to the increased flexibility of the extracellular and/or transmembrane domains of the engineered cytokine receptor switch. The longer hinge can be at least 20 amino acids, 25 amino acids, 30 amino acids, 35 amino acids, 40 amino acids, 45 amino acids, or 50 amino acids in length. Other structural features that may influence activator-independent activity include the size of the extracellular domain, the size of the transmembrane domain, and/or the size of the intracellular domain.

The structure of the engineered cytokine receptor switch (e.g., length of the hinge) can be selected to produce a desired level of activator-independent activity. Activator-independent activity can be beneficial, for example, to provide constitutive enhancement of memory phenotypes and/or lymphoid homing (e.g., in embodiments the engineered cytokine receptor switch is co-expressed with a direct CAR). Conversely, lower levels of activator-independent activity may be advantageous in situations where switchable control over immune cell activity is desired (e.g., in embodiments the engineered cytokine receptor switch is co-expressed with an indirect CAR).

### A. Activator Binding Domain

An engineered cytokine receptor switch of the present disclosure may comprise an activator binding domain. The activator binding domain may be positioned in an extracellular region of the engineered cytokine receptor switch and may be designed to bind an activator (e.g., a small molecule, a peptide, an oligonucleotide, a protein) to activate intracellular signaling through the intracellular signaling domain. In some embodiments, an activator may be selected to have low toxicity, low immunogenicity, low cross-reactivity, or combinations thereof to reduce unfavorable side effects when administered to a subject (e.g., a human subject). For example, the activator may be an exogenous activator (e.g., an exogenous small molecule, an exogenous peptide, an exogenous oligonucleotide, or an exogenous protein) that is not naturally present in a target environment (e.g., a human subject) to prevent activation of the engineered cytokine receptor switch in the absence of an external stimulus (e.g., administration of the activator), prevent cross-reactivity of the activator with other biological components, and to enable dynamic control of receptor signaling. Additional examples of activators are provided in Section II below.

The activator binding domain can be any protein, protein fragment, or peptide capable of selectively binding the activator. In some embodiments, for example, the activator binding domain may comprise an antibody (e.g., a monoclonal antibody), an antibody fragment, a single chain variable fragment (scFv), a nanobody, or a peptide. In some embodiments, an activator binding domain may comprise a fragment of an antibody (e.g., a variable fragment) that binds to a selected activator. Antibodies, antibody fragments, scFvs, and nanobodies may be produced using various methods known in the art to target a specific activator. In some embodiments, the activator binding domain is an scFv, a heavy chain variable domain (V_{H}), or a light chain variable domain (V_{L}) of an antibody, or a VHH antibody that recognizes any of the activators described herein, e.g., in Section II below. For example, the activator binding domain can be an scFv, a V_{H}, or a V_{L} of an anti-FITC antibody (e.g., a 4M5.3 anti-FITC antibody). As another example, the activator binding domain can be an scFv, a V_{H}, or a V_{L} of an anti-DOTA antibody (e.g., a C8.2.5 anti-DOTA antibody). In a further example, the activator binding domain can be an scFv, a V_{H}, or a V_{L} of an anti-MPOB antibody.

In some embodiments, the activator binding domain may be synthetic (e.g., engineered de novo to bind a specific small molecule activator or other activator type). In some embodiments, a small molecule binding domain may be humanized to reduce immunogenicity and prevent an immune reaction to the engineered cytokine receptor switch when administered to a subject (e.g., a human subject). Commercially available small molecule binding domains may be suitable for use as an activator binding domain in an engineered cytokine receptor switch.

In some embodiments, an activator binding domain may be suitable for use in an engineered cytokine receptor switch of the present disclosure if the activator binding domain does not target a small molecule produced in humans. An activator binding domain may be suitable for use in an engineered cytokine receptor switch of the present disclosure if the activator binding domain binds to a molecule that is non-toxic to humans, included in an Inactive Ingredients Database, or both.

The activator binding domain may have a molecular weight of from about 1 kDa to about 150 kDa, from about 1 kDa to about 100 kDa, from about 1 kDa to about 90 kDa, from about 1 kDa to about 80 kDa, from about 1 kDa to about 70 kDa, from about 1 kDa to about 60 kDa, from about 1 kDa to about 50 kDa, from about 1 kDa to about 40 kDa, from about 1 kDa to about 35 kDa, from about 1 kDa to about 30 kDa, from about 1 kDa to about 25 kDa, from about 1 kDa to about 10 kDa, from about 5 kDa to about 150 kDa, from about 5 kDa to about 100 kDa, from about 5 kDa to about 90 kDa, from about 5 kDa to about 80 kDa, from about 5 kDa to about 70 kDa, from about 5 kDa to about 60 kDa, from about 5 kDa to about 50 kDa, from about 5 kDa to about 40 kDa, from about 5 kDa to about 35 kDa, from about 5 kDa to about 30 kDa, from about 5 kDa to about 25 kDa, from about 5 kDa to about 10 kDa, from about 10 kDa to about 150 kDa, from about 10 kDa to about 100 kDa, from about 10 kDa to about 90 kDa, from about 10 kDa to about 80 kDa, from about 10 kDa to about 70 kDa, from about 10 kDa to about 60 kDa, from about 10 kDa to about 50 kDa, from about 10 kDa to about 40 kDa, from about 10 kDa to about 35 kDa, from about 10 kDa to about 30 kDa, from about 10 kDa to about 25 kDa, from about 20 kDa to about 150 kDa, from about 20 kDa to about 100 kDa, from about 20 kDa to about 90 kDa, from about 20 kDa to about 80 kDa, from about 20 kDa to about 70 kDa, from about 20 kDa to about 60 kDa, from about 20 kDa to about 50 kDa, from about 20 kDa to about 40 kDa, from about 20 kDa to about 35 kDa, or from about 20 kDa to about 30 kDa. For example, the activator binding domain may comprise an scFv having a molecular weight of about 20 kDa to about 35 kDa. The activator binding domain may comprise a peptide having a molecular weight of about 1 kDa to about 10 kDa.

Examples of activator binding domains that may be used in an engineered cytokine receptor switch and corresponding activators are provided in Table 2.

TABLE 2
Representative Examples of Activator Binding Domains

| Activator | | | |
|---|---|---|---|
| Binding | | | |
| Domain | SEQ ID NO | Sequence | Activator |
| anti- | SEQ ID NO: 21 | DVVMTQTPLSLPVSLGDQASISCRSSQSLVHSNGNTYLR | Fluorescein |
| fluorescein | | WYLQKPGQSPKVLIYKVSNRVSGVPDRFSGSGSGTDFTL | or |
| scFv | | KINRVEAEDLGVYFCSQSTHVPWTFGGGTKLEIKSSADD | fluorescein |
| | | AKKDAAKKDDAKKDDAKKDGGVKLDETGGGLVQPGG | derivatives |
| | | AMKLSCVTSGFTFGHYWMNWVRQSPEKGLEWVAQFRN | (e.g., FITC) |
| | | KPYNYETYYSDSVKGRFTISRDDSKSSVYLQMNNLRVED | |
| | | TGIYYCTGASYGMEYLGQGTSVTVS | |

In some embodiments, an engineered cytokine receptor switch may comprise an activator binding domain comprising at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to SEQ ID NO: 21. In some embodiments, the engineered cytokine receptor switch may comprise an activator binding domain of SEQ ID NO: 21.

### B. Intracellular Domain

An engineered cytokine receptor switch of the present disclosure may comprise an intracellular domain (also referred to herein as an “intracellular signaling domain”). The intracellular signaling domain may be positioned in an intracellular region of the engineered cytokine receptor switch and may be designed to activate intracellular signaling upon binding of an activator to an extracellular activator binding domain. Optionally, the intracellular signaling domain may exhibit activity independent of binding of the activator to the activator binding domain (activator-independent activity), as described elsewhere herein. The intracellular signaling domain may activate a cytokine signaling pathway, such as a Jak-STAT pathway. In some embodiments, activation of the cytokine signaling pathway may promote conversion of an immune cell expressing the engineered cytokine receptor switch to a memory phenotype (e.g., a central memory phenotype, a stem cell memory phenotype, an effector memory phenotype, or an effector memory re-expressing CD45RA phenotype). Alternatively or in combination, activation of the cytokine signaling pathway may upregulate expression of cell surface markers that enable homing of the immune cell to lymphoid organs (e.g., CD62L, CCR7), such as homing to the lymph nodes, spleen, thymus, and/or bone marrow.

An intracellular signaling domain may be derived from an endogenous cytokine receptor. For example, an intracellular signaling domain may be derived from an interleukin 2 receptor subunit α (IL2Rα), an interleukin 2 receptor subunit β (IL2Rβ), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (IL1R), a CD123, a CD124, an interleukin 5 receptor subunit α (IL5Rα), an interleukin 5 receptor subunit β (IL5Rβ), a CD126, a CD132, a CD129, an interleukin 11 receptor subunit α (IL11Rα), an interleukin 12 receptor subunit β1 (IL12Rβ1), an interleukin 12 receptor subunit β2 (IL12Rβ2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, or a GM-CSF.

In some embodiments, the intracellular signaling domain may comprise an intracellular domain, a fragment of an intracellular domain, or a variant of an intracellular domain of an endogenous cytokine receptor. For example, the intracellular signaling domain may comprise an intracellular domain, a fragment of an intracellular domain, or a variant of an intracellular domain of an interleukin 2 receptor subunit α (IL2Rα), an interleukin 2 receptor subunit β (IL2Rβ), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (IL1R), a CD123, a CD124, an interleukin 5 receptor subunit α (IL5Rα), an interleukin 5 receptor subunit β (IL5Rβ), a CD126, a CD132, a CD129, an interleukin 11 receptor subunit α (IL11Rα), an interleukin 12 receptor subunit β1 (IL12Rβ1), an interleukin 12 receptor subunit β2 (IL12Rβ2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, or a GM-CSF. The intracellular domain, fragment of the intracellular domain, or variant of the intracellular domain may be capable of activating the cytokine signaling pathway activated by the endogenous cytokine receptor from which it was derived.

In some embodiments, an engineered cytokine receptor switch includes a single intracellular signaling domain. Alternatively, an engineered cytokine receptor switch can include a plurality of intracellular signaling domains in tandem (e.g., two, three, four, five, or more intracellular domains in tandem). In such embodiments, some or all of the intracellular signaling domains may be the same intracellular signaling domain, or some or all of the intracellular signaling domains may be different intracellular signaling domains.

Examples of intracellular signaling domains that may be used in an engineered cytokine receptor switch are provided in Table 3.

TABLE 3
Representative Examples of Intracellular Signaling Domains

| Receptor | SEQ ID NO | Sequence |
|---|---|---|
| IL2Rα | SEQ ID NO: 29 | TWQRRQRKSRRTI |
| IL2Rβ | SEQ ID NO: 30 | NCRNTGPWLKKVLKCNTPDPSKFFSQLSSEHGGDVQKWLSSPFPSSSFSP |
| | | GGLAPEISPLEVLERDKVTQLLLQQDKVPEPASLSSNHSLTSCFTNQGYF |
| | | FFHLPDALEIEACQVYFTYDPYSEEDPDEGVAGAPTGSSPQPLQPLSGED |
| | | DAYCTFPSRDDLLLFSPSLLGGPSPPSTAPGGSGAGEERMPPSLQERVPR |
| | | DWDPQPLGPPTPGVPDLVDFQPPPELVLREAGEEVPDAGPREGVSFPWS |
| | | RPPGQGEFRALNARLPLNTDAYLSLQELQGQDPTHLV |
| IL2Rγ | SEQ ID NO: 31 | ERTMPRIPTLKNLEDLVTEYHGNFSAWSGVSKGLAESLQPDYSERLCLV |
| | | SEIPPKGGALGEGPGASPCNQHSPYWAPPCYTLKPET |
| IL7Rα | SEQ ID NO: 32 | KKRIKPIVWPSLPDHKKTLEHLCKKPRKNLNVSFNPESFLDCQIHRVDDI |
| | | QARDEVEGFLQDTFPQQLEESEKQRLGGDVQSPNCPSEDVVITPESFGRD |
| | | SSLTCLAGNVSACDAPILSSSRSLDCRESGKNGPHVYQDLLLSLGTTNST |
| | | LPPPFSLQSGILTLNPVAQGQPILTSLGSNQEEAYVTMSSFYQNQ |
| IL15Rα | SEQ ID NO: 33 | KSRQTPPLASVEMEAMEALPVTWGTSSRDEDLENCSHHL |
| IL21Rα | SEQ ID NO: 34 | SLKTHPLWRLWKKIWAVPSPERFFMPLYKGCSGDFKKWVGAPFTGSSL |
| | | ELGPWSPEVPSTLEVYSCHPPRSPAKRLQLTELQEPAELVESDGVPKPSF |
| | | WPTAQNSGGSAYSEERDRPYGLVSIDTVTVLDAEGPCTWPCSCEDDGYP |
| | | ALDLDAGLEPSPGLEDPLLDAGTTVLSCGCVSAGSPGLGGPLGSLLDRL |
| | | KPPLADGEDWAGGLPWGGRSPGGVSESEAGSPLAGLDMDTFDSGFVGS |
| | | DCSSPVECDFTSPGDEGPPRSYLRQWVVIPPPLSSPGPQAS |

In some embodiments, an engineered cytokine receptor switch may comprise an intracellular signaling domain comprising at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to any one of SEQ ID NO: 29-SEQ ID NO: 34. In some embodiments, the engineered cytokine receptor switch may comprise an intracellular signaling domain of any one of SEQ ID NO: 29-SEQ ID NO: 34.

In some embodiments, an intracellular signaling domain may comprise a sequence having at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to an intracellular domain of an endogenous cytokine receptor (e.g., an interleukin 2 receptor subunit α (IL2Rα), an interleukin 2 receptor subunit β (IL2Rβ3), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (IL1R), a CD123, a CD124, an interleukin 5 receptor subunit α (IL5Rα), an interleukin 5 receptor subunit β (IL5Rβ), a CD126, a CD132, a CD129, an interleukin 11 receptor subunit α (IL11Rα), an interleukin 12 receptor subunit β1 (IL12Rβ1), an interleukin 12 receptor subunit β2 (IL12Rβ2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, or a GM-CSF).

### C. Transmembrane Domain

An engineered cytokine receptor switch of the present disclosure may comprise a transmembrane domain. The transmembrane domain may connect an intracellular portion and an extracellular portion of the engineered cytokine receptor and may be designed to span a cell membrane and transduce a signal from an activator binding domain to an intracellular signaling domain upon binding of an activator to the activator binding domain.

A transmembrane domain may be derived from an endogenous cytokine receptor or from another type of receptor. For example, a transmembrane domain may be derived from an interleukin 2 receptor subunit α (IL2Rα), an interleukin 2 receptor subunit β (IL2Rβ), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (IL1R), a CD123, a CD124, an interleukin 5 receptor subunit α (IL5Rα), an interleukin 5 receptor subunit β (IL5Rβ), a CD126, a CD132, a CD129, an interleukin 11 receptor subunit α (IL11Rα), an interleukin 12 receptor subunit 31 (IL12Rβ1), an interleukin 12 receptor subunit β2 (IL12Rβ2), interleukin 13 receptor subunit al (IL13Rα1), a CD122, an interleukin 18 receptor (IL18R), an interleukin 23 receptor (IL23R), an interleukin 27 receptor subunit α (IL27Rα), a CD130, an immunoglobulin (e.g., an IgG1, an IgG2, an IgG3, an IgG4, an IgM, an IgA, an IgD, an IgE), a CD8, a CD28, a GM-CSF, or an erythropoietin receptor (EpoR).

In some embodiments, the transmembrane domain may comprise a transmembrane domain or a variant of a transmembrane domain of an endogenous cytokine receptor or another type of receptor. For example, the transmembrane domain may comprise a transmembrane domain or a variant of a transmembrane domain of an interleukin 2 receptor subunit α (IL2Rα), an interleukin 2 receptor subunit β (IL2Rβ), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (IL1R), a CD123, a CD124, an interleukin 5 receptor subunit α (IL5Rα), an interleukin 5 receptor subunit β (IL5Rβ), a CD126, a CD132, a CD129, an interleukin 11 receptor subunit α (IL11Rα), an interleukin 12 receptor subunit β1 (IL12Rβ1), an interleukin 12 receptor subunit β2 (IL12Rβ2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, an immunoglobulin, a CD8, a CD28, a GM-CSF, or an EpoR. The transmembrane domain, fragment of the transmembrane domain, or variant of the transmembrane domain may be capable of activating the cytokine signaling pathway activated by the endogenous cytokine receptor from which it was derived.

In some embodiments, the transmembrane domain may comprise a transmembrane domain derived from any transmembrane protein. In some embodiments, the transmembrane domain may be a synthetic transmembrane domain. For example, the transmembrane domain may comprise a synthetic transmembrane α-helix, helical bundle, or β-barrel.

Examples of transmembrane domains that may be used in an engineered cytokine receptor switch are provided in Table 4.

TABLE 4
Representative Examples of Transmembrane Domains

| Receptor | SEQ ID NO | Sequence |
|---|---|---|
| IL2Rα | SEQ ID NO: 23 | VAVAGCVFLLISVLLLSGL |
| IL2Rβ | SEQ ID NO: 24 | IPWLGHLLVGLSGAFGFIILVYLLI |
| IL2Rγ | SEQ ID NO: 25 | VVISVGSMGLIISLLCVYFWL |
| IL7Rα | SEQ ID NO: 26 | PILLTISILSFFSVALLVILACVLW |
| IL15Rα | SEQ ID NO: 27 | VAISTSTVLLCGLSAVSLLACYL |
| IL21Rα | SEQ ID NO: 28 | GWNPHLLLLLLLVIVFIPAFW |

In some embodiments, an engineered cytokine receptor switch may comprise a transmembrane domain comprising at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to any one of SEQ ID NO: 23-SEQ ID NO: 28. In some embodiments, the engineered cytokine receptor switch may comprise a transmembrane domain of any one of SEQ ID NO: 23-SEQ ID NO: 28.

In some embodiments, the transmembrane domain may comprise a sequence having at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to a transmembrane domain of an endogenous cytokine receptor or another receptor (e.g., an interleukin 2 receptor subunit α (IL2Rα), an interleukin 2 receptor subunit β (IL2Rβ), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (IL1R), a CD123, a CD124, an interleukin 5 receptor subunit α (IL5Rα), an interleukin 5 receptor subunit β (IL5Rβ), a CD126, a CD132, a CD129, an interleukin 11 receptor subunit α (IL11Rα), an interleukin 12 receptor subunit 31 (IL12Rβ1), an interleukin 12 receptor subunit β2 (IL12Rβ2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, an immunoglobulin, a CD8, a CD28, a GM-CSF, or an EpoR).

### D. Signal Peptide

An engineered cytokine receptor switch of the present disclosure may comprise a signal peptide. The signal peptide may be positioned at the N-terminus of the engineered cytokine receptor and may be designed to direct expression of the engineered cytokine receptor switch to the endoplasmic reticulum (ER). The engineered cytokine receptor may be synthesized in the ER membrane and may be trafficked to the plasma membrane as a transmembrane protein.

A signal peptide may be derived from an endogenous cytokine receptor or another type of receptor. For example, signal peptide may be derived from an interleukin 2 receptor subunit α (IL2Rα), an interleukin 2 receptor subunit β (IL2Rβ), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (IL1R), a CD123, a CD124, an interleukin 5 receptor subunit α (IL5Rα), an interleukin 5 receptor subunit β (IL5Rβ), a CD126, a CD132, a CD129, an interleukin 11 receptor subunit α (IL11Rα), an interleukin 12 receptor subunit β1 (IL12Rβ1), an interleukin 12 receptor subunit β2 (IL12Rβ2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, an immunoglobulin (e.g., an IgG1, an IgG2, an IgG3, an IgG4, an IgM, an IgA, an IgD, an IgE), a CD8, a CD28, or a GM-CSF.

In some embodiments, a signal peptide may comprise the signal peptide portion of an endogenous cytokine receptor or another receptor. For example, the signal peptide may comprise the signal peptide portion of an interleukin 2 receptor subunit α (IL2Rα), an interleukin 2 receptor subunit β (IL2Rβ), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (IL1R), a CD123, a CD124, an interleukin 5 receptor subunit α (IL5Rα), an interleukin 5 receptor subunit β (IL5Rβ3), a CD126, a CD132, a CD129, an interleukin 11 receptor subunit α (IL11Rα), an interleukin 12 receptor subunit β1 (IL12Rβ1), an interleukin 12 receptor subunit β2 (IL12Rβ2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, an immunoglobulin, a CD8, a CD28, or a GM-CSF.

In some embodiments, the signal peptide may be a signal peptide from any transmembrane or membrane-bound protein. The signal peptide may be sufficient to direct expression of the engineered cytokine receptor switch to the ER.

Examples of signal peptides that may be used in an engineered cytokine receptor switch are provided in Table 5.

TABLE 5
Representative Examples of Signal Peptides

| Receptor | SEQ ID NO | Sequence |
|---|---|---|
| IL2Rα | SEQ ID NO: 15 | MDSYLLMWGLLTFIMVPGCQA |
| IL2Rβ | SEQ ID NO: 16 | MAAPALSWRLPLLILLLPLATSWASA |
| IL2Rγ | SEQ ID NO: 17 | MLKPSLPFTSLLFLQLPLLGVG |
| IL7Rα | SEQ ID NO: 18 | MTILGTTFGMVFSLLQVVSG |
| IL15Rα | SEQ ID NO: 19 | MAPRRARGCRTLGLPALLLLLLLRPPATRG |
| IL21Rα | SEQ ID NO: 20 | MPRGWAAPLLLLLLQGGWG |

In some embodiments, an engineered cytokine receptor switch may comprise a signal peptide comprising at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to any one of SEQ ID NO: 15-SEQ ID NO: 20. In some embodiments, the engineered cytokine receptor switch may comprise a signal peptide of any one of SEQ ID NO: 15-SEQ ID NO: 20.

In some embodiments, the signal peptide may comprise a sequence having at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to a signal peptide of an endogenous cytokine receptor or another receptor (e.g., an interleukin 2 receptor subunit α (IL2Rα), an interleukin 2 receptor subunit β (IL2Rβ), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (IL1R), a CD123, a CD124, an interleukin 5 receptor subunit α (IL5Rα), an interleukin 5 receptor subunit β (IL5Rβ), a CD126, a CD132, a CD129, an interleukin 11 receptor subunit α (IL11Rα), an interleukin 12 receptor subunit β1 (IL12Rβ1), an interleukin 12 receptor subunit β2 (IL12Rβ2), interleukin 13 receptor subunit α1 (IL13Rα1), a CD122, an interleukin 18 receptor (IL18R), an interleukin 23 receptor (IL23R), an interleukin 27 receptor subunit α (IL27Rα), an immunoglobulin, a CD8, a CD28, or a CD130).

### E. Hinge

An engineered cytokine receptor switch of the present disclosure may comprise a hinge (also referred to herein as a “hinge domain”). The hinge may be positioned between the activator binding domain and the transmembrane domain and may be designed to increase the flexibility of the engineered cytokine receptor switch. Increased flexibility may reduce spatial constraints between the activator binding domain and the activator (e.g., a small molecule activator adhered to a surface), facilitating access to the activator. In some embodiments, the hinge may be engineered to provide a desired distance between the plasma membrane of a cell expressing the engineered cytokine receptor switch and an activator bound to the engineered cytokine receptor switch.

In some embodiments, the hinge may be a synthetic peptide designed to provide a desired length, flexibility, or both. In some embodiments, the hinge may be derived from an endogenous transmembrane protein. For example, the hinge may be derived from a CD8 (e.g., a CD8a), a CD3, a CD4, a CD28, a 4-1BB, a CD28, an OX40, an inducible T cell costimulatory (ICOS), a CD27, an immunoglobulin (e.g., an IgG1, an IgG2, an IgG3, an IgG4, an IgM, an IgA, an IgD, an IgE), or an EpoR. In some embodiments, the hinge may comprise a hinge of an endogenous transmembrane protein. For example, the hinge may be derived from a hinge of a CD8 (e.g., a CD8a), a CD3, a CD4, a CD28, a 4-1BB, a CD28, an OX40, an ICOS, a CD27, an immunoglobulin, or an EpoR.

Examples of hinges that may be used in an engineered cytokine receptor switch are provided in Table 6.

TABLE 6
Representative Examples of Hinges

| Hinge | SEQ ID NO | Sequence |
|---|---|---|
| CD8α Hinge | SEQ ID NO: 22 | TTTPAPRPPTPAPTIASQPLSLRPEACRPAAGGAVHTRGLDFACD |

In some embodiments, an engineered cytokine receptor switch may comprise a hinge comprising at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to SEQ ID NO: 22. In some embodiments, the engineered cytokine receptor switch may comprise a hinge of SEQ ID NO: 22.

In some embodiments, the signal peptide may comprise a sequence having at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to a hinge of an endogenous transmembrane protein (e.g., a CD8 (e.g., a CD8a), a CD3, a CD4, a CD28, a 4-1BB, a CD28, an OX40, an ICOS, a CD27, an immunoglobulin, or an EpoR).

As described herein, the length of the hinge may affect whether the engineered cytokine receptor switch exhibits activator-independent activity. For example, the hinge can be no more than 50 amino acids, 45 amino acids, 40 amino acids, 30 amino acids, 25 amino acids, 20 amino acids, 15 amino acids, 10 amino acids, 9 amino acids, 8 amino acids, 7 amino acids, 6 amino acids, 5 amino acids, 4 amino acids, 3 amino acids, 2 amino acids, or 1 amino acid in length. Alternatively or in combination, the hinge can be at least 1 amino acid, 2 amino acids, 3 amino acids, 4 amino acids, 5 amino acids, 6 amino acids, 7 amino acids, 8 amino acids, 9 amino acids, 10 amino acids, 15 amino acids, 20 amino acids, 25 amino acids, 30 amino acids, 35 amino acids, 40 amino acids, 45 amino acids, or 50 amino acids in length.

### F. Additional Components

In some embodiments, an engineered cytokine receptor switch of the present disclosure may comprise or be co-expressed with one or more additional components. Additional components that may be included in or co-expressed with an engineered cytokine receptor switch are provided in Table 7.

TABLE 7
Additional Components

| Component | SEQ ID NO | Sequence |
|---|---|---|
| P2A peptide | SEQ ID NO: 35 | GSGATNFSLLKQAGDVEENPGP |
| T2A peptide | SEQ ID NO: 36 | GSGEGRGSLLTCGDVEENPGP |
| E2A peptide | SEQ ID NO: 37 | GSGQCTNYALLKLAGDVESNPGP |
| F2A peptide | SEQ ID NO: 38 | GSGVKQTLNFDLLKLAGDVESNPGP |
| Truncated | SEQ ID NO: 39 | MPPPRLLFFLLFLTPMEVRPEEPLVVKVEEGDNAVLQCLKGTSD |
| CD19 | | GPTQQLTWSRESPLKPFLKLSLGLPGLGIHMRPLAIWLFIFNVSQ |
| | | QMGGFYLCQPGPPSEKAWQPGWTVNVEGSGELFRWNVSDLGG |
| | | LGCGLKNRSSEGPSSPSGKLMSPKLYVWAKDRPEIWEGEPPCLP |
| | | PRDSLNQSLSQDLTMAPGSTLWLSCGVPPDSVSRGPLSWTHVH |
| | | PKGPKSLLSLELKDDRPARDMWVMETGLLLPRATAQDAGKYY |
| | | CHRGNLTMSFHLEITARPVLWHWLLRTGGWKVSAVTLAYLIFC |
| | | LCSLVGILHLQRALVLRRKRKRMTDPTRRF |

In some embodiments, an engineered cytokine receptor switch may comprise or be co-expressed with a marker domain. The marker domain may be co-expressed with the cytokine receptor chain(s) of the engineered cytokine receptor switch for purposes of identifying immune cells that are expressing the engineered cytokine receptor switch (“positive cells”), enriching and purifying positive cells, acting as a conditional suicide switch for positive cells, and/or other relevant functions. The marker domain can be truncated (e.g., in the intracellular domain) such that the expressed truncated marker does not have the biological function of the native marker. The marker domain can be any cell surface molecule that is not present on natural T cells. For example, an engineered cytokine receptor switch may comprise a CD19 domain (e.g., a truncated CD19 domain of SEQ ID NO: 39), a CD20 domain (e.g., a truncated CD20 domain), a CD22 domain (e.g., a truncated CD22 domain), a CD34 domain (e.g., a truncated CD34 domain), or an EGFR domain (e.g., a truncated EGFR domain). In some embodiments, an engineered cytokine receptor switch may comprise a marker domain comprising at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to SEQ ID NO: 39. In some embodiments, the engineered cytokine receptor switch may comprise a marker domain of SEQ ID NO: 39.

Alternatively or in combination, the marker domain can be a detection marker, such as a fluorescent protein. For example, an engineered cytokine receptor switch may comprise EBFP, Sapphire, T-Sapphire, ECFP, mCFP, Cerulean, CyPet, AmCyanl, Midori-Ishi Cyan, mTFP1, GFP, EGFP, AcGFP, TurboGFP, Emerald, Azami Green, ZsGreen, EYFP, Topaz, Venus, mCitrine, yPet, PhiYFP, ZsYellow1, mBanana, Kusabira Orange, mOrange, dTomato, tdTomato, DsRed, DsRed2, DsRed-Express, DsRed-Monomer, mTangerine, mStrawberry, AsRed2, mRFP1, JREd, mCherry, HcRed1, mRaspberry, HcRed-Tandem, mPlum, or AQ143.

In some embodiments, a construct for expression of an engineered cytokine receptor switch may comprise a cleavage sequence, such as a 2A self-cleaving peptide sequence (e.g., a P2A peptide of SEQ ID NO: 35, a T2A peptide of SEQ ID NO: 36, a E2A peptide of SEQ ID NO: 37, or a F2A peptide of SEQ ID NO: 38). A 2A self-cleaving peptide sequence (also known as a “2A peptide”) may be included to link an engineered cytokine receptor switch to one or more additional engineered cytokine receptor switches for co-expression. In some embodiments, a 2A peptide may link a first engineered cytokine receptor switch to a second engineered cytokine receptor switch. For example, a 2A peptide (e.g., of any one of SEQ ID NO: 35-SEQ ID NO: 38) may link a first cytokine receptor chain (e.g., an IL2Rβ cytokine receptor chain or an IL2Rγ cytokine receptor chain) to a second cytokine receptor chain (e.g., an IL2Rβ cytokine receptor chain or an IL2Rγ cytokine receptor chain) to form a single protein that encompasses both chains of an engineered dual-chain cytokine receptor switch (e.g., a dual-chain cytokine receptor switch of SEQ ID NO: 7). After expression, the protein can be cleaved at the cleavage sequence to produce the separate two cytokine receptor chains of the dual-chain cytokine receptor switch. As another example, a 2A peptide (e.g., of any one of SEQ ID NO: 35-SEQ ID NO: 38) may be included to link a cytokine receptor chain to a marker domain (e.g., the marker domain of SEQ ID NO: 39) to form a single protein that encompasses the cytokine receptor chain and the marker domain. After expression, the protein can be cleaved at the cleavage sequence to separate the cytokine receptor chain from the marker domain.

In some embodiments, a construct for expression of an engineered cytokine receptor switch may comprise a cleavage sequence comprising at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to SEQ ID NO: 35-SEQ ID NO: 38. In some embodiments, the construct for expression of the engineered cytokine receptor switch may comprise a cleavage sequence of SEQ ID NO: 35-SEQ ID NO: 38.

Alternatively or in combination, a construct for expression of an engineered cytokine receptor switch may comprise an internal ribosome entry site (IRES) to allow for co-expression of an engineered cytokine receptor switch to an additional engineered cytokine receptor switch and/or to a marker domain. In some embodiments, a IRES may link a first engineered cytokine receptor switch to a second engineered cytokine receptor switch in an expression construct, such that the first engineered cytokine receptor switch and the second engineered cytokine receptor switch are translated as separate proteins. In some embodiments, an IRES may link an engineered cytokine receptor switch to a marker domain, such that the engineered cytokine receptor switch and the marker domain are translated as separate proteins.

### II. ACTIVATORS

An activator may activate an immune cell of the present disclosure by binding to the activator binding domain of an engineered cytokine receptor switch and activating cytokine signaling via the intracellular signaling domain. The activator may be a small molecule, a peptide, an oligonucleotide, or a protein. In some embodiments, an activator may be selected to have low toxicity, low immunogenicity, low cross-reactivity, or combinations thereof to reduce unfavorable side effects when administered to a subject (e.g., a human subject). For instance, the activator can be a molecule that is non-toxic to humans, included in an Inactive Ingredients Database, or both. In some embodiments, the activator may be an exogenous activator (e.g., an exogenous small molecule, an exogenous peptide, an exogenous oligonucleotide, or an exogenous protein) that is not naturally present in a target environment (e.g., a human subject) to prevent activation of the engineered cytokine receptor switch in the absence of an external stimulus (e.g., administration of the activator), prevent cross-reactivity of the activator with other biological components, and to enable dynamic control of receptor signaling.

Examples of small molecule activators (e.g., haptens) that may be used to activate an engineered cytokine receptor switch include fluorophores (e.g., fluorescein, fluorescein derivatives, indocyanines, indocyanine derivatives, cyanines, cyanine derivatives), chelators (e.g., DOTA), or other small molecules. For example, the fluorescein derivative may be fluorescein isothiocyanate (FITC), fluorescein 5-maleimide, fluorescein-5-carboxamide, fluorescein-6-carboxamide, or 6-FAM phosphoramidite. Additional examples of small molecules that may bind to an activator binding domain of a cytokine receptor switch include Topiramate hemisuccinate, Creatine, Acetaminophen, Ketamine, Propofol, Lidocaine, Ractopamine, Salicylate, Salicylic Acid, Sulfasalazine, Dapsone, Albendazole, Ivermectin, Levamisole, Permethrin, Pyrantel, Thiabendazole, Procainamide, Sulfamethazine, Amikacin, Amoxicillin, Ampicillin, Cefazolin, Cefuroxime, Cephalexin, Chloramphenicol, Chloramphenicol, Ciprofloxacin, Clenbuterol, Cloxacillin, Colistin A, Dicloxacillin, Enrofloxacin, Furaltadone, Gentamicin, Gentamicin, Kanamycin, Kanamycin, Kincomycin, Lincomycin, Metronidazole, Nafcillin, Nalidixic Acid, Neomycin, Neomycin, Nitrofurazone, Norfloxacin, Ofloxacin, Oxacillin, Spectinomycin, Streptomycin, Streptomycin, Sulfabenzamide, Sulfacetamide, Sulfadiazine, Sulfadimidine, Sulfametoxydiazine, Sulfanilamide, Trimethoprim, Carbamazepine, Ethosuximide, Lamotrigine, Primidone, Cetirizine, Chlorpheniramine, Diphenhydramine, Doxylamine, Promethazine, Sulfadimethoxine, Benzothiazinone, Butylated Hydroxytoluene, Tripelennamine, Chlorpromazine, Clozapine, Haloperidol, Olanzapine, Paliperidone, Quetiapine, Ribavirin, Meprobamate, Acebutolol, Atenolol, Penbutolol, Warfarin, Salmeterol, Aflatoxin B1, Tetraxetan (DOTA), MPOB, Biotin, Melamine, Methotrexate, Amphetamine, Diethylpropion, Dextromethorphan, Pseudoephedrine, Dihydrochlorothiazide, Hydrochlorothiazide, Clonazepam, Diazepam, Nitrazepam, Rhodamine B, Fluorescent Brightener Ksn, Zearalenone, Sudan Red1, Acetominophen, Acrylamide, Benzoic Acid, Benzophenone, Benzothiazine, Mercaptobenzothiazole, Erythrosine, Sudan, Tartrazine, Erythromycin, Sirolimus, Atropine, Ethyl glucuronide, Aflatoxin M1, Methocarbamol, Fentanyl, Hydromorphone, Morphine, Remifentanil, Tapentadol, Tramadol, Pregabalin, Gabapentin, Amitriptyline, Desipramine, Imipramine, Nortriptyline, Venlafaxine, Dinitrophenyl (DNP), His-Tag, PEG methoxy group, Etodolac, Ibuprofen, Ketoprofen, Meclofenamic Acid, Phenylbutazone, Acetyl Salicylic Acid, Acetamiprid, Acetochlor, Carbadazim, Carbaryl, Chlorothalonil, Chlorpyrifos, Fenpropathrin, Imazalil, Imidacloprid, Parathion, Abscisic acid, Dibutyl Phthalate, Clonazepam, Lorazepam, Oxazepam, Phenobarbital, Secobarbital, Zaleplon, Zolpidem, Trazodone, Fluoxetine, Fluvoxamine, Cortisone, Dexamethasone, Dihydrotestosterone, Fluocinolone, Methylprednisolone, Prednisolone, Stanozolol, Triamcinolone, Mazindol, Methamphetamine, Methylphenidate, Modafinil, Chrysoidine, Deoxynivalenol, Fumonisin, Microcystin Lr, Ochratoxin, Sterigmatocystin, T-2 toxin, Sildenafil, Tadalafil, Scopolamine, Florfenicol, Pirlimycin, or Sulfaquinoxaline.

In some embodiments, the activator can be an endogenous activator that is naturally present in the target environment (e.g., a human subject). The endogenous activator (e.g., an endogenous small molecular, endogenous peptide, endogenous oligonucleotide, endogenous protein) can be associated with a cancer cell (e.g., expressed by or otherwise produced by a cancer cell), a cancer microenvironment (e.g., an immunosuppressive microenvironment), or both. For example, in some embodiments, the activator is a surface antigen specific for the type of cancer to be treated, or is a portion of such a surface antigen. Examples of surface antigens that may be used to activate an engineered cytokine receptor switch include CD19, CD20, CD22, CD123, CD33, CD3, CD4, CD8, CD38, SLAMF7, BCMA, GD2, GPRC5D, MUC16, HER2, EGFR, EGFRvIII, CLL-1, CD44v6, B7-H3, EphA2, GRP78, NKG2D, CD70, folate receptor-α, or mesothelin, or a portion thereof.

In some embodiments, the activator is an immunosuppressive molecule, or is a portion of a such an immunosuppressive molecule. For example, the activator can be a molecule that induces inhibition of CAR T-cell activity, such as a ligand of an immune checkpoint family member. In some embodiments, the engineered cytokine receptor switch binds to any of the following or to a ligand of any of the following: CD2, CD95 (Fas), CTLA4 (CD152), CD172A (SIRPa), CD200R, CD223 (LAG3), CD279 (PD-1), CD272 (BTLA), CD300, CD366 (TIM3), A2aR, KIR, LPA5, TIGIT (e.g., CD155 (PVR), CD112 (PVRL2/nectin-2)), TGFβ, CD58 (LFA3), CD178 (Fas-L), CD80 (B7-1), CD86 (B7-2), CD47, CD200, LAG-3 (e.g., MHCII, FGL-1, Gal-3, LSECtin, α-syn), CD273 (PD-L2), CD274 (PD-L1), CD258 (HVEM), CD300, CD94 (NKG2A), TIM3 (e.g., Galectin 9, PtdSer, HMGB1, CEACAM1), GPR92, IL6, IL10, or adenosine.

In some embodiments, an activator may be used to activate an immune cell expressing the engineered cytokine receptor switch. For example, the activator may be used to promote conversion of the immune cell to a memory phenotype (e.g., a stem cell memory phenotype, a central memory phenotype, an effector memory phenotype, or an effector memory re-expressing CD45RA phenotype). Activation may be performed ex vivo (e.g., during immune cell manufacturing) or in vivo (e.g., during immune cell therapy treatment). In some embodiments, activation is performed both ex vivo and in vivo. In some embodiments, activation is performed ex vivo but not in vivo. In some embodiments, activation is performed in vivo but not ex vivo. In some embodiments, activation is not performed either ex vivo or in vivo, such that engineered cytokine receptor switch relies primarily or entirely on activator-independent activity for its effect.

For ex vivo activation, the activator may be conjugated (e.g., via covalent or non-covalent linkages) or otherwise attached (e.g., adsorbed, adhered) to a substrate, such as a surface (e.g., a plate surface), a bead (e.g., a polystyrene paramagnetic bead), a carrier protein (e.g., an antibody), a carrier polymer (e.g., a synthetic polymer, a biopolymer), a carrier nucleic acid (e.g., an oligonucleotide, a polynucleotide), or combinations thereof. For example, the activator may be conjugated to a carrier protein by classical stochastic cysteine and lysine conjugations, or through a site-specific conjugation technology. In some embodiments, the activator may be adhered to a surface, and immune cells expressing the engineered cytokine receptor may be added to the surface to activate the immune cells. In some embodiments, the activator may be adsorbed to beads, and the beads may be added to a suspension of immune cells to activate the immune cells. The beads may be removed prior to administration of the immune cells to a subject. Additional examples of techniques for ex vivo activation are provided in International Patent Application No. [Attorney Docket No. DCT.003WO], filed concurrently with the present application, which is incorporated herein by reference in its entirety.

For in vivo activation, the activator may be part of a bispecific agent that is administered to the subject. A bispecific agent can include an activator that binds to an engineered immune cell and a targeting moiety that binds to a target for the immune cell (e.g., a cancer cell, a cell of a lymphoid organ, or another cell type). The targeting moiety can be a carrier protein, such as an antibody, an antibody fragment, a single chain variable fragment (scFv), a nanobody, or a peptide. In some embodiments, the targeting moiety (e.g., carrier protein) may be humanized to reduce immunogenicity. The activator can be conjugated (e.g., via covalent or non-covalent linkages) to the targeting moiety to form the bispecific agent. For example, the activator may be conjugated to the targeting moiety (e.g., carrier protein) by classical stochastic cysteine and lysine conjugations, or through a site-specific conjugation technology. The bispecific agent (e.g., activator-carrier protein conjugate) may be administered to a subject who has been or will be treated with immune cells expressing the engineered cytokine receptor switch.

In some embodiments, the targeting moiety may be an antibody, antibody fragment, scFv, nanobody, peptide, etc., that binds to a tumor antigen. For example, the antigen may be CD19, CD20, CD22, CD123, CD33, CD3, CD4, CD8, CD38, SLAMF7, BCMA, GD2, GPRC5D, MUC16, HER2, EGFR, EGFRvIII, CLL-1, CD44v6, folate receptor-α, mesothelin, CD20, CD37, ROR1, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, surviving, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, IL13Rα2, B7-H3 (CD276), EPHA2, GRP78, NKG2D, or CD70.

Binding of the bispecific agent to the tumor antigen and to an engineered cytokine receptor switch may recruit an immune cell expressing the engineered cytokine receptor switch to a tumor cell. In some embodiments, the immune cell is also engineered to express a CAR for a tumor antigen of the tumor cell, which may or may not be the same as the tumor antigen recognized by the bispecific agent. Accordingly, binding of the bispecific agent to its respective tumor antigen may facilitate and/or enhance binding of the CAR to its respective tumor antigen, which in turn may facilitate killing of the tumor cell. Additional details of CARs that may be used with the engineered cytokine receptor switches described herein are provided in Section III below.

In some embodiments, the targeting moiety is a lymphoid-targeting protein that directs the immune cells to a lymphoid organ, such as a lymph node (e.g., a tumor-draining lymph node), a spleen, a thymus, or bone marrow. The lymphoid-targeting protein may comprise an antibody, an antibody fragment, a single chain variable fragment (scFv), a miniprotein, a nanobody, or a peptide. The lymphoid-targeting protein may cause the immune cell to bind an antigen presenting cell, a T-cell, a B-cell, a lymphocyte, a lymphatic endothelial cell, a B cell, a macrophage, or a lymphoid organ stroma cell. The lymphoid-targeting protein may be engineered to bind a lymphoid marker. In some embodiments, the lymphoid marker is a surface marker (e.g., a cell surface protein) expressed in one or more lymphoid organs, such as the lymph nodes (e.g., tumor-draining lymph nodes, non-tumor-draining lymph nodes), spleen, thymus, bone marrow, or combinations thereof. For example, the lymphoid marker can be a surface-exposed epitope of a cell surface or transmembrane protein. The lymphoid marker can be expressed by a cell that is resident in or otherwise associated with a lymphoid organ, such as an antigen presenting cell (e.g., a dendritic cell), a T-cell, a B-cell, a lymphocyte, a lymphatic endothelial cell, a B cell, a macrophage, or a lymphoid organ stromal cell. The lymphoid marker can be a surface marker expressed by lymphocytes that reside in lymphoid tissue, such as CD3, CD45, CD4, CD2, CD5, CD8, γδ-T-cell receptor, T19, CD45, CD205, cell-surface immunoglobulin (sIg), and L-selectin. The lymphoid marker can be a marker of a stromal cell of a lymphoid organ (e.g., lymph node stromal cells), such as PNAd, VEGFR-3, LYVE-1, Prox-1, podoplanin, CD31, MadCAM1, CXCL13, RANKL, CXCL12, APRIL, BAFF, IL-7, CCL19, CCL21, and Spns2.

Binding of the bispecific agent to the lymphoid marker and to an engineered cytokine receptor switch may recruit an immune cell expressing the engineered cytokine receptor switch to a lymphoid organ. The bispecific agent may bind the immune cell to a cell resident in or otherwise associated with a lymphoid organ, such as an antigen presenting cell (e.g., a dendritic cell), a T-cell, a B-cell, a lymphocyte, a lymphatic endothelial cell, a B cell, a macrophage, or a lymphoid organ stromal cell. The bispecific agent may activate cytokine signaling in the immune cell via the engineered cytokine receptor switch. Recruitment to the lymphoid organ may promote activation and expansion of the immune cell in the lymphoid environment. In some embodiments, once in the lymphoid organ, costimulatory molecules and adhesion molecules present in the lymphoid organ may promote conversion the engineered immune cell to a memory cell phenotype (e.g., a stem-cell memory phenotype, a central memory phenotype, an effector memory phenotype, an effector memory re-expressing CD45RA phenotype). For instance, in embodiments where the immune cell is a T-cell, the T-cells may differentiate into memory T-cells in the lymph nodes and/or other lymphoid organs, and the memory T-cells may contribute to a long-term immune response against antigens recognized by the memory T-cells. The costimulatory and adhesion molecules may also promote activation and clonal expansion of the engineered immune cell. Conversion of the engineered immune cell to a memory cell phenotype may increase the persistence and prolong the efficacy of the engineered immune cell in a subject treated with both the engineered immune cell and the bispecific agent. The lymphoid organ may provide an environment conducive to immune cell activation, facilitating immune cell differentiation and expansion prior to delivery to an immune repressive tumor microenvironment.

In some embodiments, the bispecific agent may bind to the lymphoid marker (e.g., via the lymphoid-targeting protein) with an equilibrium dissociation constant (K_{D}) of no more than 1 μM, no more than 100 nM, no more than 10 nM, or no more than 1 nM. The bispecific agent may retain the engineered immune cell in the lymphoid organ for an amount of time sufficient to activate the immune cell. For example, the bispecific agent may retain the engineered immune cell in the lymphoid organ for 6 to 96 hours, 12 to 72 hours, or 24 to 48 hours.

In some embodiments, it may be important or even necessary for the activator to be attached to a surface to effectively activate immune cells expressing the engineering cytokine receptor switch. For example, the use of such “surface-bound activators” may be beneficial for producing tissue-specific activation of the engineered immune cells. The surface can be the surface of a container (e.g., a plate, tube, bag, bioreactor, chamber, cassette, column), the surface of a bead (e.g., a microparticle, a microsphere), the surface of a tissue (e.g., a lymphoid tissue), the surface of another cell (e.g., a tumor cell, a cell of a lymphoid organ), or any other surface having a high degree of rigidity compared to the immune cell. The surface can be an organic surface, an inorganic surface, or a combination thereof. The activator can be directly attached to the surface, or can be indirectly attached to the surface via a carrier (e.g., a carrier protein, a carrier polymer, a carrier oligonucleotide, or any other bifunctional molecule capable of attaching to both the activator and to the surface). In some embodiments, a plurality of activators are attached to the surface at a sufficiently high density such that binding of two cytokine receptor chains to two proximate activators causes dimerization of the two cytokine receptor chains (e.g., homodimerization of two single-chain cytokine receptor switches or heterodimerization of the two cytokine receptor chains of a dual-chain cytokine receptor switch).

In other embodiments, however, the engineered immune cells may be activated by an activator that is not bound to any surface. For example, the activator can be provided as part of a free-floating, soluble activator-carrier complex. In some embodiments, the activator-carrier complex includes at least two activators such that binding of two cytokine receptor chains to two proximate activators causes dimerization of the two cytokine receptor chains (e.g., homodimerization of two single-chain cytokine receptor switches or heterodimerization of the two cytokine receptor chains of a dual-chain cytokine receptor switch). Moreover, the engineered immune cells may also exhibit activator-independent activity in that signaling of the engineered cytokine receptor switch may be initiated even in the absence of the activator, as described elsewhere herein.

### III. CHIMERIC ANTIGEN RECEPTORS

Provided herein are immune cells comprising an engineered cytokine receptor switch of the present disclosure and a chimeric antigen receptor (CAR). An engineered cytokine receptor switch of the present disclosure may be used in conjunction with a CAR to produce a therapeutic effect (e.g., an anti-cancer effect). In some embodiments, an engineered cytokine receptor switch may be expressed in an immune cell along with a CAR. For example, a CAR T-cell of the present disclosure may be engineered to co-express an engineered cytokine receptor switch and a CAR that binds a tumor antigen. The CAR may bind to an antigen (e.g., a tumor antigen) on a target cell to recruit the immune cell to the target cell. For example, a CAR may be engineered to bind to a surface antigen on a tumor cell via an antigen binding domain and activate intracellular signaling through an intracellular signaling domain to produce a targeted immune response against the tumor cell. The CAR may recruit the immune cell to a target cell after the immune cell has been activated. As described herein, activation of an immune cell may occur ex vivo (e.g., via binding of an activator to the engineered cytokine receptor switch during manufacturing), in vivo (e.g., via binding of an activator to the engineered cytokine receptor switch and/or activation of the immune cell in a lymphoid organ), or a combination thereof. Optionally, the CAR may recruit the immune cell to a target cell without activation of the immune cell (e.g., no activator is provided to the immune cell either ex vivo or in vivo but the engineered cytokine receptor switch may still exhibit activator-independent activity).

A CAR may comprise an extracellular domain, a transmembrane domain, and an intracellular domain. The extracellular domain of the CAR may comprise an antigen binding domain that binds specifically to an antigen. The antigen binding domain can be any protein, protein fragment, or peptide capable of selectively binding the antigen. In some embodiments, for example, the antigen binding domain may comprise an antibody (e.g., a monoclonal antibody), an antibody fragment, an scFv, a nanobody, or a peptide. In some embodiments, an antigen binding domain may comprise a fragment of an antibody (e.g., a variable fragment) that binds to a selected antigen. Antibodies, antibody fragments, scFvs, and nanobodies may be produced using various methods known in the art to target a specific antigen. In some embodiments, the antigen binding domain may comprise a VHH antibody, an scFv, a V_{H}, a V_{L}, or a ligand specific for a target antigen. The antigen binding domain of the CAR may bind to a specific epitope of the target antigen.

In some embodiments, the antigen may be a tumor antigen, such as a tumor cell surface marker, a tumor-specific antigen, or a tumor-associated antigen. For example, the antigen may be CD19, CD20, CD22, CD123, CD33, CD3, CD4, CD8, CD38, SLAMF7, BCMA, GD2, GPRC5D, MUC16, HER2, EGFR, EGFRvIII, CLL-1, CD44v6, folate receptor-α, mesothelin, CD20, CD37, ROR1, carcinoembryonic antigen (CEA), j-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, surviving, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, IL13Rα2, B7-H3 (CD276), EPHA2, GRP78, NKG2D, or CD70. In some embodiments, the CAR may be selected to target a tumor cell antigen associated with a cancer of interest. A CAR having an antigen binding domain that recognizes an antigen expressed by a target cell (e.g., a tumor antigen expressed by a cancer cell) may be referred to herein as a “direct CAR.” A direct CAR may be targeted to a target cell via direct binding of the antigen binding domain to the antigen expressed by the target cell.

In some embodiments, the antigen binding domain is a VHH antibody, an scFv, a V_{H}, or a V_{L} of an antibody, or a ligand that recognizes any of the tumor antigens described herein. For example, the antigen binding domain can be a VHH antibody, an scFv, a V_{H}, or a V_{L} of an anti-BCMA antibody (e.g., as described in U.S. Patent Publication Nos. 2020/0261501 and 2022/0127371, the disclosures of which are incorporated herein by reference in their entirety). As another example, the antigen binding domain can be a VHH antibody, an scFv, a V_{H}, or a V_{L} of an anti-CD123 antibody (e.g., as described in U.S. Patent Publication No. 2020/0254023, the disclosure of which is incorporated by reference herein in its entirety). In a further example, the antigen binding domain can be a VHH antibody, an scFv, a V_{H}, or a V_{L} of an anti-GD2 antibody.

In some embodiments, the antigen binding domain of the CAR may recognize an antigen that is not expressed by the target cell of the CAR. For instance, the antigen may be a “synthetic antigen” that is not expressed by normal cells or cancer cells of the subject. A CAR that binds to a synthetic antigen may be referred to herein as an “indirect CAR.” An indirect CAR may be targeted to a target cell via a bispecific agent including (1) the synthetic antigen and (2) a targeting moiety that binds to an antigen expressed by the target cell (e.g., a tumor antigen expressed by a tumor cell). The indirect CAR may bind indirectly to the target cell by binding of the antigen binding domain to the synthetic antigen of the bispecific agent, and by binding of the targeting moiety of the bispecific agent to the antigen expressed by the target cell. The targeting moiety and tumor antigen can be any of the embodiments described herein, e.g., in Section II above.

The synthetic antigen may be a small molecule, a peptide, an oligonucleotide, or a protein. In some embodiments, a synthetic antigen may be selected to have low toxicity, low immunogenicity, low cross-reactivity, or combinations thereof to reduce unfavorable side effects when administered to a subject (e.g., a human subject). For instance, the synthetic antigen can be a molecule that is non-toxic to humans, included in an Inactive Ingredients Database, or both. In some embodiments, the synthetic antigen may be an exogenous antigen (e.g., an exogenous small molecule, an exogenous peptide, an exogenous oligonucleotide, or an exogenous protein) that is not naturally present in a target environment (e.g., a human subject) to prevent activation of the indirect CAR in the absence of an external stimulus (e.g., administration of the bispecific agent), prevent cross-reactivity of the synthetic antigen with other biological components, and to enable dynamic control of CAR activity.

Examples of exogenous small molecules (e.g., haptens) that may be recognized by an antigen binding domain of an indirect CAR include fluorophores (e.g., fluorescein, fluorescein derivatives, indocyanines, indocyanine derivatives, cyanines, cyanine derivatives), chelators (e.g., DOTA), or other small molecules. For example, the fluorescein derivative may be fluorescein isothiocyanate (FITC), fluorescein 5-maleimide, fluorescein-5-carboxamide, fluorescein-6-carboxamide, or 6-FAM phosphoramidite. Additional examples of small molecules that may bind to an antigen binding domain of an indirect CAR include Topiramate hemisuccinate, Creatine, Acetaminophen, Ketamine, Propofol, Lidocaine, Ractopamine, Salicylate, Salicylic Acid, Sulfasalazine, Dapsone, Albendazole, Ivermectin, Levamisole, Permethrin, Pyrantel, Thiabendazole, Procainamide, Sulfamethazine, Amikacin, Amoxicillin, Ampicillin, Cefazolin, Cefuroxime, Cephalexin, Chloramphenicol, Chloramphenicol, Ciprofloxacin, Clenbuterol, Cloxacillin, Colistin A, Dicloxacillin, Enrofloxacin, Furaltadone, Gentamicin, Gentamicin, Kanamycin, Kanamycin, Kincomycin, Lincomycin, Metronidazole, Nafcillin, Nalidixic Acid, Neomycin, Neomycin, Nitrofurazone, Norfloxacin, Ofloxacin, Oxacillin, Spectinomycin, Streptomycin, Streptomycin, Sulfabenzamide, Sulfacetamide, Sulfadiazine, Sulfadimidine, Sulfametoxydiazine, Sulfanilamide, Trimethoprim, Carbamazepine, Ethosuximide, Lamotrigine, Primidone, Cetirizine, Chlorpheniramine, Diphenhydramine, Doxylamine, Promethazine, Sulfadimethoxine, Benzothiazinone, Butylated Hydroxytoluene, Tripelennamine, Chlorpromazine, Clozapine, Haloperidol, Olanzapine, Paliperidone, Quetiapine, Ribavirin, Meprobamate, Acebutolol, Atenolol, Penbutolol, Warfarin, Salmeterol, Aflatoxin B1, Tetraxetan (DOTA), MPOB, Biotin, Melamine, Methotrexate, Amphetamine, Diethylpropion, Dextromethorphan, Pseudoephedrine, Dihydrochlorothiazide, Hydrochlorothiazide, Clonazepam, Diazepam, Nitrazepam, Rhodamine B, Fluorescent Brightener Ksn, Zearalenone, Sudan Red1, Acetominophen, Acrylamide, Benzoic Acid, Benzophenone, Benzothiazine, Mercaptobenzothiazole, Erythrosine, Sudan, Tartrazine, Erythromycin, Sirolimus, Atropine, Ethyl glucuronide, Aflatoxin M1, Methocarbamol, Fentanyl, Hydromorphone, Morphine, Remifentanil, Tapentadol, Tramadol, Pregabalin, Gabapentin, Amitriptyline, Desipramine, Imipramine, Nortriptyline, Venlafaxine, Dinitrophenyl (DNP), His-Tag, PEG methoxy group, Etodolac, Ibuprofen, Ketoprofen, Meclofenamic Acid, Phenylbutazone, Acetyl Salicylic Acid, Acetamiprid, Acetochlor, Carbadazim, Carbaryl, Chlorothalonil, Chlorpyrifos, Fenpropathrin, Imazalil, Imidacloprid, Parathion, Abscisic acid, Dibutyl Phthalate, Clonazepam, Lorazepam, Oxazepam, Phenobarbital, Secobarbital, Zaleplon, Zolpidem, Trazodone, Fluoxetine, Fluvoxamine, Cortisone, Dexamethasone, Dihydrotestosterone, Fluocinolone, Methylprednisolone, Prednisolone, Stanozolol, Triamcinolone, Mazindol, Methamphetamine, Methylphenidate, Modafinil, Chrysoidine, Deoxynivalenol, Fumonisin, Microcystin Lr, Ochratoxin, Sterigmatocystin, T-2 toxin, Sildenafil, Tadalafil, Scopolamine, Florfenicol, Pirlimycin, or Sulfaquinoxaline.

In some embodiments, the antigen binding domain is a VHH antibody, an scFv, a V_{H}, or a V_{L} of an antibody, or a ligand that recognizes any of the exogenous antigens described herein. For example, the antigen binding domain can be a VHH antibody, an scFv, a V_{H}, or a V_{L} of an anti-FITC antibody (e.g., a 4M5.3 anti-FITC antibody). As another example, the antigen binding domain can be a VHH antibody, an scFv, a V_{H}, or a V_{L} of an anti-DOTA antibody (e.g., a C8.2.5 anti-DOTA antibody). In a further example, the antigen binding domain can be a VHH antibody, an scFv, a V_{H}, or a V_{L} of an anti-MPOB antibody.

In some embodiments, the antigen recognized by the indirect CAR is the same as the activator recognized by the engineered cytokine receptor switch (e.g., the antigen binding domain of the CAR can be the same as the activator binding domain of the engineered cytokine receptor switch). In such embodiments, the indirect CAR may recognize the same epitope on the activator as the engineered cytokine receptor switch, or may recognize a different epitope on the activator than the engineered cytokine receptor switch. Alternatively, the antigen recognized by the indirect CAR can be different from the activator recognized by the engineered cytokine receptor switch (e.g., the antigen binding domain of the CAR can be different than the activator binding domain of the engineered cytokine receptor switch). In some embodiments, the antigen binding domain of the indirect CAR binds to a first small molecule, and the activator binding domain of the engineered cytokine receptor switch binds to a second small molecule, where the first small molecule may be the same as or different than the second small molecule. In some embodiments, the antigen binding domain of the indirect CAR binds to a first epitope on a small molecule, and the activator binding domain of the engineered cytokine receptor switch binds to a second epitope on the small molecule, where the first epitope may be the same as or different than the second epitope.

The transmembrane domain of the CAR (e.g., a direct CAR or an indirect CAR) may link the extracellular domain to the intracellular domain. In some embodiments, the transmembrane domain may be a transmembrane domain derived from any transmembrane protein. The transmembrane domain may comprise a transmembrane region of alpha, beta, or zeta chain of the T-cell receptor a chain, T-cell receptor R chain, T-cell receptor (chain, CD28, CD3 epsilon, CD45, CD4, CD5, CD8, CD9, CD16, CD22, CD33, CD37, CD64, CD80, CD86, CD134, CD137, or CD154. For example, the transmembrane domain may comprise a CD8 transmembrane domain, including a CD8a hinge domain and/or a CD8a transmembrane domain. In some embodiments, the transmembrane domain may be synthetic. For example, a synthetic transmembrane domain may comprise mostly hydrophobic residues (e.g., glycine, leucine, isoleucine, alanine, valine, proline, methionine, phenylalanine, and tryptophan). In some embodiments, a first peptide linker (e.g., comprising glycine, serine, or combinations thereof) may connect the transmembrane domain to the extracellular domain. In some embodiments, a second peptide linker (e.g., comprising glycine, serine, or combinations thereof) may connect the transmembrane domain to the intracellular domain.

The intracellular domain of the CAR (e.g., a direct CAR or an indirect CAR), also referred to as the cytoplasmic domain, may be capable of activating a specialized immune cell function (e.g., an immune response). For example, the specialized immune cell function of a T-cell may comprise cytolytic activity, cytokine secretion, or both. In some embodiments, the intracellular domain of the CAR may comprise the intracellular domain of TCR zeta, FcR gamma, FcR beta, CD3 zeta, CD3 gamma, CD3 delta, CD3 epsilon, CD5, CD22, CD79a, CD79b, or CD66d. In some embodiments, the intracellular domain of the CAR may comprise a portion of the intracellular domain of TCR zeta, FcR gamma, FcR beta, CD3 zeta, CD3 gamma, CD3 delta, CD3 epsilon, CD5, CD22, CD79a, CD79b, or CD66d sufficient to activate the specialized immune cell function. In some embodiments, the intracellular domain of the CAR may comprise a CD137 (4-1BB) signaling domain, a CD28 signaling domain, and/or a CD3 zeta signal domain.

Binding of the antigen to the antigen binding domain of the CAR may initiate signal transduction through the transmembrane domain to the cytoplasmic domain to activate the specialized immune cell function. For example, binding of the antigen binding domain to a tumor antigen may activate the intracellular domain of the CAR to trigger cytokine release. In some embodiments, the CAR may facilitate antigen-specific cancer cell killing by binding directly or indirectly to a tumor cell surface antigen present on the cancer cell via the antigen binding domain, transducing a signal through the transmembrane domain to the intracellular domain, and activating the specialized immune cell function (e.g., cytokine release) via activation of the intracellular domain. The specialized immune cell function may kill the cancer cell or may facilitate killing of the cancer cell.

Examples of polynucleotide sequences for CARs are provided in Table 8.

TABLE 8
Representative Examples of CARs

| CAR | SEQ ID NO | Sequence |
|---|---|---|
| bb2121 (anti- | SEQ ID NO: 40 | ATGGCACTCCCCGTCACCGCCCTTCTCTTGCCCCTCGCCCTGC |
| BCMA CAR)- | | TGCTGCATGCTGCCAGGCCCGACATTGTGCTCACTCAGTCAC |
| P2A-truncated | | CTCCCAGCCTGGCCATGAGCCTGGGAAAAAGGGCCACCATC |
| CD19 | | TCCTGTAGAGCCAGTGAGTCCGTCACAATCTTGGGGAGCCAT |
| | | CTTATTCACTGGTATCAGCAGAAGCCCGGGCAGCCTCCAACC |
| | | CTTCTTATTCAGCTCGCGTCAAACGTCCAGACGGGTGTACCT |
| | | GCCAGATTTTCTGGTAGCGGGTCCCGCACTGATTTTACACTG |
| | | ACCATAGATCCAGTGGAAGAAGACGATGTGGCCGTGTATTA |
| | | TTGTCTGCAGAGCAGAACGATTCCTCGCACATTTGGTGGGGG |
| | | TACTAAGCTGGAGATTAAGGGAAGCACGTCCGGCTCAGGGA |
| | | AGCCGGGCTCCGGCGAGGGAAGCACGAAGGGGCAAATTCAG |
| | | CTGGTCCAGAGCGGACCTGAGCTGAAAAAACCCGGCGAGAC |
| | | TGTTAAGATCAGTTGTAAAGCATCTGGCTATACCTTCACCGA |
| | | CTACAGCATAAATTGGGTGAAACGGGCCCCTGGAAAGGGCC |
| | | TCAAATGGATGGGTTGGATCAATACCGAAACTAGGGAGCCT |
| | | GCTTATGCATATGACTTCCGCGGGAGATTCGCCTTTTCACTC |
| | | GAGACATCTGCCTCTACTGCTTACCTCCAAATAAACAACCTC |
| | | AAGTATGAAGATACAGCCACTTACTTTTGCGCCCTCGACTAT |
| | | AGTTACGCCATGGACTACTGGGGACAGGGAACCTCCGTTACC |
| | | GTCAGTTCCGCGGCCGCAACCACAACACCTGCTCCAAGGCCC |
| | | CCCACACCCGCTCCAACTATAGCCAGCCAACCATTGAGCCTC |
| | | AGACCTGAAGCTTGCAGGCCCGCAGCAGGAGGCGCCGTCCA |
| | | TACGCGAGGCCTGGACTTCGCGTGTGATATTTATATTTGGGC |
| | | CCCTTTGGCCGGAACATGTGGGGTGTTGCTTCTCTCCCTTGTG |
| | | ATCACTCTGTATTGTAAGCGCGGGAGAAAGAAGCTCCTGTAC |
| | | ATCTTCAAGCAGCCTTTTATGCGACCTGTGCAAACCACTCAG |
| | | GAAGAAGATGGGTGTTCATGCCGCTTCCCCGAGGAGGAAGA |
| | | AGGAGGGTGTGAACTGAGGGTGAAATTTTCTAGAAGCGCCG |
| | | ATGCTCCCGCATATCAGCAGGGTCAGAATCAGCTCTACAATG |
| | | AATTGAATCTCGGCAGGCGAGAAGAGTACGATGTTCTGGAC |
| | | AAGAGACGGGGCAGGGATCCCGAGATGGGGGGAAAGCCCC |
| | | GGAGAAAAAATCCTCAGGAGGGGTTGTACAATGAGCTGCAG |
| | | AAGGACAAGATGGCTGAAGCCTATAGCGAGATCGGAATGAA |
| | | AGGCGAAAGACGCAGAGGCAAGGGGCATGACGGTCTGTACC |
| | | AGGGTCTCTCTACAGCCACCAAGGACACTTATGATGCGTTGC |
| | | ATATGCAAGCCTTGCCACCCCGCGGATCTGGCGCCACAAACT |
| | | TCTCACTGCTGAAACAGGCCGGCGACGTCGAAGAGAATCCT |
| | | GGGCCTATGCCTCCTCCTCGGCTGCTGTTCTTCCTGCTGTTTC |
| | | TGACCCCTATGGAAGTGCGGCCCGAGGAACCTCTGGTCGTGA |
| | | AAGTTGAAGAGGGCGACAACGCCGTGCTGCAGTGTCTGAAG |
| | | GGCACATCTGATGGCCCCACACAGCAGCTGACCTGGTCTAGA |
| | | GAGAGCCCTCTGAAGCCCTTCCTGAAGCTCAGTCTGGGACTG |
| | | CCTGGACTGGGCATCCATATGAGGCCACTGGCCATCTGGCTG |
| | | TTCATCTTTAACGTGTCCCAGCAGATGGGAGGCTTCTACCTG |
| | | TGTCAGCCTGGACCTCCTAGCGAGAAAGCTTGGCAGCCTGGC |
| | | TGGACCGTGAATGTGGAAGGATCCGGCGAGCTGTTCCGGTG |
| | | GAATGTCTCTGATCTCGGCGGCCTCGGATGCGGCCTGAAGAA |
| | | TAGATCTAGCGAGGGCCCTAGCAGCCCCTCCGGAAAACTGA |
| | | TGAGCCCCAAGCTGTACGTGTGGGCCAAAGACAGACCCGAG |
| | | ATTTGGGAGGGCGAGCCTCCTTGTCTGCCACCTAGAGACAGC |
| | | CTGAATCAGAGCCTGAGCCAGGACCTGACAATGGCCCCTGG |
| | | ATCTACACTGTGGCTGAGCTGCGGAGTGCCTCCTGACAGTGT |
| | | GTCTAGAGGCCCACTGAGCTGGACACACGTGCACCCTAAGG |
| | | GCCCTAAGAGCCTGCTGAGTCTGGAACTGAAGGACGACAGG |
| | | CCCGCCAGAGATATGTGGGTCATGGAAACCGGACTGCTGCTC |
| | | CCTAGAGCCACTGCTCAAGATGCCGGCAAGTACTATTGCCAC |
| | | CGGGGCAACCTGACCATGAGCTTCCACCTGGAAATTACCGCC |
| | | AGACCAGTGCTGTGGCATTGGCTGCTTAGAACCGGCGGATG |
| | | GAAGGTGTCAGCCGTGACTCTGGCCTACCTGATCTTTTGTCT |
| | | GTGCAGCCTCGTGGGCATCCTGCATCTGCAAAGAGCCCTGGT |
| | | CCTGCGGCGGAAGCGGAAGAGAATGACCGATCCTACCAGAC |
| | | GGTTCTGATGA |
| Carvykti (anti- | SEQ ID NO: 41 | ATGGCTCTGCCTGTCACCGCTCTGCTGCTGCCTCTGGCTCTGC |
| BCMA CAR) | | TGCTGCACGCTGCTCGCCCTCAGGTCAAACTGGAAGAAAGTG |
| | | GGGGAGGCCTGGTGCAGGCAGGACGGAGCCTGCGCCTGAGC |
| | | TGCGCAGCATCCGAGCACACCTTCAGCTCCCACGTGATGGGC |
| | | TGGTTTCGGCAGGCCCCAGGCAAGGAGAGAGAGTCCGTGGC |
| | | CGTGATCGGCTGGAGGGACATCTCCACATCTTACGCCGATTC |
| | | TGTGAAGGGCCGGTTCACCATCAGCAGAGACAACGCCAAGA |
| | | AGACACTGTATCTGCAGATGAATAGCCTGAAGCCCGAGGAC |
| | | ACCGCCGTGTACTATTGCGCAGCAAGGAGAATCGACGCAGC |
| | | AGACTTTGATTCCTGGGGCCAGGGCACCCAGGTGACAGTGTC |
| | | TAGCGGAGGAGGAGGATCTGGAGGAGGAGGAAGCGGAGGA |
| | | GGAGGATCCGAGGTGCAGCTGGTGGAGTCTGGAGGCGGCCT |
| | | GGTGCAGGCCGGAGGCTCTCTGAGGCTGAGCTGTGCAGCAT |
| | | CCGGAAGAACCTTCACAATGGGCTGGTTTAGGCAGGCACCA |
| | | GGAAAGGAGAGGGAGTTCGTGGCAGCAATCAGCCTGTCCCC |
| | | TACCCTGGCCTACTATGCCGAGTCCGTGAAGGGCAGGTTTAC |
| | | CATCTCTCGCGATAACGCCAAGAATACAGTGGTGCTGCAGAT |
| | | GAACAGCCTGAAACCTGAGGACACAGCCCTGTACTATTGTGC |
| | | CGCCGATCGGAAGAGCGTGATGAGCATTAGACCCGATTATT |
| | | GGGGACAGGGCACACAGGTGACAGTGAGTAGCACTAGTACC |
| | | ACGACGCCAGCGCCGCGACCACCAACACCGGCGCCCACCAT |
| | | CGCGTCGCAGCCCCTGTCCCTGCGCCCAGAGGCGTGCCGGCC |
| | | AGCGGCGGGGGGCGCAGTGCACACGAGGGGGCTGGACTTCG |
| | | CCTGTGATATCTACATCTGGGCGCCCTTGGCCGGGACTTGTG |
| | | GGGTCCTTCTCCTGTCACTGGTTATCACCCTTTACTGCAAACG |
| | | GGGCAGAAAGAAACTCCTGTATATATTCAAACAACCATTTAT |
| | | GAGACCAGTACAAACTACTCAAGAGGAAGATGGCTGTAGCT |
| | | GCCGATTTCCAGAAGAAGAAGAAGGAGGATGTGAACTGAGA |
| | | GTGAAGTTCAGCAGGAGCGCAGACGCCCCCGCGTACCAGCA |
| | | GGGCCAGAACCAGCTCTATAACGAGCTCAATCTAGGACGAA |
| | | GAGAGGAGTACGATGTTTTGGACAAGAGACGTGGCCGGGAC |
| | | CCTGAGATGGGGGGAAAGCCGAGAAGGAAGAACCCTCAGG |
| | | AAGGCCTGTACAATGAACTGCAGAAAGATAAGATGGCGGAG |
| | | GCCTACAGTGAGATTGGGATGAAAGGCGAGCGCCGGAGGGG |
| | | CAAGGGGCACGATGGCCTTTACCAGGGTCTCAGTACAGCCA |
| | | CCAAGGACACCTACGACGCCCTTCACATGCAGGCCCTGCCCC |
| | | CTCGCTAATGA |
| anti-CD123 | SEQ ID NO: 42 | ATGGCCCTGCCAGTAACTGCTCTTCTCCTGCCACTCGCTCTTC |
| CAR | | TGCTGCACGCCGCACGGCCTCAAATTCAACTTGTGCAGAGTG |
| | | GCCCCGAGCTGAAAAAGCCCGGCGAAACAGTAAAGATCTCT |
| | | TGTAAGGCGTCTGGATATATCTTTACCAATTACGGTATGAAC |
| | | TGGGTCAAACAGGCACCCGGTAAAAGCTTTAAATGGATGGG |
| | | CTGGATAAATACTTATACCGGAGAAAGCACGTATAGCGCCG |
| | | ACTTCAAGGGTAGATTTGCGTTCAGCCTGGAAACGTCCGCCA |
| | | GCACTGCATACTTGCACATCAATGACTTGAAAAATGAGGATA |
| | | CCGCGACCTACTTTTGTGCTAGAAGTGGTGGATACGATCCCA |
| | | TGGATTACTGGGGACAGGGGACCTCTGTCACAGTCAGTTCTG |
| | | GGGGCGGCGGTTCCGGAGGTGGCGGCTCAGGTGGAGGTGGG |
| | | TCAGATATCGTGTTGACACAAAGCCCTGCTTCCCTGGCCGTC |
| | | AGCTTGGGACAGAGAGCTACTATCTCCTGTAGAGCAAGTGA |
| | | GTCTGTCGACAATTACGGCAACACCTTTATGCACTGGTACCA |
| | | GCAGAAGCCAGGCCAGCCACCGAAGCTGTTGATTTACCGGG |
| | | CCTCAAACCTCGAAAGCGGGATTCCAGCTAGATTTTCTGGCA |
| | | GCGGGAGTCGAACCGACTTCACACTGACTATTAACCCAGTGG |
| | | AAGCAGATGACGTAGCCACATATTACTGTCAGCAGAGCAAC |
| | | GAAGACCCCCCGACGTTTGGTGCTGGAACCAAACTGGAGCT |
| | | GAAAACGACAACCCCCGCTCCACGACCTCCTACTCCCGCTCC |
| | | CACAATCGCATCTCAGCCACTCTCCTTGAGGCCTGAGGCCTG |
| | | CCGACCAGCTGCAGGCGGCGCAGTACACACCAGAGGCCTCG |
| | | ACTTTGCTTGCGATATCTACATATGGGCGCCCCTGGCCGGGA |
| | | CGTGTGGGGTATTGCTCCTGAGCCTTGTGATCACTCTTTACTG |
| | | CAAAAGAGGTCGGAAAAAGCTTCTTTACATCTTTAAACAGCC |
| | | GTTCATGCGCCCGGTGCAAACAACACAGGAGGAGGATGGAT |
| | | GCAGCTGCAGGTTTCCGGAGGAGGAGGAAGGCGGGTGTGAG |
| | | CTCCGGGTAAAATTCTCCAGAAGCGCAGACGCGCCAGCCTAT |
| | | CAGCAGGGGCAGAATCAGCTCTATAACGAACTCAATCTTGG |
| | | GCGACGGGAAGAATACGATGTACTTGATAAACGCCGAGGTC |
| | | GCGACCCCGAAATGGGAGGCAAGCCTCAGAGACGCAAGAAT |
| | | CCCCAAGAAGGCCTGTATAATGAACTCCAGAAGGATAAAAT |
| | | GGCGGAGGCCTACAGCGAAATTGGTATGAAGGGAGAAAGAC |
| | | GACGCGGTAAAGGCCACGACGGCCTTTACCAGGGCCTGTCA |
| | | ACAGCTACCAAAGATACGTATGATGCACTTCACATGCAAGCC |
| | | CTGCCGCCCCGGTGATGA |
| anti-GD2 CAR | SEQ ID NO: 43 | ATGGCTCTGCCTGTCACCGCTCTGCTGCTGCCTCTGGCTCTGC |
| | | TGCTGCACGCTGCTCGCCCTCAGGTGCAGCTGCAGGAGTCTG |
| | | GCCCAGGCCTGGTGAAGCCCAGCCAGACCCTGAGCATCACC |
| | | TGCACCGTGAGCGGCTTCAGCCTGGCCAGCTACAACATCCAC |
| | | TGGGTGCGGCAGCCCCCAGGCAAGGGCCTGGAGTGGCTGGG |
| | | CGTGATCTGGGCTGGCGGCAGCACCAACTACAACAGCGCCC |
| | | TGATGAGCCGGCTGACCATCAGCAAGGACAACAGCAAGAAC |
| | | CAGGTGTTCCTGAAGATGAGCAGCCTGACAGCCGCCGACAC |
| | | CGCCGTGTACTACTGCGCCAAGCGGAGCGACGACTACAGCT |
| | | GGTTCGCCTACTGGGGCCAGGGCACCCTGGTGACCGTGAGCT |
| | | CTGGCGGAGGCGGCTCTGGCGGAGGCGGCTCTGGCGGAGGC |
| | | GGCAGCGAGAACCAGATGACCCAGAGCCCCAGCAGCTTGAG |
| | | CGCCAGCGTGGGCGACCGGGTGACCATGACCTGCAGAGCCA |
| | | GCAGCAGCGTGAGCAGCAGCTACCTGCACTGGTACCAGCAG |
| | | AAGAGCGGCAAGGCCCCAAAGGTGTGGATCTACAGCACCAG |
| | | CAACCTGGCCAGCGGCGTGACCAGCCGGTTCAGCGGCAGCG |
| | | GCAGCGGCACCGACTACACCCTGACCATCAGCAGCCTGCAG |
| | | CCCGAGGACTTCGCCACCTACTACTGCCAGCAGTACAGCGGC |
| | | TACCCCATCACCTTCGGCCAGGGCACCAAGGTGGAGATCAA |
| | | GCGGTCGGATCCCACCACGACGCCAGCGCCGCGACCACCAA |
| | | CACCGGCGCCCACCATCGCGTCGCAGCCCCTGTCCCTGCGCC |
| | | CAGAGGCGTGCCGGCCAGCGGCGGGGGGCGCAGTGCACACG |
| | | AGGGGGCTGGACTTCGCCTGTGATATCTACATCTGGGCGCCC |
| | | TTGGCCGGGACTTGTGGGGTCCTTCTCCTGTCACTGGTTATCA |
| | | CCCTTTACTGCAAACGGGGCAGAAAGAAACTCCTGTATATAT |
| | | TCAAACAACCATTTATGAGACCAGTACAAACTACTCAAGAG |
| | | GAAGATGGCTGTAGCTGCCGATTTCCAGAAGAAGAAGAAGG |
| | | AGGATGTGAACTGAGAGTGAAGTTCAGCAGGAGCGCAGACG |
| | | CCCCCGCGTACCAGCAGGGCCAGAACCAGCTCTATAACGAG |
| | | CTCAATCTAGGACGAAGAGAGGAGTACGATGTTTTGGACAA |
| | | GAGACGTGGCCGGGACCCTGAGATGGGGGGAAAGCCGAGA |
| | | AGGAAGAACCCTCAGGAAGGCCTGTACAATGAACTGCAGAA |
| | | AGATAAGATGGCGGAGGCCTACAGTGAGATTGGGATGAAAG |
| | | GCGAGCGCCGGAGGGGCAAGGGGCACGATGGCCTTTACCAG |
| | | GGTCTCAGTACAGCCACCAAGGACACCTACGACGCCCTTCAC |
| | | ATGCAGGCCCTGCCCCCTCGCTAATGA |
| anti-DOTA | SEQ ID NO: 44 | ATGGCTCTGCCAGTGACAGCTCTGCTGCTTCCTCTGGCTCTCC |
| CAR | | TGCTGCATGCCGCTAGACCTGAGCAGAAACTCATCTCTGAAG |
| | | AGGATCTGCACGTGAAACTGCAAGAGTCTGGCCCCGGACTG |
| | | GTGCAGCCTTCTCAGTCTCTCTCTCTGACCTGTACCGTGTCCG |
| | | GCTTCAGCCTGACCGATTATGGCGTGCACTGGGTTCGACAAT |
| | | CCCCAGGCAAAGGACTCGAGTGGCTGGGAGTGATTTGGAGC |
| | | GGCGGAGGCACAGCCTATAACACAGCCCTGATCAGCAGACT |
| | | GAACATCTACCGGGACAACTCCAAGAACCAGGTTTTCCTGGA |
| | | AATGAACTCCCTGCAGGCAGAGGACACCGCCATGTACTACT |
| | | GCGCCAGAAGAGGCAGCTACCCCTACAATTACTTCGACGCCT |
| | | GGGGCTGTGGCACAACCGTGACAGTTTCTAGTGGCGGAGGC |
| | | GGATCTGGTGGTGGTGGTAGCGGTGGCGGAGGATCTCAGGC |
| | | CGTGGTTATTCAAGAAAGCGCCCTGACAACCCCTCCTGGCGA |
| | | GACAGTGACACTGACATGTGGCAGCTCTACAGGCGCCGTGA |
| | | CCGCCAGCAATTACGCCAATTGGGTGCAAGAGAAGCCCGAC |
| | | CACTGCTTCACAGGCCTGATCGGCGGCCACAACAATAGACCT |
| | | CCAGGCGTGCCAGCTAGATTCAGCGGATCCCTGATCGGAGA |
| | | CAAGGCCGCTCTGACAATCGCCGGCACACAGACAGAGGACG |
| | | AGGCCATCTACTTTTGCGCCCTGTGGTACAGCGACCACTGGG |
| | | TTATCGGCGGAGGAACCAGACTGACAGTGCTGGGCACAACA |
| | | ACACCCGCACCTAGACCACCAACTCCAGCACCAACAATCGC |
| | | CTCTCAACCCCTGAGTCTGAGGCCAGAGGCATGCAGACCAG |
| | | CCGCTGGCGGTGCAGTTCACACTAGAGGACTGGACTTTGCCT |
| | | GTGACATCTACATCTGGGCCCCTCTGGCTGGAACATGTGGTG |
| | | TCCTGCTGCTGTCCCTGGTCATCACCCTGTACTGCAAGCGGG |
| | | GCAGAAAGAAACTGCTGTACATCTTCAAGCAGCCCTTCATGC |
| | | GGCCCGTGCAGACCACACAAGAGGAAGATGGCTGCTCCTGC |
| | | AGATTCCCCGAGGAAGAAGAAGGCGGCTGCGAGCTGAGAGT |
| | | GAAGTTCTCCAGATCTGCCGACGCTCCCGCCTATCAGCAGGG |
| | | ACAGAACCAGCTGTACAACGAGCTGAACCTGGGGAGAAGAG |
| | | AAGAGTACGACGTGCTGGACAAGCGGAGAGGCAGAGATCCT |
| | | GAGATGGGCGGAAAGCCCCAGCGGAGAAAGAATCCTCAAGA |
| | | GGGCCTGTATAATGAGCTGCAAAAGGACAAGATGGCCGAGG |
| | | CCTACAGCGAGATCGGAATGAAGGGCGAGCGCAGAAGAGG |
| | | AAAGGGACACGACGGACTGTACCAGGGCCTGAGCACAGCCA |
| | | CCAAGGATACCTATGACGCCCTGCACATGCAGGCCCTGCCTC |
| | | CAAGATGATGA |
| anti-FITC | SEQ ID NO: 45 | ATGGCTCTGCCTGTGACAGCTCTGCTGCTGCCTCTGGCTCTGC |
| CAR | | TTCTGCATGCCGCCAGACCTGACGTGGTCATGACACAGACAC |
| | | CTCTGAGCCTGCCTGTGTCTCTGGGAGATCAGGCCAGCATCA |
| | | GCTGCAGATCTAGCCAGAGCCTGGTGCACAGCAACGGCAAC |
| | | ACCTACCTGCGGTGGTATCTGCAGAAGCCCGGCCAGTCTCCT |
| | | AAGGTGCTGATCTACAAGGTGTCCAACAGAGTGTCCGGCGT |
| | | GCCCGATAGATTTTCTGGCAGCGGCTCTGGCACCGACTTCAC |
| | | CCTGAAGATCAATAGAGTGGAAGCCGAGGACCTGGGCGTGT |
| | | ACTTCTGTAGCCAGTCTACCCACGTGCCATGGACCTTTGGCG |
| | | GCGGAACAAAGCTGGAAATCAAGAGCAGCGCCGACGACGCC |
| | | AAGAAGGACGCCGCTAAGAAGGATGACGCCAAAAAAGACG |
| | | ATGCCAAAAAGGATGGCGGCGTGAAGCTGGACGAAACAGGC |
| | | GGAGGACTTGTTCAGCCTGGCGGAGCCATGAAGCTGAGCTG |
| | | TGTGACCAGCGGCTTCACCTTCGGCCACTACTGGATGAACTG |
| | | GGTCCGACAGAGCCCTGAGAAAGGCCTGGAATGGGTCGCCC |
| | | AGTTCAGAAACAAGCCCTACAACTACGAAACCTACTACAGC |
| | | GACAGCGTGAAGGGCAGATTCACCATCAGCCGGGACGACAG |
| | | CAAGTCCAGCGTGTACCTGCAGATGAACAACCTGCGCGTGG |
| | | AAGATACCGGCATCTACTACTGTACCGGCGCCAGCTACGGCA |
| | | TGGAATATCTCGGCCAGGGCACCAGCGTGACCGTGTCTACAA |
| | | CAACCCCTGCTCCTCGGCCTCCTACACCAGCTCCTACAATTG |
| | | CCAGCCAGCCACTGTCTCTGAGGCCCGAAGCTTGTAGACCTG |
| | | CTGCAGGCGGAGCCGTGCATACAAGAGGACTGGATTTCGCC |
| | | TGCGACTTCTGGGTGCTCGTGGTTGTTGGCGGAGTGCTGGCT |
| | | TGTTACTCCCTGCTGGTTACCGTGGCCTTCATCATCTTTTGGG |
| | | TCAAGCGGGGCAGAAAGAAGCTGCTGTACATCTTCAAGCAG |
| | | CCCTTCATGCGGCCCGTGCAGACCACACAAGAGGAAGATGG |
| | | CTGCTCCTGCAGATTCCCCGAGGAAGAAGAAGGCGGCTGCG |
| | | AGCTGAGAGTGAAGTTCAGCAGATCCGCCGACGCTCCTGCCT |
| | | ATCAGCAGGGACAGAACCAGCTGTACAACGAGCTGAACCTG |
| | | GGGAGAAGAGAAGAGTACGACGTGCTGGACAAGCGGAGAG |
| | | GCAGAGATCCTGAGATGGGCGGAAAGCCCCAGCGGAGAAAG |
| | | AATCCTCAAGAGGGCCTGTATAATGAGCTGCAGAAAGACAA |
| | | GATGGCCGAGGCCTACAGCGAGATCGGAATGAAGGGCGAGC |
| | | GCAGAAGAGGCAAGGGACACGATGGACTGTACCAGGGCCTG |
| | | AGCACCGCCACCAAGGATACCTATGATGCCCTGCACATGCA |
| | | GGCCCTGCCACCTAGATGATGA |

In some embodiments, a CAR is encoded by a sequence having at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to any one of SEQ ID NO: 40-SEQ ID NO: 45. In some embodiments, the CAR is encoded by a sequence of any one of SEQ ID NO: 40-SEQ ID NO: 45.

### IV. Vectors

The present disclosure provides polynucleotides encoding the engineered cytokine receptor switches described herein. A polynucleotide may comprise an RNA sequence or a DNA sequence encoding an engineered cytokine receptor switch and/or a CAR, or an RNA sequence or a DNA sequence reverse complementary to a sequence encoding an engineered cytokine receptor switch and/or a CAR. In some embodiments, the polynucleotide encoding the engineered cytokine receptor switch and/or CAR may be part of a polynucleotide construct (also referred to herein as a polynucleotide expression cassette) capable of expressing the engineered cytokine receptor switch and/or a CAR in a cell (e.g., an immune cell). The polynucleotide expression cassette may comprise a promoter, an open reading frame (e.g., encoding the engineered cytokine receptor switch), a 3′ untranslated region, or combinations thereof. In some embodiments, the polynucleotide expression cassette may comprise two or more open reading frames. For example, the polynucleotide expression cassette may comprise a first open reading frame encoding the engineered cytokine receptor switch and a second open reading frame encoding a CAR. The expression cassette may further comprise an origin of replication, a restriction endonuclease site, a selectable marker, or combinations thereof. The expression cassette may be capable of expressing both the engineered cytokine receptor switch and the CAR in a cell (e.g., an immune cell). In some embodiments, the cell may be a mammalian cell (e.g., a human cell). For example, the cell may be a human T-cell. A polynucleotide or polynucleotide expression cassette may be obtained using recombinant methods known in the art. Alternatively or in addition, the polynucleotide or polynucleotide expression cassette may be generated synthetically.

Also provided herein are vectors comprising a polynucleotide expression cassette capable of delivering the polynucleotide expression cassette to a target cell (e.g., an immune cell). Upon delivery, a protein encoded by the polynucleotide expression cassette (e.g., an engineered cytokine receptor switch, a CAR, or combinations thereof) may be expressed in the cell. In some embodiments, the vector may be a viral vector (e.g., an adeno-associated viral vector or a lentiviral vector). The vector may be a viral vector derived from a retrovirus, an adenovirus, an adeno-associated virus, a herpes virus, a vaccinia virus, a poxvirus, an alphavirus, a gamma retrovirus, a polyoma virus, or a lentivirus. A vector encoding an engineered cytokine receptor switch may be generated and delivered to an immune cell using standard cloning and gene delivery protocols, for example as described in Sambrook et al. (2001, Molecular Cloning: A Laboratory Manual, Cold Spring Harbor Laboratory, New York), which is herein incorporated by reference. A vector may encode a selectable marker, a reporter gene, or both to facilitate selection of cells (e.g., immune cells) successfully transfected and expressing a protein encoded by the expression cassette (e.g., the engineered cytokine receptor switch, the CAR, or both).

Alternatively, or in addition, a polynucleotide expression cassette may be introduced into a target cell using physical or chemical means. In some embodiments, a polynucleotide expression cassette may be introduced into a target cell using calcium phosphate precipitation, lipofection, particle bombardment, microinjection, electroporation, and the like. In some embodiments, the polynucleotide expression cassette may be introduced using colloidal dispersion systems (e.g., macromolecule complexes), nanocapsules, microspheres, beads, lipid-based systems (e.g., oil-in-water emulsions, micelles, mixed micelles, liposomes), and the like. Additional transfection or infection methods are described in Sambrook et al. (2001, Molecular Cloning: A Laboratory Manual, Cold Spring Harbor Laboratory, New York).

Examples of polynucleotide constructs encoding an engineered cytokine switch (SMAR) and a CAR are provided in Table 9.

TABLE 9
Representative Examples of SMAR-CAR Constructs

| SMAR-CAR | | |
|---|---|---|
| Construct | SEQ ID NO | Sequence |
| anti-FITC | SEQ ID NO: 46 | ATGGCTGCTCCTGCTCTGTCTTGGAGACTGCCCCTGCTGATTC |
| IL2Rβ SMAR- | | TGCTGCTGCCTCTGGCTACATCTTGGGCCTCTGCCGATTACA |
| F2A-anti- | | AGGATGACGACGATAAGGACGTGGTCATGACACAGACACCA |
| FITC IL2Rγ | | CTGAGCCTGCCTGTGTCTCTGGGAGATCAGGCCAGCATCAGC |
| SMAR-T2A- | | TGCAGATCCAGCCAGTCTCTGGTGCACAGCAACGGCAACAC |
| bb2121 | | CTACCTGCGGTGGTATCTGCAGAAGCCCGGCCAGTCTCCTAA |
| | | GGTGCTGATCTACAAGGTGTCCAACAGAGTGTCCGGCGTGCC |
| | | CGATAGATTTTCTGGCAGCGGCTCTGGCACCGACTTCACCCT |
| | | GAAGATCAATAGAGTGGAAGCCGAGGACCTGGGCGTGTACT |
| | | TCTGTAGCCAGTCTACCCACGTGCCATGGACCTTTGGCGGCG |
| | | GAACAAAGCTGGAAATCAAGAGCAGCGCCGACGACGCCAAG |
| | | AAGGACGCCGCTAAGAAGGATGACGCAAAGAAAGACGATG |
| | | CCAAAAAGGATGGCGGCGTGAAGCTGGACGAAACAGGCGG |
| | | AGGACTTGTTCAGCCTGGCGGAGCCATGAAGCTGAGCTGTGT |
| | | GACCAGCGGCTTCACCTTCGGCCACTACTGGATGAACTGGGT |
| | | CCGACAGAGCCCTGAGAAAGGCCTGGAATGGGTCGCCCAGT |
| | | TCAGAAACAAGCCCTACAACTACGAAACCTACTACAGCGAC |
| | | AGCGTGAAGGGCAGATTCACCATCAGCCGGGACGACAGCAA |
| | | GTCCAGCGTGTACCTGCAGATGAACAACCTGCGCGTGGAAG |
| | | ATACCGGCATCTACTACTGTACCGGCGCCAGCTACGGCATGG |
| | | AATATCTCGGCCAGGGCACCAGCGTGACCGTGTCTACAACA |
| | | ACCCCTGCTCCTCGGCCTCCTACACCAGCTCCTACAATTGCC |
| | | AGCCAGCCACTGTCTCTGAGGCCCGAAGCTTGTAGACCTGCT |
| | | GCAGGCGGAGCCGTGCATACAAGAGGACTGGATTTCGCCTG |
| | | CGACATCCCCTGGCTGGGACATCTGCTTGTTGGACTGTCTGG |
| | | CGCCTTCGGCTTCATCATCCTGGTGTATCTGCTGATCAACTGC |
| | | CGGAACACAGGCCCTTGGCTGAAGAAAGTGCTGAAGTGCAA |
| | | CACCCCTGATCCGAGCAAGTTCTTTAGCCAGCTGAGCAGCGA |
| | | GCATGGCGGCGACGTTCAGAAATGGCTGTCTAGCCCATTTCC |
| | | TAGCAGCAGCTTCAGCCCAGGTGGACTGGCCCCTGAGATTAG |
| | | CCCTCTGGAAGTGCTGGAACGGGACAAAGTGACCCAGCTGC |
| | | TCCTCCAGCAGGATAAGGTGCCAGAACCTGCCAGCCTGTCCA |
| | | GCAATCACAGCCTGACCAGCTGCTTTACCAACCAGGGCTACT |
| | | TCTTCTTCCATCTGCCTGACGCTCTGGAAATCGAGGCCTGCC |
| | | AGGTGTACTTCACCTACGATCCCTACAGCGAAGAGGACCCCG |
| | | ATGAAGGTGTTGCTGGCGCCCCTACAGGATCTTCTCCACAGC |
| | | CTCTGCAACCTCTGAGCGGCGAGGATGATGCCTACTGCACCT |
| | | TTCCAAGCAGGGACGACCTGCTCCTGTTCAGCCCATCTCTGC |
| | | TCGGAGGACCATCTCCTCCATCTACAGCTCCAGGCGGATCTG |
| | | GCGCTGGCGAGGAAAGAATGCCACCTAGCCTGCAAGAGCGG |
| | | GTGCCCAGAGATTGGGATCCTCAACCTCTCGGCCCTCCAACA |
| | | CCTGGCGTGCCAGATCTCGTGGACTTTCAGCCTCCTCCAGAG |
| | | CTGGTGCTGAGAGAAGCTGGCGAAGAAGTGCCAGACGCTGG |
| | | CCCTAGAGAGGGCGTTAGCTTTCCTTGGAGCAGACCTCCTGG |
| | | ACAGGGCGAGTTTAGGGCCCTGAATGCTAGACTGCCTCTGAA |
| | | CACCGACGCCTACCTGTCTCTGCAAGAACTGCAGGGACAAG |
| | | ACCCCACACACCTCGTTGGAAGCGGAGTGAAGCAGACCCTG |
| | | AACTTCGACCTGCTGAAACTGGCCGGCGACGTGGAAAGCAA |
| | | CCCTGGACCTATGCTGAAGCCCAGCCTGCCTTTTACCAGCCT |
| | | GCTGTTCCTGCAGCTCCCACTGCTTGGCGTGGGATACCCATA |
| | | CGATGTTCCAGATTACGCTGATGTCGTGATGACCCAGACTCC |
| | | ACTCTCTCTGCCTGTCAGCCTGGGCGATCAAGCCTCCATCTC |
| | | CTGTAGAAGCAGCCAGAGCCTGGTCCACTCCAATGGCAATA |
| | | CCTATCTCAGATGGTATCTCCAAAAGCCTGGGCAGAGCCCAA |
| | | AAGTCCTCATCTACAAAGTCTCTAATCGCGTCAGCGGAGTGC |
| | | CCGACAGGTTTAGCGGTTCTGGAAGCGGCACAGATTTCACGC |
| | | TCAAGATTAACCGCGTCGAGGCCGAAGATCTGGGAGTCTATT |
| | | TCTGCAGTCAGAGCACACATGTGCCCTGGACATTCGGCGGAG |
| | | GCACCAAGCTCGAGATCAAGTCTAGCGCCGATGATGCTAAG |
| | | AAAGATGCAGCTAAAAAGGATGATGCCAAGAAGGACGACGC |
| | | CAAGAAAGATGGCGGAGTCAAACTCGATGAGACTGGCGGAG |
| | | GCCTGGTGCAACCCGGTGGTGCTATGAAGCTGTCTTGCGTGA |
| | | CCTCCGGCTTTACATTTGGGCATTATTGGATGAATTGGGTTC |
| | | GCCAATCTCCAGAGAAGGGCCTCGAGTGGGTTGCACAGTTTC |
| | | GGAACAAACCGTACAATTACGAGACATATTACTCCGACTCCG |
| | | TGAAAGGCCGGTTCACAATCTCCCGCGACGACTCCAAGTCCT |
| | | CTGTCTATCTTCAAATGAACAATCTGAGAGTCGAGGACACGG |
| | | GGATCTACTATTGCACAGGCGCCTCTTATGGAATGGAATACC |
| | | TTGGACAGGGAACCTCTGTGACCGTCAGCACCACAACACCC |
| | | GCTCCTAGACCTCCAACTCCTGCTCCAACAATCGCCTCTCAA |
| | | CCCCTCAGCCTCAGACCTGAGGCATGTAGACCAGCAGCTGGC |
| | | GGTGCAGTTCACACCAGAGGCCTGGACTTTGCCTGTGACGTC |
| | | GTGATCAGCGTGGGCAGCATGGGCCTGATCATCTCCCTGCTG |
| | | TGTGTGTACTTTTGGCTCGAGCGGACCATGCCTCGGATCCCC |
| | | ACACTGAAGAACCTCGAGGATCTGGTCACCGAGTACCACGG |
| | | CAACTTCTCTGCTTGGAGCGGCGTGTCAAAAGGACTGGCCGA |
| | | AAGCCTGCAGCCTGACTACTCCGAGAGACTGTGCCTGGTGTC |
| | | TGAGATCCCTCCTAAAGGCGGCGCTCTCGGAGAAGGACCTG |
| | | GTGCCTCTCCATGCAATCAGCACAGCCCTTATTGGGCCCCTC |
| | | CTTGCTACACCCTGAAACCTGAGACAGGCAGCGGCGAAGGC |
| | | AGAGGCTCTCTTCTTACATGTGGCGACGTCGAAGAGAATCCC |
| | | GGACCAATGGCACTCCCCGTCACCGCCCTTCTCTTGCCCCTC |
| | | GCCCTGCTGCTGCATGCTGCCAGGCCCGACATTGTGCTCACT |
| | | CAGTCACCTCCCAGCCTGGCCATGAGCCTGGGAAAAAGGGC |
| | | CACCATCTCCTGTAGAGCCAGTGAGTCCGTCACAATCTTGGG |
| | | GAGCCATCTTATTCACTGGTATCAGCAGAAGCCCGGGCAGCC |
| | | TCCAACCCTTCTTATTCAGCTCGCGTCAAACGTCCAGACGGG |
| | | TGTACCTGCCAGATTTTCTGGTAGCGGGTCCCGCACTGATTTT |
| | | ACACTGACCATAGATCCAGTGGAAGAAGACGATGTGGCCGT |
| | | GTATTATTGTCTGCAGAGCAGAACGATTCCTCGCACATTTGG |
| | | TGGGGGTACTAAGCTGGAGATTAAGGGAAGCACGTCCGGCT |
| | | CAGGGAAGCCGGGCTCCGGCGAGGGAAGCACGAAGGGGCA |
| | | AATTCAGCTGGTCCAGAGCGGACCTGAGCTGAAAAAACCCG |
| | | GCGAGACTGTTAAGATCAGTTGTAAAGCATCTGGCTATACCT |
| | | TCACCGACTACAGCATAAATTGGGTGAAACGGGCCCCTGGA |
| | | AAGGGCCTCAAATGGATGGGTTGGATCAATACCGAAACTAG |
| | | GGAGCCTGCTTATGCATATGACTTCCGCGGGAGATTCGCCTT |
| | | TTCACTCGAGACATCTGCCTCTACTGCTTACCTCCAAATAAA |
| | | CAACCTCAAGTATGAAGATACAGCCACTTACTTTTGCGCCCT |
| | | CGACTATAGTTACGCCATGGACTACTGGGGACAGGGAACCT |
| | | CCGTTACCGTCAGTTCCGCGGCCGCAACCACAACACCTGCTC |
| | | CAAGGCCCCCCACACCCGCTCCAACTATAGCCAGCCAACCAT |
| | | TGAGCCTCAGACCTGAAGCTTGCAGGCCCGCAGCAGGAGGC |
| | | GCCGTCCATACGCGAGGCCTGGACTTCGCGTGTGATATTTAT |
| | | ATTTGGGCCCCTTTGGCCGGAACATGTGGGGTGTTGCTTCTC |
| | | TCCCTTGTGATCACTCTGTATTGTAAGCGCGGGAGAAAGAAG |
| | | CTCCTGTACATCTTCAAGCAGCCTTTTATGCGACCTGTGCAA |
| | | ACCACTCAGGAAGAAGATGGGTGTTCATGCCGCTTCCCCGAG |
| | | GAGGAAGAAGGAGGGTGTGAACTGAGGGTGAAATTTTCTAG |
| | | AAGCGCCGATGCTCCCGCATATCAGCAGGGTCAGAATCAGC |
| | | TCTACAATGAATTGAATCTCGGCAGGCGAGAAGAGTACGAT |
| | | GTTCTGGACAAGAGACGGGGCAGGGATCCCGAGATGGGGGG |
| | | AAAGCCCCGGAGAAAAAATCCTCAGGAGGGGTTGTACAATG |
| | | AGCTGCAGAAGGACAAGATGGCTGAAGCCTATAGCGAGATC |
| | | GGAATGAAAGGCGAAAGACGCAGAGGCAAGGGGCATGACG |
| | | GTCTGTACCAGGGTCTCTCTACAGCCACCAAGGACACTTATG |
| | | ATGCGTTGCATATGCAAGCCTTGCCACCCCGCTGATGA |
| anti-FITC | SEQ ID NO: 47 | ATGACCATCCTGGGCACCACCTTCGGCATGGTGTTTAGCCTG |
| IL7Rα SMAR- | | CTGCAGGTCGTGTCTGGCGATTACAAGGATGACGACGATAA |
| T2A-bb2121- | | GGACGTGGTCATGACACAGACCCCACTGTCTCTGCCTGTGTC |
| P2A- | | TCTGGGAGATCAGGCCAGCATCAGCTGCAGATCTAGCCAGA |
| Truncated | | GCCTGGTGCACAGCAACGGCAACACCTACCTGCGGTGGTATC |
| CD19 | | TGCAGAAGCCCGGCCAGTCTCCTAAGGTGCTGATCTACAAGG |
| | | TGTCCAACAGAGTGTCCGGCGTGCCCGATAGATTTTCTGGCA |
| | | GCGGCTCTGGCACCGACTTCACCCTGAAGATCAATAGAGTGG |
| | | AAGCCGAGGACCTGGGCGTGTACTTCTGTAGCCAGTCTACCC |
| | | ACGTGCCATGGACCTTTGGCGGCGGAACAAAGCTGGAAATC |
| | | AAGAGCAGCGCCGACGACGCCAAGAAGGACGCCGCTAAGA |
| | | AGGATGACGCAAAGAAAGACGATGCCAAAAAGGATGGCGG |
| | | CGTGAAGCTGGACGAAACAGGCGGAGGACTTGTTCAGCCTG |
| | | GCGGAGCCATGAAGCTGAGCTGTGTGACCAGCGGCTTCACCT |
| | | TTGGCCACTACTGGATGAACTGGGTCCGACAGAGCCCTGAG |
| | | AAAGGCCTGGAATGGGTCGCCCAGTTCAGAAACAAGCCCTA |
| | | CAACTACGAAACCTACTACAGCGACAGCGTGAAGGGCAGAT |
| | | TCACCATCAGCCGGGACGACAGCAAGTCCAGCGTGTACCTG |
| | | CAGATGAACAACCTGCGCGTGGAAGATACCGGCATCTACTA |
| | | CTGTACCGGCGCCAGCTACGGCATGGAATATCTCGGCCAGG |
| | | GCACCAGCGTGACCGTGTCTACAACAACCCCTGCTCCTCGGC |
| | | CTCCTACACCAGCTCCTACAATTGCCAGCCAGCCTCTGTCTCT |
| | | GAGGCCCGAAGCTTGTAGACCTGCTGCCGGCGGAGCTGTGC |
| | | ATACAAGAGGCCTGGATTTCGCCTGCGATCCCATCCTGCTGA |
| | | CAATCAGCATCCTGAGCTTTTTCAGCGTGGCCCTGCTGGTCA |
| | | TCCTGGCCTGTGTGCTGTGGAAGAAGCGGATCAAGCCCATCG |
| | | TGTGGCCCAGCCTGCCTGACCACAAGAAAACCCTGGAACAC |
| | | CTGTGCAAGAAGCCCCGGAAGAACCTGAACGTGTCCTTCAAT |
| | | CCCGAGAGCTTCCTGGACTGCCAGATCCACAGAGTGGACGA |
| | | CATCCAGGCCAGGGACGAAGTGGAAGGCTTTCTGCAGGACA |
| | | CATTCCCTCAGCAGCTGGAAGAGAGCGAGAAGCAGAGACTC |
| | | GGAGGCGACGTGCAGAGCCCTAATTGCCCTTCTGAGGACGTC |
| | | GTGATCACCCCTGAGAGCTTCGGCAGAGATAGCAGCCTGAC |
| | | ATGTCTGGCCGGCAATGTGTCCGCCTGTGATGCCCCTATCCT |
| | | GAGCAGCAGCAGAAGCCTGGATTGCAGAGAGAGCGGCAAG |
| | | AACGGCCCTCACGTGTACCAGGATCTGCTCCTGAGCCTGGGA |
| | | ACCACCAATAGCACACTGCCTCCACCATTCAGCCTGCAGAGC |
| | | GGCATCCTGACACTGAACCCTGTTGCTCAGGGCCAGCCAATC |
| | | CTGACAAGCCTGGGCAGCAATCAAGAAGAGGCCTACGTCAC |
| | | CATGAGCAGCTTCTACCAGAATCAAGGCTCCGGCGAAGGCA |
| | | GAGGCTCTCTGCTTACATGCGGAGATGTGGAAGAGAACCCC |
| | | GGACCTATGGCACTCCCCGTCACCGCCCTTCTCTTGCCCCTCG |
| | | CCCTGCTGCTGCATGCTGCCAGGCCCGACATTGTGCTCACTC |
| | | AGTCACCTCCCAGCCTGGCCATGAGCCTGGGAAAAAGGGCC |
| | | ACCATCTCCTGTAGAGCCAGTGAGTCCGTCACAATCTTGGGG |
| | | AGCCATCTTATTCACTGGTATCAGCAGAAGCCCGGGCAGCCT |
| | | CCAACCCTTCTTATTCAGCTCGCGTCAAACGTCCAGACGGGT |
| | | GTACCTGCCAGATTTTCTGGTAGCGGGTCCCGCACTGATTTT |
| | | ACACTGACCATAGATCCAGTGGAAGAAGACGATGTGGCCGT |
| | | GTATTATTGTCTGCAGAGCAGAACGATTCCTCGCACATTTGG |
| | | TGGGGGTACTAAGCTGGAGATTAAGGGAAGCACGTCCGGCT |
| | | CAGGGAAGCCGGGCTCCGGCGAGGGAAGCACGAAGGGGCA |
| | | AATTCAGCTGGTCCAGAGCGGACCTGAGCTGAAAAAACCCG |
| | | GCGAGACTGTTAAGATCAGTTGTAAAGCATCTGGCTATACCT |
| | | TCACCGACTACAGCATAAATTGGGTGAAACGGGCCCCTGGA |
| | | AAGGGCCTCAAATGGATGGGTTGGATCAATACCGAAACTAG |
| | | GGAGCCTGCTTATGCATATGACTTCCGCGGGAGATTCGCCTT |
| | | TTCACTCGAGACATCTGCCTCTACTGCTTACCTCCAAATAAA |
| | | CAACCTCAAGTATGAAGATACAGCCACTTACTTTTGCGCCCT |
| | | CGACTATAGTTACGCCATGGACTACTGGGGACAGGGAACCT |
| | | CCGTTACCGTCAGTTCCGCGGCCGCAACCACAACACCTGCTC |
| | | CAAGGCCCCCCACACCCGCTCCAACTATAGCCAGCCAACCAT |
| | | TGAGCCTCAGACCTGAAGCTTGCAGGCCCGCAGCAGGAGGC |
| | | GCCGTCCATACGCGAGGCCTGGACTTCGCGTGTGATATTTAT |
| | | ATTTGGGCCCCTTTGGCCGGAACATGTGGGGTGTTGCTTCTC |
| | | TCCCTTGTGATCACTCTGTATTGTAAGCGCGGGAGAAAGAAG |
| | | CTCCTGTACATCTTCAAGCAGCCTTTTATGCGACCTGTGCAA |
| | | ACCACTCAGGAAGAAGATGGGTGTTCATGCCGCTTCCCCGAG |
| | | GAGGAAGAAGGAGGGTGTGAACTGAGGGTGAAATTTTCTAG |
| | | AAGCGCCGATGCTCCCGCATATCAGCAGGGTCAGAATCAGC |
| | | TCTACAATGAATTGAATCTCGGCAGGCGAGAAGAGTACGAT |
| | | GTTCTGGACAAGAGACGGGGCAGGGATCCCGAGATGGGGGG |
| | | AAAGCCCCGGAGAAAAAATCCTCAGGAGGGGTTGTACAATG |
| | | AGCTGCAGAAGGACAAGATGGCTGAAGCCTATAGCGAGATC |
| | | GGAATGAAAGGCGAAAGACGCAGAGGCAAGGGGCATGACG |
| | | GTCTGTACCAGGGTCTCTCTACAGCCACCAAGGACACTTATG |
| | | ATGCGTTGCATATGCAAGCCTTGCCACCCCGCGGATCTGGCG |
| | | CCACAAACTTCTCACTGCTGAAACAGGCCGGCGACGTCGAA |
| | | GAGAATCCTGGGCCTATGCCTCCTCCTCGGCTGCTGTTCTTCC |
| | | TGCTGTTTCTGACCCCTATGGAAGTGCGGCCCGAGGAACCTC |
| | | TGGTCGTGAAAGTTGAAGAGGGCGACAACGCCGTGCTGCAG |
| | | TGTCTGAAGGGCACATCTGATGGCCCCACACAGCAGCTGACC |
| | | TGGTCTAGAGAGAGCCCTCTGAAGCCCTTCCTGAAGCTCAGT |
| | | CTGGGACTGCCTGGACTGGGCATCCATATGAGGCCACTGGCC |
| | | ATCTGGCTGTTCATCTTTAACGTGTCCCAGCAGATGGGAGGC |
| | | TTCTACCTGTGTCAGCCTGGACCTCCTAGCGAGAAAGCTTGG |
| | | CAGCCTGGCTGGACCGTGAATGTGGAAGGATCCGGCGAGCT |
| | | GTTCCGGTGGAATGTCTCTGATCTCGGCGGCCTCGGATGCGG |
| | | CCTGAAGAATAGATCTAGCGAGGGCCCTAGCAGCCCCTCCG |
| | | GAAAACTGATGAGCCCCAAGCTGTACGTGTGGGCCAAAGAC |
| | | AGACCCGAGATTTGGGAGGGCGAGCCTCCTTGTCTGCCACCT |
| | | AGAGACAGCCTGAATCAGAGCCTGAGCCAGGACCTGACAAT |
| | | GGCCCCTGGATCTACACTGTGGCTGAGCTGCGGAGTGCCTCC |
| | | TGACAGTGTGTCTAGAGGCCCACTGAGCTGGACACACGTGC |
| | | ACCCTAAGGGCCCTAAGAGCCTGCTGAGTCTGGAACTGAAG |
| | | GACGACAGGCCCGCCAGAGATATGTGGGTCATGGAAACCGG |
| | | ACTGCTGCTCCCTAGAGCCACTGCTCAAGATGCCGGCAAGTA |
| | | CTATTGCCACCGGGGCAACCTGACCATGAGCTTCCACCTGGA |
| | | AATTACCGCCAGACCAGTGCTGTGGCATTGGCTGCTTAGAAC |
| | | CGGCGGATGGAAGGTGTCAGCCGTGACTCTGGCCTACCTGAT |
| | | CTTTTGTCTGTGCAGCCTCGTGGGCATCCTGCATCTGCAAAG |
| | | AGCCCTGGTCCTGCGGCGGAAGCGGAAGAGAATGACCGATC |
| | | CTACCAGACGGTTCTGATGA |
| anti-FITC | SEQ ID NO: 48 | ATGACCATCCTGGGCACCACCTTCGGCATGGTGTTTAGCCTG |
| IL7Rα SMAR- | | CTGCAGGTCGTGTCTGGCGATTACAAGGATGACGACGATAA |
| T2A-Carvykti | | GGACGTGGTCATGACACAGACCCCACTGTCTCTGCCTGTGTC |
| | | TCTGGGAGATCAGGCCAGCATCAGCTGCAGATCTAGCCAGA |
| | | GCCTGGTGCACAGCAACGGCAACACCTACCTGCGGTGGTATC |
| | | TGCAGAAGCCCGGCCAGTCTCCTAAGGTGCTGATCTACAAGG |
| | | TGTCCAACAGAGTGTCCGGCGTGCCCGATAGATTTTCTGGCA |
| | | GCGGCTCTGGCACCGACTTCACCCTGAAGATCAATAGAGTGG |
| | | AAGCCGAGGACCTGGGCGTGTACTTCTGTAGCCAGTCTACCC |
| | | ACGTGCCATGGACCTTTGGCGGCGGAACAAAGCTGGAAATC |
| | | AAGAGCAGCGCCGACGACGCCAAGAAGGACGCCGCTAAGA |
| | | AGGATGACGCAAAGAAAGACGATGCCAAAAAGGATGGCGG |
| | | CGTGAAGCTGGACGAAACAGGCGGAGGACTTGTTCAGCCTG |
| | | GCGGAGCCATGAAGCTGAGCTGTGTGACCAGCGGCTTCACCT |
| | | TTGGCCACTACTGGATGAACTGGGTCCGACAGAGCCCTGAG |
| | | AAAGGCCTGGAATGGGTCGCCCAGTTCAGAAACAAGCCCTA |
| | | CAACTACGAAACCTACTACAGCGACAGCGTGAAGGGCAGAT |
| | | TCACCATCAGCCGGGACGACAGCAAGTCCAGCGTGTACCTG |
| | | CAGATGAACAACCTGCGCGTGGAAGATACCGGCATCTACTA |
| | | CTGTACCGGCGCCAGCTACGGCATGGAATATCTCGGCCAGG |
| | | GCACCAGCGTGACCGTGTCTACAACAACCCCTGCTCCTCGGC |
| | | CTCCTACACCAGCTCCTACAATTGCCAGCCAGCCTCTGTCTCT |
| | | GAGGCCCGAAGCTTGTAGACCTGCTGCCGGCGGAGCTGTGC |
| | | ATACAAGAGGCCTGGATTTCGCCTGCGATCCCATCCTGCTGA |
| | | CAATCAGCATCCTGAGCTTTTTCAGCGTGGCCCTGCTGGTCA |
| | | TCCTGGCCTGTGTGCTGTGGAAGAAGCGGATCAAGCCCATCG |
| | | TGTGGCCCAGCCTGCCTGACCACAAGAAAACCCTGGAACAC |
| | | CTGTGCAAGAAGCCCCGGAAGAACCTGAACGTGTCCTTCAAT |
| | | CCCGAGAGCTTCCTGGACTGCCAGATCCACAGAGTGGACGA |
| | | CATCCAGGCCAGGGACGAAGTGGAAGGCTTTCTGCAGGACA |
| | | CATTCCCTCAGCAGCTGGAAGAGAGCGAGAAGCAGAGACTC |
| | | GGAGGCGACGTGCAGAGCCCTAATTGCCCTTCTGAGGACGTC |
| | | GTGATCACCCCTGAGAGCTTCGGCAGAGATAGCAGCCTGAC |
| | | ATGTCTGGCCGGCAATGTGTCCGCCTGTGATGCCCCTATCCT |
| | | GAGCAGCAGCAGAAGCCTGGATTGCAGAGAGAGCGGCAAG |
| | | AACGGCCCTCACGTGTACCAGGATCTGCTCCTGAGCCTGGGA |
| | | ACCACCAATAGCACACTGCCTCCACCATTCAGCCTGCAGAGC |
| | | GGCATCCTGACACTGAACCCTGTTGCTCAGGGCCAGCCAATC |
| | | CTGACAAGCCTGGGCAGCAATCAAGAAGAGGCCTACGTCAC |
| | | CATGAGCAGCTTCTACCAGAATCAAGGCTCCGGCGAAGGCA |
| | | GAGGCTCTCTGCTTACATGCGGAGATGTGGAAGAGAACCCC |
| | | GGACCTATGGCTCTGCCTGTCACCGCTCTGCTGCTGCCTCTG |
| | | GCTCTGCTGCTGCACGCTGCTCGCCCTCAGGTCAAACTGGAA |
| | | GAAAGTGGGGGAGGCCTGGTGCAGGCAGGACGGAGCCTGCG |
| | | CCTGAGCTGCGCAGCATCCGAGCACACCTTCAGCTCCCACGT |
| | | GATGGGCTGGTTTCGGCAGGCCCCAGGCAAGGAGAGAGAGT |
| | | CCGTGGCCGTGATCGGCTGGAGGGACATCTCCACATCTTACG |
| | | CCGATTCTGTGAAGGGCCGGTTCACCATCAGCAGAGACAAC |
| | | GCCAAGAAGACACTGTATCTGCAGATGAATAGCCTGAAGCC |
| | | CGAGGACACCGCCGTGTACTATTGCGCAGCAAGGAGAATCG |
| | | ACGCAGCAGACTTTGATTCCTGGGGCCAGGGCACCCAGGTG |
| | | ACAGTGTCTAGCGGAGGAGGAGGATCTGGAGGAGGAGGAA |
| | | GCGGAGGAGGAGGATCCGAGGTGCAGCTGGTGGAGTCTGGA |
| | | GGCGGCCTGGTGCAGGCCGGAGGCTCTCTGAGGCTGAGCTG |
| | | TGCAGCATCCGGAAGAACCTTCACAATGGGCTGGTTTAGGCA |
| | | GGCACCAGGAAAGGAGAGGGAGTTCGTGGCAGCAATCAGCC |
| | | TGTCCCCTACCCTGGCCTACTATGCCGAGTCCGTGAAGGGCA |
| | | GGTTTACCATCTCTCGCGATAACGCCAAGAATACAGTGGTGC |
| | | TGCAGATGAACAGCCTGAAACCTGAGGACACAGCCCTGTAC |
| | | TATTGTGCCGCCGATCGGAAGAGCGTGATGAGCATTAGACCC |
| | | GATTATTGGGGACAGGGCACACAGGTGACAGTGAGTAGCAC |
| | | TAGTACCACGACGCCAGCGCCGCGACCACCAACACCGGCGC |
| | | CCACCATCGCGTCGCAGCCCCTGTCCCTGCGCCCAGAGGCGT |
| | | GCCGGCCAGCGGCGGGGGGCGCAGTGCACACGAGGGGGCTG |
| | | GACTTCGCCTGTGATATCTACATCTGGGCGCCCTTGGCCGGG |
| | | ACTTGTGGGGTCCTTCTCCTGTCACTGGTTATCACCCTTTACT |
| | | GCAAACGGGGCAGAAAGAAACTCCTGTATATATTCAAACAA |
| | | CCATTTATGAGACCAGTACAAACTACTCAAGAGGAAGATGG |
| | | CTGTAGCTGCCGATTTCCAGAAGAAGAAGAAGGAGGATGTG |
| | | AACTGAGAGTGAAGTTCAGCAGGAGCGCAGACGCCCCCGCG |
| | | TACCAGCAGGGCCAGAACCAGCTCTATAACGAGCTCAATCT |
| | | AGGACGAAGAGAGGAGTACGATGTTTTGGACAAGAGACGTG |
| | | GCCGGGACCCTGAGATGGGGGGAAAGCCGAGAAGGAAGAA |
| | | CCCTCAGGAAGGCCTGTACAATGAACTGCAGAAAGATAAGA |
| | | TGGCGGAGGCCTACAGTGAGATTGGGATGAAAGGCGAGCGC |
| | | CGGAGGGGCAAGGGGCACGATGGCCTTTACCAGGGTCTCAG |
| | | TACAGCCACCAAGGACACCTACGACGCCCTTCACATGCAGG |
| | | CCCTGCCCCCTCGCTAATGA |
| anti-FITC | SEQ ID NO: 49 | ATGACCATCCTGGGCACCACCTTCGGCATGGTGTTTAGCCTG |
| IL7Rα SMAR- | | CTGCAGGTCGTGTCTGGCGATTACAAGGATGACGACGATAA |
| T2A-anti- | | GGACGTGGTCATGACACAGACCCCACTGTCTCTGCCTGTGTC |
| CD123 CAR | | TCTGGGAGATCAGGCCAGCATCAGCTGCAGATCTAGCCAGA |
| | | GCCTGGTGCACAGCAACGGCAACACCTACCTGCGGTGGTATC |
| | | TGCAGAAGCCCGGCCAGTCTCCTAAGGTGCTGATCTACAAGG |
| | | TGTCCAACAGAGTGTCCGGCGTGCCCGATAGATTTTCTGGCA |
| | | GCGGCTCTGGCACCGACTTCACCCTGAAGATCAATAGAGTGG |
| | | AAGCCGAGGACCTGGGCGTGTACTTCTGTAGCCAGTCTACCC |
| | | ACGTGCCATGGACCTTTGGCGGCGGAACAAAGCTGGAAATC |
| | | AAGAGCAGCGCCGACGACGCCAAGAAGGACGCCGCTAAGA |
| | | AGGATGACGCAAAGAAAGACGATGCCAAAAAGGATGGCGG |
| | | CGTGAAGCTGGACGAAACAGGCGGAGGACTTGTTCAGCCTG |
| | | GCGGAGCCATGAAGCTGAGCTGTGTGACCAGCGGCTTCACCT |
| | | TTGGCCACTACTGGATGAACTGGGTCCGACAGAGCCCTGAG |
| | | AAAGGCCTGGAATGGGTCGCCCAGTTCAGAAACAAGCCCTA |
| | | CAACTACGAAACCTACTACAGCGACAGCGTGAAGGGCAGAT |
| | | TCACCATCAGCCGGGACGACAGCAAGTCCAGCGTGTACCTG |
| | | CAGATGAACAACCTGCGCGTGGAAGATACCGGCATCTACTA |
| | | CTGTACCGGCGCCAGCTACGGCATGGAATATCTCGGCCAGG |
| | | GCACCAGCGTGACCGTGTCTACAACAACCCCTGCTCCTCGGC |
| | | CTCCTACACCAGCTCCTACAATTGCCAGCCAGCCTCTGTCTCT |
| | | GAGGCCCGAAGCTTGTAGACCTGCTGCCGGCGGAGCTGTGC |
| | | ATACAAGAGGCCTGGATTTCGCCTGCGATCCCATCCTGCTGA |
| | | CAATCAGCATCCTGAGCTTTTTCAGCGTGGCCCTGCTGGTCA |
| | | TCCTGGCCTGTGTGCTGTGGAAGAAGCGGATCAAGCCCATCG |
| | | TGTGGCCCAGCCTGCCTGACCACAAGAAAACCCTGGAACAC |
| | | CTGTGCAAGAAGCCCCGGAAGAACCTGAACGTGTCCTTCAAT |
| | | CCCGAGAGCTTCCTGGACTGCCAGATCCACAGAGTGGACGA |
| | | CATCCAGGCCAGGGACGAAGTGGAAGGCTTTCTGCAGGACA |
| | | CATTCCCTCAGCAGCTGGAAGAGAGCGAGAAGCAGAGACTC |
| | | GGAGGCGACGTGCAGAGCCCTAATTGCCCTTCTGAGGACGTC |
| | | GTGATCACCCCTGAGAGCTTCGGCAGAGATAGCAGCCTGAC |
| | | ATGTCTGGCCGGCAATGTGTCCGCCTGTGATGCCCCTATCCT |
| | | GAGCAGCAGCAGAAGCCTGGATTGCAGAGAGAGCGGCAAG |
| | | AACGGCCCTCACGTGTACCAGGATCTGCTCCTGAGCCTGGGA |
| | | ACCACCAATAGCACACTGCCTCCACCATTCAGCCTGCAGAGC |
| | | GGCATCCTGACACTGAACCCTGTTGCTCAGGGCCAGCCAATC |
| | | CTGACAAGCCTGGGCAGCAATCAAGAAGAGGCCTACGTCAC |
| | | CATGAGCAGCTTCTACCAGAATCAAGGCTCCGGCGAAGGCA |
| | | GAGGCTCTCTGCTTACATGCGGAGATGTGGAAGAGAACCCC |
| | | GGACCTATGGCCCTGCCAGTAACTGCTCTTCTCCTGCCACTC |
| | | GCTCTTCTGCTGCACGCCGCACGGCCTCAAATTCAACTTGTG |
| | | CAGAGTGGCCCCGAGCTGAAAAAGCCCGGCGAAACAGTAAA |
| | | GATCTCTTGTAAGGCGTCTGGATATATCTTTACCAATTACGG |
| | | TATGAACTGGGTCAAACAGGCACCCGGTAAAAGCTTTAAAT |
| | | GGATGGGCTGGATAAATACTTATACCGGAGAAAGCACGTAT |
| | | AGCGCCGACTTCAAGGGTAGATTTGCGTTCAGCCTGGAAACG |
| | | TCCGCCAGCACTGCATACTTGCACATCAATGACTTGAAAAAT |
| | | GAGGATACCGCGACCTACTTTTGTGCTAGAAGTGGTGGATAC |
| | | GATCCCATGGATTACTGGGGACAGGGGACCTCTGTCACAGTC |
| | | AGTTCTGGGGGCGGCGGTTCCGGAGGTGGCGGCTCAGGTGG |
| | | AGGTGGGTCAGATATCGTGTTGACACAAAGCCCTGCTTCCCT |
| | | GGCCGTCAGCTTGGGACAGAGAGCTACTATCTCCTGTAGAGC |
| | | AAGTGAGTCTGTCGACAATTACGGCAACACCTTTATGCACTG |
| | | GTACCAGCAGAAGCCAGGCCAGCCACCGAAGCTGTTGATTT |
| | | ACCGGGCCTCAAACCTCGAAAGCGGGATTCCAGCTAGATTTT |
| | | CTGGCAGCGGGAGTCGAACCGACTTCACACTGACTATTAACC |
| | | CAGTGGAAGCAGATGACGTAGCCACATATTACTGTCAGCAG |
| | | AGCAACGAAGACCCCCCGACGTTTGGTGCTGGAACCAAACT |
| | | GGAGCTGAAAACGACAACCCCCGCTCCACGACCTCCTACTCC |
| | | CGCTCCCACAATCGCATCTCAGCCACTCTCCTTGAGGCCTGA |
| | | GGCCTGCCGACCAGCTGCAGGCGGCGCAGTACACACCAGAG |
| | | GCCTCGACTTTGCTTGCGATATCTACATATGGGCGCCCCTGG |
| | | CCGGGACGTGTGGGGTATTGCTCCTGAGCCTTGTGATCACTC |
| | | TTTACTGCAAAAGAGGTCGGAAAAAGCTTCTTTACATCTTTA |
| | | AACAGCCGTTCATGCGCCCGGTGCAAACAACACAGGAGGAG |
| | | GATGGATGCAGCTGCAGGTTTCCGGAGGAGGAGGAAGGCGG |
| | | GTGTGAGCTCCGGGTAAAATTCTCCAGAAGCGCAGACGCGC |
| | | CAGCCTATCAGCAGGGGCAGAATCAGCTCTATAACGAACTC |
| | | AATCTTGGGCGACGGGAAGAATACGATGTACTTGATAAACG |
| | | CCGAGGTCGCGACCCCGAAATGGGAGGCAAGCCTCAGAGAC |
| | | GCAAGAATCCCCAAGAAGGCCTGTATAATGAACTCCAGAAG |
| | | GATAAAATGGCGGAGGCCTACAGCGAAATTGGTATGAAGGG |
| | | AGAAAGACGACGCGGTAAAGGCCACGACGGCCTTTACCAGG |
| | | GCCTGTCAACAGCTACCAAAGATACGTATGATGCACTTCACA |
| | | TGCAAGCCCTGCCGCCCCGGTGATGA |
| anti-FITC | SEQ ID NO: 50 | ATGACCATCCTGGGCACCACCTTCGGCATGGTGTTTAGCCTG |
| IL7Rα SMAR- | | CTGCAGGTCGTGTCTGGCGATTACAAGGATGACGACGATAA |
| T2A-anti-GD2 | | GGACGTGGTCATGACACAGACCCCACTGTCTCTGCCTGTGTC |
| CAR | | TCTGGGAGATCAGGCCAGCATCAGCTGCAGATCTAGCCAGA |
| | | GCCTGGTGCACAGCAACGGCAACACCTACCTGCGGTGGTATC |
| | | TGCAGAAGCCCGGCCAGTCTCCTAAGGTGCTGATCTACAAGG |
| | | TGTCCAACAGAGTGTCCGGCGTGCCCGATAGATTTTCTGGCA |
| | | GCGGCTCTGGCACCGACTTCACCCTGAAGATCAATAGAGTGG |
| | | AAGCCGAGGACCTGGGCGTGTACTTCTGTAGCCAGTCTACCC |
| | | ACGTGCCATGGACCTTTGGCGGCGGAACAAAGCTGGAAATC |
| | | AAGAGCAGCGCCGACGACGCCAAGAAGGACGCCGCTAAGA |
| | | AGGATGACGCAAAGAAAGACGATGCCAAAAAGGATGGCGG |
| | | CGTGAAGCTGGACGAAACAGGCGGAGGACTTGTTCAGCCTG |
| | | GCGGAGCCATGAAGCTGAGCTGTGTGACCAGCGGCTTCACCT |
| | | TTGGCCACTACTGGATGAACTGGGTCCGACAGAGCCCTGAG |
| | | AAAGGCCTGGAATGGGTCGCCCAGTTCAGAAACAAGCCCTA |
| | | CAACTACGAAACCTACTACAGCGACAGCGTGAAGGGCAGAT |
| | | TCACCATCAGCCGGGACGACAGCAAGTCCAGCGTGTACCTG |
| | | CAGATGAACAACCTGCGCGTGGAAGATACCGGCATCTACTA |
| | | CTGTACCGGCGCCAGCTACGGCATGGAATATCTCGGCCAGG |
| | | GCACCAGCGTGACCGTGTCTACAACAACCCCTGCTCCTCGGC |
| | | CTCCTACACCAGCTCCTACAATTGCCAGCCAGCCTCTGTCTCT |
| | | GAGGCCCGAAGCTTGTAGACCTGCTGCCGGCGGAGCTGTGC |
| | | ATACAAGAGGCCTGGATTTCGCCTGCGATCCCATCCTGCTGA |
| | | CAATCAGCATCCTGAGCTTTTTCAGCGTGGCCCTGCTGGTCA |
| | | TCCTGGCCTGTGTGCTGTGGAAGAAGCGGATCAAGCCCATCG |
| | | TGTGGCCCAGCCTGCCTGACCACAAGAAAACCCTGGAACAC |
| | | CTGTGCAAGAAGCCCCGGAAGAACCTGAACGTGTCCTTCAAT |
| | | CCCGAGAGCTTCCTGGACTGCCAGATCCACAGAGTGGACGA |
| | | CATCCAGGCCAGGGACGAAGTGGAAGGCTTTCTGCAGGACA |
| | | CATTCCCTCAGCAGCTGGAAGAGAGCGAGAAGCAGAGACTC |
| | | GGAGGCGACGTGCAGAGCCCTAATTGCCCTTCTGAGGACGTC |
| | | GTGATCACCCCTGAGAGCTTCGGCAGAGATAGCAGCCTGAC |
| | | ATGTCTGGCCGGCAATGTGTCCGCCTGTGATGCCCCTATCCT |
| | | GAGCAGCAGCAGAAGCCTGGATTGCAGAGAGAGCGGCAAG |
| | | AACGGCCCTCACGTGTACCAGGATCTGCTCCTGAGCCTGGGA |
| | | ACCACCAATAGCACACTGCCTCCACCATTCAGCCTGCAGAGC |
| | | GGCATCCTGACACTGAACCCTGTTGCTCAGGGCCAGCCAATC |
| | | CTGACAAGCCTGGGCAGCAATCAAGAAGAGGCCTACGTCAC |
| | | CATGAGCAGCTTCTACCAGAATCAAGGCTCCGGCGAAGGCA |
| | | GAGGCTCTCTGCTTACATGCGGAGATGTGGAAGAGAACCCC |
| | | GGACCTATGGCTCTGCCTGTCACCGCTCTGCTGCTGCCTCTG |
| | | GCTCTGCTGCTGCACGCTGCTCGCCCTCAGGTGCAGCTGCAG |
| | | GAGTCTGGCCCAGGCCTGGTGAAGCCCAGCCAGACCCTGAG |
| | | CATCACCTGCACCGTGAGCGGCTTCAGCCTGGCCAGCTACAA |
| | | CATCCACTGGGTGCGGCAGCCCCCAGGCAAGGGCCTGGAGT |
| | | GGCTGGGCGTGATCTGGGCTGGCGGCAGCACCAACTACAAC |
| | | AGCGCCCTGATGAGCCGGCTGACCATCAGCAAGGACAACAG |
| | | CAAGAACCAGGTGTTCCTGAAGATGAGCAGCCTGACAGCCG |
| | | CCGACACCGCCGTGTACTACTGCGCCAAGCGGAGCGACGAC |
| | | TACAGCTGGTTCGCCTACTGGGGCCAGGGCACCCTGGTGACC |
| | | GTGAGCTCTGGCGGAGGCGGCTCTGGCGGAGGCGGCTCTGG |
| | | CGGAGGCGGCAGCGAGAACCAGATGACCCAGAGCCCCAGCA |
| | | GCTTGAGCGCCAGCGTGGGCGACCGGGTGACCATGACCTGC |
| | | AGAGCCAGCAGCAGCGTGAGCAGCAGCTACCTGCACTGGTA |
| | | CCAGCAGAAGAGCGGCAAGGCCCCAAAGGTGTGGATCTACA |
| | | GCACCAGCAACCTGGCCAGCGGCGTGACCAGCCGGTTCAGC |
| | | GGCAGCGGCAGCGGCACCGACTACACCCTGACCATCAGCAG |
| | | CCTGCAGCCCGAGGACTTCGCCACCTACTACTGCCAGCAGTA |
| | | CAGCGGCTACCCCATCACCTTCGGCCAGGGCACCAAGGTGG |
| | | AGATCAAGCGGTCGGATCCCACCACGACGCCAGCGCCGCGA |
| | | CCACCAACACCGGCGCCCACCATCGCGTCGCAGCCCCTGTCC |
| | | CTGCGCCCAGAGGCGTGCCGGCCAGCGGCGGGGGGCGCAGT |
| | | GCACACGAGGGGGCTGGACTTCGCCTGTGATATCTACATCTG |
| | | GGCGCCCTTGGCCGGGACTTGTGGGGTCCTTCTCCTGTCACT |
| | | GGTTATCACCCTTTACTGCAAACGGGGCAGAAAGAAACTCCT |
| | | GTATATATTCAAACAACCATTTATGAGACCAGTACAAACTAC |
| | | TCAAGAGGAAGATGGCTGTAGCTGCCGATTTCCAGAAGAAG |
| | | AAGAAGGAGGATGTGAACTGAGAGTGAAGTTCAGCAGGAGC |
| | | GCAGACGCCCCCGCGTACCAGCAGGGCCAGAACCAGCTCTA |
| | | TAACGAGCTCAATCTAGGACGAAGAGAGGAGTACGATGTTT |
| | | TGGACAAGAGACGTGGCCGGGACCCTGAGATGGGGGGAAAG |
| | | CCGAGAAGGAAGAACCCTCAGGAAGGCCTGTACAATGAACT |
| | | GCAGAAAGATAAGATGGCGGAGGCCTACAGTGAGATTGGGA |
| | | TGAAAGGCGAGCGCCGGAGGGGCAAGGGGCACGATGGCCTT |
| | | TACCAGGGTCTCAGTACAGCCACCAAGGACACCTACGACGC |
| | | CCTTCACATGCAGGCCCTGCCCCCTCGCTAATGA |
| anti-FITC | SEQ ID NO: 51 | ATGACCATCCTGGGCACCACCTTCGGCATGGTGTTTAGCCTG |
| IL7Rα SMAR- | | CTGCAGGTCGTGTCTGGCGATTACAAGGATGACGACGATAA |
| T2A-anti- | | GGACGTGGTCATGACACAGACCCCACTGTCTCTGCCTGTGTC |
| DOTA CAR | | TCTGGGAGATCAGGCCAGCATCAGCTGCAGATCTAGCCAGA |
| | | GCCTGGTGCACAGCAACGGCAACACCTACCTGCGGTGGTATC |
| | | TGCAGAAGCCCGGCCAGTCTCCTAAGGTGCTGATCTACAAGG |
| | | TGTCCAACAGAGTGTCCGGCGTGCCCGATAGATTTTCTGGCA |
| | | GCGGCTCTGGCACCGACTTCACCCTGAAGATCAATAGAGTGG |
| | | AAGCCGAGGACCTGGGCGTGTACTTCTGTAGCCAGTCTACCC |
| | | ACGTGCCATGGACCTTTGGCGGCGGAACAAAGCTGGAAATC |
| | | AAGAGCAGCGCCGACGACGCCAAGAAGGACGCCGCTAAGA |
| | | AGGATGACGCAAAGAAAGACGATGCCAAAAAGGATGGCGG |
| | | CGTGAAGCTGGACGAAACAGGCGGAGGACTTGTTCAGCCTG |
| | | GCGGAGCCATGAAGCTGAGCTGTGTGACCAGCGGCTTCACCT |
| | | TTGGCCACTACTGGATGAACTGGGTCCGACAGAGCCCTGAG |
| | | AAAGGCCTGGAATGGGTCGCCCAGTTCAGAAACAAGCCCTA |
| | | CAACTACGAAACCTACTACAGCGACAGCGTGAAGGGCAGAT |
| | | TCACCATCAGCCGGGACGACAGCAAGTCCAGCGTGTACCTG |
| | | CAGATGAACAACCTGCGCGTGGAAGATACCGGCATCTACTA |
| | | CTGTACCGGCGCCAGCTACGGCATGGAATATCTCGGCCAGG |
| | | GCACCAGCGTGACCGTGTCTACAACAACCCCTGCTCCTCGGC |
| | | CTCCTACACCAGCTCCTACAATTGCCAGCCAGCCTCTGTCTCT |
| | | GAGGCCCGAAGCTTGTAGACCTGCTGCCGGCGGAGCTGTGC |
| | | ATACAAGAGGCCTGGATTTCGCCTGCGATCCCATCCTGCTGA |
| | | CAATCAGCATCCTGAGCTTTTTCAGCGTGGCCCTGCTGGTCA |
| | | TCCTGGCCTGTGTGCTGTGGAAGAAGCGGATCAAGCCCATCG |
| | | TGTGGCCCAGCCTGCCTGACCACAAGAAAACCCTGGAACAC |
| | | CTGTGCAAGAAGCCCCGGAAGAACCTGAACGTGTCCTTCAAT |
| | | CCCGAGAGCTTCCTGGACTGCCAGATCCACAGAGTGGACGA |
| | | CATCCAGGCCAGGGACGAAGTGGAAGGCTTTCTGCAGGACA |
| | | CATTCCCTCAGCAGCTGGAAGAGAGCGAGAAGCAGAGACTC |
| | | GGAGGCGACGTGCAGAGCCCTAATTGCCCTTCTGAGGACGTC |
| | | GTGATCACCCCTGAGAGCTTCGGCAGAGATAGCAGCCTGAC |
| | | ATGTCTGGCCGGCAATGTGTCCGCCTGTGATGCCCCTATCCT |
| | | GAGCAGCAGCAGAAGCCTGGATTGCAGAGAGAGCGGCAAG |
| | | AACGGCCCTCACGTGTACCAGGATCTGCTCCTGAGCCTGGGA |
| | | ACCACCAATAGCACACTGCCTCCACCATTCAGCCTGCAGAGC |
| | | GGCATCCTGACACTGAACCCTGTTGCTCAGGGCCAGCCAATC |
| | | CTGACAAGCCTGGGCAGCAATCAAGAAGAGGCCTACGTCAC |
| | | CATGAGCAGCTTCTACCAGAATCAAGGCTCCGGCGAAGGCA |
| | | GAGGCTCTCTGCTTACATGCGGAGATGTGGAAGAGAACCCC |
| | | GGACCTATGGCTCTGCCAGTGACAGCTCTGCTGCTTCCTCTG |
| | | GCTCTCCTGCTGCATGCCGCTAGACCTGAGCAGAAACTCATC |
| | | TCTGAAGAGGATCTGCACGTGAAACTGCAAGAGTCTGGCCC |
| | | CGGACTGGTGCAGCCTTCTCAGTCTCTCTCTCTGACCTGTACC |
| | | GTGTCCGGCTTCAGCCTGACCGATTATGGCGTGCACTGGGTT |
| | | CGACAATCCCCAGGCAAAGGACTCGAGTGGCTGGGAGTGAT |
| | | TTGGAGCGGCGGAGGCACAGCCTATAACACAGCCCTGATCA |
| | | GCAGACTGAACATCTACCGGGACAACTCCAAGAACCAGGTT |
| | | TTCCTGGAAATGAACTCCCTGCAGGCAGAGGACACCGCCAT |
| | | GTACTACTGCGCCAGAAGAGGCAGCTACCCCTACAATTACTT |
| | | CGACGCCTGGGGCTGTGGCACAACCGTGACAGTTTCTAGTGG |
| | | CGGAGGCGGATCTGGTGGTGGTGGTAGCGGTGGCGGAGGAT |
| | | CTCAGGCCGTGGTTATTCAAGAAAGCGCCCTGACAACCCCTC |
| | | CTGGCGAGACAGTGACACTGACATGTGGCAGCTCTACAGGC |
| | | GCCGTGACCGCCAGCAATTACGCCAATTGGGTGCAAGAGAA |
| | | GCCCGACCACTGCTTCACAGGCCTGATCGGCGGCCACAACA |
| | | ATAGACCTCCAGGCGTGCCAGCTAGATTCAGCGGATCCCTGA |
| | | TCGGAGACAAGGCCGCTCTGACAATCGCCGGCACACAGACA |
| | | GAGGACGAGGCCATCTACTTTTGCGCCCTGTGGTACAGCGAC |
| | | CACTGGGTTATCGGCGGAGGAACCAGACTGACAGTGCTGGG |
| | | CACAACAACACCCGCACCTAGACCACCAACTCCAGCACCAA |
| | | CAATCGCCTCTCAACCCCTGAGTCTGAGGCCAGAGGCATGCA |
| | | GACCAGCCGCTGGCGGTGCAGTTCACACTAGAGGACTGGAC |
| | | TTTGCCTGTGACATCTACATCTGGGCCCCTCTGGCTGGAACA |
| | | TGTGGTGTCCTGCTGCTGTCCCTGGTCATCACCCTGTACTGCA |
| | | AGCGGGGCAGAAAGAAACTGCTGTACATCTTCAAGCAGCCC |
| | | TTCATGCGGCCCGTGCAGACCACACAAGAGGAAGATGGCTG |
| | | CTCCTGCAGATTCCCCGAGGAAGAAGAAGGCGGCTGCGAGC |
| | | TGAGAGTGAAGTTCTCCAGATCTGCCGACGCTCCCGCCTATC |
| | | AGCAGGGACAGAACCAGCTGTACAACGAGCTGAACCTGGGG |
| | | AGAAGAGAAGAGTACGACGTGCTGGACAAGCGGAGAGGCA |
| | | GAGATCCTGAGATGGGCGGAAAGCCCCAGCGGAGAAAGAAT |
| | | CCTCAAGAGGGCCTGTATAATGAGCTGCAAAAGGACAAGAT |
| | | GGCCGAGGCCTACAGCGAGATCGGAATGAAGGGCGAGCGCA |
| | | GAAGAGGAAAGGGACACGACGGACTGTACCAGGGCCTGAGC |
| | | ACAGCCACCAAGGATACCTATGACGCCCTGCACATGCAGGC |
| | | CCTGCCTCCAAGATGATGA |
| anti-DOTA | SEQ ID NO: 52 | ATGACCATCCTGGGCACCACCTTCGGCATGGTGTTTAGCCTG |
| IL7Rα SMAR- | | CTGCAGGTCGTGTCTGGCGATTACAAGGATGACGACGATAA |
| T2A-anti- | | GCACGTGAAACTGCAAGAGTCTGGCCCCGGACTGGTGCAGC |
| FITC CAR | | CTTCTCAGTCTCTCTCTCTGACCTGTACCGTGTCCGGCTTCAG |
| | | CCTGACCGATTATGGCGTGCACTGGGTTCGACAATCCCCAGG |
| | | CAAAGGACTCGAGTGGCTGGGAGTGATTTGGAGCGGCGGAG |
| | | GCACAGCCTATAACACAGCCCTGATCAGCAGACTGAACATCT |
| | | ACCGGGACAACTCCAAGAACCAGGTTTTCCTGGAAATGAAC |
| | | TCCCTGCAGGCAGAGGACACCGCCATGTACTACTGCGCCAG |
| | | AAGAGGCAGCTACCCCTACAATTACTTCGACGCCTGGGGCTG |
| | | TGGCACAACCGTGACAGTTTCTAGTGGCGGAGGCGGATCTG |
| | | GTGGTGGTGGTAGCGGTGGCGGAGGATCTCAGGCCGTGGTT |
| | | ATTCAAGAAAGCGCCCTGACAACCCCTCCTGGCGAGACAGT |
| | | GACACTGACATGTGGCAGCTCTACAGGCGCCGTGACCGCCA |
| | | GCAATTACGCCAATTGGGTGCAAGAGAAGCCCGACCACTGC |
| | | TTCACAGGCCTGATCGGCGGCCACAACAATAGACCTCCAGG |
| | | CGTGCCAGCTAGATTCAGCGGATCCCTGATCGGAGACAAGG |
| | | CCGCTCTGACAATCGCCGGCACACAGACAGAGGACGAGGCC |
| | | ATCTACTTTTGCGCCCTGTGGTACAGCGACCACTGGGTTATC |
| | | GGCGGAGGAACCAGACTGACAGTGCTGGGCACAACAACCCC |
| | | TGCTCCTCGGCCTCCTACACCAGCTCCTACAATTGCCAGCCA |
| | | GCCTCTGTCTCTGAGGCCCGAAGCTTGTAGACCTGCTGCCGG |
| | | CGGAGCTGTGCATACAAGAGGCCTGGATTTCGCCTGCGATCC |
| | | CATCCTGCTGACAATCAGCATCCTGAGCTTTTTCAGCGTGGC |
| | | CCTGCTGGTCATCCTGGCCTGTGTGCTGTGGAAGAAGCGGAT |
| | | CAAGCCCATCGTGTGGCCCAGCCTGCCTGACCACAAGAAAA |
| | | CCCTGGAACACCTGTGCAAGAAGCCCCGGAAGAACCTGAAC |
| | | GTGTCCTTCAATCCCGAGAGCTTCCTGGACTGCCAGATCCAC |
| | | AGAGTGGACGACATCCAGGCCAGGGACGAAGTGGAAGGCTT |
| | | TCTGCAGGACACATTCCCTCAGCAGCTGGAAGAGAGCGAGA |
| | | AGCAGAGACTCGGAGGCGACGTGCAGAGCCCTAATTGCCCT |
| | | TCTGAGGACGTCGTGATCACCCCTGAGAGCTTCGGCAGAGAT |
| | | AGCAGCCTGACATGTCTGGCCGGCAATGTGTCCGCCTGTGAT |
| | | GCCCCTATCCTGAGCAGCAGCAGAAGCCTGGATTGCAGAGA |
| | | GAGCGGCAAGAACGGCCCTCACGTGTACCAGGATCTGCTCCT |
| | | GAGCCTGGGAACCACCAATAGCACACTGCCTCCACCATTCAG |
| | | CCTGCAGAGCGGCATCCTGACACTGAACCCTGTTGCTCAGGG |
| | | CCAGCCAATCCTGACAAGCCTGGGCAGCAATCAAGAAGAGG |
| | | CCTACGTCACCATGAGCAGCTTCTACCAGAATCAAGGCTCCG |
| | | GCGAAGGCAGAGGCTCTCTGCTTACATGCGGAGATGTGGAA |
| | | GAGAACCCCGGACCTATGGCTCTGCCTGTGACAGCTCTGCTG |
| | | CTGCCTCTGGCTCTGCTTCTGCATGCCGCCAGACCTGACGTG |
| | | GTCATGACACAGACACCTCTGAGCCTGCCTGTGTCTCTGGGA |
| | | GATCAGGCCAGCATCAGCTGCAGATCTAGCCAGAGCCTGGT |
| | | GCACAGCAACGGCAACACCTACCTGCGGTGGTATCTGCAGA |
| | | AGCCCGGCCAGTCTCCTAAGGTGCTGATCTACAAGGTGTCCA |
| | | ACAGAGTGTCCGGCGTGCCCGATAGATTTTCTGGCAGCGGCT |
| | | CTGGCACCGACTTCACCCTGAAGATCAATAGAGTGGAAGCC |
| | | GAGGACCTGGGCGTGTACTTCTGTAGCCAGTCTACCCACGTG |
| | | CCATGGACCTTTGGCGGCGGAACAAAGCTGGAAATCAAGAG |
| | | CAGCGCCGACGACGCCAAGAAGGACGCCGCTAAGAAGGATG |
| | | ACGCCAAAAAAGACGATGCCAAAAAGGATGGCGGCGTGAA |
| | | GCTGGACGAAACAGGCGGAGGACTTGTTCAGCCTGGCGGAG |
| | | CCATGAAGCTGAGCTGTGTGACCAGCGGCTTCACCTTCGGCC |
| | | ACTACTGGATGAACTGGGTCCGACAGAGCCCTGAGAAAGGC |
| | | CTGGAATGGGTCGCCCAGTTCAGAAACAAGCCCTACAACTA |
| | | CGAAACCTACTACAGCGACAGCGTGAAGGGCAGATTCACCA |
| | | TCAGCCGGGACGACAGCAAGTCCAGCGTGTACCTGCAGATG |
| | | AACAACCTGCGCGTGGAAGATACCGGCATCTACTACTGTACC |
| | | GGCGCCAGCTACGGCATGGAATATCTCGGCCAGGGCACCAG |
| | | CGTGACCGTGTCTACAACAACCCCTGCTCCTCGGCCTCCTAC |
| | | ACCAGCTCCTACAATTGCCAGCCAGCCACTGTCTCTGAGGCC |
| | | CGAAGCTTGTAGACCTGCTGCAGGCGGAGCCGTGCATACAA |
| | | GAGGACTGGATTTCGCCTGCGACTTCTGGGTGCTCGTGGTTG |
| | | TTGGCGGAGTGCTGGCTTGTTACTCCCTGCTGGTTACCGTGG |
| | | CCTTCATCATCTTTTGGGTCAAGCGGGGCAGAAAGAAGCTGC |
| | | TGTACATCTTCAAGCAGCCCTTCATGCGGCCCGTGCAGACCA |
| | | CACAAGAGGAAGATGGCTGCTCCTGCAGATTCCCCGAGGAA |
| | | GAAGAAGGCGGCTGCGAGCTGAGAGTGAAGTTCAGCAGATC |
| | | CGCCGACGCTCCTGCCTATCAGCAGGGACAGAACCAGCTGT |
| | | ACAACGAGCTGAACCTGGGGAGAAGAGAAGAGTACGACGTG |
| | | CTGGACAAGCGGAGAGGCAGAGATCCTGAGATGGGCGGAAA |
| | | GCCCCAGCGGAGAAAGAATCCTCAAGAGGGCCTGTATAATG |
| | | AGCTGCAGAAAGACAAGATGGCCGAGGCCTACAGCGAGATC |
| | | GGAATGAAGGGCGAGCGCAGAAGAGGCAAGGGACACGATG |
| | | GACTGTACCAGGGCCTGAGCACCGCCACCAAGGATACCTAT |
| | | GATGCCCTGCACATGCAGGCCCTGCCACCTAGATGATGA |

In some embodiments, a polynucleotide construct includes a sequence having at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to any one of SEQ ID NO: 46-SEQ ID NO: 52. In some embodiments, the polynucleotide construct has a sequence of any one of SEQ ID NO: 46-SEQ ID NO: 52.

### V. ENGINEERED IMMUNE CELLS

An engineered cytokine receptor switch, as described herein, may be expressed in an immune cell (e.g., a lymphocyte or macrophage) to produce an engineered immune cell. An engineered immune cell, as described herein, may comprise an immune cell (e.g., a lymphocyte or macrophage) engineered to express one or more exogenous receptors (e.g., a CAR, an engineered cytokine receptor switch, or both). A polynucleotide expression cassette encoding the engineered cytokine receptor switch may be delivered to the immune cell using a vector, as described herein, thereby engineering the immune cell to express the engineered cytokine receptor switch. In some embodiments, the immune cell may be further engineered to express an additional component, such as a CAR or a second engineered cytokine receptor switch. The immune cell engineered to express a cytokine receptor switch may be a T-cell, a regulatory T-cell, a B-cell, a natural killer (NK) cell, a FcεRIγ deficient NK cell (g-NK cell), a neutrophil, an eosinophil, a macrophage, a γδ T-cell, or other immune cell type.

Immune cells expressing the engineered cytokine receptor switch may be activated by contacting the cells with an activator. The activator may be attached to a substrate, such as a surface, a bead, a carrier protein, a carrier polymer, a carrier nucleic acid, or combinations thereof. Alternatively or in addition, the activator may be part of a bispecific agent, e.g., the activator may be conjugated to a targeting moiety that binds to a target (e.g., a cancer cell, a cell of a lymphoid organ, or another cell type). Activating the immune cells may promote conversion to memory cell phenotypes. As a result, an activated population of engineered immune cells may have a higher proportion of stem-cell memory phenotypes and central memory phenotypes than a population of engineered immune cells that has not been activated or a population of immune cells that has not been engineered to express a cytokine receptor switch. Activating the immune cells may alternatively or additionally promote homing to lymphoid organs (e.g., lymph nodes, spleen, thymus, and/or bone marrow), where the lymphoid environment may facilitate activation, expansion, and/or conversion to memory cell phenotypes.

In some embodiments, activation may be performed ex vivo. For example, activation may be performed on engineered immune cells prior to administration to a subject, e.g., by administration of an activator bound to a substrate, such as a surface (e.g., a plate surface), a bead (e.g., a polystyrene paramagnetic bead), a carrier protein (e.g., an antibody), a carrier polymer (e.g., a synthetic polymer, a biopolymer), a carrier nucleic acid (e.g., an oligonucleotide, a polynucleotide), or combinations thereof. The immune cells can be exposed to the activator for at least 1 hour, 2 hours, 3 hours, 4 hours, 5 hours, 6 hours, 7 hours, 8 hours, 9 hours, 10 hours, 11 hours, 12 hours, or 24 hours. Ex vivo activation may be performed at least 1 hour, 2 hours, 3 hours, 4 hours, 5 hours, 6 hours, 7 hours, 8 hours, 9 hours, 10 hours, 11 hours, 12 hours, or 24 hours before administration of the engineered immune cells to the subject. The concentration of the activator for ex vivo activation can be within a range from 1 nM to 1000 nM, 1 nM to 500 nM, 1 nM to 100 nM, 1 nM to 50 nM, 1 nM to 10 nM, 10 nM to 1000 n, 10 nM to 500 nM, 10 nM to 100 nM, 10 nM to 50 nM, 50 nM to 1000 nM, 50 nM to 500 nM, 50 nM to 100 nM, 100 nM to 1000 nM, 100 nM to 500 nM, or 500 nM to 1000 nM. Following ex vivo activation, the substrate-bound activator may be removed from the immune cells, e.g., by cleaving the activator from the substrate (e.g., via disulfide reduction, pH-based cleavage, photocleavage, protease cleavage) and/or mechanical disruption.

In some embodiments, activation may be performed in vivo. For example, an activator may be administered to a subject who has received or will receive the engineered immune cells. In some embodiments, a bispecific agent including the activator conjugated to a targeting moiety (e.g., a carrier protein such as an antibody) is administered to a subject who has received or will receive the engineered immune cells. A bispecific agent may be used to recruit an engineered immune cell of the present disclosure to a lymphoid organ by binding to an activator binding domain of an engineered cytokine receptor switch. In some embodiments, providing a bispecific agent to a subject who has been treated with an immune cell expressing an engineered cytokine receptor switch may recruit the immune cell to the lymphoid organ of the subject. The bispecific agent may further activate the immune cell. Activation of the immune cell by the bispecific agent may be facilitated by the lymphoid environment.

The dosage of the activator (e.g., a bispecific agent including the activator) can be within a range from 0.1 mg/kg to 10 mg/kg, 0.1 mg/kg to 5 mg/kg, 0.1 mg/kg to 2 mg/kg, 0.1 mg/kg to 1 mg/kg, 0.1 mg/kg to 0.5 mg/kg, 0.5 mg/kg to 10 mg/kg, 0.5 mg/kg to 5 mg/kg, 0.5 mg/kg to 2 mg/kg, 0.5 mg/kg to 1 mg/kg, 1 mg/kg to 10 mg/kg, 1 mg/kg to 5 mg/kg, 1 mg/kg to 2 mg/kg, 2 mg/kg to 10 mg/kg, 2 mg/kg to 5 mg/kg, or 5 mg/kg to 10 mg/kg. In some embodiments, the activator (e.g., a bispecific agent including the activator) is administered to the subject at least 30 minutes, 1 hour, 2 hours, 3 hours, 4 hours, 5 hours, 6 hours, 7 hours, 8 hours, 9 hours, 10 hours, 11 hours, 12 hours, 24 hours, 36 hours, or 48 hours before the subject receives the engineered immune cells. Alternatively or in combination, the activator (e.g., a bispecific agent including the activator) is administered to the subject concurrently with the engineered immune cells. Alternatively or in combination, the activator (e.g., a bispecific agent including the activator) is administered to the subject at least 30 minutes, 1 hour, 2 hours, 3 hours, 4 hours, 5 hours, 6 hours, 7 hours, 8 hours, 9 hours, 10 hours, 11 hours, 12 hours, 24 hours, 36 hours, or 48 hours after the subject receives the engineered immune cells.

In some embodiments, the activator (e.g., a bispecific agent including the activator) is administered to the subject a single time. Alternatively, the activator (e.g., a bispecific agent including the activator) may be administered to the subject multiple times, e.g., two, three, four, five, or more times. The administration frequency can be at any suitable time interval, such as daily, weekly, biweekly, monthly, yearly, etc. In such embodiments, the bispecific agent that is administered to the subject may be the same for some or all of the administrations, or may be different for some or all of the administrations. For instance, a first bispecific agent including the activator and a targeting moiety for a lymphoid organ may be provided to the subject at a first time point to recruit the engineered immune cells to a lymphoid organ; and a second bispecific agent including the activator and a targeting moiety for a tumor antigen may be provided to the subject at a second, later time point to recruit the engineered immune cells to a tumor cell.

In some embodiments, activation is performed both ex vivo and in vivo. In some embodiments, activation is performed ex vivo but not in vivo. In some embodiments, activation is performed in vivo but not ex vivo. In some embodiments, no activation is performed either ex vivo or in vivo, e.g., if the immune cells exhibit activator-independent activity.

### A. Collection

An immune cell to be engineered (e.g., to express a cytokine receptor switch, a CAR, or both) may be obtained from a subject. Immune cells may be obtained from blood (e.g., peripheral blood mononuclear cells), bone marrow, lymph node tissue, cord blood, thymus tissue, tissue from a site of infection, ascites, pleural effusion, spleen tissue, tumors, or combinations thereof collected from the subject. Immune cells may be collected from a subject using any technique known in the art (e.g., Ficoll separation or apheresis). In some embodiments, immune cells collected from a subject may comprise T-cells, monocytes, granulocytes, B-cells, other nucleated white blood cells, red blood cells, platelets, or combinations thereof.

In some embodiments, immune cells (e.g., T-cells, regulatory T-cells, B-cells, NK cells, macrophages, γδ T-cells, or combinations thereof) may be collected from a donor. The cells may be obtained from blood (e.g., peripheral blood mononuclear cells), bone marrow, lymph node tissue, cord blood, thymus tissue, tissue from a site of infection, ascites, pleural effusion, spleen tissue, tumors, or combinations thereof collected from the subject. Immune cells (e.g., precursors to engineered immune cells of the present disclosure) may be collected from a subject using any technique known in the art (e.g., Ficoll separation). In some cases, the precursor cells are collected from blood, for example through apheresis, leukapheresis, or buffy coat preparation.

In some embodiments, precursor cells (e.g., cells to be engineered to express a cytokine receptor switch, a CAR, or both) are immune cells collected from a donor. The immune cells can be of a single type, or can be a heterogeneous collection of cells. The immune cells can include T-cells, B-cells, natural killer cells (NK cells), FcεRIγ deficient NK cells (g-NK cells), macrophages, monocytes, basophils, eosinophils, neutrophils, megakaryocytes, thrombocytes, or combinations thereof. In some cases, the immune cells are CD4⁺ or CD8⁺ T cells. In some cases, the immune cells are naïve T and/or naïve B cells.

The immune cells can be enriched for specific cell types. For many of the methods disclosed herein, blood-derived immune cells are separated from other whole blood components, for example through monocyte depletion, centrifugation, filtration, or clotting. The immune cells can also be subjected to positive or negative selection for certain cell types. In many cases, immune cells from a donor are separated from other peripheral blood mononuclear cells (PBMCs) through negative selection for surface markers expressed by non-target cells, such as CD25, CD45, CD103, or FOXP3. In specific cases, the immune cells are depleted of memory (e.g., central memory T cells, effector memory T cells, virtual memory T cells, memory B cells, etc.) and/or effector cells. In some cases, the immune cells are enriched for a particular type of T or B cell, such as a γδ T-cell, a T_{H}1 cell, a T_{H}2 cell, a T_{H}17 cell, a T_{H}22 cell, a T helper cell, a T regulatory cell, or a combination thereof. As a nonlimiting example, immune cell enrichment can include selectively binding one or more cell type-specific surface markers on a magnetically separatable bead or on a column. In some cases, the immune cells are of a single cell type.

In some cases, the immune cells are obtained through leukapheresis, bone marrow biopsy, or a combination thereof. In some cases, the immune cells comprise at least about 10%, at least about 20%, at least about 30%, at least about 40%, at least about 50%, at least about 60%, at least about 70%, or at least about 80% naïve T cells and naïve B cells as a percentage of total cell population prior to contacting the immune cells with the activator.

The precursor cells can also include a totipotent, pluripotent, multipotent, or oligopotent cell. In many such cases, the precursor cells include immune precursor cells, such as common myeloid progenitor cells, granulocyte progenitor cells, myeloblasts, monocytes, common lymphoid progenitor cells, lymphoid progenitor cells, progenitor B cells, or combinations thereof. In some cases, the precursor cells include a stem cell, such as a tetraploid reprogrammed cell, an induced pluripotent stem cell, an embryoblast, a lymphoid stem cell, or a myeloid stem cell.

### B. Transfection

The immune cells may be isolated and transfected with a polynucleotide expression cassette encoding the engineered cytokine receptor switch, thereby engineering the immune cell to express the cytokine receptor switch. The engineered immune cells may be cultured and formulated for delivery to a subject. In some embodiments, the subject may be the donor.

The polynucleotide expression cassette can encode an engineered protein such as a CAR and/or an engineered cytokine receptor switch. The polynucleotide expression cassette can also encode elements for gene editing, nucleases, reverse transcriptases, integrases, recombinases, and combinations thereof. As non-limiting examples, a nuclease encoded by the polynucleotide expression cassette can include a zinc finger nuclease, a transcription activator-like effector nuclease (TALEN), a Cas3 nuclease, a Cas9 nuclease, a CRISPR/Cas12 nuclease, a CRISPR/Cas14 nuclease, a Fok1 nuclease, or a combination thereof. The polynucleotide expression cassette can also encode additional transcripts and proteins which further affect cell phenotype, such as small interfering RNA (siRNA), microRNA (miRNA), short hairpin RNA (shRNA), ribozymes, transcription factors, or immunomodulatory elements.

The immune cells can be transfected with a polynucleotide expression cassette encoding the engineered cytokine receptor switch and/or CAR, thereby engineering the immune cell to express the cytokine receptor switch and/or CAR. The polynucleotide expression cassette can be a plasmid, a cosmid, a viral vector, or a combination thereof. The polynucleotide expression cassette can be naked or delivered via a nonviral vector (e.g., liposomes or lipid nanoparticles). The expression cassette can have one or more control sequences which affect expression of the engineered cytokine receptor switch. The expression cassette can also include one or more selection markers.

The polynucleotide expression cassette may comprise an RNA sequence or a DNA sequence encoding an engineered cytokine receptor switch and/or CAR, or an RNA sequence or a DNA sequence reverse complementary to a sequence encoding an engineered cytokine receptor switch and/or CAR. In some embodiments, the polynucleotide encoding the engineered cytokine receptor switch and/or CAR may be part of a polynucleotide expression cassette capable of expressing the engineered cytokine receptor switch and/or CAR in a cell (e.g., an immune cell). The polynucleotide expression cassette may comprise a promoter, an open reading frame (e.g., encoding the engineered cytokine receptor switch and/or CAR), a 3′ untranslated region, or combinations thereof. In some embodiments, the polynucleotide expression cassette may comprise two or more open reading frames. For example, the polynucleotide expression cassette may comprise a first open reading frame encoding the engineered cytokine receptor switch and a second open reading frame encoding a CAR. The expression cassette may further comprise an origin of replication, a restriction endonuclease site, a selectable marker, or combinations thereof. The expression cassette may be capable of expressing both the engineered cytokine receptor switch and the CAR in a cell (e.g., an immune cell). In some embodiments, the cell may be a mammalian cell (e.g., a human cell). For example, the cell may be a human T-cell. A polynucleotide or polynucleotide expression cassette may be obtained using recombinant methods known in the art. Alternatively or in addition, the polynucleotide or polynucleotide expression cassette may be generated synthetically.

Also provided herein are vectors comprising the polynucleotide expression cassette. The vector may be capable of delivering the polynucleotide expression cassette to a target cell (e.g., an immune cell). Upon delivery, a protein encoded by the polynucleotide expression cassette (e.g., an engineered cytokine receptor switch, a CAR, or combinations thereof) may be expressed in the cell. In some cases, the polynucleotide expression cassette is encoded within a viral vector. In some cases, the viral vector is a lentiviral vector, an adeno-associated viral vector, a vaccinia viral vector, a poxvirus viral vector, a herpes viral vector, an alphavirus viral vector, gamma retrovirus, a polyoma viral vector, or a combination thereof. In some cases, the viral vector is a gamma retrovirus, an adeno-associated viral vector or a lentiviral vector. In some cases, the viral vector is a lentiviral vector. In some cases, the viral vector has a titer of between about 10⁶ and about 10⁹ virions per ml. In some cases, transfection includes delivery of nonviral vectors (e.g., in a lipid or chitosan nanoparticle or with a colloidal dispersion system), for example mRNA encoding an engineered cytokine receptor switch, a CAR, a transcription factor, or a nuclease (e.g., a zing finger protein, a TAL-effector domain protein, or a CRISPR/Cas nuclease); DNA (e.g., DNA encoding an engineered cytokine receptor switch) for targeted recombination; a nuclease, signaling molecule, or transcription factor; siRNA; miRNA; or a combination thereof. In some cases, between about 10% and about 30%, between about 10% and about 50%, between about 20% and about 60%, between about 40% and about 90%, or between about 60% and about 95% of naïve T cells and B cells from among the immune cell population are transfected.

A vector encoding an engineered cytokine receptor switch may be generated and delivered to an immune cell using standard cloning and gene delivery protocols, for example as described in Sambrook et al. (2001, Molecular Cloning: A Laboratory Manual, Cold Spring Harbor Laboratory, New York), which is herein incorporated by reference. A vector may encode a selectable marker, a reporter gene, or both to facilitate selection of cells (e.g., immune cells) successfully transfected and expressing a protein encoded by the expression cassette (e.g., the engineered cytokine receptor switch, the CAR, or both).

Alternatively, or in addition thereto, a polynucleotide expression cassette may be introduced into a target cell using physical or chemical means. In some embodiments, a polynucleotide expression cassette may be introduced into a target cell using calcium phosphate precipitation, lipofection, particle bombardment, microinjection, electroporation, and the like. In some embodiments, the polynucleotide expression cassette may be introduced using colloidal dispersion systems (e.g., macromolecule complexes), nanocapsules, microspheres, beads, lipid-based systems (e.g., oil-in-water emulsions, micelles, mixed micelles, liposomes), and the like. Additional transfection or infection methods are described in Sambrook et al. (2001, Molecular Cloning: A Laboratory Manual, Cold Spring Harbor Laboratory, New York).

In some cases, the immune cells are contacted to a receptor agonist concurrently with and/or prior to transfection. In some cases, the receptor agonist is selected from the group consisting of granulocyte macrophage-colony stimulating factor (GM-CSF), stem cell factor (SCF), interleukin-1 (IL1), interleukin-2 (IL2), interleukin-β (IL3), a CD3 agonist, a CD4 agonist, a CD8 agonist, a CD16 agonist, a CD23 agonist, a CD28 agonist, a CD47 agonist, a CD80 agonist, a CD113 agonist, a CD131 agonist, a CD137 agonist, an HLA-E agonist, a 41BBL agonist, and a combination thereof. In some cases, the receptor agonist is selected from the group consisting of a CD3 agonist, a CD23 agonist, a CD28 agonist, an IL2 receptor agonist, and a combination thereof. In some cases, the CD3 agonist is an antigen. In some cases, the receptor agonist is coupled to a substrate. In some cases, the substrate comprises a peptide, an antibody, a minibody, a nanobody, a fragment antigen-binding, a nanoparticle, a microparticle, a polymer matrix, a surface, a surface functionalization (e.g., a dextran polymer functionalized with the activator), a carbon nanomaterial, a quantum dot, a surface, or a combination thereof. In some cases, the substrate is contacted to the immune cells at a ratio of between about 50:1 and about 1:1 (substrate to immune cells). In some cases, the immune cells are contacted with a CD3 agonist and a CD28 agonist prior to transfection. In some cases, the immune cells are contacted with IL2 prior to transfection. In some cases, the immune cells are contacted with IL2 during transfection. In some cases, the immune cells are expanded by about 2-fold, about 5-fold, about 10-fold, about 25-fold, about 50-fold, or about 100-fold prior to transfection and the contacting to the substrate. In some cases, the immune cells are expanded by between about 2-fold and about 10-fold, by between about 2-fold and about 25-fold, by between about 5-fold and about 50-fold, or by between about 10-fold and about 100-fold prior to transfection and the contacting to the substrate.

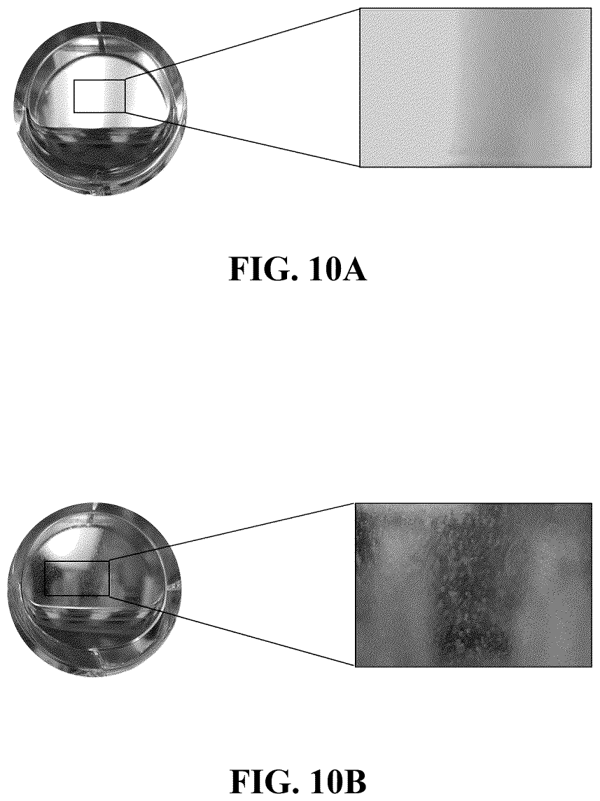

Following transfection, the immune cells can be fractionated on a substrate. In some cases, the immune cells are collected on a substrate that is functionalized with an activator (e.g., a species which binds to an activator binding domain of an engineered cytokine receptor switch), which may also be used to perform ex vivo activation of the immune cells as described herein. As depicted in FIG. 10A and FIG. 10B, which shows immune cells engineered to express an engineered cytokine receptor switch grown in an unfunctionalized well and a fluorescein-functionalized well, respectively, activator functionalized substrates (e.g., test tube surfaces, cell culture flasks, cell culture bags, cell culture plates, G-rex bioreactors, CentriCult chambers, cassettes, column materials, beads such as magnetically separatable beads) can selectively bind cells which express engineered cytokine receptor switches. Alternatively, or in addition thereto, immune cells can be collected on substrates functionalized with agents which bind engineered (e.g., CAR) or native (e.g., phenotype-specific immune cell markers, such as CD45RA or CCR7) receptors. A method can comprise separating immune cells which express a particular receptor (e.g., an engineered cytokine receptor switch) from immune cells which do not express the receptor by binding the receptor to a substrate. Unbound cells can then be washed or removed, while the receptor-expressing immune cells can be collected from the substrate. Alternatively, the substrate (along with bound immune cells) can be separated from unbound immune cells, for example through gravimetric or magnetic separation.

In some cases, immune cells are fractionated by cell phenotype. During such processes, subsets of the engineered immune cell population which express a particular cell marker (e.g., CCR7 or CD45RA) can be captured and separated from subsets of the engineered immune cell population which do not express the cell marker. For example, T cells can be separated from non-T cells of the immune cell population through CD3 affinity collection. In some cases, a method includes selecting memory T cells from an immune cell population, for example by collecting CCR7+ and/or CCR7+/CD45RA+ cells from the immune cell population.

### C. Immune Cell Phenotypes

Immune cells (e.g., immune cells engineered to express an engineered cytokine receptor switch) can be engineered and cultured to achieve a target ratio of phenotypes. When challenged with antigens, immune cells often differentiate towards terminal effector phenotypes, which typically have high potencies but relatively short lifespans in vivo in the absence of antigen. While such immune cells can have pronounced activities against acute diseases, rapid immune cell death following partial disease clearance can allow for disease recurrence. Memory cells typically exhibit lower potencies than terminal effector cells, but comprise longer in vivo lifespans, as well as a greater ability to divide, often yielding a high persistence in the absence of their target antigens. Accordingly, controlling effector and memory phenotype ratios can be important for tailoring a treatment to a subject. As disclosed herein, in many cases, a combination of effector and memory phenotypes is preferred for treating and managing diseases. In some embodiments, a desired ratio of effector and memory phenotypes may be achieved by mixing populations of immune cells expressing an engineered cytokine receptor switch. For example, an amount of a first population of cells expressing an engineered cytokine receptor switch that has been activated using an ex vivo activation method described herein may be combined with an amount of a second population of cells expressing an engineered cytokine receptor switch that has not been activated such that the resulting population of cells contains the desired ratio of effector cells and memory cells.

In some embodiments, a target ratio of phenotypes may alternatively or additionally be achieved by targeting immune cells to a lymphoid organ, as described herein. In some embodiments, a phenotype ratio may be controlled by administering an amount of a bispecific agent to target a desired portion of the engineered immune cells to the lymphoid organ. Increasing a dose of the bispecific agent administered to a patient may increase the fraction of engineered immune cells that target to the lymphoid organ, thereby increasing the proportion of memory cell phenotypes in the immune cell population. Decreasing a dose of the bispecific agent administered to a patient may decrease the fraction of engineered immune cells that target to the lymphoid organ, thereby increasing the proportion of effector cell phenotypes in the immune cell population.

In some embodiments, a ratio of effector and memory phenotypes in an immune cell population is between about 20:1 and about 1:20, between about 5:1 and about 1:5, or between about 2:1 and about 1:2. In some cases, a ratio of effector and memory phenotypes in an immune cell population is between about 100:1 and about 10:1, between about 50:1 and about 5:1, between about 25:1 and about 5:1, between about 15:1 and about 3:1, between about 10:1 and about 2:1, between about 5:1 and about 3:2, or between about 4:1 and about 3:2. In some cases, a ratio of effector and memory phenotypes in an immune cell population is between about 1:100 and about 1:10, between about 1:50 and about 1:5, between about 1:25 and about 1:5, between about 1:15 and about 1:3, between about 1:10 and about 1:2, between about 1:5 and about 2:3, or between about 1:4 and about 2:3. In some cases, at least about 10%, at least about 20%, at least about 30%, at least about 40%, at least about 50%, at least about 60%, at least about 70%, at least about 80%, at least about 90%, at least about 95%, at least about 98%, or at least about 99% of the immune cell population has a memory phenotype. In some cases, at least about 10%, at least about 20%, at least about 30%, at least about 40%, at least about 50%, at least about 60%, at least about 70%, at least about 80%, or at least about 90% of the immune cell population has an effector phenotype.

In some cases, at least about 40% of T cells of the immune cell population are central memory cells. In some cases, between about 40% and about 85% of T cells of the immune cell population are central memory cells. In some cases, between about 40% and about 75% of T cells of the immune cell population are central memory cells. In some cases, between about 45% and about 75% of T cells of the immune cell population are central memory cells. In some cases, between about 50% and about 75% of T cells of the immune cell population are central memory cells. In some cases, between about 60% and about 90% of T cells of the immune cell population are central memory cells.

In some cases, between about 3% and about 30% of T cells of the immune cell population are stem memory T cells. In some cases, between about 4% and about 25% of T cells of the immune cell population are stem memory T cells. In some cases, between about 4% and about 20% of T cells of the immune cell population are stem memory T cells. In some cases, between about 3% and about 15% of T cells of the immune cell population are stem memory T cells.

In some cases, between about 5% and about 60% of T cells of the immune cell population are effector memory T cells. In some cases, between about 5% and about 50% of T cells of the immune cell population are effector memory T cells. In some cases, between about 8% and about 40% of T cells of the immune cell population are effector memory T cells. In some cases, between about 8% and about 35% of T cells of the immune cell population are effector memory T cells. In some cases, between about 5% and about 35% of T cells of the immune cell population are effector memory T cells. In some cases, between about 5% and about 25% of T cells of the immune cell population are effector memory T cells. In some cases, between about 5% and about 20% of T cells of the immune cell population are effector memory T cells.

In some cases, less than about 20% of T cells of the immune cell population are terminally differentiated effector memory T cells. In some cases, less than about 15% of T cells of the immune cell population are terminally differentiated effector memory T cells. In some cases, less than about 10% of T cells of the immune cell population are terminally differentiated effector memory T cells. In some cases, less than about 8% of T cells of the immune cell population are terminally differentiated effector memory T cells. In some cases, less than about 6% of T cells of the immune cell population are terminally differentiated effector memory T cells. In some cases, less than about 4% of T cells of the immune cell population are terminally differentiated effector memory T cells. In some cases, between about 0.5% and about 20% of T cells of the immune cell population are terminally differentiated effector memory T cells. In some cases, between about 0.5% and about 10% of T cells of the immune cell population are terminally differentiated effector memory T cells. In some cases, between about 0.5% and about 6% of T cells of the immune cell population are terminally differentiated effector memory T cells.

### VI. COMPOSITIONS COMPRISING ENGINEERED IMMUNE CELLS

Further provided herein are compositions comprising engineered immune cells suitable for administration to a subject in need thereof. The engineered immune cells or precursor cells thereof may be cultured and formulated for delivery to a subject (e.g., a donor of precursors of the engineered immune cells). In some cases, precursor cells are cultured prior to transfection with a polynucleotide encoding a cytokine receptor switch. In such cases, the precursor cells can be activated, differentiated, and or expanded. The cells can be further cultured following transfection, for example, to further expand the cells or to affect terminal differentiation. In some cases, precursor cells are transfected with the cytokine receptor switch, a CAR, a transcription factor, or a combination thereof prior to culturing. The engineered immune cells can express an engineered cytokine receptor switch, and optionally can further express a CAR. In some cases, the engineered cytokine receptor switch is not activated prior to administration to the subject.

An engineered immune cell composition can be formulated with a solution tolerated by the immune cells. The immune cells are formulated with a solution of biological origin, such as plasma; a synthetic solution, such as saline, Ringer's solution, dextrose solution, phosphate buffered saline; water; or a combination thereof. The formulation can also include a nonaqueous vehicle, such as ethyl oleate or a fatty acid triglyceride.

An engineered immune cell composition can include a pharmaceutically acceptable carrier, diluent, or excipient. In some cases, the composition comprises a viscosity-enhancing agent (e.g., sodium carboxymethylcellulose, dextran, or glycerol). In some cases, the composition comprises an isotonicity imparting agent, such as sodium chloride, potassium chloride, or monosodium phosphate. In some cases, the composition comprises a stabilizing agent, such as carboxymethyl cellulose, alginate, polyethylene glycol, or a polyol. In some cases, the composition comprises a preservative, such as thimerosal, m- or o-cresol, formalin or benzyl alcohol. In some cases, the composition comprises an adjuvant, such as aluminum hydroxide. In some cases, the composition comprises a buffer, such as bicarbonate, TRIS, HEPES, MOPS, CHES, CHAPS, or phosphate buffered saline.

Engineered immune cell compositions can either be liquid injectables or solids which can be taken up in a suitable liquid as a suspension or solution for injection. Thus, in a non-liquid formulation, the excipient can comprise, for example, dextrose, human serum albumin, and/or preservatives to which sterile water or saline can be added prior to administration.

A composition can comprise engineered immune cells (e.g., engineered immune cells of a therapeutic composition for administration to a subject) with a ratio of effector and memory phenotypes of between about 20:1 and about 1:20, between about 5:1 and about 1:5, or between about 2:1 and about 1:2. In some cases, the engineered immune cells have a ratio of effector and memory phenotypes of between about 100:1 and about 10:1, between about 50:1 and about 5:1, between about 25:1 and about 5:1, between about 15:1 and about 3:1, between about 10:1 and about 2:1, between about 5:1 and about 3:2, or between about 4:1 and about 3:2. In some cases, the engineered immune cells have a ratio of effector and memory phenotypes of between about 1:100 and about 1:10, between about 1:50 and about 1:5, between about 1:25 and about 1:5, between about 1:15 and about 1:3, between about 1:10 and about 1:2, between about 1:5 and about 2:3, or between about 1:4 and about 2:3. In some cases, the engineered immune cells comprise at least about 50%, at least about 60%, at least about 70%, at least about 80%, at least about 90%, at least about 95%, at least about 98%, or at least about 99% memory phenotype cells. In some cases, at least about 50%, at least about 60%, at least about 70%, at least about 80%, at least about 90%, at least about 95%, at least about 98%, or at least about 99% of T cells of the engineered immune cells are memory T cells. In some cases, at least about 50%, at least about 60%, at least about 70%, at least about 80%, at least about 90%, at least about 95%, at least about 98%, or at least about 99% of B cells of the engineered immune cells are memory B cells.

### VII. THERAPEUTIC METHODS

The engineered cytokine receptor switches of the present disclosure may be used to treat a condition in a subject. Immune cells (e.g., lymphocytes, macrophages, or combinations thereof) engineered to express a cytokine receptor switch may be administered to a subject to treat a disease. In some embodiments, the immune cell may be further engineered to express an additional component, such as a CAR. For example, the immune cell may be a CAR T-cell engineered to express a CAR and a cytokine receptor switch.

The engineered immune cells may be activated (e.g., ex vivo, in vivo, or both) to promote formation of memory phenotypes (e.g., a stem-cell memory phenotype, a central memory phenotype, or combinations thereof) which increase immune cell persistence in the subject. Increased persistence of the engineered immune cells in the subject may improve patient outcome by reducing the chance of disease recurrence (e.g., cancer recurrence). The engineered immune cells may be administered to a subject having a condition, and the engineered immune cells may treat the condition. For example, the engineered immune cells may be CAR T-cells, and administration of the CAR T-cells to the subject may treat a cancer in the subject by targeting and killing cancer cells expressing an antigen that binds to a CAR expressed by the CAR T-cell. In some embodiments, the engineered immune cells may be re-activated in vivo by administering the activator to the subject. Re-activation of the engineered immune cells may treat or prevent disease recurrence. Alternatively, the engineered immune cells may not be activated ex vivo, may not be activated in vivo, or both (e.g., relying partially or entirely on activator-independent activity for the desired therapeutic effect).

An engineered immune cell may be re-activated one or more times after being administrated to the subject. In some embodiments, the engineered immune cell may be re-activated about 1 day, about 2 days, about 3 days, about 4 days, about 5 days, about 6 days, about 7 days, about 8 days, about 9 days, about 10 days, about 11 days, about 12 days, about 13 days, about 14 days, about 16 days, about 18 days, about 20 days, about 22 days, about 24 days, about 26 days, about 28 days, about 35 days, about 42 days, about 49 days, about 56 days, or combinations thereof, after being administered to the subject. In some embodiments, the engineered immune cell may be re-activated about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 21 months, about 24 months, about 27 months, about 30 months, about 33 months, about 36 months, or combinations thereof, after being administered to the subject. In some embodiments, the engineered immune cell may be re-activated about 1 year, about 2 years, about 3 years, about 4 years, about 5 years, about 6 years, about 7 years, about 8 years, about 9 years, about 10 years, or combinations thereof, after being administered to the subject.

In some embodiments, the engineered immune cell may be re-activated one or more times from about 7 days to about 56 days, from about 14 days to about 56 days, from about 21 days to about 56 days, from about 28 days to about 56 days, from about 35 days to about 56 days, from about 42 days to about 56 days, from about 49 days to about 56 days, from about 1 month to about 36 months, from about 2 months to about 36 months, from about 3 months to about 36 months, from about 4 months to about 36 months, from about 5 months to about 36 months, from about 6 months to about 36 months, from about 12 months to about 36 months, from about 18 months to about 36 months, from about 24 months to about 36 months, from about 30 months to about 36 months, from about 1 year to about 10 years, from about 2 years to about 10 years, from about 3 years to about 10 years, from about 4 years to about 10 years, from about 5 years to about 10 years, from about 1 years to about 8 years, from about 2 years to about 8 years, from about 3 years to about 8 years, from about 4 years to about 8 years, from about 5 years to about 8 years, from about 1 years to about 6 years, from about 2 years to about 6 years, from about 3 years to about 6 years, from about 4 years to about 6 years, or from about 5 years to about 6 years after being administered to the subject.

Immune cells expressing an engineered cytokine receptor switch of the preset disclosure, and optionally further expressing a CAR (e.g., a direct CAR or an indirect CAR), may be used to treat a number of different conditions. In some embodiments, an engineered immune cell may be used to treat a cancer, such as acute myeloid leukemia, multiple myeloma, ovarian cancer, mesothelioma, non-Hodgkin lymphoma, acute lymphoblastic leukemia, mantle cell lymphoma, follicular lymphoma, glioma (e.g., diffuse midline glioma), pancreatic cancer, prostate cancer, or gastric cancer.

In some embodiments, an immune cell engineered to express an engineered cytokine receptor switch and a direct CAR that directly recognizes a tumor antigen may be used to treat a cancer. For example, an immune cell engineered to express a cytokine receptor switch and an anti-CD123 CAR or an anti-CD33 CAR may be used to treat acute myeloid leukemia. In another example, an immune cell engineered to express a cytokine receptor switch and an anti-BCMA CAR or an anti-GPRCSD CAR may be used to treat multiple myeloma. In another example, an immune cell engineered to express a cytokine receptor switch and an anti-MUC16 CAR, an anti-HER2 CAR, an anti-mesothelin CAR, an anti-folate receptor-α CAR may be used to treat ovarian cancer. In another example, an immune cell engineered to express a cytokine receptor switch and an anti-mesothelin CAR may be used to treat mesothelioma. In a further example, an immune cell engineered to express a cytokine receptor switch and an anti-GD2 CAR may be used to treat glioma.

In some embodiments, an immune cell engineered to express an engineered cytokine receptor switch and an indirect CAR that recognizes a bispecific agent that binds to a tumor antigen may be used to treat a cancer. For example, an immune cell engineered to express a cytokine receptor switch and an indirect CAR that recognizes a bispecific agent including an anti-CD123 antibody or an anti-CD33 antibody may be used to treat acute myeloid leukemia. In another example, an immune cell engineered to express a cytokine receptor switch and an indirect CAR that recognizes a bispecific agent including an anti-BCMA antibody or an anti-GPRC5D antibody may be used to treat multiple myeloma. In another example, an immune cell engineered to express a cytokine receptor switch and an indirect CAR that recognizes a bispecific agent including an anti-MUC16 antibody, an anti-HER2 antibody, an anti-mesothelin antibody, or an anti-folate receptor-α antibody may be used to treat ovarian cancer. In another example, an immune cell engineered to express a cytokine receptor switch and an indirect CAR that recognizes a bispecific agent including an anti-mesothelin antibody may be used to treat mesothelioma. In a further example, an immune cell engineered to express a cytokine receptor switch and an indirect CAR that recognizes a bispecific agent including an anti-GD2 antibody may be used to treat glioma.

In some cases, between about 10⁵ and about 5×10⁷ immune cells are administered to the subject. In some cases, between about 5×10⁴ and about 3×10⁷ memory immune cells are administered to the subject. In some cases, between about 5×10⁴ and about 3×10⁷ effector immune cells are administered to the subject. In some cases, a method of treatment comprises administering a first dose of engineered immune cells comprising at least 50% effector phenotype immune cells, and a second dose of engineered immune cells comprising at least 50% memory phenotype immune cells. In some cases, a method of treatment comprises administering a first dose of engineered immune cells comprising a greater number of effector phenotype immune cells than memory phenotype immune cells, and a second dose of engineered immune cells comprising a greater number of memory phenotype immune cells than effector phenotype immune cells. In some cases, the first dose of engineered immune cells and the second dose of engineered immune cells express an engineered cytokine receptor switch. In some cases, the first dose of engineered immune cells and the second dose of engineered immune cells are subjected to different activation conditions prior to administration. In some cases, the second dose of engineered immune cells is administered at least 1 day, at least 3 days, at least 7 days, at least 14 days, at least 28 days, or at least 60 days after the first dose of engineered immune cells.

Immune cells expressing an engineered cytokine receptor switch may persist in a subject longer than an immune cell that does not express an engineered cytokine receptor switch. For example, a CAR T-cell expressing an engineered cytokine receptor switch may persist in a subject longer than a CAR T-cell that does not express an engineered cytokine receptor switch. In some embodiments, an immune cell expressing an engineered cytokine receptor switch may persist in a subject for at least about 2 times, at least about 3 times, at least about 4 times, at least about 5 times, at least about 6 times, at least about 7 times, at least about 8 times, at least about 9 times, at least about 10 times, at least about 15 times, at least about 20 times, at least about 25 times, at least about 50 times, or at least about 100 times as long as an immune cell that does not express an engineered cytokine receptor switch.

In some embodiments, an immune cell expressing an engineered cytokine receptor switch may persist in a subject for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 16 days, at least about 18 days, at least about 20 days, at least about 22 days, at least about 24 days, at least about 26 days, at least about 28 days, at least about 35 days, at least about 42 days, at least about 49 days, at least about 56 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 21 months, at least about 24 months, at least about 27 months, at least about 30 months, at least about 33 months, at least about 36 months, at least about 1 year, at least about 2 years, at least about 3 years, at least about 4 years, at least about 5 years, at least about 6 years, at least about 7 years, at least about 8 years, at least about 9 years, or at least about 10 years after being administered to the subject.

A composition of the present disclosure (e.g., an immune cell expressing an engineered cytokine receptor switch and a CAR) may be used in a method of treating cancer in a subject. The compositions described herein may promote activation, differentiation, or clonal expansion of an engineered immune cell (e.g., an immune cell expressing an engineered cytokine receptor switch and a CAR) by recruiting the engineered immune cell to a lymphoid organ (e.g., the lymph nodes, spleen, thymus, bone marrow) via a bispecific agent (e.g., a composition comprising an activator linked to a lymphoid-targeting protein). A bispecific agent may bind to an engineered cytokine receptor switch expressed by the engineered immune cell via the activator, and the lymphoid-targeting protein of the bispecific agent may bind to a lymphoid marker, recruiting the engineered immune cell to the lymphoid organ.

The lymphoid organ may comprise an environment conducive to immune cell activation, differentiation, and clonal expansion. In some embodiments, binding the activator to the engineered cytokine receptor switch may activate cytokine signaling through the engineered cytokine receptor switch, promoting differentiation of the immune cell into a memory cell phenotype. Differentiation of the immune cell into memory cell phenotypes may increase persistence of the immune cells in the subject, increasing the duration of an anti-tumor response produced by the immune cell (e.g., by binding of the CAR to a target antigen). Clonal expansion of the immune cell may increase the immune cell population, including populations of effector cell phenotypes, memory cell phenotypes, or both. Increasing the population of the effector cell phenotype may increase an acute anti-tumor response produced by the immune cell.

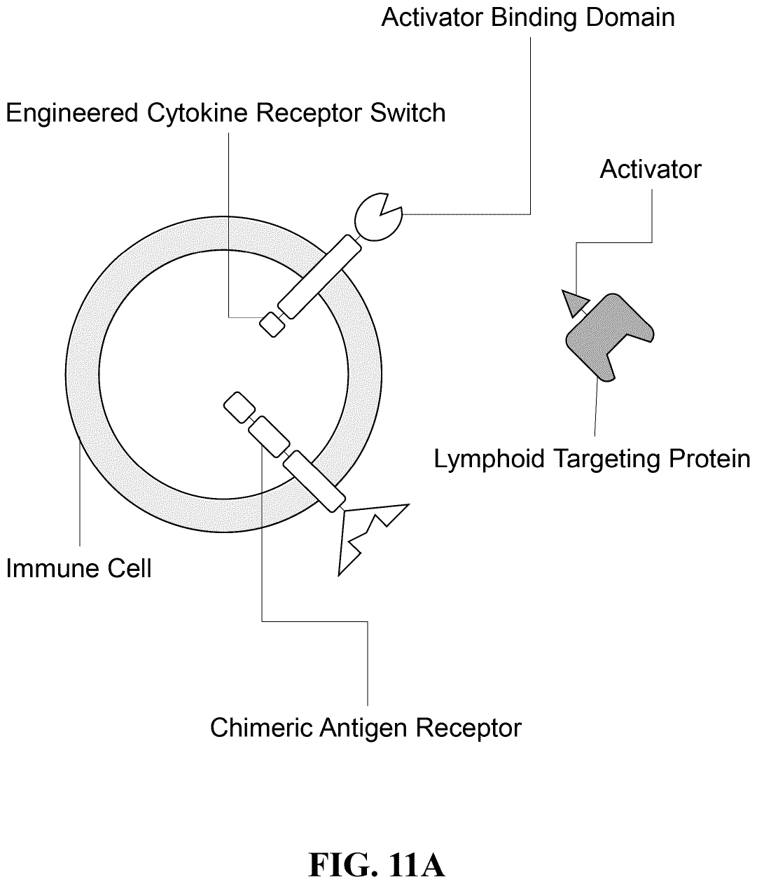

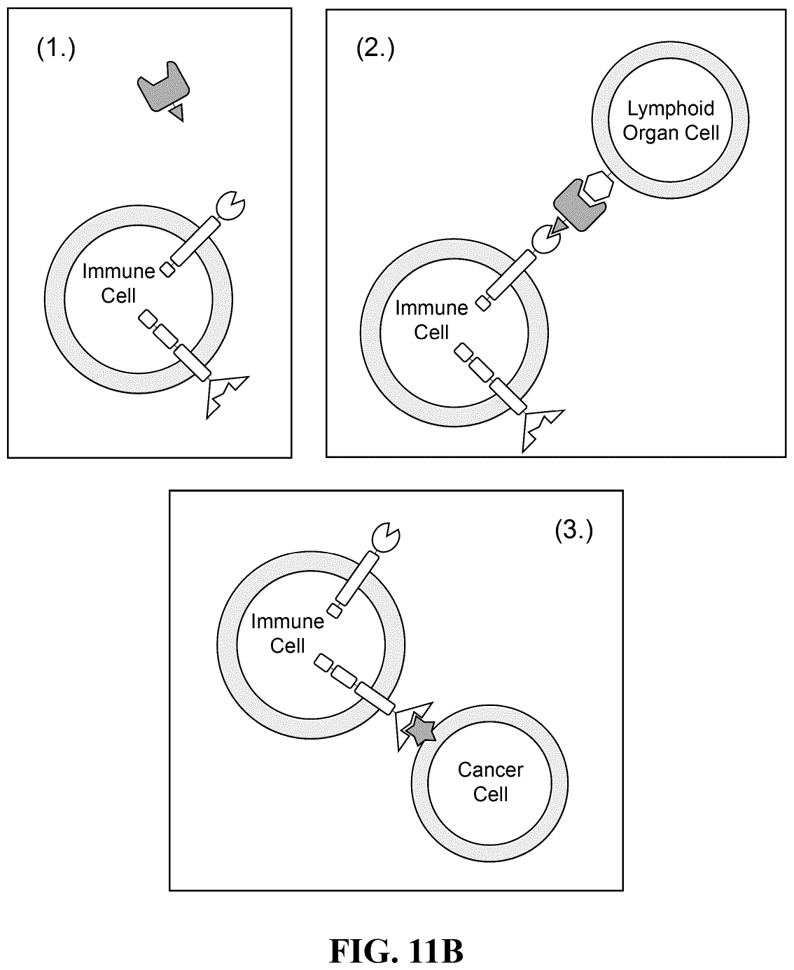

As shown in FIG. 11A and FIG. 11B (panel 1), an immune cell expressing an engineered cytokine receptor switch (e.g., any one of SEQ ID NO: 1-SEQ ID NO: 7) and a CAR may be administered to a subject having cancer. For example, the subject may have acute myeloid leukemia, multiple myeloma, ovarian cancer, mesothelioma, non-Hodgkin lymphoma, acute lymphoblastic leukemia, mantle cell lymphoma, follicular lymphoma, glioma, pancreatic cancer, prostate cancer, or gastric cancer. The subject may be further administered a bispecific agent comprising an activator that binds to the engineered cytokine receptor switch and a lymphoid-targeting protein, as shown in FIG. 11A and FIG. 11B (panel 1). The bispecific agent may recruit the immune cell to the lymphoid organ by binding to a lymphoid marker expressed by a cell in or associated with the lymphoid organ (e.g., a lymphocyte or a lymphoid organ stromal cell), as shown in FIG. 11B (panel 2). The binding of the bispecific agent to the immune cell can activate cytokine signaling through the engineered cytokine receptor switch. Additionally, in the lymphoid organ, the immune cell may proliferate and differentiation, forming populations of effector cells and memory cells. The effector cells may promote tumor cell killing upon recruitment to a tumor cell via the CAR, as shown in FIG. 11B (panel 3). The memory cells may persist in the subject, promoting long-term anti-cancer effects.

In some embodiments, recruitment to the lymphoid organ via a bispecific agent may promote expression of CCR7, CD45RA, CD95, or combinations thereof in the engineered immune cell. In some embodiments, the bispecific agent may bind to the lymphoid marker (e.g., via the lymphoid-targeting protein) with an equilibrium dissociation constant (K_{D}) of no more than 1 μM, no more than 100 nM, no more than 10 nM, or no more than 1 nM. The engineered immune cell may be retained in the lymphoid organ for an amount of time sufficient to activate the immune cell. For example, the engineered immune cell may be retained in the lymphoid organ for 6 to 96 hours, 12 to 72 hours, or 24 to 48 hours.

In some embodiments, a method of treating a cancer in a subject is provided. The method can include administering to the subject an immune cell population including immune cells expressing an engineered cytokine receptor switch and a CAR (e.g., a direct CAR or an indirect CAR). The engineered cytokine receptor switch can include an activator binding domain that binds an activator (e.g., an exogenous small molecule), a signal peptide, a hinge domain, a transmembrane domain, and/or an intracellular domain. In some embodiments, the immune cells are activated ex vivo with the activator before being administered to the subject, e.g., by exposing the immune cells to the activator on a substrate. The ex vivo activation can cause conversion of the immune cells to a memory phenotype, upregulation of lymphoid homing markers on the immune cell, or a combination thereof. In other embodiments, the immune cells are not activated ex vivo before being administered to the subject.

The method can further include activating the immune cells in vivo concurrently with or after the immune cells are administered to the subject, e.g., by administering the activator to the subject concurrently with or after the immune cells are administered to the subject. The in vivo activation can cause conversion of the immune cells to a memory phenotype, upregulation of lymphoid homing markers on the immune cell, or a combination thereof. In some embodiments, a bispecific agent including the activator is administered to the subject. The bispecific agent can include a targeting moiety conjugated to the activator. The targeting moiety can recognize a target for the immune cell, such as a cancer cell, a cell of a lymphoid organ, or another cell type. In some embodiments, the targeting moiety is a lymphoid-targeting protein that directs the immune cell to a lymphoid organ. In some embodiments, the targeting moiety binds a tumor antigen. Optionally, the activator can be administered multiple times, e.g., to re-activate the immune cells in vivo. In other embodiments, the immune cells are not activated in vivo.

In some embodiments, the method includes causing conversion of the immune cells to a memory phenotype, upregulation of lymphoid homing markers on the immune cell, or a combination thereof, without activating the immune cells with the activator. In some embodiments, the method includes causing conversion of the immune cells to a memory phenotype, upregulation of lymphoid homing markers on the immune cell, or a combination thereof, via activator-independent activity of the engineered cytokine receptor switch. The activator-independent activity can include dimerization of the engineered cytokine receptor switch without binding of the activator to the engineered cytokine receptor switch and/or interactions of the engineered cytokine receptor switch another receptor on the immune cell (e.g., the CAR).

In some embodiments, the method includes administering an activator to the subject after the immune cells are administered to the subject, where the activator is administered after a delay period sufficient for activator-independent activity of the engineered cytokine receptor switch to occur. For example, the delay period can be at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 16 days, at least about 18 days, at least about 20 days, at least about 22 days, at least about 24 days, at least about 26 days, at least about 28 days, at least about 35 days, at least about 42 days, at least about 49 days, at least about 56 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. The administration of the activator can cause in vivo activation of the engineered cytokine receptor switch after the engineered cytokine receptor switch has exhibited activator-independent activity. In some embodiments, the activator-independent activity causes conversion of the immune cells to a memory phenotype and/or upregulation of lymphoid homing markers on the immune cell, and the administration of the activator enhances the conversion of the immune cells to a memory phenotype and/or the upregulation of lymphoid homing markers.

In embodiments where the immune cell is engineered to express an indirect CAR, the method can further include administering a bispecific agent to the subject, where the bispecific agent includes a synthetic antigen recognized by the indirect CAR, and a targeting moiety that recognizes a tumor antigen on a cancer cell. The bispecific agent can be administered concurrently with or after the immune cells are administered to the subject. The dosage of the bispecific agent can be within a range from 0.1 mg/kg to 10 mg/kg, 0.1 mg/kg to 5 mg/kg, 0.1 mg/kg to 2 mg/kg, 0.1 mg/kg to 1 mg/kg, 0.1 mg/kg to 0.5 mg/kg, 0.5 mg/kg to 10 mg/kg, 0.5 mg/kg to 5 mg/kg, 0.5 mg/kg to 2 mg/kg, 0.5 mg/kg to 1 mg/kg, 1 mg/kg to 10 mg/kg, 1 mg/kg to 5 mg/kg, 1 mg/kg to 2 mg/kg, 2 mg/kg to 10 mg/kg, 2 mg/kg to 5 mg/kg, or 5 mg/kg to 10 mg/kg.

In some embodiments, the method includes administering a first bispecific agent to the subject, and administering a second bispecific agent to the subject. For example, the first bispecific agent can include an activator for an engineered cytokine receptor switch and a first targeting moiety; and the second bispecific agent can include a synthetic antigen for an indirect CAR and a second targeting moiety. The activator can be the same as the synthetic antigen, or the activator can be different than the synthetic antigen. The first targeting moiety can be the same as the second targeting moiety, or the first targeting moiety can be different than the second targeting moiety. The first targeting moiety can recognize the same epitope as the second targeting moiety, or can recognize a different epitope than the second targeting moiety. The dosage of the first targeting moiety can be the same as, greater than, or less than the dosage of the second targeting moiety.

As another example, the first bispecific agent can include an activator for an engineered cytokine receptor switch and a first targeting moiety; and the second bispecific agent can include the activator for the engineered cytokine receptor switch and a second targeting moiety. The first targeting moiety can be different than the second targeting moiety. For instance, the first targeting moiety can recognize a first cell type (e.g., a cell of a lymphoid organ) and the second targeting moiety can recognize a second cell type (e.g., a cancer cell). The dosage of the first targeting moiety can be the same as, greater than, or less than the dosage of the second targeting moiety.

The first bispecific agent may be administered before, concurrently with, and/or after the second bispecific agent. In some embodiments, the first bispecific agent is administered at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 16 days, at least about 18 days, at least about 20 days, at least about 22 days, at least about 24 days, at least about 26 days, at least about 28 days, at least about 35 days, at least about 42 days, at least about 49 days, at least about 56 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months before the second bispecific agent is administered.

In some embodiments, the second bispecific agent is administered at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 16 days, at least about 18 days, at least about 20 days, at least about 22 days, at least about 24 days, at least about 26 days, at least about 28 days, at least about 35 days, at least about 42 days, at least about 49 days, at least about 56 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months before the first bispecific agent is administered.

### VIII. EXAMPLES

The present technology is further illustrated by the following non-limiting examples.

### Example 1: Development of Engineered Cytokine Receptor Switches for Immune Cell Activation

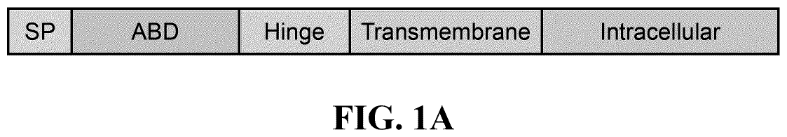

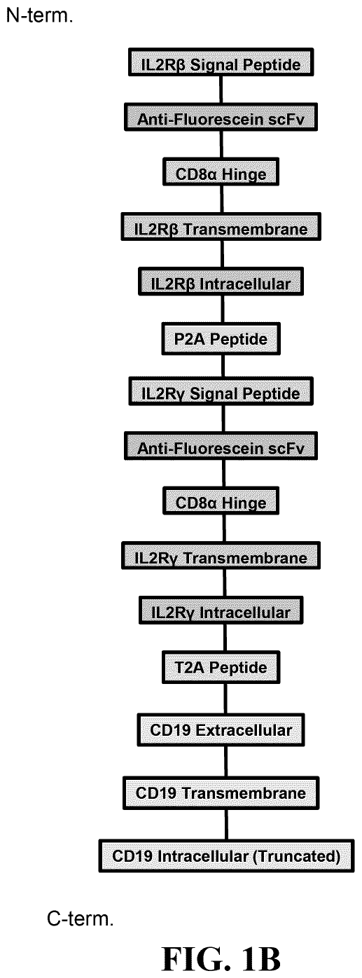

This example describes development of engineered cytokine receptor switches for immune cell activation. Cytokine receptors were engineered to be activated by a fluorescein isothiocyanate (FITC) small molecule activator. An intracellular signaling domain and transmembrane domain (TM) of an endogenous cytokine receptor were fused to a CD8a hinge, an activator binding domain (ABD) for a small molecule activator, such as an anti-FITC single-chain variable fragment (scFv), and a signal peptide (SP) of an endogenous cytokine receptor, as illustrated in FIG. 1A. The engineered cytokine receptors were based on an IL2Rα, IL2Rβ, IL2Rγ, IL7Rα, IL15Rα, or IL21Rα cytokine receptor. Sequences of the engineered cytokine receptors are provided in Table 1.

SEQ ID NO: 1 contained an IL2Rα signaling peptide (SEQ ID NO: 15), an anti-FITC scFv (SEQ ID NO: 21), a CD8 hinge (SEQ ID NO: 22), an IL2Rα transmembrane domain (SEQ ID NO: 23), and an IL2Rα intracellular signaling domain (SEQ ID NO: 29). SEQ ID NO: 2 contained an IL2Rβ signal peptide (SEQ ID NO: 16), an anti-FITC scFv (SEQ ID NO: 21), a CD8 hinge (SEQ ID NO: 22), an IL2Rβ transmembrane domain (SEQ ID NO: 24), and an IL2Rβ intracellular signaling domain (SEQ ID NO: 30). SEQ ID NO: 3 contained an IL2Rγ signal peptide (SEQ ID NO: 17), an anti-FITC scFv (SEQ ID NO: 21), a CD8 hinge (SEQ ID NO: 22), an IL2Rγ transmembrane domain (SEQ ID NO: 25), and an IL2Rγ intracellular signaling domain (SEQ ID NO: 31). SEQ ID NO: 4 contained an IL7Rα signal peptide (SEQ ID NO: 18), an anti-FITC scFv (SEQ ID NO: 21), a CD8 hinge (SEQ ID NO: 22), an IL7Rα transmembrane domain (SEQ ID NO: 26), and an IL7Rα intracellular signaling domain (SEQ ID NO: 32). SEQ ID NO: 5 contained an IL15Rα signal peptide (SEQ ID NO: 19), an anti-FITC scFv (SEQ ID NO: 21), a CD8 hinge (SEQ ID NO: 22), an IL15Rα transmembrane domain (SEQ ID NO: 27), and an IL15Rα intracellular signaling domain (SEQ ID NO: 33). SEQ ID NO: 6 contained an IL21Rα signal peptide (SEQ ID NO: 20), an anti-FITC scFv (SEQ ID NO: 21), a CD8 hinge (SEQ ID NO: 22), an IL21Rα transmembrane domain (SEQ ID NO: 28), and an IL21Rα intracellular signaling domain (SEQ ID NO: 34).

As shown in FIG. 1B, a dual-chain engineered cytokine receptor switch of SEQ ID NO: 7 contained an IL2Rβ signal peptide (SEQ ID NO: 16), an anti-FITC scFv (SEQ ID NO: 21), a CD8 hinge (SEQ ID NO: 22), an IL2Rβ transmembrane domain (SEQ ID NO: 24), an IL2Rβ intracellular signaling domain (SEQ ID NO: 30), a P2A peptide (SEQ ID NO: 35), an IL2Rγ signal peptide (SEQ ID NO: 17), an anti-FITC scFv (SEQ ID NO: 21), a CD8 hinge (SEQ ID NO: 22), an IL2Rγ transmembrane domain (SEQ ID NO: 25), an IL2Rγ intracellular signaling domain (SEQ ID NO: 31), a T2A peptide (SEQ ID NO: 36), and a truncated CD19 (SEQ ID NO: 39). The truncated CD19 included the entire extracellular and transmembrane domains and a truncated intracellular domain (cytoplasmic tail) to abrogate signaling. After translation, cleavage occurred at the P2A peptide and T2A peptide sequences, thus producing three separate proteins: (1) an engineered IL2Rβ cytokine receptor chain containing the IL2Rβ signal peptide (SEQ ID NO: 16), the anti-FITC scFv (SEQ ID NO: 21), the CD8 hinge (SEQ ID NO: 22), the IL2Rβ transmembrane domain (SEQ ID NO: 24), and the IL2Rβ intracellular signaling domain (SEQ ID NO: 30); (2) an engineered IL2Rγ cytokine receptor chain containing the IL2Rγ signal peptide (SEQ ID NO: 17), the anti-FITC scFv (SEQ ID NO: 21), the CD8 hinge (SEQ ID NO: 22), the IL2Rγ transmembrane domain (SEQ ID NO: 25), and the IL2Rγ intracellular signaling domain (SEQ ID NO: 31); and (3) the truncated CD19 (SEQ ID No: 39).

### Example 2: Controlling T-Cell Phenotype Using Small Molecule-Activated Cytokine Receptor Switches

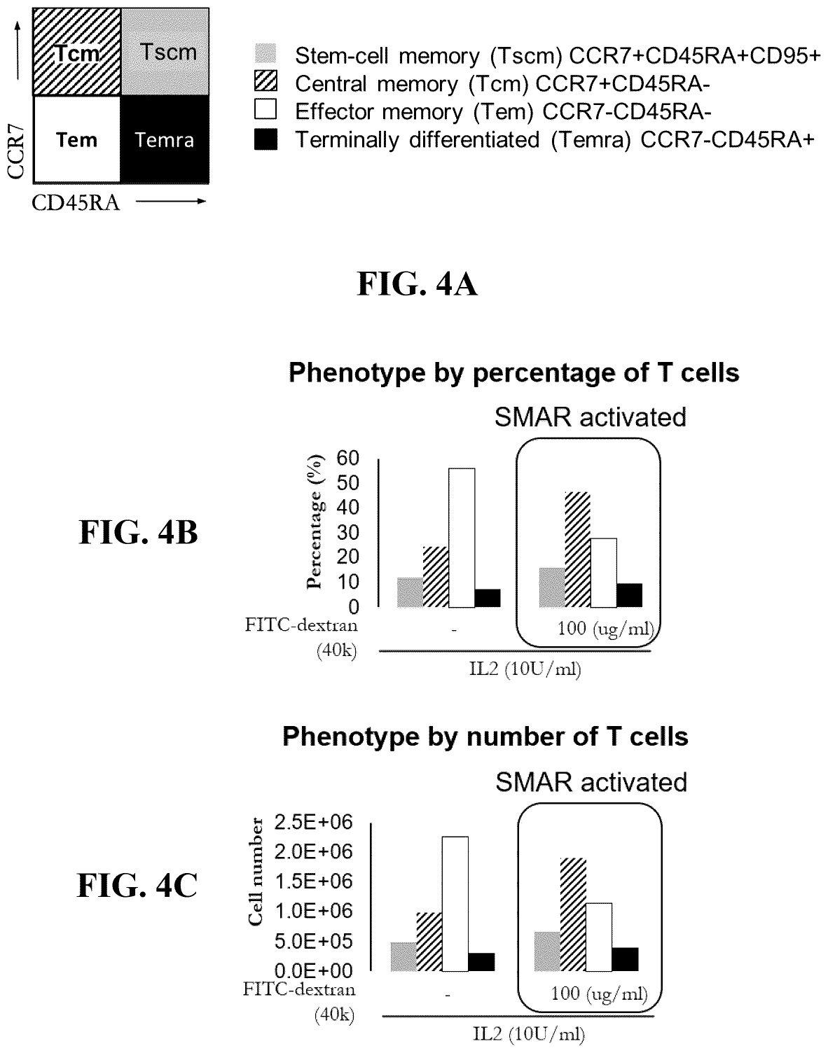

This example describes controlling T-cell phenotype using small molecule-activated cytokine receptor switches. After activation with CD3/CD28 Dynabeads, an engineered cytokine receptor switch derived from IL7Rα (“IL7Rα SMAR,” SEQ ID NO: 4) was transfected into T-cells in the presence of IL2 (10 U/mL). A portion of the IL2-treated cells were also treated with FITC conjugated to dextran (FITC-dextran, 100 μg/mL) to activate the engineered cytokine receptors and promote conversion from effector memory to central memory cell phenotype. The phenotype distribution of each population of T-cells was determined by assaying for phenotypic markers. As illustrated in FIG. 4A, cells positive for CCR7, CD45RA, and CD95 were identified as having a stem-cell memory (T_{scm}) phenotype, cells positive for CCR7 but negative for CD45RA were identified as having a central memory (T_{cm}) phenotype, cells negative for both CCR7 and CD45RA were identified has having an effector memory (Tₑₘ) phenotype, and cells positive for CD45RA but negative for CCR7 were identified as having an effector memory re-expressing CD45RA (Tₑₘᵣₐ) phenotype.

T-cells treated with IL2 and the FITC-dextran small molecule activator contained a higher portion of central memory cells than the population of cells treated with IL2 alone. As show in FIG. 4B (by percent of cells) and FIG. 4C (by number of cells), most of the T-cells treated with IL2 and FITC-dextran had a central memory phenotype, while most of the T-cells treated with IL2 alone had an effector memory phenotype. The T-cells treated with IL2 and FITC-dextran also showed an increase in stem-cell memory phenotype compared to the T-cells treated with IL2 alone. These data indicate that the IL7Rα SMAR promoted conversion from effector memory phenotype to central memory and stem-cell memory phenotypes when activated by FITC.

### Example 3: CAR T-Cell Mediated Tumor Cell Killing Facilitated by Engineered Cytokine Receptor Switches Following Tumor Re-Challenge

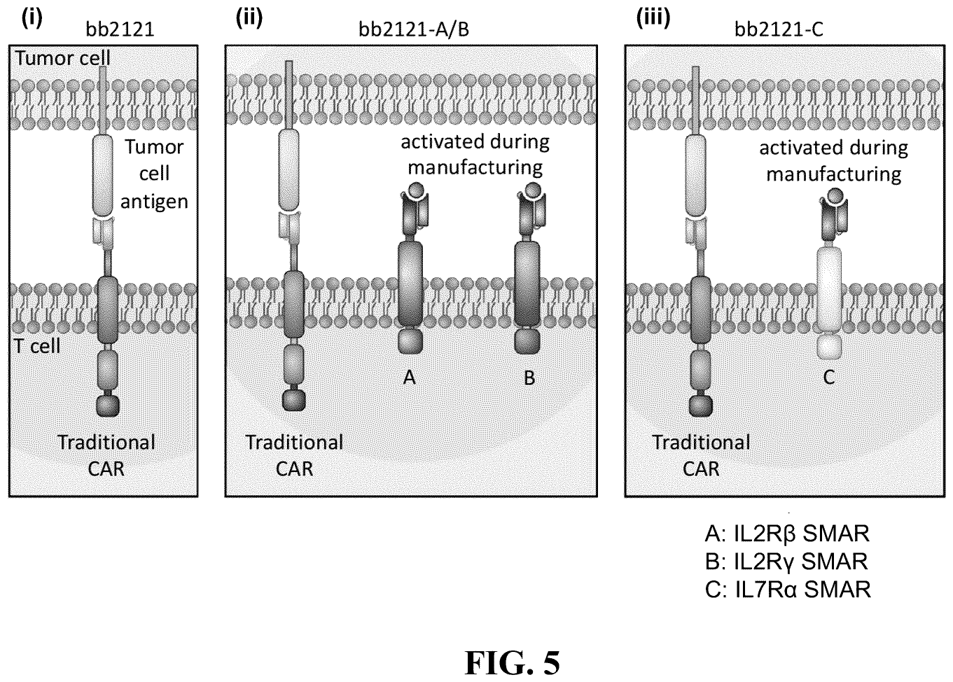

This example describes CAR T-cell mediated tumor cell killing facilitated by engineered cytokine receptor switches following tumor re-challenge. The tumor re-challenge assay was designed to assess proliferation and long-term tumor-killing function of CAR T-cells expressing a CAR with or without co-expression of an engineered cytokine receptor switch. T-cells were transfected to express a BCMA-targeting CAR (“bb2121 CAR”) and either (i) no engineered cytokine receptor switch, (ii) a dual-chain engineered cytokine receptor switch with a first chain derived from IL2R (“IL2RβSMAR,” SEQ ID NO: 2) (A) and a second chain derived from IL2Rγ (“IL2Rγ SMAR,” SEQ ID NO: 3) (B), or (iii) the IL7Rα SMAR (C), as illustrated in FIG. 5. The CAR T-cells were treated with FITC-dextran to activate the engineered cytokine receptor switches, if present.

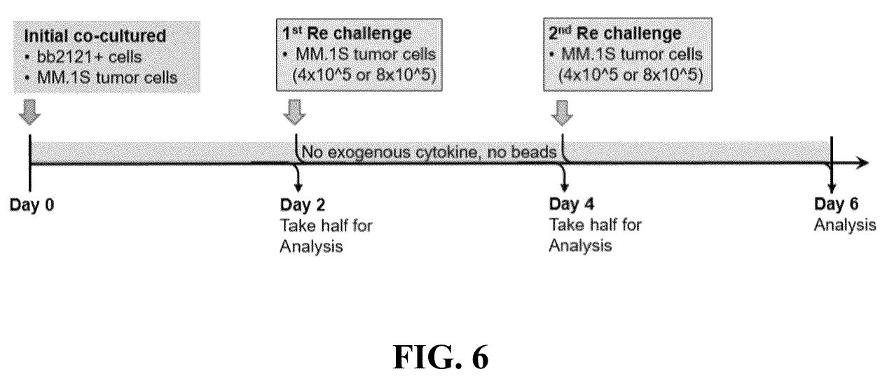

The activated CAR T-cells were co-cultured on day 0 with multiple myeloma tumor cells (MM.1S tumor cells) at an effector to target ratio (E/T) of 1/4 CAR⁺ cells to tumor cells by combining 1×10⁵ CAR⁺ cells and 4×10⁵ MM.1S target cells, or of 1/8 by combining 1×10⁵ CAR⁺ cells and 8×10⁵ MM.1S target cells. As illustrated in the assay timeline in FIG. 6, tumor rechallenges were performed on day 2 (I^{st} Rechallenge) and day 4 (2^{nd} rechallenge) by removing cells for analysis and adding either 4×10⁵ MM.1S target cells (E/T ratio of 1/4) or 8×10⁵ MM.1S target cells (E/T ratio of 1/8) to the CAR⁺ cells. Cells from the second rechallenge were removed on day 6 for analysis.

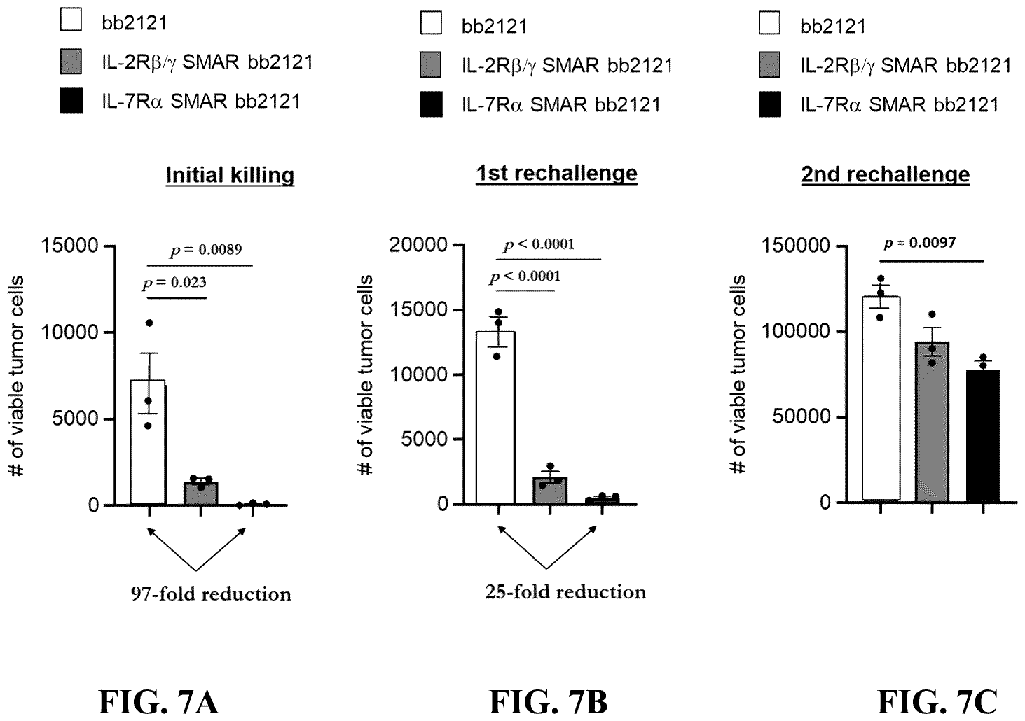

Long-term tumor-killing function of the assayed CAR T-cell populations was evaluated by quantifying the number of viable tumor cells in the culture 2 days after each tumor cell introduction. T-cells expressing both the bb2121 CAR and the IL7Rα SMAR (right) showed a 97-fold reduction in the number of viable tumor cells compared to the T-cells expressing only the bb2121 CAR (left) in the initial tumor cell killing measured on day 2 (FIG. 7A; E/T ratio of 1/8). T-cells expressing the bb2121 CAR, the IL2Rβ SMAR, and the IL2Rγ SMAR (middle) also showed a significant reduction in tumor cells in the initial tumor cell killing relative to the T-cells expressing only the bb2121 CAR. In the first tumor rechallenge, measured on day 4 (FIG. 7B; E/T ratio of 1/8), the T-cells expressing both the bb2121 CAR and the IL7Rα SMAR (right) showed a 25-fold reduction in the number of viable tumor cells compared to the T-cells expressing only the bb2121 CAR (left). The reduction in tumor cells in the first rechallenge T-cells expressing the bb2121 CAR, the IL2Rβ SMAR, and the IL2Rγ SMAR (middle) also showed a significant reduction in tumor cells in the initial tumor cell killing relative to the T-cells expressing only the bb2121 CAR was also significant. As measured on day 6, the T-cells expressing both the bb2121 CAR and the IL7Rα SMAR (right) showed a significant reduction in the number of viable tumor cells compared to the T-cells expressing only the bb2121 CAR (left) in the second tumor rechallenge (FIG. 7C; E/T ratio of 1/8). The error bars in FIG. 7A, FIG. 7B, and FIG. 7C represent ±standard error of the mean (SEM), and p values were determined using a one-way ANOVA with Tukey post hoc test. These data demonstrate that both the single-chain and dual-chain engineered cytokine receptors promoted long-term tumor-killing function of CAR T-cells.

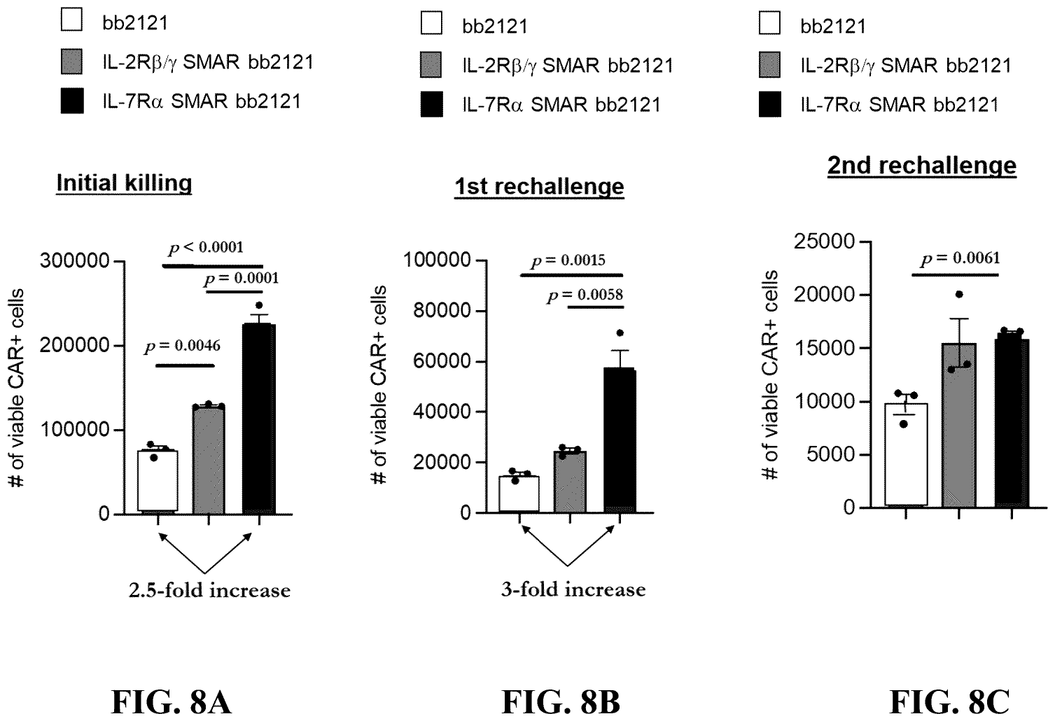

T-cell proliferation was evaluated by quantifying the number of viable CAR+(bb2121+) T-cells following the initial killing, first tumor rechallenge, and second tumor rechallenge, measured on days 2, 4, and 6, respectively. T-cells expressing both the bb2121 CAR and the IL7Rα SMAR (right) showed a 2.5-fold increase in the number of viable CAR⁺ cells compared to the T-cells expressing only the bb2121 CAR (left) in the initial tumor cell killing (FIG. 8A; E/T ratio of 1/8). This represented an increase in the number of viable CAR⁺ cells over the starting number of viable CAR⁺ cells (100,000 cells) in cells expressing the single-chain engineered cytokine receptor switch. T-cells expressing the bb2121 CAR, the IL2Rβ SMAR, and the IL2Rγ SMAR (middle) also showed a significant increase in the number of viable CAR⁺ cells in the initial tumor cell killing relative to the T-cells expressing only the bb2121 CAR. In the first tumor rechallenge (FIG. 8B; E/T ratio of 1/8), the T-cells expressing both the bb2121 CAR and the IL7Rα SMAR (right) showed a 3-fold increase in the number of viable CAR⁺ cells compared to the T-cells expressing only the bb2121 CAR (left). The T-cells expressing both the bb2121 CAR and the IL7Rα SMAR (right) showed a significant increase in the number of viable CAR⁺ cells compared to the T-cells expressing only the bb2121 CAR (left) in the second tumor rechallenge (FIG. 8C; E/T ratio of 1/8). The error bars in FIG. 8A, FIG. 8B, and FIG. 8C represent ±standard error of the mean (SEM), and p values were determined using a one-way ANOVA with Tukey post hoc test. These data demonstrate that both the single-chain and dual-chain engineered cytokine receptors promoted CAR T-cell proliferation and survival, suggesting that they enhanced generation of memory CAR T-cells, improving durability and long-term survivability of the CAR T-cells.

### Example 4: In Vivo Tumor-Killing Properties of CAR T-Cells Expressing an Engineered Cytokine Receptor Switch

This example describes study designs for investigating the in vivo tumor-killing properties of CAR T-cells expressing an engineered cytokine receptor switch. T-cells are transfected to co-express (1) a bb2121 CAR and the IL7Rα SMAR (“SMAR1-bb2121”), (2) to co-express the bb2121 CAR, the IL2Rβ SMAR, and the IL2Rγ SMAR (“SMAR2-bb2121”), or (3) to express the bb2121 CAR alone (“bb2121”).

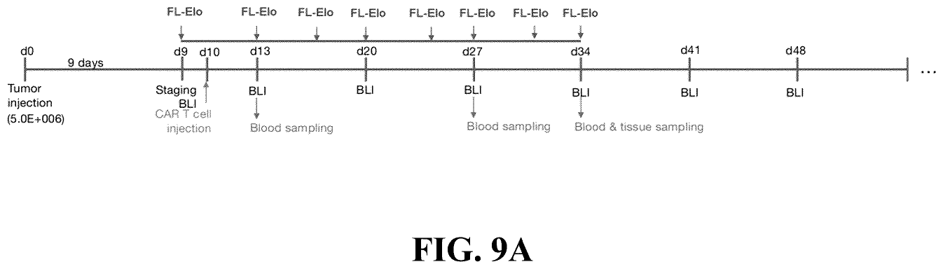

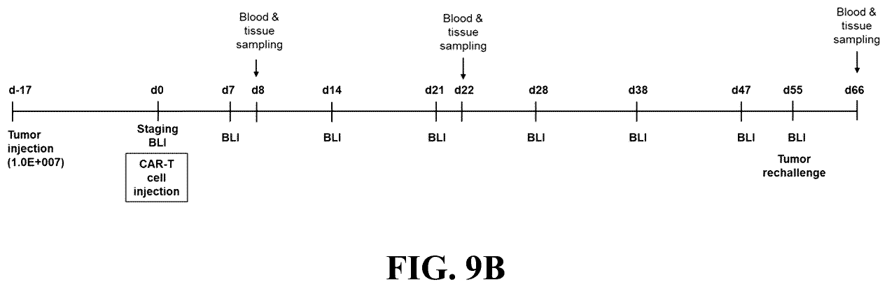

FIG. 9A illustrates a timeline for a first study design to investigate tumor killing with in vivo activation of the engineered cytokine receptor switch. As indicated in the timeline provided in FIG. 9A, on day 0 (“d0”) of the study, 5×10⁶ tumor cells are injected into each mouse. On day 9 (“d9”), mice with size-matched tumors are randomized into different groups (Table 10) and are treated with FITC-conjugated anti-CS1 elotuzumab biosimilar as the SMAR activator (“FL-elo”). On day 10 (“d10”), mice receive either bb2121 CAR T cells, SMAR1-bb2121 T cells, or SMAR2-bb2121 T cells. The SMAR activator is injected into mice in the appropriate sample groups on days 13, 17, 20, 24, 27, 31, and 34 (“d13,” “d17,” “d20,” “d24,” “d27,” “d31,” and “d34,” respectively). Bioluminescence imaging of the tumor is performed on days 9, 13, 20, 27, 34, 41, and 48 (“d9,” “d13,” “d20,” “d27,” “d34,” “d41,” and “d48,” respectively). Blood sampling is performed on days 13, 27, and 34 (“d13,” “d27,” and “d34,” respectively). Tissue sampling is performed on day 34 (“d34”).

TABLE 10
Study Group Conditions

| | | CAR | Antibody |
|---|---|---|---|
| Group | Description | T-Cell Dose | Dose |
| 1 | Control | None | None |
| 2 | bb2121 | 2 × 10⁶ | None |
| 3 | bb2121 | 5 × 10⁶ | None |
| 4 | bb2121 + FL-elo | 5 × 10⁶ | 1.0 mg/kg FL-elo |
| 5 | SMAR1-bb2121 | 2 × 10⁶ | None |
| 6 | SMAR1-bb2121 | 5 × 10⁶ | None |
| 7 | SMAR1-bb2121 + FL-elo | 5 × 10⁶ | 1.0 mg/kg FL-elo |
| 8 | SMAR2-bb2121 | 2 × 10⁶ | None |
| 9 | SMAR2-bb2121 | 5 × 10⁶ | None |
| 10 | SMAR2-bb2121 + FL-elo | 5 × 10⁶ | 1.0 mg/kg FL-elo |
| 11 | bb2121 + FL-elo | 5 × 10⁶ | 1.0 mg/kg FL-elo |
| 12 | SMAR1-bb2121 + FL-elo | 5 × 10⁶ | 1.0 mg/kg FL-elo |
| 13 | SMAR2-bb2121 + FL-elo | 5 × 10⁶ | 1.0 mg/kg FL-elo |

Groups receiving the fluorescein-antibody conjugate (i.e., groups 4, 7, 10, 11, 12, and 13) are injected with 1.0 mg/kg of FL-elo twice per week for 3 to 4 weeks (i.e., on days 13, 17, 20, 24, 27, 31, and 34). Bioluminescent imaging (BLI) of each mouse is performed on day 9, prior to CAR T-cell injection, and then weekly on days 13, 20, 27, 34, 41, and 48 to evaluate tumor progression. Blood sampling is performed on days 13, 27, and 34, and tissue sampling is performed on day 34.

FIG. 9B illustrates a timeline for a second study design to investigate tumor killing without in vivo activation of the engineered cytokine receptor switch. As indicated in the timeline provided in FIG. 9B, 17 days before initiation of the treatment phase of the study (“d-17”), 1×10⁷ tumor cells are injected into each mouse. On day 0 (“d0”), mice with size-matched tumors are randomized into different groups (Table 11) and receive either bb2121 CAR T-cells, SMAR1-bb2121 T cells, or SMAR2-bb2121 T cells. Bioluminescence imaging of the tumor is performed on days 0, 7, 14, 21, 28, 38, 47, and 55 (“d0,” “d7,” “d14,” “d21,” “d28,” “d38,” “d47,” and “d55,” respectively). Blood and tissue sampling is performed on days 8, 22, and 66 (“d8,” “d22,” and “d66,” respectively). Mice are rechallenged with tumor cells on day 55 (“d55”).

TABLE 11
Study Group Conditions

| Group | Description | CAR T-Cell Dose |
|---|---|---|
| 1 | Control | None |
| 2 | bb2121 | 2 × 10⁶ |
| 3 | bb2121 | 5 × 10⁶ |
| 4 | SMAR1-bb2121 | 2 × 10⁶ |
| 5 | SMAR1-bb2121 | 5 × 10⁶ |
| 6 | SMAR2-bb2121 | 2 × 10⁶ |
| 7 | SMAR2-bb2121 | 5 × 10⁶ |

### Example 5: In Vivo T-Cell Phenotype Conversion Facilitated by an Engineered Cytokine Receptor Switch

This example describes in vivo T-cell phenotype conversion facilitated by an engineered cytokine receptor switch. T-cells are transfected to express a CAR and an engineered cytokine receptor switch (e.g., a single-chain receptor switch of any one of SEQ ID NO: 1-SEQ ID NO: 6, a dual-chain receptor switch including SEQ ID NO: 2 and SEQ ID NO: 3, or of SEQ ID NO: 7) capable of being activated by a small molecule activator (e.g., fluorescein, a fluorescein derivative, or DOTA). The resulting CAR T-cells are injected into a subject having cancer. Phenotype conversion of the CAR T-cells is stimulated in vivo by injecting a small molecule activator-antibody conjugate into the subject having cancer. Administration of the small molecule activator-antibody conjugate activates the engineered cytokine receptor switch and promotes conversion of the CAR T-cells into memory T-cells, which persist in the subject long-term, and prevents formation of terminal effector CAR T-cells. The small molecule activator-antibody conjugate is administered to the subject weekly or twice weekly until the cancer is treated. In the event of cancer recurrence, the subject is re-administered the small molecule activator-antibody conjugate weekly or twice weekly until the cancer enters remission.

### Example 6: Treatment of Acute Myeloid Leukemia Using CAR T-Cells Expressing an Engineered Cytokine Receptor Switch

This example describes treatment of acute myeloid leukemia (AML) in a subject in need thereof using CAR T-cells expressing an engineered cytokine receptor switch. T-cells collected from the subject are transfected to co-express a CAR targeting CD123, CD33, or other AML marker, and an engineered cytokine receptor switch (e.g., a single-chain receptor switch of any one of SEQ ID NO: 1-SEQ ID NO: 6; a dual-chain receptor switch including SEQ ID NO: 2 and SEQ ID NO: 3, or of SEQ ID NO: 7). The CAR T-cells are treated with a small molecule activator (e.g., fluorescein, a fluorescein derivative, or DOTA) to activate the cytokine receptor switch and promote conversion of the T-cells to memory cell phenotypes. The activated CAR T-cells are administered to the subject having AML, and the CAR T-cells target and kill the tumor cells, thereby treating the AML. The activated CAR T-cells persist in the subject, enabling long-term cancer-targeting and preventing recurrence of the AML. In the event of recurrence, the small molecule activator conjugated to a protein substrate is administered to the subject to re-activate the CAR T-cells and treat the recurrent AML.

### Example 7: Treatment of Multiple Myeloma Using CAR T-Cells Expressing an Engineered Cytokine Receptor Switch

This example describes treatment of multiple myeloma in a subject in need thereof using CAR T-cells expressing an engineered cytokine receptor switch. T-cells collected from the subject are transfected to co-express a CAR targeting BCMA, GPRC5D, or other multiple myeloma marker, and an engineered cytokine receptor switch (e.g., a single-chain receptor switch of any one of SEQ ID NO: 1-SEQ ID NO: 6; a dual-chain receptor switch including SEQ ID NO: 2 and SEQ ID NO: 3, or of SEQ ID NO: 7). The CAR T-cells are treated with a small molecule activator (e.g., fluorescein, a fluorescein derivative, or DOTA) to activate the cytokine receptor switch and promote conversion of the T-cells to memory cell phenotypes. The activated CAR T-cells are administered to the subject having multiple myeloma, and the CAR T-cells target and kill the tumor cells, thereby treating the multiple myeloma. The activated CAR T-cells persist in the subject, enabling long-term cancer-targeting and preventing recurrence of the multiple myeloma. In the event of recurrence, the small molecule activator conjugated to a protein substrate is administered to the subject to re-activate the CAR T-cells and treat the recurrent multiple myeloma.

### Example 8: Treatment of Ovarian Cancer Using CAR T-Cells Expressing an Engineered Cytokine Receptor Switch

This example describes treatment of ovarian cancer in a subject in need thereof using CAR T-cells expressing an engineered cytokine receptor switch. T-cells collected from the subject are transfected to co-express a CAR targeting mesothelin, MUC16, HER2, EGFR, folate receptor-α, or other ovarian cancer marker, and an engineered cytokine receptor switch (e.g., a single-chain receptor switch of any one of SEQ ID NO: 1-SEQ ID NO: 6; a dual-chain receptor switch including SEQ ID NO: 2 and SEQ ID NO: 3, or of SEQ ID NO: 7). The CAR T-cells are treated with a small molecule activator (e.g., fluorescein, a fluorescein derivative, or DOTA) to activate the cytokine receptor switch and promote conversion of the T-cells to memory cell phenotypes. The activated CAR T-cells are administered to the subject having ovarian cancer, and the CAR T-cells target and kill the tumor cells, thereby treating the ovarian cancer. The activated CAR T-cells persist in the subject, enabling long-term cancer-targeting and preventing recurrence of the ovarian cancer. In the event of recurrence, the small molecule activator conjugated to a protein substrate is administered to the subject to re-activate the CAR T-cells and treat the recurrent ovarian cancer.

### Example 9: Treatment of Mesothelioma Using CAR T-Cells Expressing an Engineered Cytokine Receptor Switch

This example describes treatment of mesothelioma in a subject in need thereof using CAR T-cells expressing an engineered cytokine receptor switch. T-cells collected from the subject are transfected to co-express a CAR targeting mesothelin or other mesothelioma marker, and an engineered cytokine receptor switch (e.g., a single-chain receptor switch of any one of SEQ ID NO: 1-SEQ ID NO: 6; a dual-chain receptor switch including SEQ ID NO: 2 and SEQ ID NO: 3, or of SEQ ID NO: 7). The CAR T-cells are treated with a small molecule activator (e.g., fluorescein, a fluorescein derivative, or DOTA) to activate the cytokine receptor switch and promote conversion of the T-cells to memory cell phenotypes. The activated CAR T-cells are administered to the subject having mesothelioma, and the CAR T-cells target and kill the tumor cells, thereby treating the mesothelioma. The activated CAR T-cells persist in the subject, enabling long-term cancer-targeting and preventing recurrence of the mesothelioma. In the event of recurrence, the small molecule activator conjugated to a protein substrate is administered to the subject to re-activate the CAR T-cells and treat the recurrent mesothelioma.

### Example 10: Recruitment of Engineered T-Cells to Lymph Nodes to Promote Differentiation to Memory T-Cells

This example describes recruitment of engineered T-cells to lymph nodes. T-cells engineered to express a CAR and an engineered cytokine receptor switch are intravenously administered to a subject. The engineered cytokine receptor switch expressed by the engineered T-cells includes an activator binding domain that binds to a fluorescein or a fluorescein derivative. A lymph node targeting agent containing an anti-PNAd scFv or an anti-CD205 scFv conjugated to fluorescein or a fluorescein derivative is administered to the subject prior to the administration of the engineered T cells. The anti-PNAd scFv or anti-CD205 scFv of the lymph node targeting agent binds to PNAd or CD205 in the lymph nodes, respectively, and the fluorescein or fluorescein derivative of the lymph node targeting agent binds to the engineered T-cell via the activator binding domain of the engineered cytokine receptor switch. The lymph node targeting agent recruits the engineered T-cell to the lymph nodes. In the lymph nodes, the engineered T-cells differentiate into memory T-cells.

### Example 11: Treatment of Cancer by Targeting Engineered T-Cells to Lymph Nodes

This example describes treatment of cancer by targeting engineered T-cells to lymph nodes. T-cells collected from a subject having cancer are engineered to express a CAR and an engineered cytokine receptor switch. The CAR is designed to bind a cell surface marker associated with the cancer. The engineered cytokine receptor switch includes an activator binding domain that binds to a fluorescein or a fluorescein derivative and an intracellular cytokine signaling domain that is activated upon binding of the activator binding domain to fluorescein. The engineered T-cells are administered to the subject.

A lymph node targeting agent containing an anti-PNAd scFv or an anti-CD205 scFv conjugated to fluorescein or a fluorescein derivative is also administered to the subject. The anti-CDPNAd scFv or anti-CD205 scFv of the lymph node targeting agent binds to PNAd or CD205 in the lymph nodes, respectively, and the fluorescein or fluorescein derivative of the lymph node targeting agent binds to the engineered T-cell via the small molecule binding domain of the engineered cytokine receptor switch. The lymph node targeting agent recruits the engineered T-cell to the lymph nodes. In the lymph nodes, the engineered T-cells differentiate into memory T-cells.

The memory T-cells persist in the subject and continue to replicate to produce effector T-cells that target the cancer in the subject via the CAR, killing the cancer. The differentiated memory T-cells persist in the subject longer than the engineered T-cells that did not differentiate into memory T-cells, providing a long-term cancer killing effect, and preventing relapse of the cancer.

### Example 12: Design of Lymph Node Targeting Agents

This example describes design of lymph node targeting agents. Miniproteins are engineered de novo to bind to a lymph node surface marker. The lymph node surface marker is CD3, CD45, CD4, CD2, CD5, CD8, T6-T-cell receptor, T19, CD45, cell-surface immunoglobulin (sIg), L-selectin, PNAd, VEGFR-3, LYVE-1, Prox-1, podoplanin, CD31, MadCAM1, CXCL13, RANKL, CXCL12, APRIL, BAFF, IL-7, CCL19, CCL21, or Spns2. An engineered miniprotein binds to the corresponding lymph node surface markers with nanomolar affinity. An identified lymph node-binding miniprotein is conjugated to a small molecule activator. The small molecule activator is a fluorescein, a fluorescein derivative, or a tetraxetan (DOTA). The resulting lymph node targeting agent is capable of recruiting engineered T-cells expressing an engineered cytokine receptor with a small molecule binding domain to the lymph nodes.

### Example 13: Constructs for Expression of Engineered Cytokine Receptor Switches and/or CARs in Immune Cells

This example describes constructs that may be used to express engineered cytokine receptor switches (SMARs) and/or CARs in an immune cell.

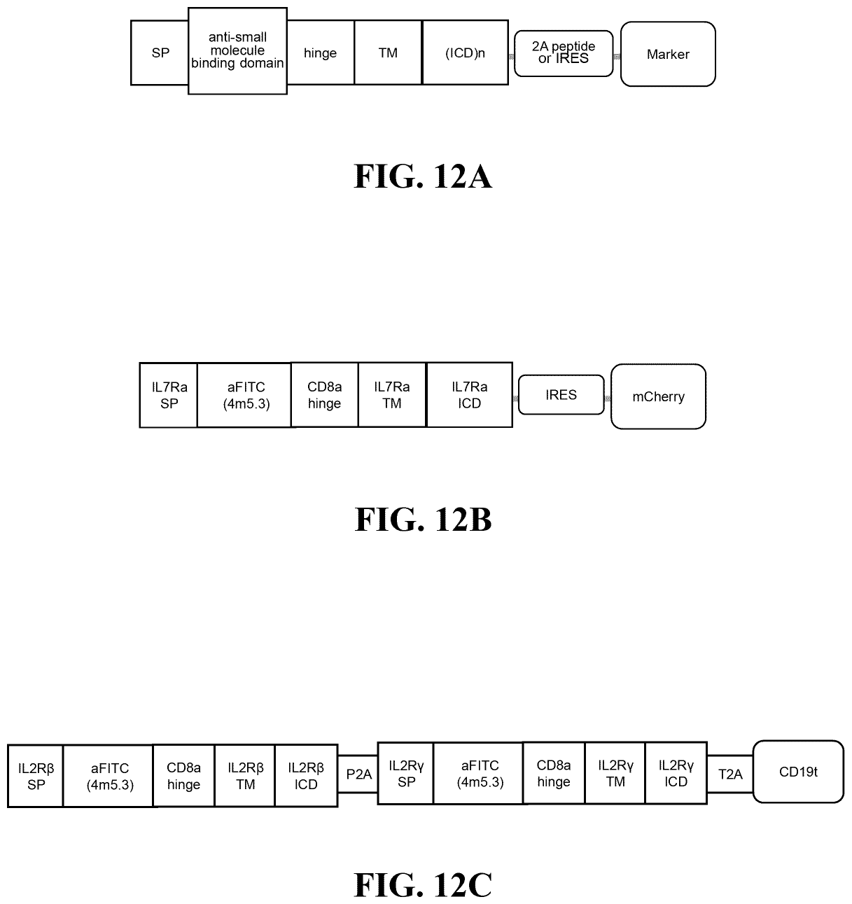

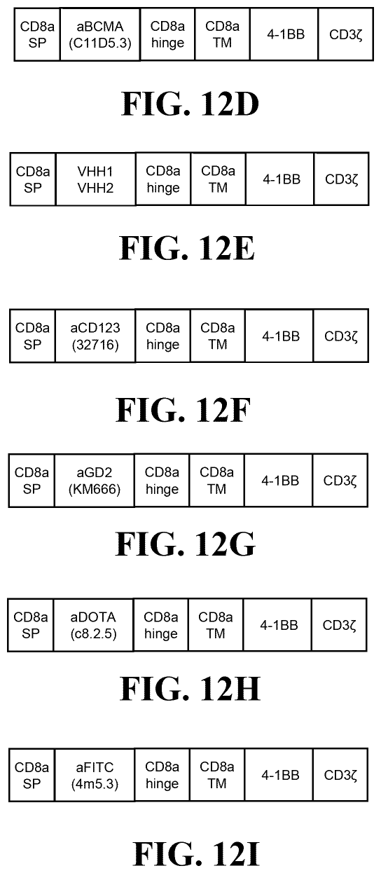

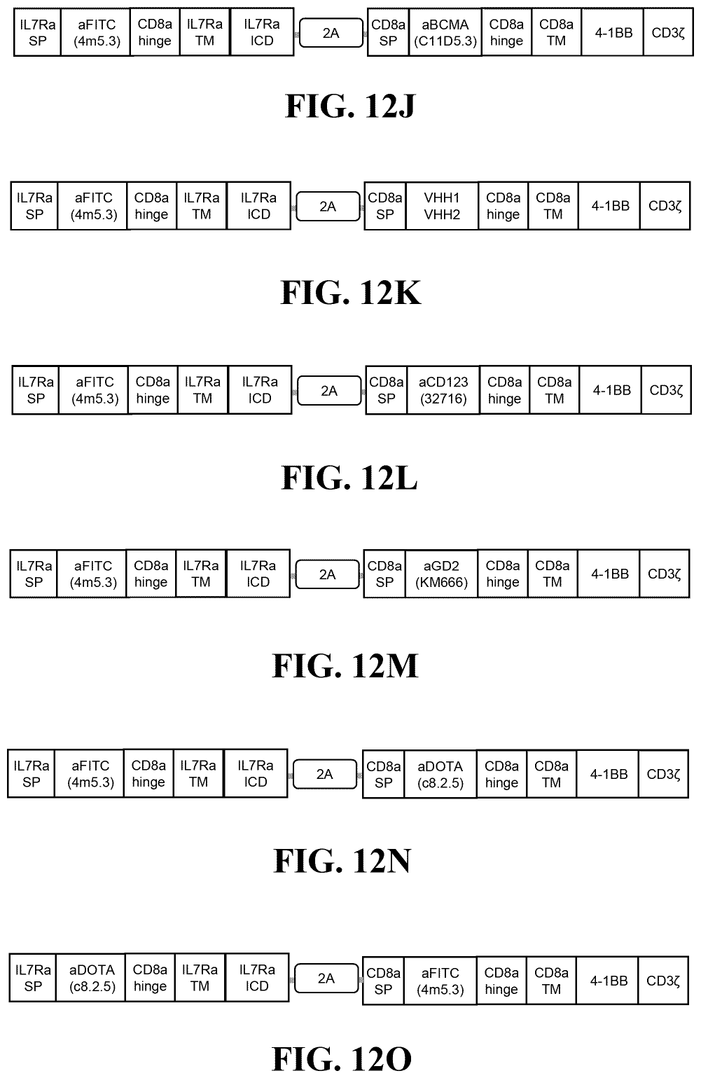

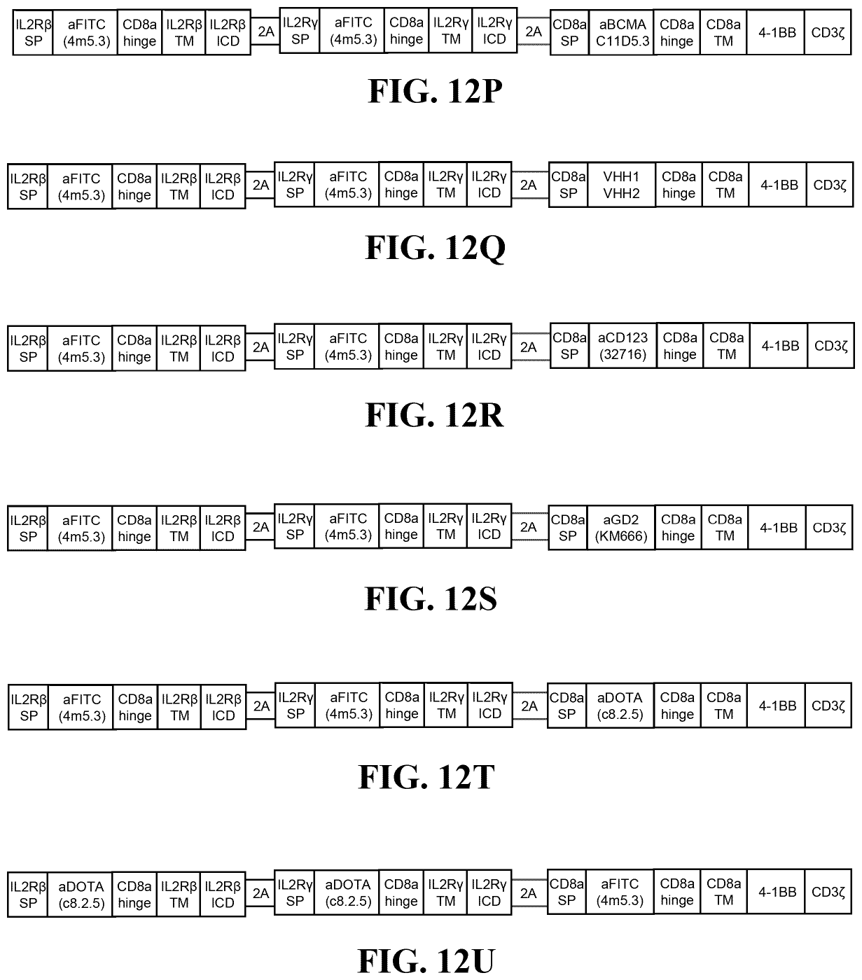

FIG. 12A is a schematic illustration of a domain layout of a construct for expression of a SMAR. As shown in FIG. 12A, the SMAR includes a signal peptide (SP), an anti-small molecule binding domain, a hinge, a transmembrane domain (TM), and an intracellular domain (ICD)n. The construct also includes a 2A peptide or an IRES, and a marker. The signal peptide can be a signal peptide of IL2Rα, IL2Rβ, IL2Rγ, IL7Rα, IL15Rα, IL21Rα, IgG1, GM-CSF, etc. The anti-small molecule binding domain can be an scFv, V_{H}, or V_{L} of an anti-FITC antibody (e.g., 4M5.3), an anti-DOTA antibody (e.g., C8.2.5), an anti-MPOB antibody, etc. The hinge can be a hinge domain of CD8a, CD28, IgG1, IgG4, EpoR, etc. The transmembrane domain can be a transmembrane domain of IL2Rα, IL2Rβ, IL2Rγ, IL7Rα, IL15Rα, IL21Rα, IgG1, GM-CSF, EpoR, etc. The intracellular domain can be a single intracellular domain of IL2Rα, IL2Rβ, IL2Rγ, IL7Rα, IL15Rα, IL21Rα, GM-CSF, etc. Alternatively, the intracellular domain can be multiple intracellular domains of any of the above in tandem, such as two or more of the same intracellular domain or a combination of two or more different intracellular domains. The marker domain can be a truncated CD19 domain, a truncated CD34 domain, a fluorescent protein, etc.

FIG. 12B is a schematic illustration of a domain layout of an example construct for expression of an IL7Rα SMAR. As shown in FIG. 12B, the IL7Rα SMAR includes an IL7Rα signal peptide, an anti-FITC scFv, a CD8a hinge, an IL7Rα transmembrane domain, and an IL7Rα intracellular domain. The construct also includes an IRES and an mCherry marker domain.

FIG. 12C is a schematic illustration of a domain layout of an example construct for expression of an IL2Rβ/γ SMAR. As shown in FIG. 12C, the IL2Rβ/γ SMAR includes a first cytokine receptor chain including an IL2Rβ signal peptide, an anti-FITC scFv, a CD8a hinge, an IL2Rβ transmembrane domain, and an IL2Rβ intracellular domain. The IL2Rβ/γ SMAR includes a second cytokine receptor chain including an IL2Rγ signal peptide, an anti-FITC scFv, a CD8a hinge, an IL2Rγ transmembrane domain, and an IL2Rγ intracellular domain. The first and second cytokine receptor chains are connected by a P2A peptide sequence. The construct also includes a T2A peptide sequence and a truncated CD19 marker domain.

FIG. 12D is a schematic illustration of a domain layout of an example construct for expression of an anti-BCMA CAR (bb2121). As shown in FIG. 12D, the anti-BCMA CAR includes a CD8a signal peptide, an anti-BCMA scFv (C11D5.3), a CD8a hinge, a CD8a transmembrane domain, a 4-1BB intracellular domain, and a CD3 (intracellular domain.

FIG. 12E is a schematic illustration of a domain layout of an example construct for expression of an anti-BCMA CAR (Carvykti). As shown in FIG. 12E, the anti-BCMA CAR includes a CD8a signal peptide, an anti-BCMA VHH1 and VHH2, a CD8a hinge, a CD8a transmembrane domain, a 4-1BB intracellular domain, and a CD3 (intracellular domain.

FIG. 12F is a schematic illustration of a domain layout of an example construct for expression of an anti-CD123 CAR. As shown in FIG. 12F, the anti-CD123 CAR includes a CD8a signal peptide, an anti-CD123 scFv (32716), a CD8a hinge, a CD8a transmembrane domain, a 4-1BB intracellular domain, and a CD3 (intracellular domain.

FIG. 12G is a schematic illustration of a domain layout of an example construct for expression of an anti-GD2 CAR. As shown in FIG. 12G, the anti-GD2 CAR includes a CD8a signal peptide, an anti-GD2 scFv (KM666), a CD8a hinge, a CD8a transmembrane domain, a 4-1BB intracellular domain, and a CD3 (intracellular domain.

FIG. 12H is a schematic illustration of a domain layout of an example construct for expression of an anti-DOTA indirect CAR. As shown in FIG. 12H, the anti-DOTA indirect CAR includes a CD8a signal peptide, an anti-DOTA scFv (c8.2.5), a CD8a hinge, a CD8a transmembrane domain, a 4-1BB intracellular domain, and a CD3 (intracellular domain.

FIG. 12I is a schematic illustration of a domain layout of an example construct for expression of an anti-FITC indirect CAR. As shown in FIG. 12I, the anti-FITC indirect CAR includes a CD8a signal peptide, an anti-FITC scFv (4m5.3), a CD8a hinge, a CD8a transmembrane domain, a 4-1 in intracellular domain, and a CD3ζ intracellular domain.

FIG. 12J is a schematic illustration of a domain layout of an example construct for expression of an IL7Rα SMAR and an anti-BCMA CAR (bb2121). As shown in FIG. 12J, the construct includes the IL7Rα SMAR of FIG. 12B and the anti-BCMA CAR of FIG. 12D linked to each other via a 2A peptide.

FIG. 12K is a schematic illustration of a domain layout of an example construct for expression of an IL7Rα SMAR and an anti-BCMA CAR (Carvykti). As shown in FIG. 12K, the construct includes the IL7Rα SMAR of FIG. 12B and the anti-BCMA CAR of FIG. 12E linked to each other via a 2A peptide.

FIG. 12L is a schematic illustration of a domain layout of an example construct for expression of an IL7Rα SMAR and an anti-CD123 CAR. As shown in FIG. 12L, the construct includes the IL7Rα SMAR of FIG. 12B and the anti-CD123 CAR of FIG. 12F linked to each other via a 2A peptide.

FIG. 12M is a schematic illustration of a domain layout of an example construct for expression of an IL7Rα SMAR and an anti-GD2 CAR. As shown in FIG. 12M, the construct includes the IL7Rα SMAR of FIG. 12B and the anti-GD2 CAR of FIG. 12G linked to each other via a 2A peptide.

FIG. 12N is a schematic illustration of a domain layout of an example construct for expression of an IL7Rα SMAR and an anti-DOTA indirect CAR. As shown in FIG. 12N, the construct includes the IL7Rα SMAR of FIG. 12B and the anti-DOTA indirect CAR of FIG. 12H linked to each other via a 2A peptide.

FIG. 12O is a schematic illustration of a domain layout of an example construct for expression of an IL7Rα SMAR and an anti-FITC indirect CAR. As shown in FIG. 12O, the construct includes the IL7Rα SMAR of FIG. 12B (except that the anti-FITC scFv is replaced with an anti-DOTA scFv (c8.2.5)) and the anti-FITC indirect CAR of FIG. 12I linked to each other via a 2A peptide.

FIG. 12P is a schematic illustration of a domain layout of an example construct for expression of an IL2Rβ/γ SMAR and an anti-BCMA CAR (bb2121). As shown in FIG. 12P, the construct includes the first and second cytokine receptor chains of the IL2Rβ/γ SMAR of FIG. 12C and the anti-BCMA CAR of FIG. 12D linked to each other via 2A peptides.

FIG. 12Q is a schematic illustration of a domain layout of an example construct for expression of an IL2Rβ/γ SMAR and an anti-BCMA CAR (Carvykti). As shown in FIG. 12Q, the construct the first and second cytokine receptor chains of the IL2Rβ/γ SMAR of FIG. 12C and the anti-BCMA CAR of FIG. 12E linked to each other via 2A peptides.

FIG. 12R is a schematic illustration of a domain layout of an example construct for expression of an IL2Rβ/γ SMAR and an anti-CD123 CAR. As shown in FIG. 12R, the construct includes the first and second cytokine receptor chains of the IL2Rβ/γ SMAR of FIG. 12C and the anti-CD123 CAR of FIG. 12F linked to each other via 2A peptides.

FIG. 12S is a schematic illustration of a domain layout of an example construct for expression of an IL2Rβ/γ SMAR and an anti-GD2 CAR. As shown in FIG. 12S, the construct includes the first and second cytokine receptor chains of the IL2Rβ/γ SMAR of FIG. 12C and the anti-GD2 CAR of FIG. 12G linked to each other via 2A peptides.

FIG. 12T is a schematic illustration of a domain layout of an example construct for expression of an IL2Rβ/γ SMAR and an anti-DOTA indirect CAR. As shown in FIG. 12T, the construct includes the first and second cytokine receptor chains of the IL2Rβ/γ SMAR of FIG. 12C and the anti-DOTA indirect CAR of FIG. 12H linked to each other via 2A peptides.

FIG. 12U is a schematic illustration of a domain layout of an example construct for expression of an IL2Rβ/γ SMAR and an anti-FITC indirect CAR. As shown in FIG. 12U, the construct includes the first and second cytokine receptor chains of the IL2Rβ/γ SMAR of FIG. 12C (except that the anti-FITC scFvs are replaced with anti-DOTA scFvs (c8.2.5)) and the anti-FITC indirect CAR of FIG. 12I linked to each other via 2A peptides.

### Example 14: Activator-Independent Activity of Immune Cells with an Engineered Cytokine Receptor Switch and a CAR

This example describes investigation of activator-independent activity in immune cells expressing IL7Rα SMAR constructs and an anti-BCMA CAR (Carvykti).

The following IL7Rα SMAR constructs were prepared by modifying the full length IL7Rα SMAR (“IL7Rα (WT) SMAR”, SEQ ID NO: 4): (1) an IL7Rα SMAR in which the 272th-459th amino acids of the IL7Rα intracellular domain were truncated, leaving a non-functional 7 amino acid tail (“IL7Rα (tICD) SMAR”), (2) an IL7Rα SMAR in which the V_{H} of the anti-FITC scFv was deleted (“IL7Rα (tVH) SMAR”), and (3) an IL7Rα SMAR in which the CD8a hinge was replaced with a GSG linker (“IL7Rα (GSG) SMAR”).

The polynucleotide constructs encoding the modified IL7Rα SMARs and the Carvykti CAR are provided in Table 12.

TABLE 12
Modified IL7Rα SMAR-Carvykti Constructs

| SMAR-CAR | | |
|---|---|---|
| Construct | SEQ ID NO | Sequence |
| anti-FITC | SEQ ID NO: 53 | ATGACCATCCTGGGCACCACCTTCGGCATGGTGTTTAGCCTG |
| IL7Rα SMAR | | CTGCAGGTCGTGTCTGGCGATTACAAGGATGACGACGATAA |
| (tICD)-T2A- | | GGACGTGGTCATGACACAGACCCCACTGTCTCTGCCTGTGTC |
| Carvykti | | TCTGGGAGATCAGGCCAGCATCAGCTGCAGATCTAGCCAGA |
| | | GCCTGGTGCACAGCAACGGCAACACCTACCTGCGGTGGTATC |
| | | TGCAGAAGCCCGGCCAGTCTCCTAAGGTGCTGATCTACAAGG |
| | | TGTCCAACAGAGTGTCCGGCGTGCCCGATAGATTTTCTGGCA |
| | | GCGGCTCTGGCACCGACTTCACCCTGAAGATCAATAGAGTGG |
| | | AAGCCGAGGACCTGGGCGTGTACTTCTGTAGCCAGTCTACCC |
| | | ACGTGCCATGGACCTTTGGCGGCGGAACAAAGCTGGAAATC |
| | | AAGAGCAGCGCCGACGACGCCAAGAAGGACGCCGCTAAGA |
| | | AGGATGACGCAAAGAAAGACGATGCCAAAAAGGATGGCGG |
| | | CGTGAAGCTGGACGAAACAGGCGGAGGACTTGTTCAGCCTG |
| | | GCGGAGCCATGAAGCTGAGCTGTGTGACCAGCGGCTTCACCT |
| | | TTGGCCACTACTGGATGAACTGGGTCCGACAGAGCCCTGAG |
| | | AAAGGCCTGGAATGGGTCGCCCAGTTCAGAAACAAGCCCTA |
| | | CAACTACGAAACCTACTACAGCGACAGCGTGAAGGGCAGAT |
| | | TCACCATCAGCCGGGACGACAGCAAGTCCAGCGTGTACCTG |
| | | CAGATGAACAACCTGCGCGTGGAAGATACCGGCATCTACTA |
| | | CTGTACCGGCGCCAGCTACGGCATGGAATATCTCGGCCAGG |
| | | GCACCAGCGTGACCGTGTCTACAACAACCCCTGCTCCTCGGC |
| | | CTCCTACACCAGCTCCTACAATTGCCAGCCAGCCTCTGTCTCT |
| | | GAGGCCCGAAGCTTGTAGACCTGCTGCCGGCGGAGCTGTGC |
| | | ATACAAGAGGCCTGGATTTCGCCTGCGATCCCATCCTGCTGA |
| | | CAATCAGCATCCTGAGCTTTTTCAGCGTGGCCCTGCTGGTCA |
| | | TCCTGGCCTGTGTGCTGTGGAAGAAGCGGATCAAGCCCATCG |
| | | GCTCCGGCGAAGGCAGAGGCTCTCTGCTTACATGCGGAGAT |
| | | GTGGAAGAGAACCCCGGACCTATGGCTCTGCCTGTCACCGCT |
| | | CTGCTGCTGCCTCTGGCTCTGCTGCTGCACGCTGCTCGCCCTC |
| | | AGGTCAAACTGGAAGAAAGTGGGGGAGGCCTGGTGCAGGCA |
| | | GGACGGAGCCTGCGCCTGAGCTGCGCAGCATCCGAGCACAC |
| | | CTTCAGCTCCCACGTGATGGGCTGGTTTCGGCAGGCCCCAGG |
| | | CAAGGAGAGAGAGTCCGTGGCCGTGATCGGCTGGAGGGACA |
| | | TCTCCACATCTTACGCCGATTCTGTGAAGGGCCGGTTCACCA |
| | | TCAGCAGAGACAACGCCAAGAAGACACTGTATCTGCAGATG |
| | | AATAGCCTGAAGCCCGAGGACACCGCCGTGTACTATTGCGC |
| | | AGCAAGGAGAATCGACGCAGCAGACTTTGATTCCTGGGGCC |
| | | AGGGCACCCAGGTGACAGTGTCTAGCGGAGGAGGAGGATCT |
| | | GGAGGAGGAGGAAGCGGAGGAGGAGGATCCGAGGTGCAGC |
| | | TGGTGGAGTCTGGAGGCGGCCTGGTGCAGGCCGGAGGCTCT |
| | | CTGAGGCTGAGCTGTGCAGCATCCGGAAGAACCTTCACAAT |
| | | GGGCTGGTTTAGGCAGGCACCAGGAAAGGAGAGGGAGTTCG |
| | | TGGCAGCAATCAGCCTGTCCCCTACCCTGGCCTACTATGCCG |
| | | AGTCCGTGAAGGGCAGGTTTACCATCTCTCGCGATAACGCCA |
| | | AGAATACAGTGGTGCTGCAGATGAACAGCCTGAAACCTGAG |
| | | GACACAGCCCTGTACTATTGTGCCGCCGATCGGAAGAGCGTG |
| | | ATGAGCATTAGACCCGATTATTGGGGACAGGGCACACAGGT |
| | | GACAGTGAGTAGCACTAGTACCACGACGCCAGCGCCGCGAC |
| | | CACCAACACCGGCGCCCACCATCGCGTCGCAGCCCCTGTCCC |
| | | TGCGCCCAGAGGCGTGCCGGCCAGCGGCGGGGGGCGCAGTG |
| | | CACACGAGGGGGCTGGACTTCGCCTGTGATATCTACATCTGG |
| | | GCGCCCTTGGCCGGGACTTGTGGGGTCCTTCTCCTGTCACTG |
| | | GTTATCACCCTTTACTGCAAACGGGGCAGAAAGAAACTCCTG |
| | | TATATATTCAAACAACCATTTATGAGACCAGTACAAACTACT |
| | | CAAGAGGAAGATGGCTGTAGCTGCCGATTTCCAGAAGAAGA |
| | | AGAAGGAGGATGTGAACTGAGAGTGAAGTTCAGCAGGAGCG |
| | | CAGACGCCCCCGCGTACCAGCAGGGCCAGAACCAGCTCTAT |
| | | AACGAGCTCAATCTAGGACGAAGAGAGGAGTACGATGTTTT |
| | | GGACAAGAGACGTGGCCGGGACCCTGAGATGGGGGGAAAG |
| | | CCGAGAAGGAAGAACCCTCAGGAAGGCCTGTACAATGAACT |
| | | GCAGAAAGATAAGATGGCGGAGGCCTACAGTGAGATTGGGA |
| | | TGAAAGGCGAGCGCCGGAGGGGCAAGGGGCACGATGGCCTT |
| | | TACCAGGGTCTCAGTACAGCCACCAAGGACACCTACGACGC |
| | | CCTTCACATGCAGGCCCTGCCCCCTCGCTAATGA |
| anti-FITC | SEQ ID NO: 54 | ATGACCATCCTGGGCACCACCTTCGGCATGGTGTTTAGCCTG |
| IL7Rα SMAR | | CTGCAGGTCGTGTCTGGCGATTACAAGGATGACGACGATAA |
| (tVH)-T2A- | | GGACGTGGTCATGACACAGACCCCACTGTCTCTGCCTGTGTC |
| Carvykti | | TCTGGGAGATCAGGCCAGCATCAGCTGCAGATCTAGCCAGA |
| | | GCCTGGTGCACAGCAACGGCAACACCTACCTGCGGTGGTATC |
| | | TGCAGAAGCCCGGCCAGTCTCCTAAGGTGCTGATCTACAAGG |
| | | TGTCCAACAGAGTGTCCGGCGTGCCCGATAGATTTTCTGGCA |
| | | GCGGCTCTGGCACCGACTTCACCCTGAAGATCAATAGAGTGG |
| | | AAGCCGAGGACCTGGGCGTGTACTTCTGTAGCCAGTCTACCC |
| | | ACGTGCCATGGACCTTTGGCGGCGGAACAAAGCTGGAAATC |
| | | ACAACAACCCCTGCTCCTCGGCCTCCTACACCAGCTCCTACA |
| | | ATTGCCAGCCAGCCTCTGTCTCTGAGGCCCGAAGCTTGTAGA |
| | | CCTGCTGCCGGCGGAGCTGTGCATACAAGAGGCCTGGATTTC |
| | | GCCTGCGATCCCATCCTGCTGACAATCAGCATCCTGAGCTTT |
| | | TTCAGCGTGGCCCTGCTGGTCATCCTGGCCTGTGTGCTGTGG |
| | | AAGAAGCGGATCAAGCCCATCGTGTGGCCCAGCCTGCCTGA |
| | | CCACAAGAAAACCCTGGAACACCTGTGCAAGAAGCCCCGGA |
| | | AGAACCTGAACGTGTCCTTCAATCCCGAGAGCTTCCTGGACT |
| | | GCCAGATCCACAGAGTGGACGACATCCAGGCCAGGGACGAA |
| | | GTGGAAGGCTTTCTGCAGGACACATTCCCTCAGCAGCTGGAA |
| | | GAGAGCGAGAAGCAGAGACTCGGAGGCGACGTGCAGAGCC |
| | | CTAATTGCCCTTCTGAGGACGTCGTGATCACCCCTGAGAGCT |
| | | TCGGCAGAGATAGCAGCCTGACATGTCTGGCCGGCAATGTGT |
| | | CCGCCTGTGATGCCCCTATCCTGAGCAGCAGCAGAAGCCTGG |
| | | ATTGCAGAGAGAGCGGCAAGAACGGCCCTCACGTGTACCAG |
| | | GATCTGCTCCTGAGCCTGGGAACCACCAATAGCACACTGCCT |
| | | CCACCATTCAGCCTGCAGAGCGGCATCCTGACACTGAACCCT |
| | | GTTGCTCAGGGCCAGCCAATCCTGACAAGCCTGGGCAGCAA |
| | | TCAAGAAGAGGCCTACGTCACCATGAGCAGCTTCTACCAGA |
| | | ATCAAGGCTCCGGCGAAGGCAGAGGCTCTCTGCTTACATGCG |
| | | GAGATGTGGAAGAGAACCCCGGACCTATGGCTCTGCCTGTC |
| | | ACCGCTCTGCTGCTGCCTCTGGCTCTGCTGCTGCACGCTGCTC |
| | | GCCCTCAGGTCAAACTGGAAGAAAGTGGGGGAGGCCTGGTG |
| | | CAGGCAGGACGGAGCCTGCGCCTGAGCTGCGCAGCATCCGA |
| | | GCACACCTTCAGCTCCCACGTGATGGGCTGGTTTCGGCAGGC |
| | | CCCAGGCAAGGAGAGAGAGTCCGTGGCCGTGATCGGCTGGA |
| | | GGGACATCTCCACATCTTACGCCGATTCTGTGAAGGGCCGGT |
| | | TCACCATCAGCAGAGACAACGCCAAGAAGACACTGTATCTG |
| | | CAGATGAATAGCCTGAAGCCCGAGGACACCGCCGTGTACTA |
| | | TTGCGCAGCAAGGAGAATCGACGCAGCAGACTTTGATTCCTG |
| | | GGGCCAGGGCACCCAGGTGACAGTGTCTAGCGGAGGAGGAG |
| | | GATCTGGAGGAGGAGGAAGCGGAGGAGGAGGATCCGAGGT |
| | | GCAGCTGGTGGAGTCTGGAGGCGGCCTGGTGCAGGCCGGAG |
| | | GCTCTCTGAGGCTGAGCTGTGCAGCATCCGGAAGAACCTTCA |
| | | CAATGGGCTGGTTTAGGCAGGCACCAGGAAAGGAGAGGGAG |
| | | TTCGTGGCAGCAATCAGCCTGTCCCCTACCCTGGCCTACTAT |
| | | GCCGAGTCCGTGAAGGGCAGGTTTACCATCTCTCGCGATAAC |
| | | GCCAAGAATACAGTGGTGCTGCAGATGAACAGCCTGAAACC |
| | | TGAGGACACAGCCCTGTACTATTGTGCCGCCGATCGGAAGA |
| | | GCGTGATGAGCATTAGACCCGATTATTGGGGACAGGGCACA |
| | | CAGGTGACAGTGAGTAGCACTAGTACCACGACGCCAGCGCC |
| | | GCGACCACCAACACCGGCGCCCACCATCGCGTCGCAGCCCCT |
| | | GTCCCTGCGCCCAGAGGCGTGCCGGCCAGCGGGGGGGGCG |
| | | CAGTGCACACGAGGGGGCTGGACTTCGCCTGTGATATCTACA |
| | | TCTGGGCGCCCTTGGCCGGGACTTGTGGGGTCCTTCTCCTGT |
| | | CACTGGTTATCACCCTTTACTGCAAACGGGGCAGAAAGAAA |
| | | CTCCTGTATATATTCAAACAACCATTTATGAGACCAGTACAA |
| | | ACTACTCAAGAGGAAGATGGCTGTAGCTGCCGATTTCCAGA |
| | | AGAAGAAGAAGGAGGATGTGAACTGAGAGTGAAGTTCAGCA |
| | | GGAGCGCAGACGCCCCCGCGTACCAGCAGGGCCAGAACCAG |
| | | CTCTATAACGAGCTCAATCTAGGACGAAGAGAGGAGTACGA |
| | | TGTTTTGGACAAGAGACGTGGCCGGGACCCTGAGATGGGGG |
| | | GAAAGCCGAGAAGGAAGAACCCTCAGGAAGGCCTGTACAAT |
| | | GAACTGCAGAAAGATAAGATGGCGGAGGCCTACAGTGAGAT |
| | | TGGGATGAAAGGCGAGCGCCGGAGGGGCAAGGGGCACGAT |
| | | GGCCTTTACCAGGGTCTCAGTACAGCCACCAAGGACACCTAC |
| | | GACGCCCTTCACATGCAGGCCCTGCCCCCTCGCTAATGA |
| anti-FITC | SEQ ID NO: 55 | ATGACCATCCTGGGCACCACCTTCGGCATGGTGTTTAGCCTG |
| IL7Rα SMAR | | CTGCAGGTCGTGTCTGGCGATTACAAGGATGACGACGATAA |
| (GSG)-T2A- | | GGACGTGGTCATGACACAGACCCCACTGTCTCTGCCTGTGTC |
| Carvykti | | TCTGGGAGATCAGGCCAGCATCAGCTGCAGATCTAGCCAGA |
| | | GCCTGGTGCACAGCAACGGCAACACCTACCTGCGGTGGTATC |
| | | TGCAGAAGCCCGGCCAGTCTCCTAAGGTGCTGATCTACAAGG |
| | | TGTCCAACAGAGTGTCCGGCGTGCCCGATAGATTTTCTGGCA |
| | | GCGGCTCTGGCACCGACTTCACCCTGAAGATCAATAGAGTGG |
| | | AAGCCGAGGACCTGGGCGTGTACTTCTGTAGCCAGTCTACCC |
| | | ACGTGCCATGGACCTTTGGCGGCGGAACAAAGCTGGAAATC |
| | | AAGAGCAGCGCCGACGACGCCAAGAAGGACGCCGCTAAGA |
| | | AGGATGACGCAAAGAAAGACGATGCCAAAAAGGATGGCGG |
| | | CGTGAAGCTGGACGAAACAGGCGGAGGACTTGTTCAGCCTG |
| | | GCGGAGCCATGAAGCTGAGCTGTGTGACCAGCGGCTTCACCT |
| | | TTGGCCACTACTGGATGAACTGGGTCCGACAGAGCCCTGAG |
| | | AAAGGCCTGGAATGGGTCGCCCAGTTCAGAAACAAGCCCTA |
| | | CAACTACGAAACCTACTACAGCGACAGCGTGAAGGGCAGAT |
| | | TCACCATCAGCCGGGACGACAGCAAGTCCAGCGTGTACCTG |
| | | CAGATGAACAACCTGCGCGTGGAAGATACCGGCATCTACTA |
| | | CTGTACCGGCGCCAGCTACGGCATGGAATATCTCGGCCAGG |
| | | GCACCAGCGTGACCGTGTCTGGATCTGGCCCCATCCTGCTGA |
| | | CAATCAGCATCCTGAGCTTTTTCAGCGTGGCCCTGCTGGTCA |
| | | TCCTGGCCTGTGTGCTGTGGAAGAAGCGGATCAAGCCCATCG |
| | | TGTGGCCCAGCCTGCCTGACCACAAGAAAACCCTGGAACAC |
| | | CTGTGCAAGAAGCCCCGGAAGAACCTGAACGTGTCCTTCAAT |
| | | CCCGAGAGCTTCCTGGACTGCCAGATCCACAGAGTGGACGA |
| | | CATCCAGGCCAGGGACGAAGTGGAAGGCTTTCTGCAGGACA |
| | | CATTCCCTCAGCAGCTGGAAGAGAGCGAGAAGCAGAGACTC |
| | | GGAGGCGACGTGCAGAGCCCTAATTGCCCTTCTGAGGACGTC |
| | | GTGATCACCCCTGAGAGCTTCGGCAGAGATAGCAGCCTGAC |
| | | ATGTCTGGCCGGCAATGTGTCCGCCTGTGATGCCCCTATCCT |
| | | GAGCAGCAGCAGAAGCCTGGATTGCAGAGAGAGCGGCAAG |
| | | AACGGCCCTCACGTGTACCAGGATCTGCTCCTGAGCCTGGGA |
| | | ACCACCAATAGCACACTGCCTCCACCATTCAGCCTGCAGAGC |
| | | GGCATCCTGACACTGAACCCTGTTGCTCAGGGCCAGCCAATC |
| | | CTGACAAGCCTGGGCAGCAATCAAGAAGAGGCCTACGTCAC |
| | | CATGAGCAGCTTCTACCAGAATCAAGGCTCCGGCGAAGGCA |
| | | GAGGCTCTCTGCTTACATGCGGAGATGTGGAAGAGAACCCC |
| | | GGACCTATGGCTCTGCCTGTCACCGCTCTGCTGCTGCCTCTG |
| | | GCTCTGCTGCTGCACGCTGCTCGCCCTCAGGTCAAACTGGAA |
| | | GAAAGTGGGGGAGGCCTGGTGCAGGCAGGACGGAGCCTGCG |
| | | CCTGAGCTGCGCAGCATCCGAGCACACCTTCAGCTCCCACGT |
| | | GATGGGCTGGTTTCGGCAGGCCCCAGGCAAGGAGAGAGAGT |
| | | CCGTGGCCGTGATCGGCTGGAGGGACATCTCCACATCTTACG |
| | | CCGATTCTGTGAAGGGCCGGTTCACCATCAGCAGAGACAAC |
| | | GCCAAGAAGACACTGTATCTGCAGATGAATAGCCTGAAGCC |
| | | CGAGGACACCGCCGTGTACTATTGCGCAGCAAGGAGAATCG |
| | | ACGCAGCAGACTTTGATTCCTGGGGCCAGGGCACCCAGGTG |
| | | ACAGTGTCTAGCGGAGGAGGAGGATCTGGAGGAGGAGGAA |
| | | GCGGAGGAGGAGGATCCGAGGTGCAGCTGGTGGAGTCTGGA |
| | | GGCGGCCTGGTGCAGGCCGGAGGCTCTCTGAGGCTGAGCTG |
| | | TGCAGCATCCGGAAGAACCTTCACAATGGGCTGGTTTAGGCA |
| | | GGCACCAGGAAAGGAGAGGGAGTTCGTGGCAGCAATCAGCC |
| | | TGTCCCCTACCCTGGCCTACTATGCCGAGTCCGTGAAGGGCA |
| | | GGTTTACCATCTCTCGCGATAACGCCAAGAATACAGTGGTGC |
| | | TGCAGATGAACAGCCTGAAACCTGAGGACACAGCCCTGTAC |
| | | TATTGTGCCGCCGATCGGAAGAGCGTGATGAGCATTAGACCC |
| | | GATTATTGGGGACAGGGCACACAGGTGACAGTGAGTAGCAC |
| | | TAGTACCACGACGCCAGCGCCGCGACCACCAACACCGGCGC |
| | | CCACCATCGCGTCGCAGCCCCTGTCCCTGCGCCCAGAGGCGT |
| | | GCCGGCCAGCGGCGGGGGGCGCAGTGCACACGAGGGGGCTG |
| | | GACTTCGCCTGTGATATCTACATCTGGGCGCCCTTGGCCGGG |
| | | ACTTGTGGGGTCCTTCTCCTGTCACTGGTTATCACCCTTTACT |
| | | GCAAACGGGGCAGAAAGAAACTCCTGTATATATTCAAACAA |
| | | CCATTTATGAGACCAGTACAAACTACTCAAGAGGAAGATGG |
| | | CTGTAGCTGCCGATTTCCAGAAGAAGAAGAAGGAGGATGTG |
| | | AACTGAGAGTGAAGTTCAGCAGGAGCGCAGACGCCCCCGCG |
| | | TACCAGCAGGGCCAGAACCAGCTCTATAACGAGCTCAATCT |
| | | AGGACGAAGAGAGGAGTACGATGTTTTGGACAAGAGACGTG |
| | | GCCGGGACCCTGAGATGGGGGGAAAGCCGAGAAGGAAGAA |
| | | CCCTCAGGAAGGCCTGTACAATGAACTGCAGAAAGATAAGA |
| | | TGGCGGAGGCCTACAGTGAGATTGGGATGAAAGGCGAGCGC |
| | | CGGAGGGGCAAGGGGCACGATGGCCTTTACCAGGGTCTCAG |
| | | TACAGCCACCAAGGACACCTACGACGCCCTTCACATGCAGG |
| | | CCCTGCCCCCTCGCTAATGA |

Activated T-cells were transduced using lentiviral vectors expressing IL7Rα (WT) SMAR-Carvykti (SEQ ID NO: 48), IL7Rα (tICD) SMAR-Carvykti (SEQ ID NO: 53), IL7Rα˜(tVH) SMAR-Carvykti (SEQ ID NO: 54), or IL7Rα (GSG) SMAR-Carvykti (SEQ ID NO: 55), and their tumor killing ability was tested in a MM.1S tumor rechallenge assay. Briefly, the same CAR+ rate of different groups of SMAR-Carvykti-expressing T-cells were co-cultured with MM.1S-GFP cells in an L/T ratio of 1/20. Every 2-3 days, fluorescent images of tumor cells were taken, then additional MM.1S-GFP cells were added to the co-culture. T-cell activation with FITC was not performed prior to the tumor killing assay.

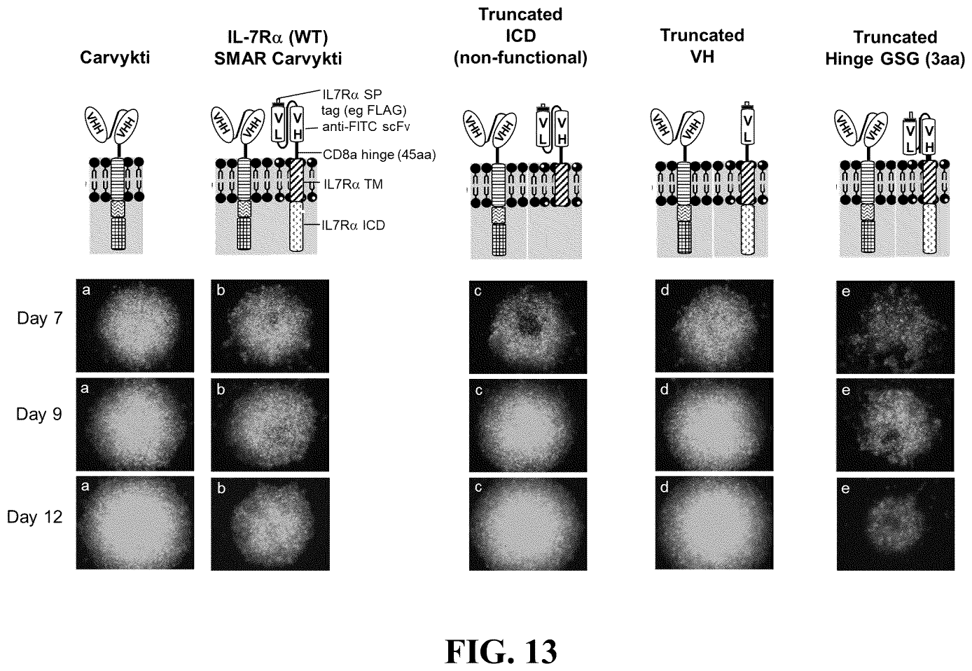

FIG. 13 illustrates the tested constructs (top) and the fluorescent images taken at day 7, 9, and 12 after co-culture (bottom) for cells expressing Carvykti alone (images labeled “a”), Carvykti with the IL,7Rα (WT) SMAR (images labeled “b”), Carvykti with the IL,7Rα (tICD) SMAR (images labeled “c”), Carvykti with the IL,7Rα (tVH) SMAR (images labeled “d”), and Carvykti with the IL,7Rα (GSG) SMAR (images labeled “e”). In FIG. 13, increased brightness corresponds to poor tumor cell killing, and reduced brightness corresponds to strong tumor cell killing. Cells that co-expressed the IL7Rα (WT) SMAR exhibited increased tumor cell killing at days 7, 9, and 12 compared to cells expressing Carvykti alone. Killing was reduced compared to the Caryvkti only control when the IL7Rα intracellular domain was truncated and when the anti-FITC scFv was truncated. Killing was improved on days 7, 9, and 12 compared to the Carvykti only control when the hinge was shortened to 3 amino acids (the hinge is 45 amino acids in the full length IL7Rα SMAR). Without wishing to be bound by theory, these results suggest that the scFv domain of the IL7Rα SAMR may interact with the extracellular domain of Carvykti, which may explain the activator-independent activity of the IL7Rα SMAR in the presence of Carvykti. Moreover, these results indicate that the adjustments to the hinge (e.g., length, rigidity) may be used to increase or decrease the extent of activator-independent activity.

### Example 15: In Vitro Tumor Rechallenge of Immune Cells with Engineered Cytokine Receptor Switches

This example describes studies investigating the activity of immune cells expressing engineered cytokine receptor switches in an in vitro tumor rechallenge assay.

T-cells were transfected to express a BCMA-targeting CAR and either (i) no engineered cytokine receptor switch (“bb2121”), (ii) an IL2Rβ/7 engineered cytokine receptor switch (“IL2Rβ/γ SMAR bb2121”), or (iii) an IL7Rα engineered cytokine receptor switch (“IL7Rα SMAR bb2121”). The CAR T-cells were treated with FITC-dextran to activate the engineered cytokine receptor switches, if present. The activated CAR T-cells were co-cultured on day 0 with multiple myeloma tumor cells (MM.1S tumor cells) at an effector to target ratio (E/T) of 1/4 CAR⁺ cells to tumor cells by combining 1×10⁵ CAR⁺ cells and 4×10⁵ MM.1S target cells. Tumor rechallenges were performed on day 2 (1^{st} Rechallenge) and day 4 (2^{nd} rechallenge) by removing cells for analysis and adding 4×10⁵ MM.1S target cells (E/T ratio of 1/4) to the CAR⁺ cells. Cells from the second rechallenge were removed on day 6 for analysis.

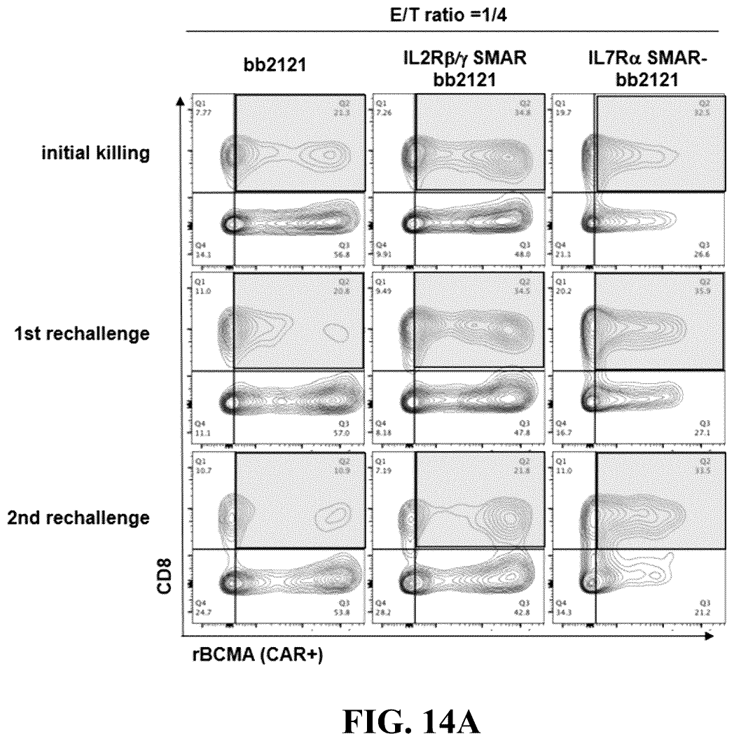

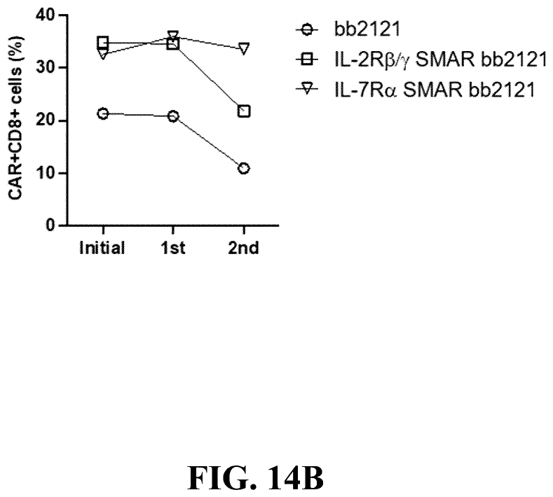

FIGS. 14A and 14B illustrate flow cytometry results for the in vitro tumor rechallenge assay performed for bb2121, IL2Rβ/7 SMAR bb2121, and IL7Rα SMAR bb2121 CAR T-cells. As shown in FIGS. 14A and 14B, cells expressing a bb2121 CAR with either a IL2Rβ/γ SMAR or a IL7Rα SMAR exhibited increased amounts and persistence of CD8+ cells, compared to cells expressing the bb2121 CAR only. These results demonstrate that the IL7Rα SMAR increased the persistence of CD8+ CAR T cells during tumor rechallenge.

### Example 16: In Vitro Tumor Rechallenge of Immune Cells with Engineered Cytokine Receptor Switches without Activation

This example describes studies investigating the activity of immune cells expressing engineered cytokine receptor switches in an in vitro tumor rechallenge assay, without activation of the switches during manufacturing.

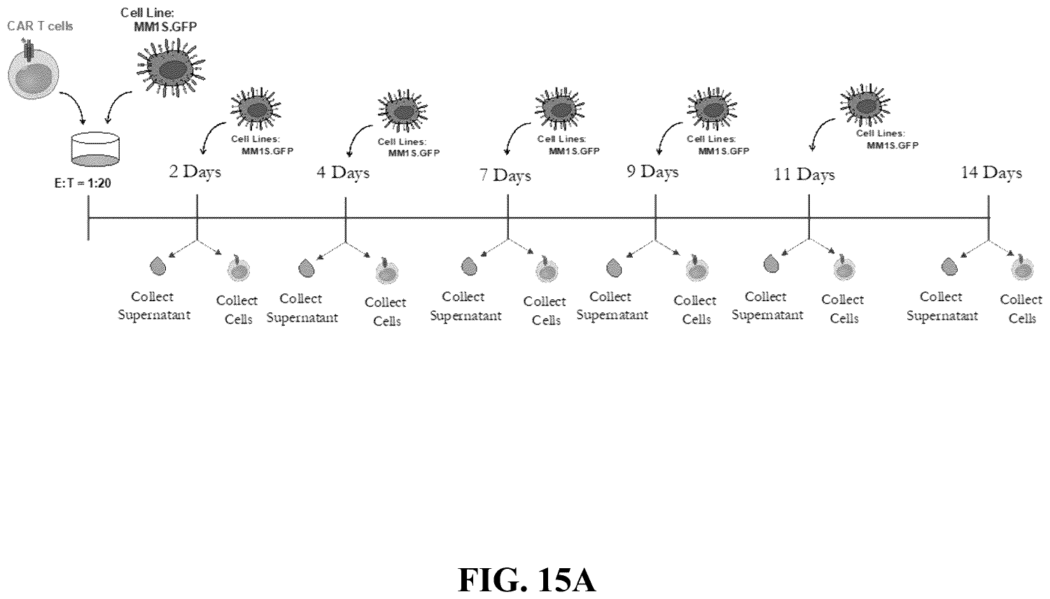

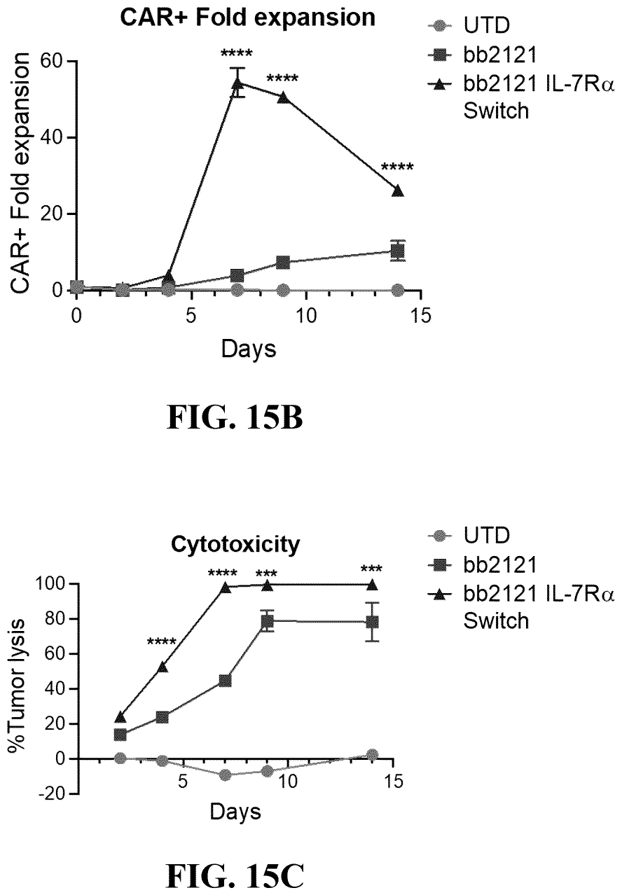

An in vitro tumor rechallenge assay was performed using T-cells from a different donor than the preceding Examples. T-cells were transfected to express a BCMA-targeting CAR and either (i) no engineered cytokine receptor switch (“bb2121”) or (ii) an IL7Rα engineered cytokine receptor switch (“IL7Rα SMAR bb2121”). The CAR T-cells were not activated with FITC during manufacturing. The timeline for the rechallenge assay is shown in FIG. 15A. Briefly, the activated CAR T-cells were co-cultured on day 0 with GFP-expressing MM.1S tumor cells in an effector to target ratio (E/T) of 1:20 CAR⁺ cells to tumor cells. Tumor rechallenges were performed on days 2, 4, 7 9, and 11. Cells and supernatant were collected on days 2, 4, 7, 9, 11, and 14 for analysis.

FIGS. 15B and 15B are graphs illustrating CAR T-cell expansion (FIG. 15B) and tumor cell killing (FIG. 15C) in the tumor rechallenge assay. CAR T-cells including the IL-7Rα SMAR demonstrated superior tumor cell killing and T-cell expansion than CAR T-cells expressing the bb2121 CAR alone and untransfected control T-cells (UTD). These results corroborate the previous data shown in Examples 3 and 15, thereby demonstrating that the previous results are reproducible and independent of donor cell population. Notably, the IL7Rα SMAR was not activated with FITC during manufacturing, which suggests that the observed effects are attributable to activator-independent activity of the IL7Rα SMAR.

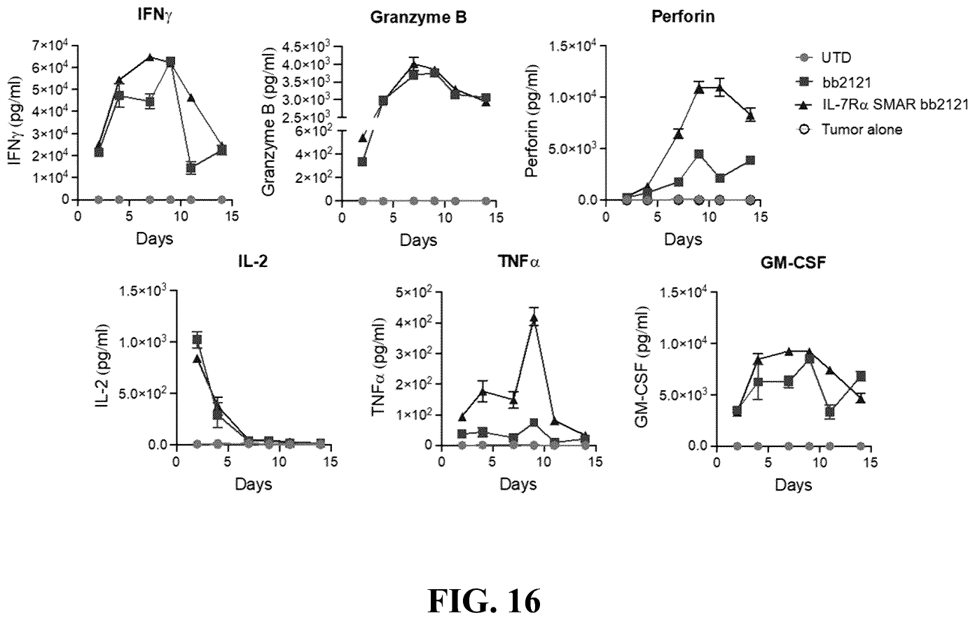

FIG. 16 is a series of graphs showing the effect of an IL7Rα SMAR on cytokine expression in bb2121 CAR T-cells. The levels of IFNγ, granzyme B, perforin, IL2, TNFα, and GM-CSF were assayed in supernatant samples collected from untransfected control T-cells, bb2121 CAR T-cells, IL7Rα SMAR bb2121 CAR T-cells, and tumor cells without T-cells. As shown in FIG. 16, the IL7Rα SMAR bb2121 CAR T-cells had higher levels of perforin and TNFα compared to the bb2121 CAR T-cells, which is consistent with the superior tumor killing observed with the IL7Rα SMAR bb2121 CAR T-cells. No significant differences in the levels of IFNγ, granzyme B, IL2, and GM-CSF in the IL7Rα SMAR bb2121 CAR T-cells versus the bb2121 CAR T-cells were observed.

### Example 17: Control of T-Cell Phenotypes in Immune Cells with Engineered Cytokine Receptor Switches and an Anti-BCMA CAR (Carvykti)

This example describes studies investigating the control of cell phenotypes in immune cells expressing engineered cytokine receptor switches and an anti-BCMA CAR (Carvykti).

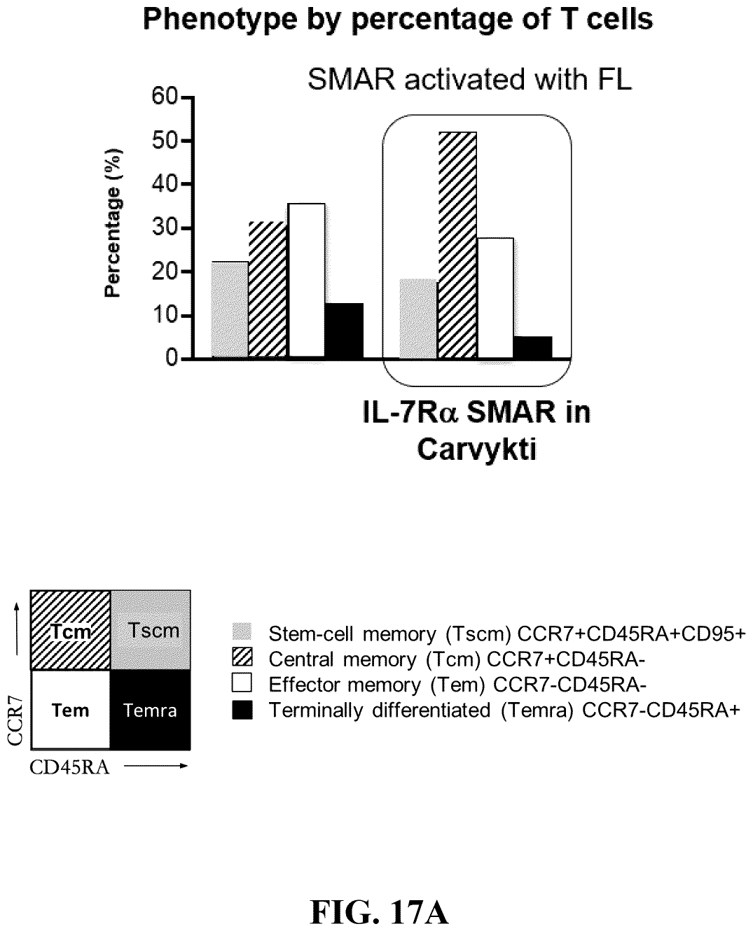

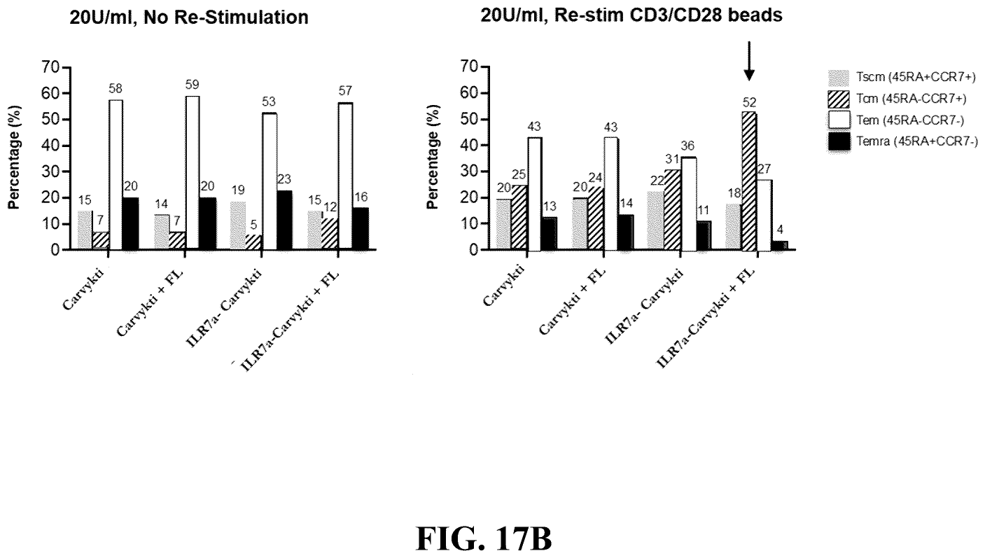

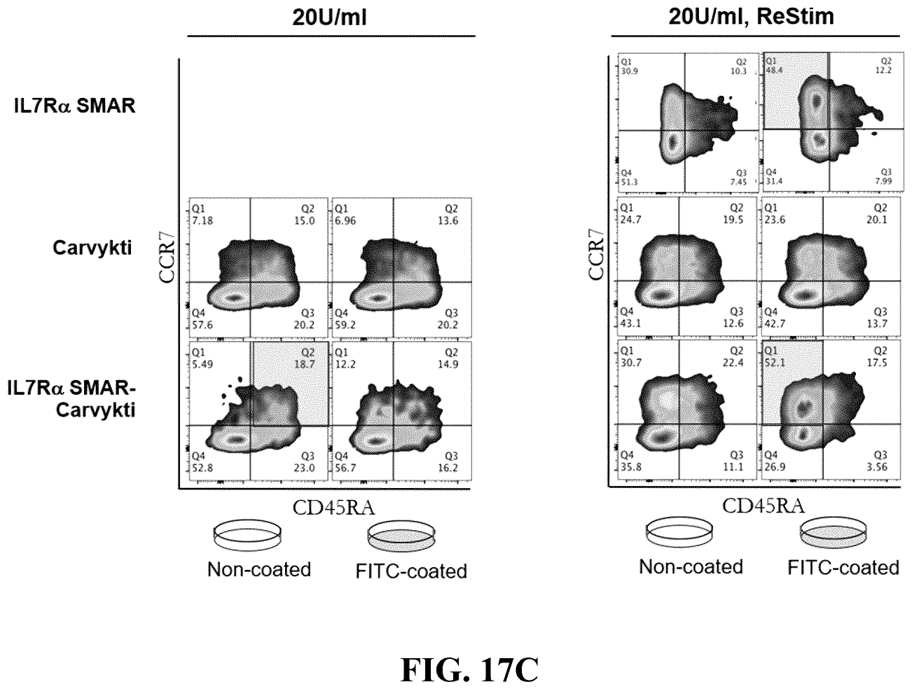

T-cells were transfected to express a BCMA-targeting CAR (Carvykti). After stimulation with CD3/CD28 Dynabeads, an engineered cytokine receptor switch derived from IL7Rα (“IL7Rα SMAR,” SEQ ID NO: 4) was transfected into T-cells in the presence of IL2 (100 U/mL). A portion of the IL2-treated cells were grown on a FITC-dextran-coated surface to activate the engineered cytokine receptors and promote conversion from effector memory to central memory cell phenotype. The phenotype distribution of each population of T-cells was determined by assaying for phenotypic markers. As illustrated in FIG. 17A (bottom), cells positive for CCR7, CD45RA, and CD95 were identified as having a stem-cell memory (T_{scm}) phenotype, cells positive for CCR7 but negative for CD45RA were identified as having a central memory (T_{cm}) phenotype, cells negative for both CCR7 and CD45RA were identified has having an effector memory (Tₑₘ) phenotype, and cells positive for CD45RA but negative for CCR7 were identified as having an effector memory re-expressing CD45RA (Tₑₘₐ) phenotype.

T-cells treated with IL2 and the FITC-dextran small molecule activator contained a higher portion of central memory cells than the population of cells treated with IL2 alone. As shown in FIG. 17A (by percent of cells), most of the T-cells treated with IL2 and FITC-dextran (bars at right, in box) had a central memory phenotype, while most of the T-cells treated with IL2 alone (bars at left) had an effector memory phenotype. These data indicate that the IL7Rα SMAR promoted conversion from effector memory phenotype to central memory phenotypes when activated by FITC.

FIG. 17B is a series of graphs showing a comparison of T-cell phenotypes between populations with CD3/CD28 re-stimulation (right) and without re-stimulation (left). Both groups were also treated with 20 U/mL IL2. The cell populations that were tested were T-cells with the Carvykti CAR alone (“Carvykti”), T-cells with the Carvykti CAR alone that were treated with FITC-dextran (“Carvykti+FL”), T-cells with the Carvykti CAR and the IL7Rα SMAR (“IL7Rα-Carvykti”), and T-cells with the Carvykti CAR and the IL7Rα SMAR that were treated with FITC-dextran (“IL7Rα-Carvykti+FL”). As shown in the left graph in FIG. 17B, in the absence of CD3/CD28 re-stimulation, the majority of the cells in all populations had an effector memory phenotype. As shown in the right graph in FIG. 17B, after CD3/CD28 re-stimulation, which was performed 7 days after the initial CD3/CD28 stimulation, most of T-cells with the Carvykti CAR and the IL7Rα SMAR that were treated with FITC-dextran had a central memory phenotype, whereas the majority of the cells in the other populations had an effector memory phenotype. These results indicate that IL7Rα SMAR activation in Carvykti CAR T-cells induced conversion into a central memory phenotype. T-cells with the Carvykti CAR and the IL7Rα SMAR that were not treated with FITC-dextran were primarily effector memory phenotypes, which suggests that there was little or no FITC-independent activity in these cells.

FIG. 17C is a series of flow cytometry plots showing a comparison of T-cell phenotypes between populations with CD3/CD28 re-stimulation (right) and without re-stimulation (left), and with FITC activation (“FITC-coated”) and without FITC activation (“non-coated”). All groups were also treated with 20 U/mL IL2. The cell populations that were tested were T-cells with the IL7Rα SMAR alone (“IL7Rα SMAR”), T-cells with the Carvykti CAR alone (“Carvykti”), and T-cells with the IL7Rα SMAR and the Carvykti CAR (“IL7Rα SMAR-Carvykti”). As shown in FIG. 17C, activation of the IL7Rα SMAR with FITC generated Carvykti CAR T-cells having a memory phenotype ex vivo.

### Example 18: In Vitro Tumor Cell Killing by Immune Cells with Engineered Cytokine Receptor Switches and an Anti-BCMA CAR (Carvykti)

This example describes studies investigating the in vitro tumor cell killing by immune cells expressing engineered cytokine receptor switches and an anti-BCMA CAR (Carvykti).

T-cells were transfected to express a BCMA-targeting CAR (Carvykti). After stimulation with CD3/CD28 Dynabeads, an engineered cytokine receptor switch derived from IL7Rα (“IL7Rα SMAR,” SEQ ID NO: 4) was transduced into T-cells in the presence of IL2 (100 U/mL). A portion of the IL2-treated cells were grown on a FITC-dextran-coated surface to activate the engineered cytokine receptors and promote conversion from effector memory to central memory cell phenotype. Cells were expanded in the presence of 20 U/mL IL2. CD3/CD28 restimulation was performed 7 days after the initial stimulation.

Tumor killing ability was tested in a MM.1S tumor rechallenge assay. Briefly, the same CAR⁺ rate of different groups of T-cells were co-cultured with MM.1S-GFP cells in an E/T ratio of 1/20. Every 2-3 days, fluorescent images of tumor cells were taken, then additional MM.1S-GFP cells were added to the co-culture. No treatments with IL2, FITC activation, or CD3/CD28 Dynabeads were performed during the tumor rechallenge assay.

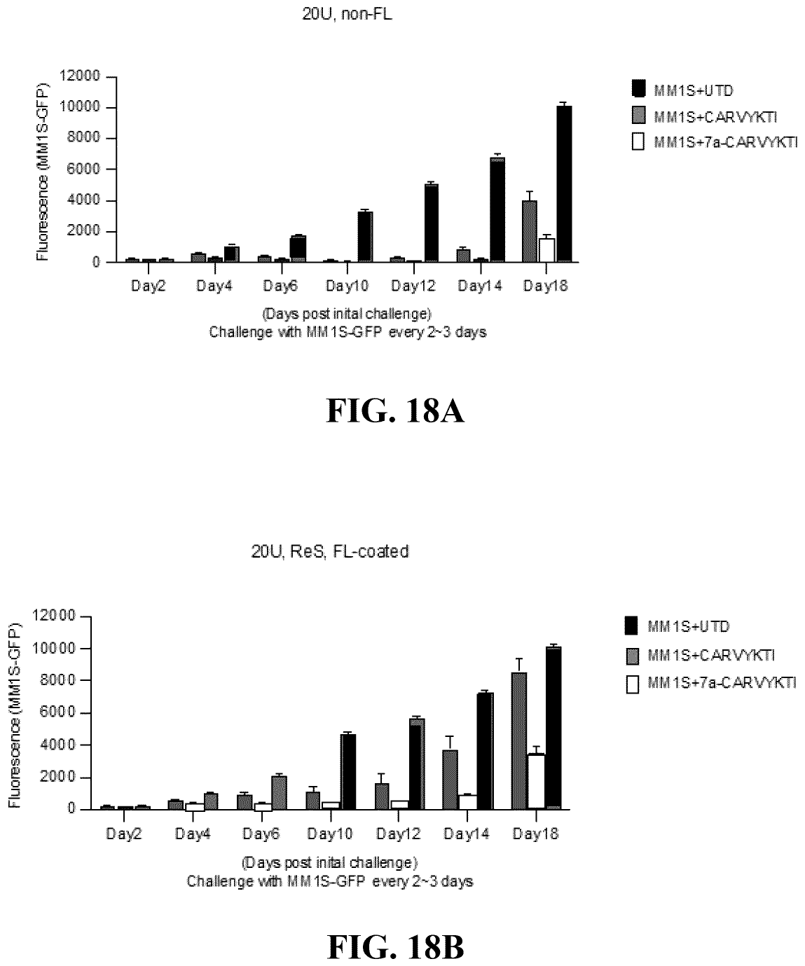

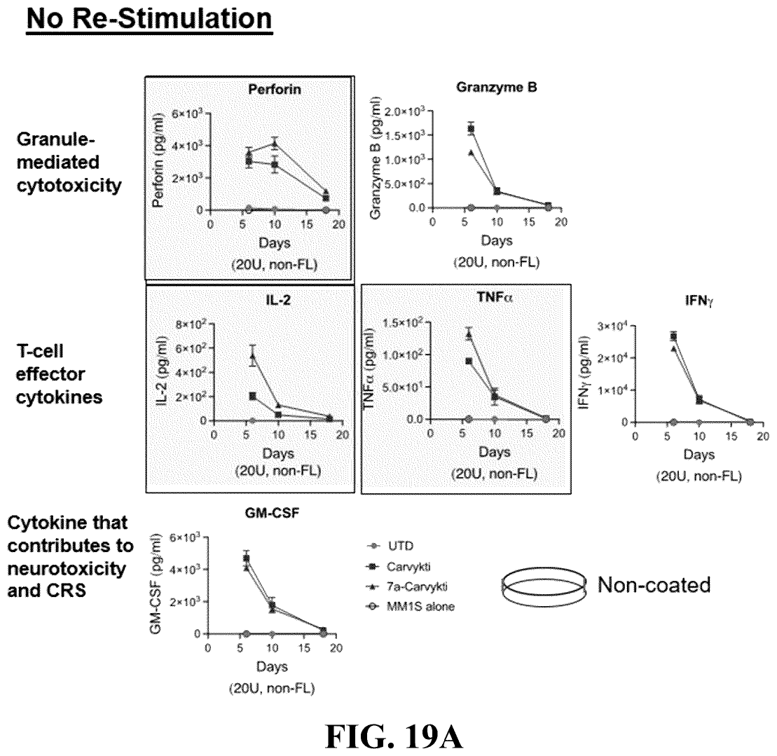

FIG. 18A is a graph illustrating tumor cell killing by T-cell populations manufactured on non-coated plates in the absence of CD3/CD28 restimulation, and FIG. 18B is a graph illustrating tumor cell killing by T-cell populations manufactured on FITC-coated plates with CD3/CD28 re-stimulation. The graphs show fluorescence of GFP-expressing MM.1S cells co-cultured with untransfected control T-cells (“MM1S+UTD”), with T-cells expressing the Carvykti CAR alone (“MM1S+CARVYKTI”), or with T-cells expressing the IL7Rα SMAR and the Carvykti CAR (“MMS+7a-CARVYKTI”); lower fluorescence values indicate greater tumor cell killing. As shown in FIGS. 18A and 18B, T-cells including the IL7Rα SMAR demonstrated superior tumor cell killing compared to T-cells including the Carvykti CAR alone. Moreover, as shown in FIG. 18A, tumor cell killing was observed even without FITC-activation, which suggests that both FITC-independent activity and FITC-mediated activation of the SMAR can enhance tumor killing under the study conditions. The reduced efficacy with CD3/CD28 restimulation may be attributable to faster T-cell exhaustion.

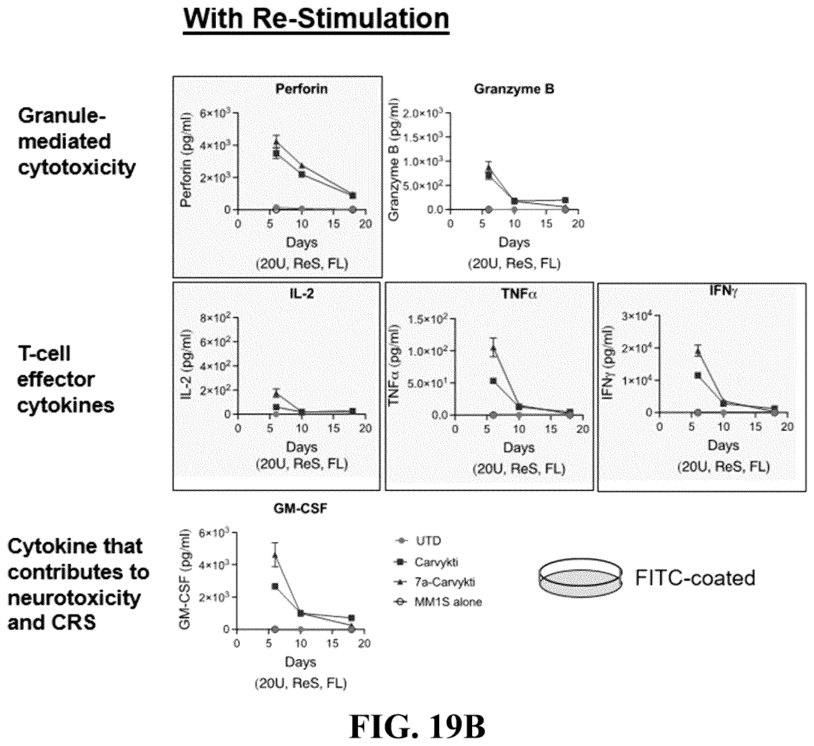

FIG. 19A is a series of graphs illustrating cytokine production during tumor rechallenge by cells manufactured without CD3/CD28 re-stimulation and without FITC activation, and FIG. 19B is a series of graphs illustrating cytokine production during tumor rechallenge by cells manufactured with CD3/CD28 re-stimulation and FITC activation. Levels of perforin, granzyme B, IL2, TNFα, and GM-CSF were measured in samples collected from the media of coculture of tumor cells with untransfected control T-cells (“UTD”), Carvykti CAR T-cells (“Carvykti”), IL7Rα SMAR Carvykti CAR T-cells (“7a-Carvykti”), and tumor cells without T-cells (“MM1S alone”). As shown in FIGS. 19A and 19B, IL2, TNFα, IFNγ, and perforin were upregulated at early time points in the in vitro tumor rechallenge assay in cells expressing the IL7Rα SMAR, both with and without FITC activation, which suggests the involvement of FITC-independent activity of the SMAR.

### Example 19: In Vivo Anti-Tumor Activity of Immune Cells with Engineered Cytokine Receptor Switches and an Anti-BCMA CAR (Bb2121)

This example describes a dose finding study with T-cells expressing an anti-BMCA CAR (bb2121) alone (“bb2121”) and with an IL7Rα SMAR (“IL7Rα SMAR bb2121”) in an MM.1S-luc-GFP disseminated model. The studies were conducted to validate anti-tumor activity using an internally generated MM1.S-luc-GFP cell line. It was contemplated that differentiation between bb2121 alone and IL7Rα SMAR bb2121 in vivo may be observed by using a more aggressive MM.1S tumor model in combination with fewer CAR-T cells.

Mice were acquired from Charles River Lab. The mice were female mice of strain NCG, and were 7-8 weeks old at the start of treatment. 28 animals were inoculated, and 28 were used in the study.

The MM.1S-luc-GFP cells used in this Example were generated in-house. The cells were maintained in RPMI 1640 medium supplemented with 10% FBS. The passage number of the frozen cells was P3 and the passage number of the inoculated cells was P6.

MM.1S cells were inoculated into 6-7-week-old female NCG mice in a volume of 200 μL per mouse. Cell inoculums were prepared using sterile DPBS at 2.5 million/mouse, or 12.5 million/mL. MM.1S-luc-GFP cells were aseptically injected intravenously into the right lateral tail vein of the mice using a 26 G 5/8 needle. Six days after MM.1S cell inoculations, mice were imaged and randomized into seven groups as shown in Table 13 below. The average bioluminescence signal was 4×10⁸ photons/second (total flux) for each treatment group after randomization.

On Day 0 (start of treatment, six days after MM.1S inoculation), frozen bb2121 vials alone and IL7Rα SMAR bb2121 vials were pooled and prepared for CAR-T injections. Cells were injected intravenously in amounts of 0.5×10⁶, 2×10⁶, or 5×10⁶ CAR⁺ T cells per mouse.

TABLE 13
Study Groups

| | | Number of | Number |
|---|---|---|---|
| Group | Treatment | CAR+ T Cells | of Mice |
| 1 | Vehicle | N/A | 4 |
| 2 | bb2121 | 0.5 × 10⁶ | 4 |
| 3 | IL7Rα-bb2121 | 0.5 × 10⁶ | 4 |
| 4 | bb2121 | 2 × 10⁶ | 4 |
| 5 | IL7Rα-bb2121 | 2 × 10⁶ | 4 |
| 6 | bb2121 | 5 × 10⁶ | 4 |
| 7 | IL7Rα-bb2121 | 5 × 10⁶ | 4 |

Ventral and dorsal images were acquired using an IVIS Lumina one day before treatment, then weekly to monitor tumor growth. The bioluminescence signals were calculated on each day of imaging using Living Image software. The sum of ventral and dorsal images was used for the calculations of mean BLI. At the end of the study, data was log transformed and analyzed using an unpaired t-test for statistical analysis.

Persistence measurements from blood were collected from 5×10⁶ per mouse groups for both bb2121 alone and IL7Rα SMAR bb2121. Specifically, 100 μL of whole blood was collected from each mouse into EDTA-coated tubes, and 500 μL of ACK lysis buffer was added to each. Samples were vortexed and left to sit for 5 minutes before being centrifuged at 1200 rpm for 5 minutes. Samples were lysed and centrifuged a further two times to remove red blood cells (RBCs). Cells were then resuspended in 2 mL BD Pharmingen Stain Buffer and passed through 70 μm cell strainers. Finally, samples were centrifuged at 1200 rpm for 5 minutes and resuspended in Bambankers freezing media (Nippon Genetics Europe Gmbh).

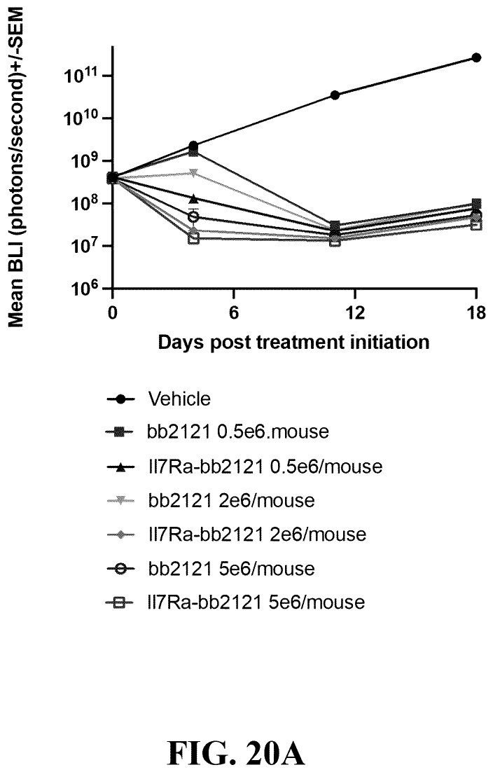

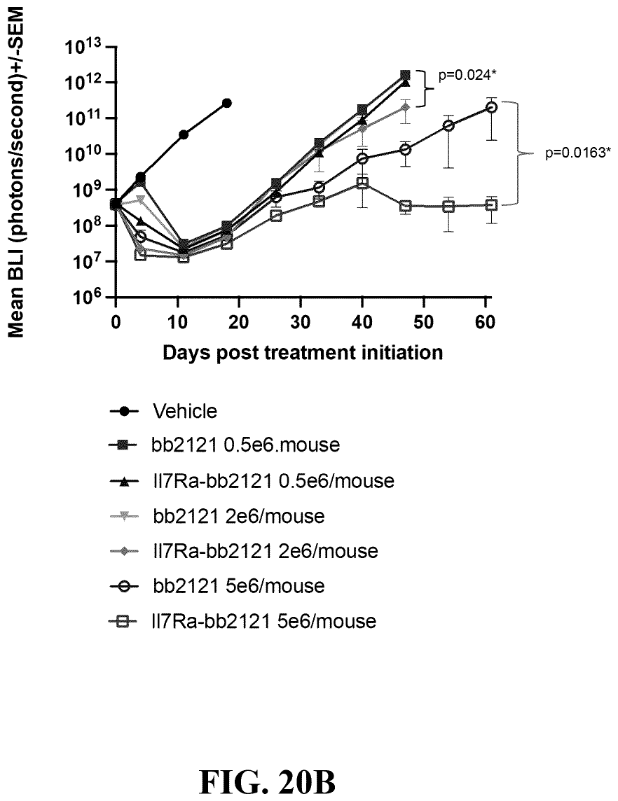

FIG. 20A is a graph illustrating tumor cell killing in the various study groups through Day 18 (lower bioluminescence intensity (BLI) corresponds to higher killing). All treatment groups exhibited significant killing compared to the vehicle only control, as indicated in Table 14 below.

TABLE 14
Day 18 Results

| | | Mean BLI and p values |
|---|---|---|
| | Treatment | compared to Vehicle |
| Group | (once per week) | on Day 8 |
| 1 | Vehicle (PBS) | 2.7 × 10¹¹ |
| 2 | bb2121 (0.5 × 10⁶) | 9.7 × 10⁷ p < 0.0001 |
| 3 | IL7Rα-bb2121 (0.5 × 10⁶) | 7.5 × 10⁷ p < 0.0001 |
| 4 | bb2121 (2 × 10⁶) | 1.0 × 10⁸ p < 0.0001 |
| 5 | IL7Rα-bb2121 (2 × 10⁶) | 4.8 × 10⁷ p < 0.0001 |
| 6 | bb2121 (5 × 10⁶) | 5.3 × 10⁷ p < 0.0001 |
| 7 | IL7Rα-bb2121 (5 × 10⁶) | 3.1 × 10⁷ p < 0.0001 |

FIG. 20B is a graph illustrating tumor cell killing in the various study groups through Day 60 (lower BLI corresponds to higher killing). As shown in FIG. 10B, co-expression of IL7Rα SMAR with the bb2121 CAR demonstrated superior tumor cell killing in the MM.1S mouse model compared to the bb2121 CAR alone.

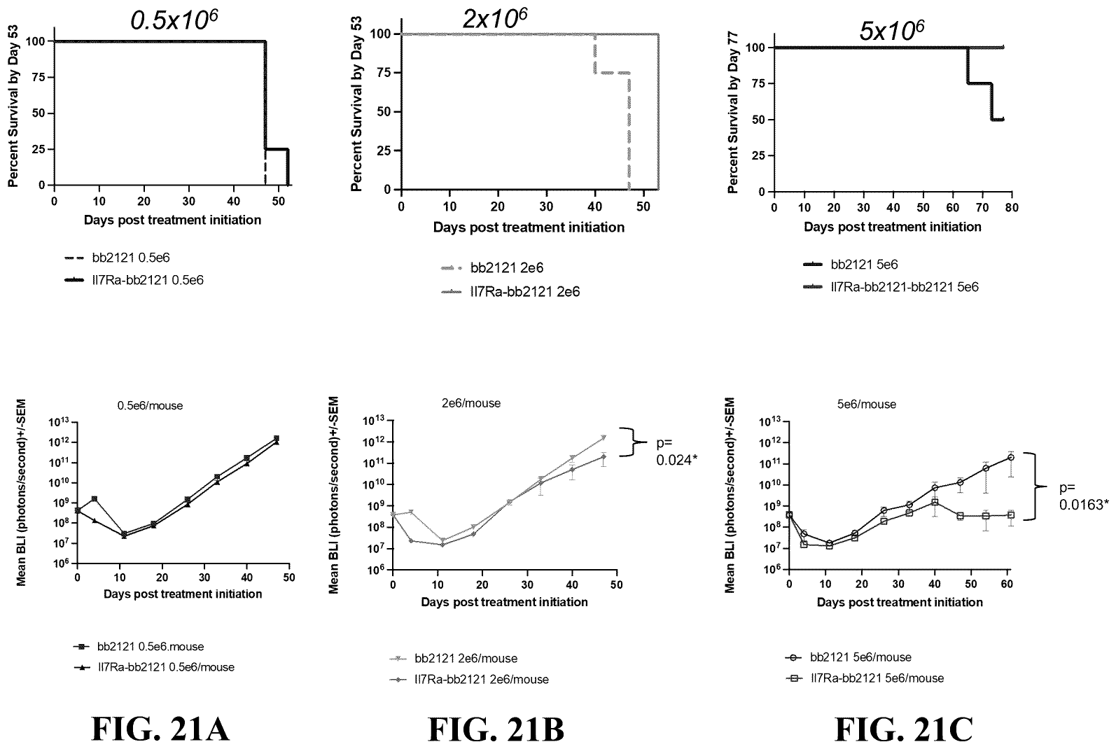

FIGS. 21A-21C are graphs illustrating survival (top) and tumor cell killing (bottom) in mice treated with 0.5×10⁶ (FIG. 21A), 2×10⁶ (FIG. 21B), and 5×10⁶ (FIG. 21C) bb2121 or IL7Rα-bb2121 cells. As shown in FIG. 21A, at Day 52, there was 0% survival in the bb2121 group and 25% survival in the IL7Rα-bb2121 group. As shown in FIG. 21B, at Day 53, there was 0% survival in the bb2121 group and 100% survival in the IL7Rα-bb2121 group. As shown in FIG. 12C, at Day 77, there was 50% survival in the bb2121 group and 100% survival in the IL7Rα-bb2121. These data indicated that superior survival was observed in mice treated with IL7Rα-bb2121 at higher doses (2×10⁶ and 5×10⁶) compared to mice treated with bb2121 only.

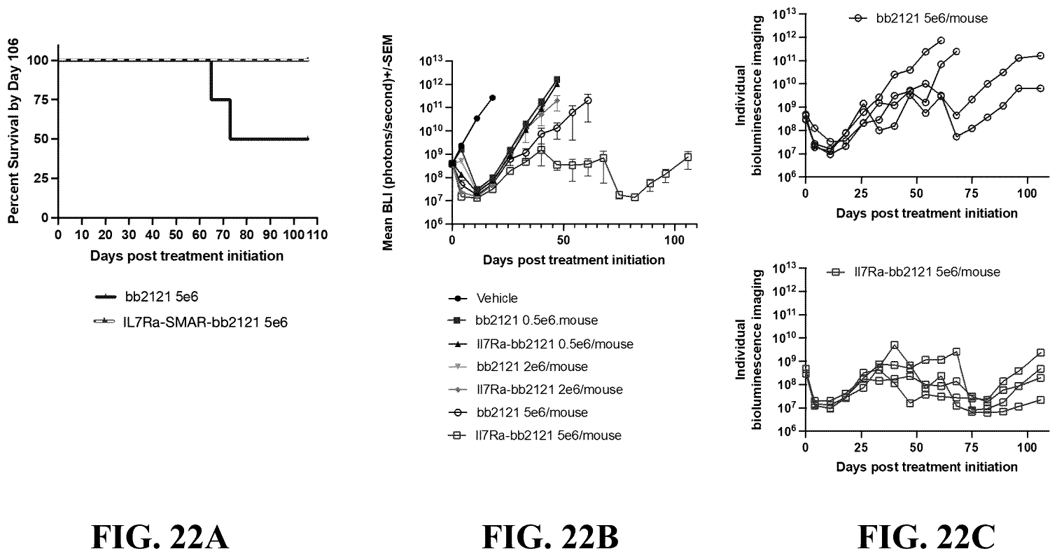

FIGS. 22A-22C are graphs illustrating survival (FIG. 22A), tumor cell killing (FIGS. 22B and 22C) at Day 105. As shown in FIG. 22A, at Day 105, there was 50% survival in the bb2121 5×10⁶ group versus 100% survival in the IL7Rα-bb2121 5×10⁶ group. As shown in FIG. 22B (showing BLI across each study group) and FIG. 22C (showing BLI for individual mice in the bb2121 5×10⁶ group (top) and the IL7Rα-bb2121 5×10⁶ group (bottom)), IL7Rα-bb2121 exhibited superior tumor cell killing compared to bb2121 alone. These results demonstrate improved survival and tumor control in mice treated with IL7Rα SMAR bb2121 compared to bb2121 alone.

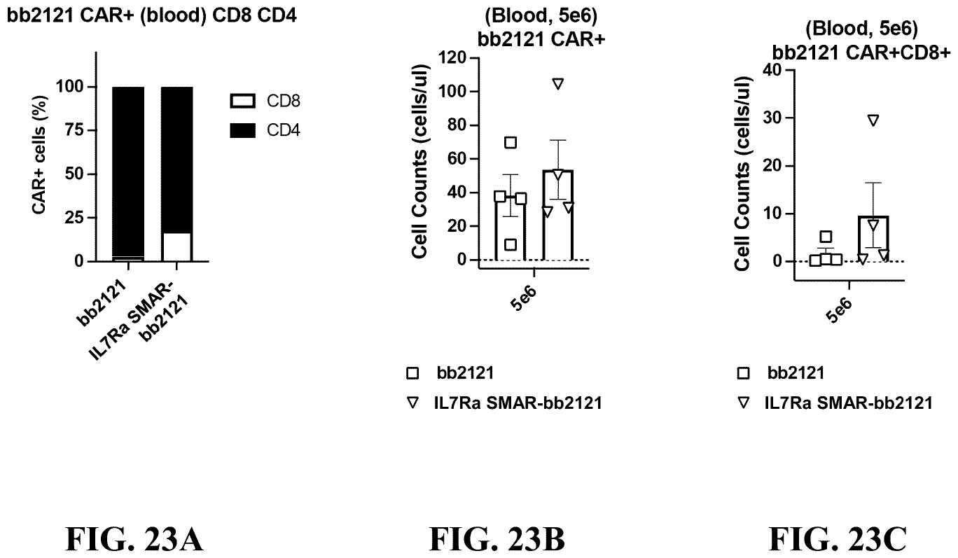

FIGS. 23A-23C are graphs illustrating percentages of CD8+/CD4+ cells (FIG. 23A), CAR+ cell counts (FIG. 23B), and CAR+ CD8+ cell counts (FIG. 23C) at Day 106. As shown in FIG. 23A-23C, bb2121 CAR T-cells with the IL7Rα SMAR increased the persistence of CAR+ CD8+ cells in vivo compared to cells without the SMAR.

Overall, the results of this example indicate that, compared to cells expressing the bb2121 CAR alone, cells expressing the IL7Rα SMAR with the bb2121 CAR demonstrated superior tumor killing at the 5×10⁶ dose, increased survival benefit, greater CAR-T cell expansion, greater memory phenotype, and improved persistence and durability. Moreover, these effects were observed without ex vivo or in vivo activation of the IL7Rα SMAR, which suggests that activator-independent activity is capable of eliciting phenotypic changes sufficient to produce a potent anti-tumor response. It is hypothesized that anti-tumor efficacy may be further enhanced with ex vivo and/or in vivo activation of the IL7Rα SMAR.

### Example 20: In Vivo Tumor Rechallenge Assay with Immune Cells with Engineered Cytokine Receptor Switches and an Anti-BCMA CAR (Bb2121)

This example describes a study to determine the anti-tumor activity of bb2121 CAR T-cells and IL7Rα SMAR bb2121 CAR T-cells in an MM1.S-luc-GFP disseminated tumor model in which mice were inoculated with a slow-growing tumor cell line, then rechallenged with a fast growing cell line. Blood and tissue samples were generated from satellite mice to test for changes in persistence and phenotypic changes over time. It was contemplated that differentiation between bb2121 alone and IL7Rα SMAR bb2121 in vivo may be observed by using a more aggressive MM1.S tumor model in combination with fewer CAR-T cells, based on previous dose finding studies.

Mice were acquired from Charles River Lab. The mice were female mice of strain NCG, and were 9 weeks old at the start of treatment. 72 animals were inoculated, and 70 were used in the study.

Mice were inoculated with MM.1S cells, which is a BCMA+ human multiple myeloma line. In the initial inoculation, the MM.1S-luc-GFP cells used were acquired from BPS Bioscience (San Diego, CA). The cells were maintained in RPMI 1640 medium supplemented with 10% FBS and gentamycin. The passage number of the frozen cells was P3 and the passage number of the inoculated cells was P7. 1×10⁷ cells in sterile DPBS (Gibco) were injected intravenously into the right lateral tail vein of each mouse in a volume of 200 μL using a 26 G 5/8 needle. 17 days after MM.1S inoculation, mice were imaged and randomized into three groups as shown in Table 15 below. The average bioluminescence signal for each treatment group was 2.5×10⁸ photons/second (total flux).

On Day 0 (start of treatment, 17 days after MM.1S inoculation), fresh CAR-T cells, either expressing the bb2121 CAR alone (“bb2121”) or co-expressing the IL7Rα SMAR with the bb2121 CAR (“IL7Rα SMAR bb2121”), were injected intravenously, 5×10⁶ per mouse.

TABLE 15
Study Groups

| | | | Number | Number |
|---|---|---|---|---|
| | Treatment/Route of | | of | of CAR+ |
| Group | Administration | Schedule | Mice | cells |
| 1 | Vehicle (PBS) | Once per week | 10 | N/A |
| 2 | bb2121 | Once per week | 10 | 5 × 10⁶ |
| 3 | IL7Rα SMAR | Once per week | 10 | 5 × 10⁶ |
| | bb2121 | | | |

Mice were re-challenged on Day 55 with MM.1S-luc-GFP cells generated in-house. Rechallenge was performed with this more aggressive MM.1S cell line, since superior anti-tumor activity was observed with both bb2121 and IL7Rα SMAR bb2121. These MM.1S-luc-GFP cells were maintained in RPMI 1640 medium supplemented with 10% FBS. The passage number of the frozen cells was P4 and the passage number of the inoculated cells was P7. 2×10⁶ cells were injected per mouse in a volume of 200 μL as described above.

Ventral and dorsal images were acquired on an IVIS Lumina one day before treatment, then weekly to monitor tumor growth. The bioluminescence signals were calculated on each day of imaging using Living Image software. The sum of the ventral and dorsal images were used for the calculations of mean BLI. At the end of the study, data was log transformed and analyzed using an unpaired t-test for statistical analysis.

Persistence measurements from blood, spleen, and lymph nodes were collected from satellite mice on Days 8, 22, and 66. Specifically, 100 μL of whole blood was collected from each mouse into EDTA-coated tubes, and 500 μL of ACK lysis buffer was added to each. Samples were vortexed and left to sit for 5 minutes before being centrifuged at 1200 rpm for 5 minutes. Samples were lysed and centrifuged a further two times to remove red blood cells (RBCs). Cells were then resuspended in 2 mL BD Pharmingen Stain Buffer and passed through 70 μm cell strainers. Finally, samples were centrifuged at 1200 rpm for 5 minutes and resuspended in Bambankers freezing media (Nippon Genetics Europe Gmbh).

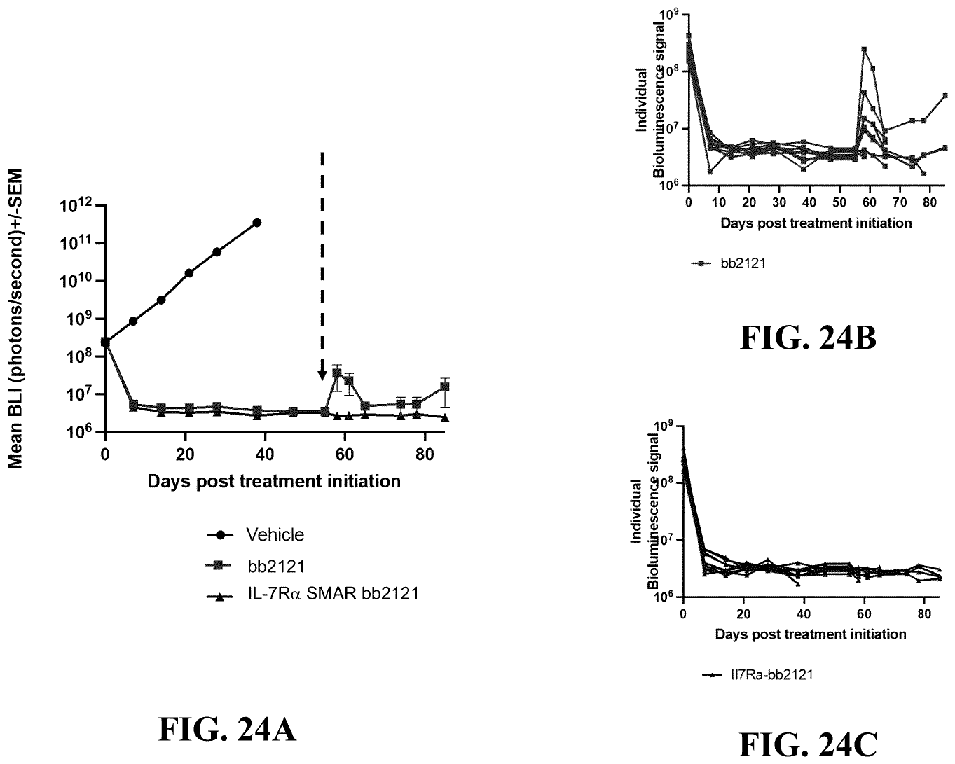

FIGS. 24A-24C are graphs illustrating tumor cell killing across the various study groups (FIG. 24A; arrow indicates the MM.1S rechallenge on Day 55), in individual mice in the bb2121 group (FIG. 24B), and in individual mice in the IL7Rα SMAR bb2121 group (FIG. 24C) (lower BLI corresponds to higher killing). As shown in FIG. 24A, both bb2121 and IL7Rα SMAR bb2121 exhibited significant tumor cell killing compared to the vehicle only control. After the rechallenge on Day 55, a greater tumor burden was observed in mice treated with bb2121 alone versus mice treated with IL7Rα SMAR bb2121 (p=0.0063 at Day 58, p=0.0133 at Day 61, and p=0.0745 at Day 85). A complete response was observed (tumor was undetectable) in 1/10 mice treated with IL7Rα SMAR bb2121 on Day 55. Moreover, as shown in FIGS. 24B and 24C, greater biological variability was observed in the bb2121 group versus a more consistent response in the IL7Rα SMAR bb2121 group.

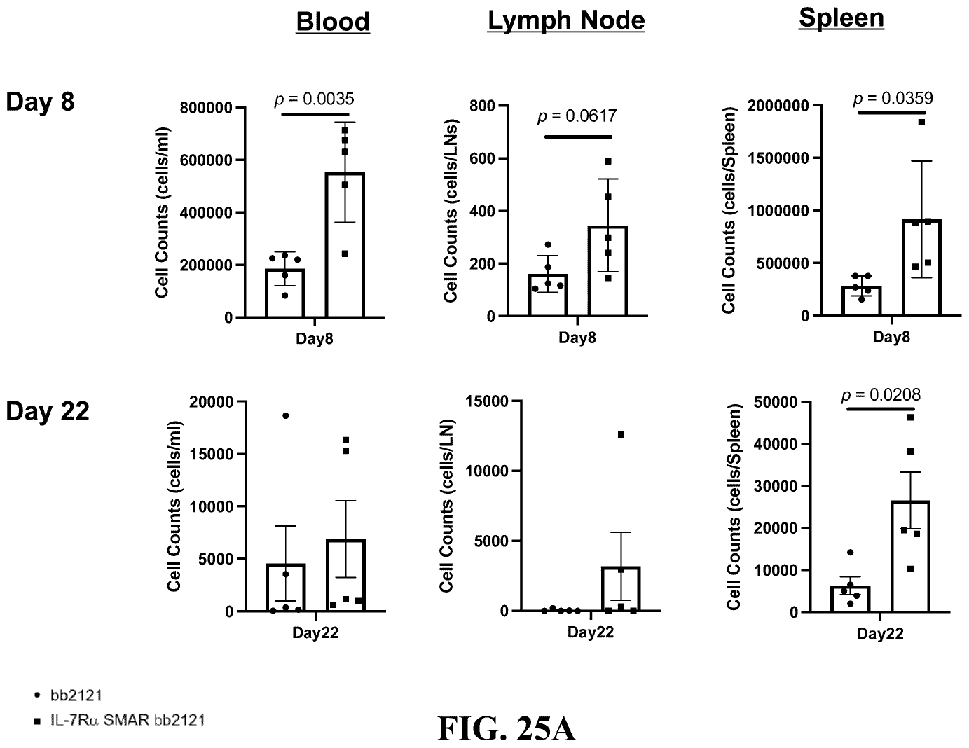

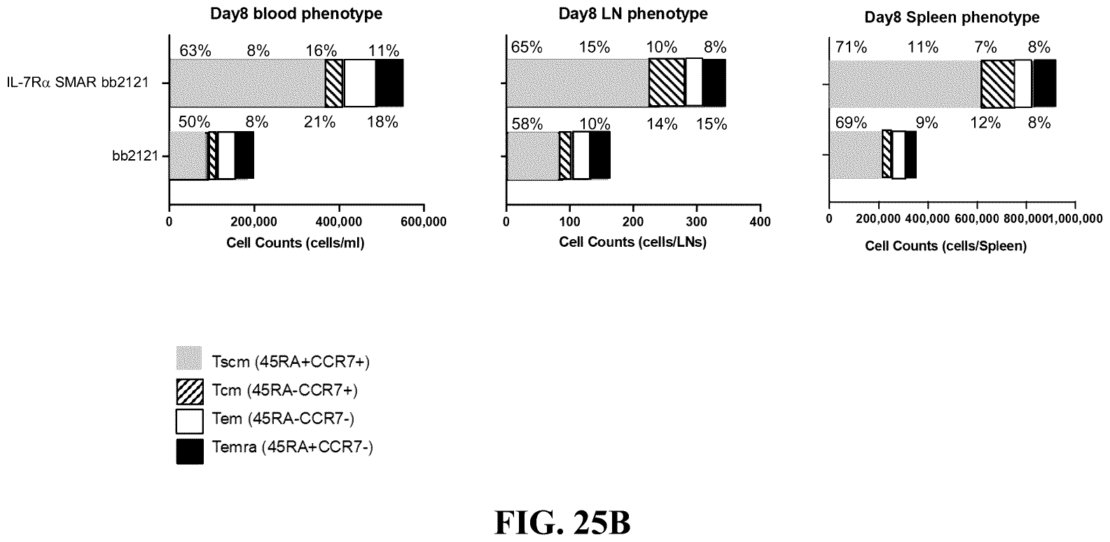

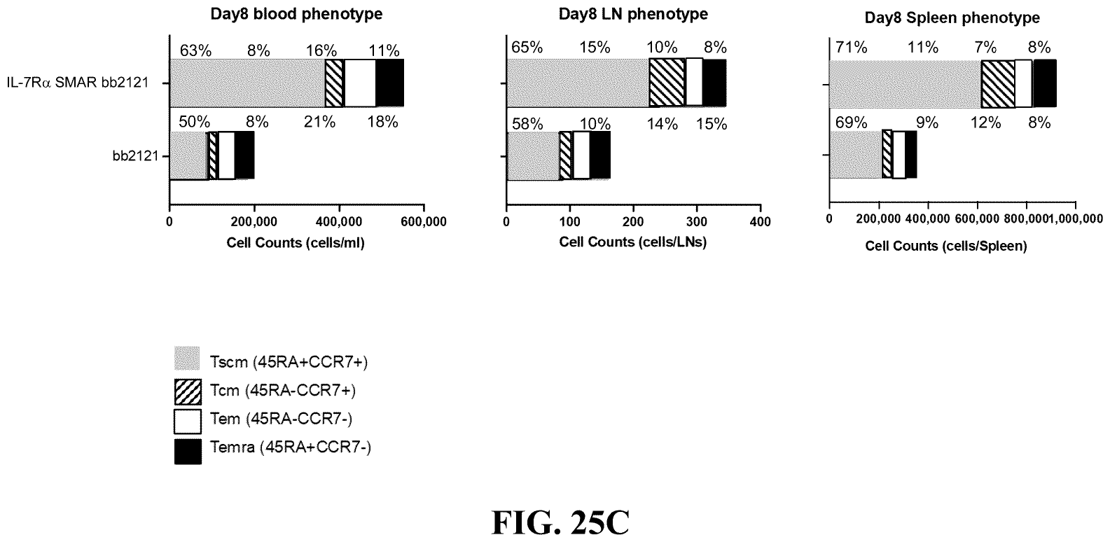

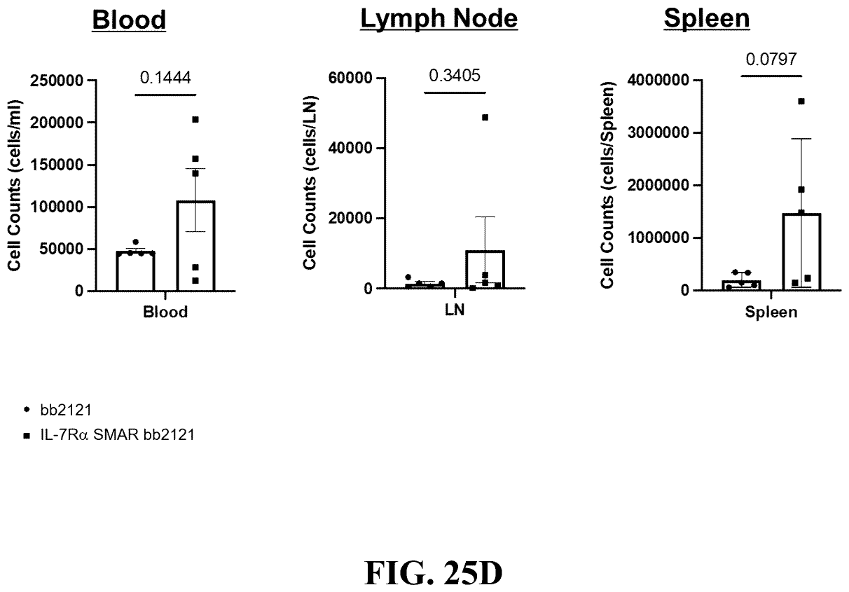

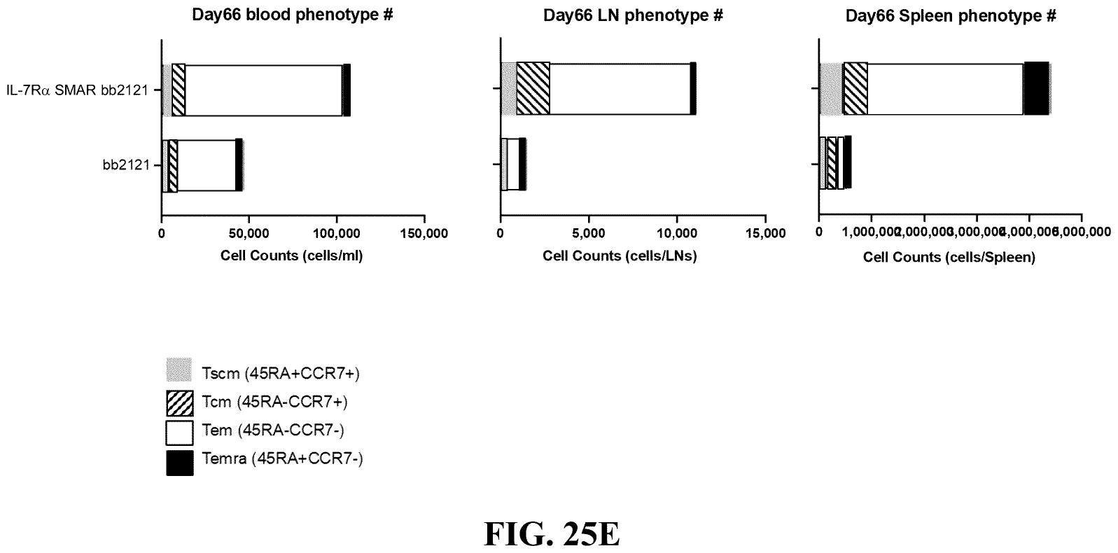

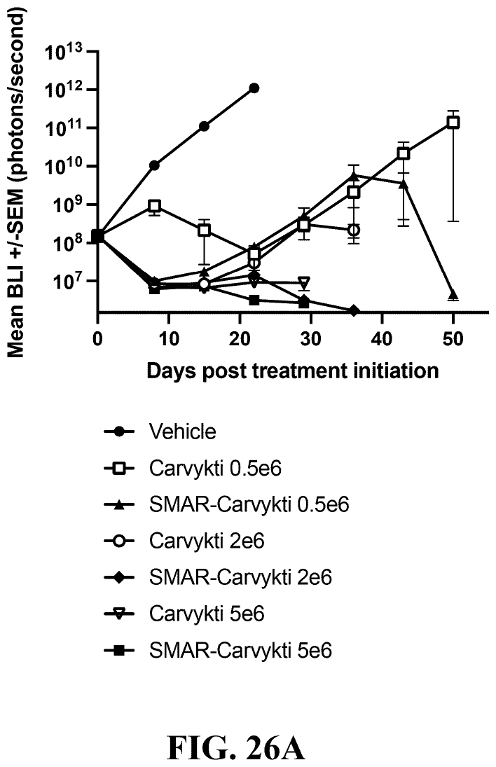

FIG. 25A is a series of graphs illustrating cell counts in the blood, lymph nodes, and spleen at Day 8 (top) and Day 22 (bottom). As shown in FIG. 25A, mice treated with IL7Rα SMAR bb2121 showed greater cell expansion in the blood, lymph nodes, and spleen compared to mice treated with bb2121 alone.

FIG. 25B is a series of graphs illustrating cell phenotypes in the blood (left), lymph nodes (center), and spleen (right) at Day 8. FIG. 25C is a series of graphs illustrating cell phenotypes in the blood (left), lymph nodes (center), and spleen (right) at Day 22. As shown in FIGS. 25B and 25C, on Day 8 and Day 22, mice treated with IL7Rα SMAR bb2121 demonstrated increased numbers of memory phenotype cells in the blood, lymph nodes, and spleen compared to mice treated with bb2121 alone.

FIG. 25D is a series of graphs illustrating cell counts in the blood, lymph nodes, and spleen at Day 66 (11 days after rechallenge), and FIG. 25E is a series of graphs illustrating cell phenotypes in the blood (left), lymph nodes (center), and spleen (right) at Day 66. As shown in FIG. 25D, after rechallenge, mice treated with IL7Rα SMAR bb2121 demonstrated increased numbers cells in the blood, lymph nodes, and spleen compared to mice treated with bb2121 alone. As shown in FIG. 25E, both cohorts demonstrated effector memory cell phenotypes, showing the impact of immune memory after rechallenge in producing effector cells.

Overall, the results of this example indicate that, compared to cells expressing the bb2121 CAR alone, cells expressing the IL7Rα SMAR with the bb2121 CAR demonstrated superior tumor killing after rechallenge, greater CAR-T cell expansion, greater persistence, and greater memory phenotype. Moreover, these effects were observed without ex vivo or in vivo activation of the IL7Rα SMAR, which suggests that activator-independent activity is capable of eliciting phenotypic changes sufficient to produce a potent anti-tumor response. It is hypothesized that anti-tumor efficacy may be further enhanced with ex vivo and/or in vivo activation of the IL7Rα SMAR.

### Example 21: In Vivo Anti-Tumor Activity of Immune Cells with Engineered Cytokine Receptor Switches and an Anti-BCMA CAR (Carvykti)

This example describes studies to investigate the anti-tumor activity of T-cells expressing an anti-BCMA CAR (Carvykti) alone (“Carvykti”) and with an IL7Rα SMAR (“SMAR-Carvykti” or “IL7Rα SMAR Carvykti”) in an MM1.S-luc-GFP disseminated model. It was contemplated that differentiation between Carvykti and IL-7Rα SMAR Carvykti in vivo may be observed by using a more aggressive MM1.S tumor model in combination with less CAR-T cells based on previous dose finding studies.

Mice were acquired from Charles River Lab. The mice were female mice of strain NCG, and were 7 weeks old at the start of treatment. 35 animals were inoculated, and 40 were used in the study.

The MM.1S-luc-GFP cells used in this example were generated in-house. The cells were maintained in RPMI 1640 medium supplemented with 10% FBS. The passage number of the frozen cells was P3 and the passage number of the inoculated cells was P7.

On the day of MM.1S tumor cell injections, cell inoculums were prepared using sterile DPBS at 1 million/mouse or 5 million/mL. MM.1S-luc-GFP cells were aseptically injected intravenously into the right lateral tail vein using a 26 G 5/8 needle. Six days after MM1.S cell inoculations, mice were imaged and randomized into seven groups, as shown in Table 16 below. The average BLI (photons/second) was 1.5×10⁸ total flux.

On Day 0 (start of treatment, 6 days after MM.1S inoculation), frozen vials of Carvykti or IL7Rα SMAR Carvykti cells were pooled together for cell injections on Day 0. Cells were injected intravenously at amounts of 0.5×10⁶, 2×10⁶, or 5×10⁶ CAR⁺ T cells per mouse.

TABLE 16
Study Groups

| | | | Number | Number |
|---|---|---|---|---|
| | | | of | of CAR+ |
| Group | Treatment | Schedule | Mice | T cells |
| 1 | Vehicle | Once per week | 5 | N/A |
| 2 | Carvykti | Once per week | 5 | 0.5 × 10⁶ |
| 3 | SMAR-Carvykti | Once per week | 5 | 0.5 × 10⁶ |
| 4 | Carvykti | Once per week | 5 | 2 × 10⁶ |
| 5 | SMAR-Carvykti | Once per week | 5 | 2 × 10⁶ |
| 6 | Carvykti | Once per week | 5 | 5 × 10⁶ |
| 7 | SMAR-Carvykti | Once per week | 5 | 5 × 10⁶ |

Ventral and dorsal images were acquired on using an IVIS Lumina one day before treatment, then weekly to monitor tumor growth. The bioluminescence signals were calculated on each day of imaging using Living Image software. The sum of the ventral and dorsal images was used for the calculations of mean BLI. At the end of the study, data was log transformed and analyzed using an unpaired t-test for statistical analysis.

Persistence measurements from blood were collected from 5×10⁶ per mouse groups for both bb2121 alone and IL7Rα SMAR bb2121. Specifically, 100 μL of whole blood was collected from each mouse into EDTA-coated tubes, and 500 μL of ACK lysis buffer was added to each. Samples were vortexed and left to sit for 5 minutes before being centrifuged at 1200 rpm for 5 minutes. Samples were lysed and centrifuged a further two times to remove red blood cells (RBCs). Cells were then resuspended in 2 mL BD Pharmingen Stain Buffer and passed through 70 μm cell strainers. Finally, samples were centrifuged at 1200 rpm for 5 minutes and resuspended in Bambankers freezing media (Nippon Genetics Europe Gmbh).

Bone marrow was analyzed in mice from the 0.5×10⁶ group only on Day 53. Two femurs were collected from each mouse and flushed with cold FACS buffer into centrifuge tubes. The solution was then aspirated via pipette to break up clumps of bone marrow before being centrifuged at 1200 rpm for 5 minutes. The samples were then resuspended in 2 mL BD stain buffer and passed through 70 μm cell strainers. Finally, the samples were centrifuged at 1200 rpm for 5 minutes and resuspended in Bambankers freezing media.

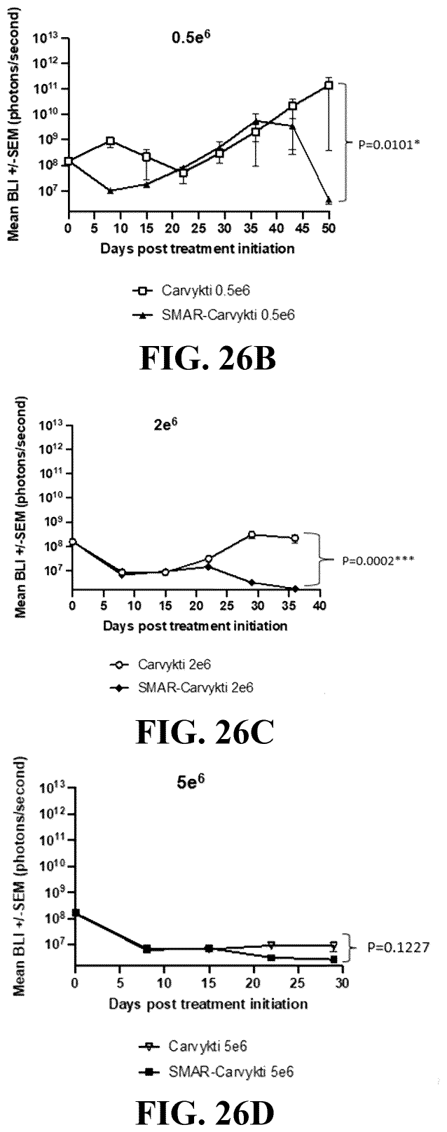

FIG. 26A is a graph illustrating tumor cell killing in the various study groups through Day 50 (lower bioluminescence intensity (BLI) corresponds to higher killing). Due to Graft vs Host Disease (GvHD), the 2×10⁶ and 5×10⁶ SMAR-Carvykti groups were terminated earlier than planned (Day 29 for the 5×10⁶ group, Day 36 for the 2×10⁶ group; more severe symptoms of GvHD occurred with SMAR-Carvykti). All treatment groups exhibited significant killing compared to the vehicle only control, as indicated in Table 17 below.

TABLE 17
Day 22 Results

| | | | P value |
|---|---|---|---|
| | | | compared to |
| | | Mean BLI on | Vehicle on |
| Group | Treatment | Day 22 | Day 22 |
| 1 | Vehicle | 1.11 × 10¹² | |
| 2 | Carvykti (0.5 × 10⁶) | 5.18 × 10⁷ | p < 0.0001 |
| 3 | SMAR-Carvykti (0.5 × 10⁶) | 7.90 × 10⁷ | p < 0.0001 |
| 4 | Carvykti (2 × 10⁶) | 3.02 × 10⁷ | p < 0.0001 |
| 5 | SMAR-Carvykti (2 × 10⁶) | 1.142 × 10⁷ | p < 0.0001 |
| 6 | Carvykti (5 × 10⁶) | 9.17 × 10⁶ | p < 0.0001 |
| 7 | SMAR-Carvykti (5 × 10⁶) | 3.16 × 10⁶ | p < 0.0001 |

FIGS. 26B-26D are graphs illustrating tumor cell killing in the 0.5×10⁶ (FIG. 26B), 2×10⁶ (FIG. 26C), and 5×10⁶ (FIG. 26D) groups. As shown in FIGS. 26B and 26C, IL7Rα SMAR Carvykti demonstrated superior in vivo tumor control compared to Carvykti alone at doses of 0.5×10⁶ and 2×10⁶ cells.

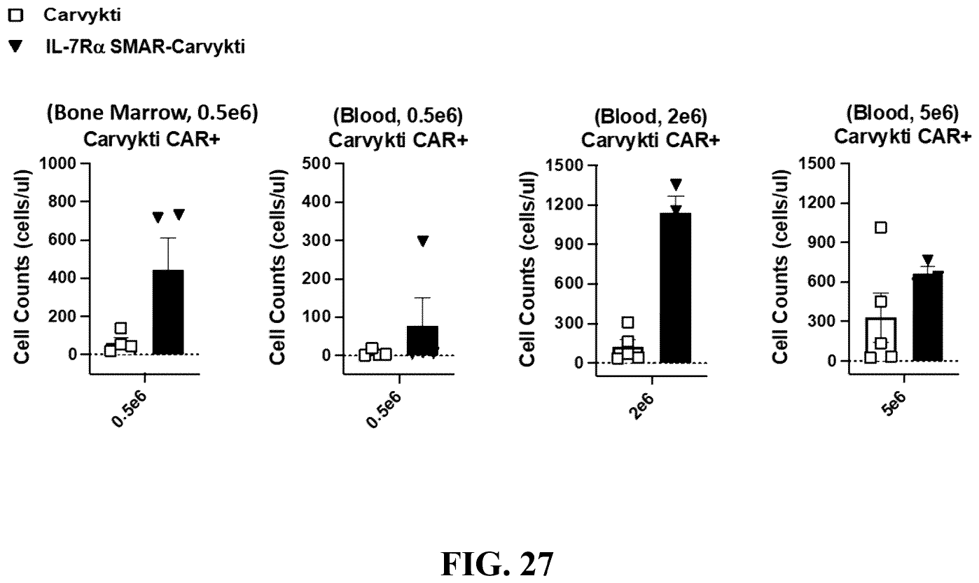

FIG. 27 is a series of graphs illustrating CAR+ cell counts in the bone marrow (0.5×10⁶ group only, Day 53, leftmost graph) and blood (0.5×10⁶ group, Day 53, second to left graph; 2×10⁶, Day 36, second to right graph; 5×10⁶, Day 29, rightmost graph). As shown in FIG. 27, IL7Rα SMAR Carvykti increased cell expansion in the bone marrow and blood compared to Carvykti alone.

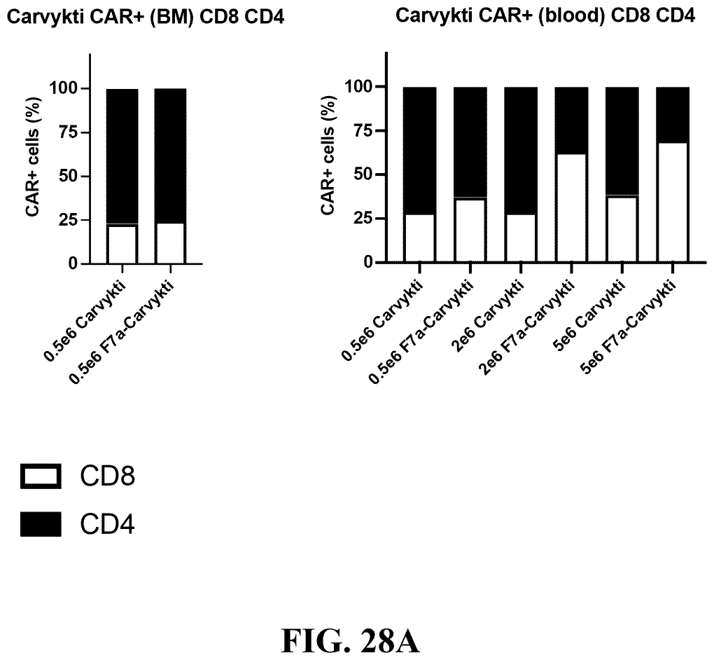

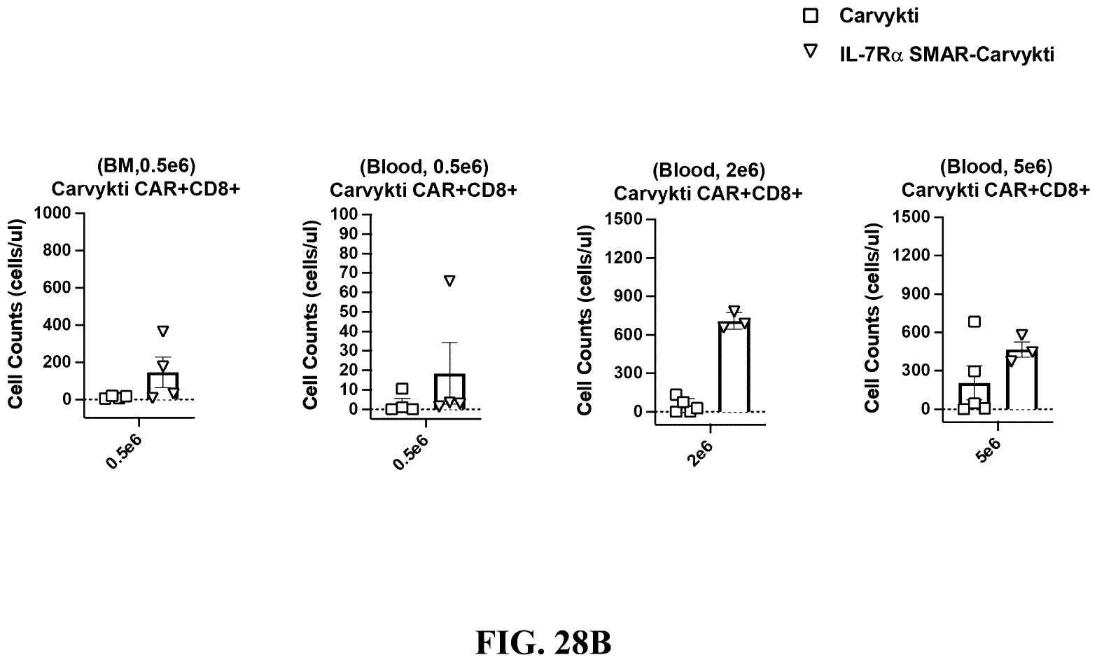

FIG. 28A is a series of graphs illustrating percentages of CD8+/CD4+ cells in the bone marrow (“BM,” left) and blood (right), and FIG. 28B is a series of graphs illustrating CAR+CD8+ cell counts in the bone marrow and blood. Data for the 0.5×10⁶ was obtained at Day 53, data for the 2×10⁶ group was obtained at Day 36, and data for the 5×10⁶ group was obtained at Day 29. These results demonstrate that IL7Rα SMAR Carvykti increased the persistence of CD8+ cells in the bone marrow and blood compared to Carvykti alone.

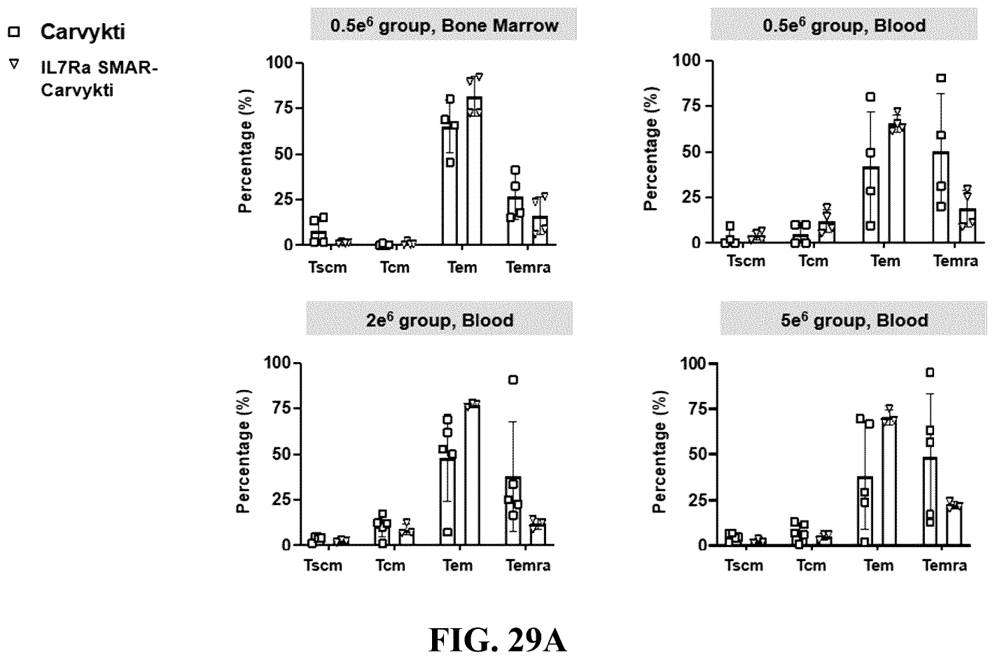

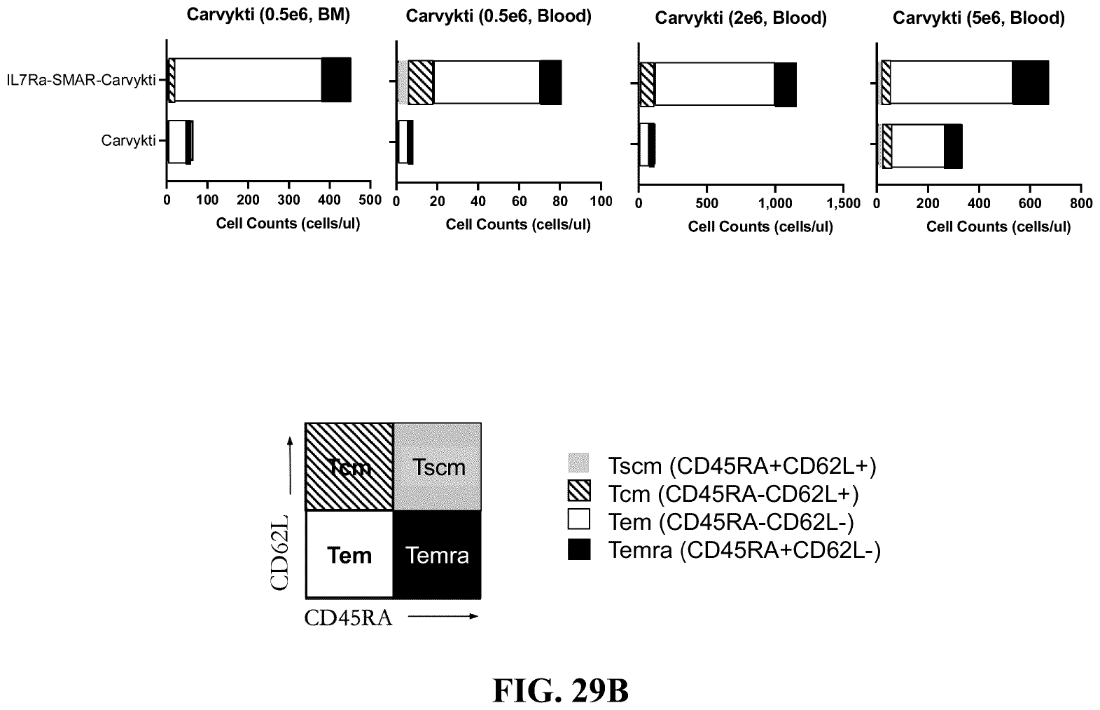

FIGS. 29A and 29B are a series of graphs illustrating cell phenotypes by percentage (FIG. 29A) and by cell counts (FIG. 29B) in the bone marrow and blood. Data for the 0.5×10⁶ group was obtained at Day 53, data for the 2×10⁶ group was obtained at Day 36, and data for the 5×10⁶ group was obtained at Day 29. As illustrated in FIG. 29B (bottom), cells positive for CD45RA and CD62L were identified as having a stem-cell memory (T_{scm}) phenotype, cells positive for CD62L but negative for CD45RA were identified as having a central memory (T_{cm}) phenotype, cells negative for both CD62L and CD45RA were identified has having an effector memory (T_{cm}) phenotype, and cells positive for CD45RA but negative for CD62L were identified as having an effector memory re-expressing CD45RA (Tₑₘᵣₐ) phenotype. As shown in FIGS. 29A and 29B, IL7Rα SMAR Carvykti promoted the Tₑₘ phenotype but reduced the Tₑₘᵣₐ phenotype. Taken together, the data shown in FIGS. 27-29B suggest that the IL7Rα SMAR increased persistence of the CAR T-cells in the MM.1S tumor model.

The results from the bb2121 and the Carvykti studies described herein suggest that the presence of the SMAR increases CAR T-cell persistence and antitumor efficacy. Differences in the results across these studies may be attributable to the different study timelines. For instance, in the bb2121 studies, tumor rechallenge was performed 47 days after the initial killing. At this stage, most of the tumor cells had been killed and thus most of the CAR T-cells exhibited a T_{cm} phenotype. In the Carvykti studies, CAR T-cells were analyzed during the initial phase of tumor killing. Therefore, most of the CAR T-cells were still active and thus exhibited a Tₑₘ phenotype.

### IX. ADDITIONAL EXAMPLES

Additional examples of aspects of the present technology are described below as numbered clauses (1, 2, 3, etc.) for convenience. These are provided as examples and do not limit the subject technology.
1. A composition comprising:
an immune cell population, wherein the immune cell population comprises immune cells
   expressing a cytokine receptor switch comprising:
   an activator binding domain,
   a signal peptide,
   a hinge domain,
   a transmembrane domain, and
   an intracellular domain, wherein the intracellular domain comprises a cytokine receptor intracellular domain,
wherein at least 20% of the immune cells in the immune cell population have a memory phenotype.
2. The composition of Clause 1, wherein at least 30%, at least 40%, or at least 50% of the immune cells in the immune cell population have a memory phenotype.
3. The composition of Clause 1 or 2, wherein not less than 20% and not more than 90%, not less than 30% and not more than 80%, not less than 40% and not more than 70% of the immune cells in the immune cell population have a memory phenotype.
4. The composition of any one of Clauses 1-3, wherein at least 10% of the immune cells in the immune cell population have an effector phenotype.
5. The composition of any one of Clauses 1-4, wherein at least 20%, at least 30%, at least 40%, at least 50%, or at least 60% of the immune cells in the immune cell population have an effector phenotype.
6. The composition of Clause 4 or 5, wherein immune cells having the memory phenotype persist for longer than immune cells having the effector phenotype.
7. The composition of any one of Clauses 4-6, wherein the immune cells having the effector phenotype have a stronger anti-tumor potency than the immune cells having the memory phenotype.
8. The composition of any one of Clauses 1-7, wherein the intracellular domain is derived from an endogenous cytokine receptor intracellular domain.
9. The composition of Clause 8, wherein the endogenous cytokine receptor intracellular domain is an IL2Rα intracellular domain, an IL2Rβ intracellular domain, an IL2Rγ intracellular domain, an IL4Rα intracellular domain, an IL7Rα intracellular domain, an IL9Rα intracellular domain, an IL15Rα intracellular domain, or an IL21Rα intracellular domain.
10. The composition of Clause 8 or 9, wherein the intracellular domain comprises a sequence having at least 80%, at least 90%, at least 95%, at least 97%, or 100% sequence identity to the endogenous cytokine receptor intracellular domain.
11. The composition of any one of Clauses 1-10, wherein the intracellular domain comprises a sequence having at least 80%, at least 90%, at least 95%, at least 97%, or 100% sequence identity to any one of SEQ ID NO: 29-SEQ ID NO: 34.
12. The composition of any one of Clauses 1-11, wherein the intracellular domain comprises a sequence of any one of SEQ ID NO: 29-SEQ ID NO: 34.
13. The composition of any one of Clauses 1-12, wherein the intracellular domain comprises or is derived from an intracellular domain of any of the following: IL2Rα, IL2Rβ, IL2Rγ, IL4Rα, IL7Rα, IL15Rα, IL21Rα, IL1R, CD123, CD124, IL5Rα, IL5Rβ, CD126, CD132, CD129, IL11Rα, IL12Rβ1, IL12Rβ2, IL13Rα1, CD122, IL18R, IL23R, IL27Rα, CD130, or GM-CSF.
14. The composition of any one of Clauses 1-13, wherein the intracellular domain comprises a single intracellular domain.
15. The composition of any one of Clauses 1-13, wherein the intracellular domain comprises a plurality of intracellular domains in tandem.
16. The composition of any one of Clauses 1-15, wherein the transmembrane domain is a cytokine receptor transmembrane domain.
17. The composition of any one of Clauses 1-16, wherein the transmembrane domain is derived from an endogenous cytokine receptor transmembrane domain.
18. The composition of Clause 17, wherein the endogenous cytokine receptor transmembrane domain is an IL2Rα transmembrane domain, an IL2Rβ transmembrane domain, an IL2Rγ transmembrane domain, an IL4Rα transmembrane domain, an IL7Rα transmembrane domain, an IL9Rα transmembrane domain, an IL15Rα transmembrane domain, or an IL21Rα transmembrane domain.
19. The composition of Clause 17 or 18, wherein the transmembrane domain comprises a sequence having at least 80%, at least 90%, at least 95%, at least 97%, or 100% sequence identity to the endogenous cytokine receptor transmembrane domain.
20. The composition of any one of Clauses 1-19, wherein the transmembrane domain comprises a sequence having at least 80%, at least 90%, at least 95%, at least 97%, or 100% sequence identity to any one of SEQ ID NO: 23-SEQ ID NO: 28.
21. The composition of any one of Clauses 1-20, wherein the transmembrane domain comprises a sequence of any one of SEQ ID NO: 23-SEQ ID NO: 28.
22. The composition of any one of Clauses 1-21, wherein the transmembrane domain comprises or is derived from a transmembrane domain of any of the following: IL2Rα, IL2Rβ, IL2Rγ, IL4Rα, IL7Rα, IL15Rα, IL21Rα, IL1R, CD123, CD124, IL5Rα, IL5Rβ, CD126, CD132, CD129, IL11Rα, IL12Rβ1, IL12Rβ2, IL13Rα1, CD122, IL18R, IL23R, IL27Rα, CD130, an immunoglobulin, CD8, CD28, GM-CSF, or EpoR.
23. The composition of any one of Clauses 1-22, wherein the hinge domain is derived from an endogenous hinge domain.
24. The composition of Clause 23, wherein the endogenous hinge domain is a CD8 hinge, a CD3 hinge, a CD4 hinge, or a CD28 hinge.
25. The composition of Clause 23 or 24, wherein the hinge domain comprises a sequence having at least 80%, at least 90%, at least 95%, at least 97%, or 100% sequence identity to the endogenous hinge domain.
26. The composition of any one of Clauses 1-25, wherein the hinge domain comprises a sequence having at least 80%, at least 90%, at least 95%, at least 97%, or 100% sequence identity to SEQ ID NO: 22.
27. The composition of any one of Clauses 1-26, wherein the hinge domain comprises a sequence of SEQ ID NO: 22.
28. The composition of any one of Clauses 1-27, wherein the hinge domain comprises or is derived from a hinge domain of any of the following: CD8, CD3, CD4, CD28, 4-1BB, CD28, OX40, ICOS, CD27, an immunoglobulin, or EpoR.
29. The composition of any one of Clauses 1-28, wherein the hinge domain has a length greater than or equal to 1 amino acid, 2 amino acids, 3 amino acids, 4 amino acids, 5 amino acids, 6 amino acids, 7 amino acids, 8 amino acids, 9 amino acids, 10 amino acids, 15 amino acids, 20 amino acids, 25 amino acids, 30 amino acids, 35 amino acids, 40 amino acids, 45 amino acids, or 50 amino acids.
30. The composition of any one of Clauses 1-29, wherein the hinge domain has a length less than or equal to 50 amino acids, 45 amino acids, 40 amino acids, 30 amino acids, 25 amino acids, 20 amino acids, 15 amino acids, 10 amino acids, 9 amino acids, 8 amino acids, 7 amino acids, 6 amino acids, 5 amino acids, 4 amino acids, 3 amino acids, 2 amino acids, or 1 amino acid.
31. The composition of any one of Clauses 1-30, wherein the signal peptide is a cytokine receptor signal peptide.
32. The composition of any one of Clauses 1-31, wherein the signal peptide is derived from an endogenous cytokine signal peptide.
33. The composition of Clause 32, wherein the endogenous cytokine receptor signal peptide is an IL2Rα signal peptide, an IL2Rβ signal peptide, an IL2Rγ signal peptide, an IL4Rα signal peptide, an IL7Rα signal peptide, an IL9Rα signal peptide, an IL15Rα signal peptide, or an IL21Rα signal peptide.
34. The composition of Clause 32 or 33, wherein the signal peptide comprises a sequence having at least 80%, at least 90%, at least 95%, at least 97%, or 100% sequence identity to the endogenous cytokine receptor signal peptide.
35. The composition of any one of Clauses 1-34, wherein the signal peptide comprises a sequence having at least 80%, at least 90%, at least 95%, at least 97%, or 100% sequence identity to any one of SEQ ID NO: 15-SEQ ID NO: 20.
36. The composition of any one of Clauses 1-35, wherein the signal peptide comprises a sequence of any one of SEQ ID NO: 15-SEQ ID NO: 20.
37. The composition of any one of Clauses 1-36, wherein the signal peptide comprises or is derived from a signal peptide of any of the following: IL2Rα, IL2Rβ, IL2Rγ, IL4Rα, IL7Rα, IL15Rα, IL21Rα, IL1R, CD123, CD124, IL5Rα, IL5Rβ, CD126, CD132, CD129, IL11Rα, IL12Rβ1, IL12Rβ2, IL13Rα1, CD122, IL18R, IL23R, IL27Rα, CD130, an immunoglobulin, CD8, CD28, or GM-CSF.
38. The composition of any one of Clauses 1-37, wherein the activator binding domain comprises a single-chain variable fragment (scFv), a peptide, or a nanobody.
39. The composition of Clause 38, wherein the peptide comprises a molecular weight of from 1 kDa to 10 kDa.
40. The composition of any one of Clauses 1-39, wherein the activator binding domain comprises an scFv.
41. The composition of any one of Clauses 1-40, wherein the activator binding domain comprises a sequence having at least 80%, at least 90%, at least 95%, at least 97%, or 100% sequence identity to SEQ ID NO: 21.
42. The composition of any one of Clauses 1-41, wherein the activator binding domain comprises a sequence of SEQ ID NO: 21.
43. The composition of any one of Clauses 1-42, wherein the activator binding domain is humanized.
44. The composition of any one of Clauses 1-43, wherein the activator binding domain is capable of binding an activator.
45. The composition of Clause 44, wherein the activator is non-immunogenic.
46. The composition of Clause 44 or 45, wherein the activator is an exogenous activator.
47. The composition of any one of Clauses 44-46, wherein the activator is a cancer cell surface antigen comprising CD19, CD20, CD22, CLL-1, CD123, CD33, CD3, CD4, CD8, CD38, CD44v6, SLAMF7, BCMA, GD2, GPRC5D, MUC16, HER2, EGFR, EGFRvIII, folate receptor-α, B7-H3, EphA2, GRP78, NKG2D, CD70, or mesothelin.
48. The composition of any one of Clauses 44-47, wherein the activator is an immunosuppressive molecule or a portion thereof.
49. The composition of Clause 48, wherein the immunosuppressive molecule induces inhibition of CAR T-cell activity.
50. The composition of Clause 48 or 49, wherein the activator is or is a ligand of any of the following: CD2, CD95 (Fas), CTLA4 (CD152), CD172A (SIRPa), CD200R, CD223 (LAG3), CD279 (PD-1), CD272 (BTLA), CD300, CD366 (TIM3), A2aR, KIR, LPA5, TIGIT, TGFβ, CD58 (LFA3), CD178 (Fas-L), CD80 (B7-1), CD86 (B7-2), CD47, CD200, LAG-3, CD273 (PD-L2), CD274 (PD-L1), CD258 (HVEM), CD300, CD94 (NKG2A), TIM3, GPR92, IL6, IL10, or adenosine.
51. The composition of any one of Clauses 44-50, wherein the activator has low toxicity.
52. The composition of any one of Clauses 44-51, wherein the activator has low cross-reactivity.
53. The composition of any one of Clauses 44-52, wherein the activator is a small molecule, a peptide, or an oligonucleotide.
54. The composition of any one of Clauses 44-53, wherein the activator is selected from the group consisting of fluorescein, fluorescein isothiocyanate (FITC), fluorescein 5-maleimide, fluorescein-5-carboxamide, fluorescein-6-carboxamide, 6-FAM phosphoramidite, topiramate hemisuccinate, creatine, acetaminophen, ketamine, propofol, lidocaine, ractopamine, salicylate, salicylic acid, sulfasalazine, dapsone, albendazole, ivermectin, levamisole, permethrin, pyrantel, thiabendazole, procainamide, sulfamethazine, amikacin, amoxicillin, ampicillin, cefazolin, cefuroxime, cephalexin, chloramphenicol, chloramphenicol, ciprofloxacin, clenbuterol, cloxacillin, colistin A, dicloxacillin, enrofloxacin, furaltadone, gentamicin, gentamicin, kanamycin, kanamycin, kincomycin, lincomycin, metronidazole, nafcillin, nalidixic acid, neomycin, neomycin, nitrofurazone, norfloxacin, ofloxacin, oxacillin, spectinomycin, streptomycin, streptomycin, sulfabenzamide, sulfacetamide, sulfadiazine, sulfadimidine, sulfametoxydiazine, sulfanilamide, trimethoprim, carbamazepine, ethosuximide, lamotrigine, primidone, cetirizine, chlorpheniramine, diphenhydramine, doxylamine, promethazine, sulfadimethoxine, benzothiazinone, butylated hydroxytoluene, tripelennamine, chlorpromazine, clozapine, haloperidol, olanzapine, paliperidone, quetiapine, ribavirin, meprobamate, acebutolol, atenolol, penbutolol, warfarin, salmeterol, aflatoxin B1, tetraxetan (DOTA), 4-[(6-methylpyrazin-2-yl) oxy]benzoate (MPOB), biotin, melamine, methotrexate, amphetamine, diethylpropion, dextromethorphan, pseudoephedrine, dihydrochlorothiazide, hydrochlorothiazide, clonazepam, diazepam, nitrazepam, rhodamine B, fluorescent brightener Ksn, zearalenone, Sudan Red1, acetominophen, acrylamide, benzoic acid, benzophenone, benzothiazine, mercaptobenzothiazole, erythrosine, Sudan, tartrazine, erythromycin, sirolimus, atropine, ethyl glucuronide, aflatoxin M1, methocarbamol, fentanyl, hydromorphone, morphine, remifentanil, tapentadol, tramadol, pregabalin, gabapentin, amitriptyline, desipramine, imipramine, nortriptyline, venlafaxine, dinitrophenyl, His-tag, PEG methoxy group, etodolac, ibuprofen, ketoprofen, meclofenamic acid, phenylbutazone, acetyl salicylic acid, acetamiprid, acetochlor, carbadazim, carbaryl, chlorothalonil, chlorpyrifos, fenpropathrin, imazalil, imidacloprid, parathion, abscisic acid, dibutyl phthalate, clonazepam, lorazepam, oxazepam, phenobarbital, secobarbital, zaleplon, zolpidem, trazodone, fluoxetine, fluvoxamine, cortisone, dexamethasone, dihydrotestosterone, fluocinolone, methylprednisolone, prednisolone, stanozolol, triamcinolone, mazindol, methamphetamine, methylphenidate, modafinil, chrysoidine, deoxynivalenol, fumonisin, microcystin Lr, ochratoxin, sterigmatocystin, T-2 toxin, sildenafil, tadalafil, scopolamine, florfenicol, pirlimycin, and sulfaquinoxaline.
55. The composition of any one of Clauses 44-54, wherein the activator is a small molecule.
56. The composition of Clause 55, wherein the small molecule is fluorescein, a fluorescein derivative, or tetraxetan (DOTA).
57. The composition of Clause 56, wherein the fluorescein derivative is fluorescein isothiocyanate (FITC), fluorescein 5-maleimide, fluorescein-5-carboxamide, fluorescein-6-carboxamide, or 6-FAM phosphoramidite.
58. The composition of any one of Clauses 44-57, wherein the intracellular domain is in an active conformation when the activator binding domain is bound to the activator.
59. The composition of Clause 58, wherein the active conformation of the intracellular domain is capable of activating a cytokine signaling pathway.
60. The composition of Clause 59, wherein the cytokine signaling pathway comprises a Jak-STAT pathway.
61. The composition of Clause 59 or 60, wherein the activation of the cytokine signaling pathway causes conversion to a memory phenotype, upregulation of lymphoid homing markers, or a combination thereof.
62. The composition of any one of Clauses 44-61, further comprising the activator.
63. The composition of Clause 62, further comprising a bispecific agent comprising the activator and a targeting moiety.
64. The composition of Clause 63, wherein the activator is conjugated to the targeting moiety.
65. The composition of Clause 63 or 64, wherein the targeting moiety comprises an antibody, antibody fragment, scFv, nanobody, or peptide.
66. The composition of any one of Clauses 63-65, wherein the targeting moiety binds to a tumor antigen.
67. The composition of Clause 66, wherein the tumor antigen is selected from the group consisting of CD19, CD20, CD22, CD123, CD33, CD3, CD4, CD8, CD38, SLAMF7, BCMA, GD2, GPRC5D, MUC16, HER2, EGFR, EGFRvIII, CLL-1, CD44v6, folate receptor-α, mesothelin, CD20, CD37, ROR1, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, surviving, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, IL13Rα2, B7-H3 (CD276), EPHA2, GRP78, NKG2D, and CD70.
68. The composition of any one of Clauses 63-67, wherein the targeting moiety binds to a lymphoid marker.
69. The composition of Clause 68, wherein the lymphoid marker is expressed on a lymph node, spleen, thymus, bone marrow, or a combination thereof.
70. The composition of Clause 68 or 69, wherein the lymphoid marker is selected from the group consisting of PNAd, VEGFR-3, LYVE-1, Prox-1, podoplanin, CD31, MadCAM1, CXCL13, RANKL, CXCL12, APRIL, BAFF, IL-7, CCL19, CCL21, and Spns2.
71. The composition of Clause 62, wherein the activator is conjugated to a substrate.
72. The composition of Clause 71, wherein the substrate comprises a surface, a bead, a carrier protein, a carrier polymer, a carrier nucleic acid, or a combination thereof.
73. The composition of any one of Clauses 1-72, wherein the intracellular domain is in an inactive conformation when the activator binding domain is unbound.
74. The composition of any one of Clauses 1-73, wherein the cytokine receptor switch exhibits activator-independent activity when the activator binding domain is unbound.
75. The composition of Clause 74, wherein the cytokine receptor switch is capable of dimerizing to another receptor when the activator binding domain is unbound.
76. The composition of Clause 75, wherein the other receptor is another cytokine receptor switch, an endogenous cytokine receptor, or a chimeric antigen receptor.
77. The composition of any one of Clauses 74-76, wherein the activator-independent activity causes conversion to a memory phenotype, upregulation of lymphoid homing markers, or a combination thereof.
78. The composition of any one of Clauses 1-77, wherein the cytokine receptor switch comprises a sequence having at least 80%, at least 90%, at least 95%, at least 97%, or 100% sequence identity to any one of SEQ ID NO: 1-SEQ ID NO: 7.
79. The composition of any one of Clauses 1-78, wherein the cytokine receptor switch comprises a sequence of any one of SEQ ID NO: 1-SEQ ID NO: 7.
80. The composition of any one of Clauses 1-79, wherein the cytokine receptor switch is encoded by a sequence having at least 80%, at least 90%, at least 95%, at least 97%, or 100% sequence identity to any one of SEQ ID NO: 8-SEQ ID NO: 14.
81. The composition of any one of Clauses 1-80, wherein the cytokine receptor switch is encoded by a sequence of any one of SEQ ID NO: 8-SEQ ID NO: 14.
82. The composition of any one of Clauses 1-81, further comprising a polynucleotide expression cassette encoding the cytokine receptor switch.
83. The composition of any one of Clauses 1-82, further comprising a vector encoding the cytokine receptor switch.
84. The composition of Clause 83, wherein the vector is a viral vector.
85. The composition of Clause 84, wherein the viral vector is a lentiviral vector, an adeno-associated viral vector, a vaccinia viral vector, a poxvirus viral vector, a herpes viral vector, an alphavirus viral vector, a gamma retrovirus viral vector, a polyoma viral vector, or a combination thereof.
86. The composition of any one of Clauses 1-85, wherein the immune cells comprise T-cells, B-cells, natural killer cells (NK cells), FcFRIγ deficient NK cells (g-NK cells), neutrophils, eosinophils, macrophages, γδ T-cells, or combinations thereof.
87. The composition of any one of Clauses 1-86, wherein the memory phenotype comprises a stem-cell memory phenotype, a central memory phenotype, an effector memory phenotype, an effector memory re-expressing CD45RA phenotype, or a combination thereof.
88. The composition of any one of Clauses 1-87, wherein the immune cells further express a chimeric antigen receptor (CAR).
89. The composition of Clause 88, wherein the CAR comprises an antigen binding domain, a transmembrane domain, and an intracellular domain.
90. The composition of Clause 89, wherein the antigen binding domain comprises an antibody, an antibody fragment, an scFv, a nanobody, or a peptide.
91. The composition of Clause 89 or 90, wherein the CAR is an indirect CAR and the antigen binding domain binds a synthetic antigen.
92. The composition of Clause 91, wherein the synthetic antigen is a small molecule, a peptide, an oligonucleotide, or a protein.
93. The composition of Clause 91 or 92, wherein the synthetic antigen is different than an activator bound by the activator binding domain.
94. The composition of Clause 91 or 92, wherein the synthetic antigen is the same as the activator bound by the activator binding domain.
95. The composition of any one of Clauses 91-94, wherein the synthetic antigen is selected from the group consisting of fluorescein, fluorescein isothiocyanate (FITC), fluorescein 5-maleimide, fluorescein-5-carboxamide, fluorescein-6-carboxamide, 6-FAM phosphoramidite, topiramate hemisuccinate, creatine, acetaminophen, ketamine, propofol, lidocaine, ractopamine, salicylate, salicylic acid, sulfasalazine, dapsone, albendazole, ivermectin, levamisole, permethrin, pyrantel, thiabendazole, procainamide, sulfamethazine, amikacin, amoxicillin, ampicillin, cefazolin, cefuroxime, cephalexin, chloramphenicol, chloramphenicol, ciprofloxacin, clenbuterol, cloxacillin, colistin A, dicloxacillin, enrofloxacin, furaltadone, gentamicin, gentamicin, kanamycin, kanamycin, kincomycin, lincomycin, metronidazole, nafcillin, nalidixic acid, neomycin, neomycin, nitrofurazone, norfloxacin, ofloxacin, oxacillin, spectinomycin, streptomycin, streptomycin, sulfabenzamide, sulfacetamide, sulfadiazine, sulfadimidine, sulfametoxydiazine, sulfanilamide, trimethoprim, carbamazepine, ethosuximide, lamotrigine, primidone, cetirizine, chlorpheniramine, diphenhydramine, doxylamine, promethazine, sulfadimethoxine, benzothiazinone, butylated hydroxytoluene, tripelennamine, chlorpromazine, clozapine, haloperidol, olanzapine, paliperidone, quetiapine, ribavirin, meprobamate, acebutolol, atenolol, penbutolol, warfarin, salmeterol, aflatoxin B1, tetraxetan (DOTA), 4-[(6-methylpyrazin-2-yl) oxy]benzoate (MPOB), biotin, melamine, methotrexate, amphetamine, diethylpropion, dextromethorphan, pseudoephedrine, dihydrochlorothiazide, hydrochlorothiazide, clonazepam, diazepam, nitrazepam, rhodamine B, fluorescent brightener Ksn, zearalenone, Sudan Red1, acetominophen, acrylamide, benzoic acid, benzophenone, benzothiazine, mercaptobenzothiazole, erythrosine, Sudan, tartrazine, erythromycin, sirolimus, atropine, ethyl glucuronide, aflatoxin M1, methocarbamol, fentanyl, hydromorphone, morphine, remifentanil, tapentadol, tramadol, pregabalin, gabapentin, amitriptyline, desipramine, imipramine, nortriptyline, venlafaxine, dinitrophenyl, His-tag, PEG methoxy group, etodolac, ibuprofen, ketoprofen, meclofenamic acid, phenylbutazone, acetyl salicylic acid, acetamiprid, acetochlor, carbadazim, carbaryl, chlorothalonil, chlorpyrifos, fenpropathrin, imazalil, imidacloprid, parathion, abscisic acid, dibutyl phthalate, clonazepam, lorazepam, oxazepam, phenobarbital, secobarbital, zaleplon, zolpidem, trazodone, fluoxetine, fluvoxamine, cortisone, dexamethasone, dihydrotestosterone, fluocinolone, methylprednisolone, prednisolone, stanozolol, triamcinolone, mazindol, methamphetamine, methylphenidate, modafinil, chrysoidine, deoxynivalenol, fumonisin, microcystin Lr, ochratoxin, sterigmatocystin, T-2 toxin, sildenafil, tadalafil, scopolamine, florfenicol, pirlimycin, and sulfaquinoxaline.
96. The composition of any one of Clauses 91-95, further comprising a bispecific agent comprising the synthetic antigen conjugated to a targeting moiety.
97. The composition of Clause 96, wherein the targeting moiety comprises an antibody, antibody fragment, scFv, nanobody, or peptide.
98. The composition of Clause 96 or 97, wherein the targeting moiety binds to a tumor antigen.
99. The composition of Clause 98, wherein the tumor antigen is selected from the group consisting of CD19, CD20, CD22, CD123, CD33, CD3, CD4, CD8, CD38, SLAMF7, BCMA, GD2, GPRC5D, MUC16, HER2, EGFR, EGFRvIII, CLL-1, CD44v6, folate receptor-α, mesothelin, CD20, CD37, ROR1, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, surviving, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, IL13Rα2, B7-H3 (CD276), EPHA2, GRP78, NKG2D, and CD70.
100. The composition of Clause 78 or 79, wherein the CAR is a direct CAR and the antigen binding domain binds a tumor antigen.
101. The composition of Clause 85, wherein the tumor antigen is selected from the group consisting of CD19, CD20, CD22, CD123, CD33, CD3, CD4, CD8, CD38, SLAMF7, BCMA, GD2, GPRC5D, MUC16, HER2, EGFR, EGFRvIII, CLL-1, CD44v6, folate receptor-α, mesothelin, CD20, CD37, ROR1, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, surviving, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, IL13Rα2, B7-H3 (CD276), EPHA2, GRP78, NKG2D, and CD70.
102. The composition of Clause 88, wherein the CAR binds CD19, CD20, CD22, CD123, CD33, CLL-1, CD38, CD3, CD4, CD8, SLAMF7, BCMA, GD2, GPRC5D, MUC16, HER2, EGFR, EGFRvII, CD44v6, B7-H3, EphA2, GRP78, NKG2D, CD70, folate receptor-α, or mesothelin.
103. The composition of any one of Clauses 88-102, wherein the CAR is encoded by a sequence having at least 80%, at least 90%, at least 95%, at least 97%, or 100% sequence identity to any one of SEQ ID NO: 40-SEQ ID NO: 45.
104. The composition of any one of Clauses 88-103, wherein the CAR is encoded by a sequence of any one of SEQ ID NO: 40-SEQ ID NO: 45.
105. The composition of any one of Clauses 88-104, wherein the cytokine receptor switch and the CAR are encoded by a sequence having at least 80%, at least 90%, at least 95%, at least 97%, or 100% sequence identity to any one of SEQ ID NO: 46-SEQ ID NO: 52.
106. The composition of any one of Clauses 88-105, wherein the cytokine receptor switch and the CAR are encoded by a sequence of any one of SEQ ID NO: 46-SEQ ID NO: 52.
107. A pharmaceutical composition comprising the composition of any one of Clauses 1-106 and a pharmaceutically acceptable carrier.
108. A method of activating an immune cell, the method comprising contacting the composition of any one of any one of Clauses 1-106 with an activator and binding the activator to the activator binding domain of the cytokine receptor switch, thereby activating the immune cells.
109. The method of Clause 108, wherein the immune cells comprise T-cells, B-cells, natural killer cells, (NK cells), FcεRIγ deficient NK cells (g-NK cells), neutrophils, eosinophils macrophages, γδ T-cells, or combinations thereof.
110. The method of Clause 108 or 109, wherein the method is performed ex vivo.
111. The method of Clause 108 or 109, wherein the method is performed in vivo.
112. The method of any one of Clauses 108-111, wherein the activator is conjugated to a substrate.
113. The method of Clause 112, wherein the substrate is a surface, a bead, a carrier protein, or a carrier nucleic acid.
114. The method of Clause 113, wherein the carrier protein comprises a peptide, an scFv, or a nanobody.
115. A method of treating a cancer in a subject, the method comprising administering to the subject an immune cell activated using the method of any one of Clauses 108-114.
116. A method of treating cancer in a subject, the method comprising administering to the subject the composition of any one of Clauses 1-106.
117. A method of treating cancer in a subject, the method comprising administering to the subject the pharmaceutical composition of Clause 107.
118. A method of treating a cancer in a subject, the method comprising:
administering to the subject an immune cell population comprising immune cells
   expressing a chimeric antigen receptor (CAR) and a cytokine receptor switch,
   wherein the cytokine receptor switch comprises:
   an activator binding domain that binds an activator,
   a signal peptide,
   a hinge domain,
   a transmembrane domain, and
   an intracellular domain, wherein the intracellular domain comprises a cytokine receptor intracellular domain,
   wherein at least 20% of the immune cells in the immune cell population have a memory phenotype, thereby treating the cancer.
119. The method of Clause 118, further comprising recruiting the immune cells to a cancer cell by binding the CAR to a target antigen on the cancer cell.
120. The method of Clause 118, further comprising recruiting the immune cell to a cancer cell by administering a bispecific agent to the subject, wherein the bispecific agent comprises a targeting moiety that binds to a target antigen on the cancer cell and a synthetic antigen, and wherein the CAR binds to the synthetic antigen.
121. The method of Clause 119 or 120, further comprising activating an immune response against the cancer cell.
122. The method of any one of Clauses 119-121, further comprising killing the cancer cell.
123. The method of any one of Clauses 118-122, further comprising activating the immune cells by contacting the activator binding domain to the activator.
124. The method of Clause 123, wherein activating the immune cells is performed in vivo.
125. The method of Clause 123 or 124, wherein activating the immune cells is performed ex vivo.
126. The method of any one of Clauses 123-125, further comprising administering a bispecific agent to the subject, wherein the bispecific agent comprises a targeting moiety and the activator.
127. The method of Clause 126, wherein the targeting moiety binds to a tumor antigen.
128. The method of Clause 127, wherein the tumor antigen is selected from the group consisting of CD19, CD20, CD22, CD123, CD33, CD3, CD4, CD8, CD38, SLAMF7, BCMA, GD2, GPRC5D, MUC16, HER2, EGFR, EGFRvIII, CLL-1, CD44v6, folate receptor-α, mesothelin, CD20, CD37, ROR1, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, surviving, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, IL13Rα2, B7-H3 (CD276), EPHA2, GRP78, NKG2D, and CD70.
129. The method of Clause 126, wherein the targeting moiety binds to a lymphoid marker.
130. The method of Clause 129, wherein the lymphoid marker is selected from the group consisting of PNAd, VEGFR-3, LYVE-1, Prox-1, podoplanin, CD31, MadCAM1, CXCL13, RANKL, CXCL12, APRIL, BAFF, IL-7, CCL19, CCL21, and Spns2.
131. The method of any one of Clauses 118-122, wherein the activator binding domain is not contacted with the activator.
132. The method of Clause 131, wherein the cytokine receptor switch exhibits activator-independent activity causing conversion to the memory phenotype, upregulation of a lymphoid homing marker, or a combination thereof.
133. The method of Clause 131 or 132, wherein the cytokine receptor switch dimerizes with another receptor in the absence of the activator.
134. The method of Clause 133, wherein the other receptor is another cytokine receptor switch, an endogenous cytokine receptor, or the CAR.
135. The method of any one of Clauses 118-134, wherein the cancer is acute myeloid leukemia, multiple myeloma, ovarian cancer, mesothelioma, non-Hodgkin lymphoma, acute lymphoblastic leukemia, mantle cell lymphoma, follicular lymphoma, glioma, pancreatic cancer, prostate cancer, or gastric cancer.
136. The method of any one of Clauses 118-135, wherein the immune cells are recruited to a target antigen on a cancer cell, and the target antigen comprises CD19, CD20, CD22, CLL-1, CD123, CD33, CD3, CD4, CD8, CD38, SLAMF7, BCMA, GD2, GPRC5D, MUC16, HER2, EGFR, EGFRvII, CD44v6, B7-H3, EphA2, GRP78, NKG2D, CD70, folate receptor-α, or mesothelin.
137. The method of any one of Clauses 118-136, wherein immune cells having the memory phenotype persist in the subject for longer than immune cells having an effector phenotype.
138. The method of any one of Clauses 118-137, wherein immune cells having the memory phenotype convert to immune cells having an effector phenotype.
139. The method of Clause 137 or 138, wherein the immune cells having the effector phenotype have a stronger anti-tumor potency against the cancer than the immune cells having the memory phenotype.
140. The method of any one of Clauses 118-139, wherein treating the cancer comprises preventing recurrence of the cancer.
141. The method of any one of Clauses 118-140, further comprising administering the activator to the subject to re-activate the immune cells.
142. The method of Clause 141, wherein the immune cells are re-activated multiple times.
143. The method of any one of Clauses 118-142, wherein the immune cells are re-activated between one week and one year after administering the immune cells.
144. The method of any one of Clauses 118-143, wherein the immune cells persist in the subject for at least 1 month, at least 2 months, at least 3 months, at least 4 months, at least 5 months, at least 6 months, at least 7 months, at least 8 months, at least 9 months, at least 10 months, at least 11 months, or at least 12 months.
145. The method of any one of Clauses 118-144, wherein the immune cells persist in the subject at least 2 times, at least 3 times, or at least 5 times as long as an immune cell that does not express the engineered cytokine receptor switch.
146. The method of any one of Clauses 118-145, further comprising upregulating expression of a lymphoid homing marker on the immune cells.
147. The method of Clause 146, wherein the lymphoid homing marker comprises CD62L, CCR7, or a combination thereof.
148. The method of any one of Clauses 118-147, further comprising: administering a first bispecific agent to the subject, and administering a second bispecific agent to the subject.
149. The method of Clause 148, wherein the first bispecific agent comprises the activator and a first targeting moiety, and the second bispecific agent comprises a synthetic antigen for the CAR and a second targeting moiety.
150. The method of Clause 148, wherein the first bispecific agent comprises the activator and a first targeting moiety, and the second bispecific agent comprises the activator and a second targeting moiety.
151. The method of Clause 149 or 150, wherein the first targeting moiety and the second targeting moiety are different.
152. The method of Clause 149 or 150, wherein the first targeting moiety and the second targeting moiety are the same.
153. The method of any one of Clauses 148-152, wherein the first bispecific agent is administered at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 16 days, at least about 18 days, at least about 20 days, at least about 22 days, at least about 24 days, at least about 26 days, at least about 28 days, at least about 35 days, at least about 42 days, at least about 49 days, at least about 56 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months before the second bispecific agent.
154. The method of any one of Clauses 148-152, wherein the first bispecific agent is administered concurrently with the second bispecific agent.
155. The method of any one of Clauses 148-152, wherein the first bispecific agent is administered at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 16 days, at least about 18 days, at least about 20 days, at least about 22 days, at least about 24 days, at least about 26 days, at least about 28 days, at least about 35 days, at least about 42 days, at least about 49 days, at least about 56 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months after the second bispecific agent.
156. The method of any one of Clauses 118-155, wherein the intracellular domain comprises or is derived from an intracellular domain of any of the following: IL2Rα, IL2Rβ, IL2Rγ, IL4Rα, IL7Rα, IL15Rα, IL21Rα, IL1R, CD123, CD124, IL5Rα, IL5Rβ, CD126, CD132, CD129, IL11Rα, IL12Rβ1, IL12Rβ2, IL13Rα1, CD122, IL18R, IL23R, IL27Rα, CD130, or GM-CSF.
157. The method of any one of Clauses 118-156, wherein the transmembrane domain comprises or is derived from a transmembrane domain of any of the following: IL2Rα, IL2R, IL2Rγ, IL4Rα, IL7Rα, IL15Rα, IL21Rα, IL1R, CD123, CD124, IL5Rα, IL5Rβ, CD126, CD132, CD129, IL11Rα, IL12Rβ1, IL12Rβ2, IL13Rα1, CD122, IL18R, IL23R, IL27Rα, CD130, an immunoglobulin, CD8, CD28, GM-CSF, or EpoR.
158. The method of any one of Clauses 118-157, wherein the hinge domain comprises or is derived from a hinge domain of any of the following: CD8, CD3, CD4, CD28, 4-1BB, CD28, OX40, ICOS, CD27, an immunoglobulin, or EpoR.
159. The method of any one of Clauses 118-158, wherein the signal peptide comprises or is derived from a signal peptide of any of the following: IL2Rα, IL2Rβ, IL2Rγ, IL4Rα, IL7Rα, IL15Rα, IL21Rα, IL1R, CD123, CD124, IL5Rα, IL5Rβ, CD126, CD132, CD129, IL11Rα, IL12Rβ1, IL12Rβ2, IL13Rα1, CD122, IL18R, IL23R, IL27Rα, CD130, an immunoglobulin, CD8, CD28, or GM-CSF.
160. The method of any one of Clauses 118-159, wherein the activator is selected from the group consisting of fluorescein, fluorescein isothiocyanate (FITC), fluorescein 5-maleimide, fluorescein-5-carboxamide, fluorescein-6-carboxamide, 6-FAM phosphoramidite, topiramate hemisuccinate, creatine, acetaminophen, ketamine, propofol, lidocaine, ractopamine, salicylate, salicylic acid, sulfasalazine, dapsone, albendazole, ivermectin, levamisole, permethrin, pyrantel, thiabendazole, procainamide, sulfamethazine, amikacin, amoxicillin, ampicillin, cefazolin, cefuroxime, cephalexin, chloramphenicol, chloramphenicol, ciprofloxacin, clenbuterol, cloxacillin, colistin A, dicloxacillin, enrofloxacin, furaltadone, gentamicin, gentamicin, kanamycin, kanamycin, kincomycin, lincomycin, metronidazole, nafcillin, nalidixic acid, neomycin, neomycin, nitrofurazone, norfloxacin, ofloxacin, oxacillin, spectinomycin, streptomycin, streptomycin, sulfabenzamide, sulfacetamide, sulfadiazine, sulfadimidine, sulfametoxydiazine, sulfanilamide, trimethoprim, carbamazepine, ethosuximide, lamotrigine, primidone, cetirizine, chlorpheniramine, diphenhydramine, doxylamine, promethazine, sulfadimethoxine, benzothiazinone, butylated hydroxytoluene, tripelennamine, chlorpromazine, clozapine, haloperidol, olanzapine, paliperidone, quetiapine, ribavirin, meprobamate, acebutolol, atenolol, penbutolol, warfarin, salmeterol, aflatoxin B1, tetraxetan (DOTA), 4-[(6-methylpyrazin-2-yl) oxy]benzoate (MPOB), biotin, melamine, methotrexate, amphetamine, diethylpropion, dextromethorphan, pseudoephedrine, dihydrochlorothiazide, hydrochlorothiazide, clonazepam, diazepam, nitrazepam, rhodamine B, fluorescent brightener Ksn, zearalenone, Sudan Red1, acetominophen, acrylamide, benzoic acid, benzophenone, benzothiazine, mercaptobenzothiazole, erythrosine, Sudan, tartrazine, erythromycin, sirolimus, atropine, ethyl glucuronide, aflatoxin M1, methocarbamol, fentanyl, hydromorphone, morphine, remifentanil, tapentadol, tramadol, pregabalin, gabapentin, amitriptyline, desipramine, imipramine, nortriptyline, venlafaxine, dinitrophenyl, His-tag, PEG methoxy group, etodolac, ibuprofen, ketoprofen, meclofenamic acid, phenylbutazone, acetyl salicylic acid, acetamiprid, acetochlor, carbadazim, carbaryl, chlorothalonil, chlorpyrifos, fenpropathrin, imazalil, imidacloprid, parathion, abscisic acid, dibutyl phthalate, clonazepam, lorazepam, oxazepam, phenobarbital, secobarbital, zaleplon, zolpidem, trazodone, fluoxetine, fluvoxamine, cortisone, dexamethasone, dihydrotestosterone, fluocinolone, methylprednisolone, prednisolone, stanozolol, triamcinolone, mazindol, methamphetamine, methylphenidate, modafinil, chrysoidine, deoxynivalenol, fumonisin, microcystin Lr, ochratoxin, sterigmatocystin, T-2 toxin, sildenafil, tadalafil, scopolamine, florfenicol, pirlimycin, and sulfaquinoxaline.
161. The method of any one of Clauses 118-160, wherein the CAR binds to a synthetic antigen selected from the group consisting of fluorescein, fluorescein isothiocyanate (FITC), fluorescein 5-maleimide, fluorescein-5-carboxamide, fluorescein-6-carboxamide, 6-FAM phosphoramidite, topiramate hemisuccinate, creatine, acetaminophen, ketamine, propofol, lidocaine, ractopamine, salicylate, salicylic acid, sulfasalazine, dapsone, albendazole, ivermectin, levamisole, permethrin, pyrantel, thiabendazole, procainamide, sulfamethazine, amikacin, amoxicillin, ampicillin, cefazolin, cefuroxime, cephalexin, chloramphenicol, chloramphenicol, ciprofloxacin, clenbuterol, cloxacillin, colistin A, dicloxacillin, enrofloxacin, furaltadone, gentamicin, gentamicin, kanamycin, kanamycin, kincomycin, lincomycin, metronidazole, nafcillin, nalidixic acid, neomycin, neomycin, nitrofurazone, norfloxacin, ofloxacin, oxacillin, spectinomycin, streptomycin, streptomycin, sulfabenzamide, sulfacetamide, sulfadiazine, sulfadimidine, sulfametoxydiazine, sulfanilamide, trimethoprim, carbamazepine, ethosuximide, lamotrigine, primidone, cetirizine, chlorpheniramine, diphenhydramine, doxylamine, promethazine, sulfadimethoxine, benzothiazinone, butylated hydroxytoluene, tripelennamine, chlorpromazine, clozapine, haloperidol, olanzapine, paliperidone, quetiapine, ribavirin, meprobamate, acebutolol, atenolol, penbutolol, warfarin, salmeterol, aflatoxin B1, tetraxetan (DOTA), 4-[(6-methylpyrazin-2-yl) oxy]benzoate (MPOB), biotin, melamine, methotrexate, amphetamine, diethylpropion, dextromethorphan, pseudoephedrine, dihydrochlorothiazide, hydrochlorothiazide, clonazepam, diazepam, nitrazepam, rhodamine B, fluorescent brightener Ksn, zearalenone, Sudan Red1, acetominophen, acrylamide, benzoic acid, benzophenone, benzothiazine, mercaptobenzothiazole, erythrosine, Sudan, tartrazine, erythromycin, sirolimus, atropine, ethyl glucuronide, aflatoxin M1, methocarbamol, fentanyl, hydromorphone, morphine, remifentanil, tapentadol, tramadol, pregabalin, gabapentin, amitriptyline, desipramine, imipramine, nortriptyline, venlafaxine, dinitrophenyl, His-tag, PEG methoxy group, etodolac, ibuprofen, ketoprofen, meclofenamic acid, phenylbutazone, acetyl salicylic acid, acetamiprid, acetochlor, carbadazim, carbaryl, chlorothalonil, chlorpyrifos, fenpropathrin, imazalil, imidacloprid, parathion, abscisic acid, dibutyl phthalate, clonazepam, lorazepam, oxazepam, phenobarbital, secobarbital, zaleplon, zolpidem, trazodone, fluoxetine, fluvoxamine, cortisone, dexamethasone, dihydrotestosterone, fluocinolone, methylprednisolone, prednisolone, stanozolol, triamcinolone, mazindol, methamphetamine, methylphenidate, modafinil, chrysoidine, deoxynivalenol, fumonisin, microcystin Lr, ochratoxin, sterigmatocystin, T-2 toxin, sildenafil, tadalafil, scopolamine, florfenicol, pirlimycin, and sulfaquinoxaline.
162. The method of any one of Clauses 118-160, wherein the CAR binds to a tumor antigen selected from the group consisting of CD19, CD20, CD22, CD123, CD33, CD3, CD4, CD8, CD38, SLAMF7, BCMA, GD2, GPRC5D, MUC16, HER2, EGFR, EGFRvII, CLL-1, CD44v6, folate receptor-α, mesothelin, CD20, CD37, ROR1, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, surviving, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, IL13Rα2, B7-H3 (CD276), EPHA2, GRP78, NKG2D, and CD70.
163. A method of treating a cancer in a subject, the method comprising administering to the subject a composition of any one of Clauses 1-106, thereby treating the cancer.
164. A method for treating a subject, the method comprising:
administering a first dose of immune cells comprising a greater number of effector phenotype immune cells than memory phenotype immune cells, and
administering a second dose of immune cells comprising a greater number of memory phenotype immune cells than effector phenotype immune cells.
165. The method of Clause 164, wherein the first dose of immune cells and the second dose of immune cells express an engineered cytokine receptor switch.
166. The method of Clause 164 or 165, wherein the first dose of immune cells and the second dose of immune cells express a chimeric antigen receptor.
167. The method of any one of Clauses 165-166, wherein the second dose of immune cells is administered at least 1 day after the first dose of immune cells.
168. The method of any one of Clauses 164-167, wherein the first dose of immune cells comprises the composition of any one of Clauses 1-106.
169. The method of any one of Clauses 164-168, wherein the second dose of immune cells comprises the composition of any one of Clauses 1-106.
170. A composition comprising:
an engineered immune cell comprising a cytokine receptor switch, or a polynucleotide
   encoding the cytokine receptor switch, wherein the cytokine receptor switch
   comprises:
   activator binding domain with affinity for an activator,
   a signal peptide,
   a hinge domain,
   a transmembrane domain, and
   an intracellular domain comprising a cytokine receptor intracellular domain; and
a bispecific agent comprising the activator linked to a targeting protein, wherein the
   targeting protein has affinity for a lymphoid marker.
171. The composition of Clause 170, wherein the lymphoid marker comprises CD3, CD45, CD4, CD2, CD5, CD8, γδ-T-cell receptor, T19, CD45, cell-surface immunoglobulin (sIg), L-selectin, PNAd, VEGFR-3, LYVE-1, Prox-1, podoplanin, CD31, MadCAM1, CXCL13, RANKL, CXCL12, APRIL, BAFF, IL-7, CCL19, CCL21, or Spns2.
172. The composition of Clause 170 or 171, wherein the targeting protein comprises a miniprotein, a peptide, an antibody, an antibody fragment, a single-chain variable fragment (scFv), or a nanobody.
173. The composition of any one of Clauses 170-172, wherein the targeting protein comprises a miniprotein.
174. The composition of any one of Clauses 170-173, wherein the activator is non-immunogenic.
175. The composition of any one of Clauses 170-174, wherein the activator is an exogenous activator.
176. The composition of any one of Clauses 170-175, wherein the activator has low toxicity.
177. The composition of any one of Clauses 170-176, wherein the activator has low cross-reactivity.
178. The composition of any one of Clauses 170-177, wherein the activator is a small molecule, a peptide, or an oligonucleotide.
179. The composition of any one of Clauses 170-178, wherein the activator is selected from the group consisting of fluorescein, fluorescein isothiocyanate (FITC), fluorescein 5-maleimide, fluorescein-5-carboxamide, fluorescein-6-carboxamide, 6-FAM phosphoramidite, topiramate hemisuccinate, creatine, acetaminophen, ketamine, propofol, lidocaine, ractopamine, salicylate, salicylic acid, sulfasalazine, dapsone, albendazole, ivermectin, levamisole, permethrin, pyrantel, thiabendazole, procainamide, sulfamethazine, amikacin, amoxicillin, ampicillin, cefazolin, cefuroxime, cephalexin, chloramphenicol, chloramphenicol, ciprofloxacin, clenbuterol, cloxacillin, colistin A, dicloxacillin, enrofloxacin, furaltadone, gentamicin, gentamicin, kanamycin, kanamycin, kincomycin, lincomycin, metronidazole, nafcillin, nalidixic acid, neomycin, neomycin, nitrofurazone, norfloxacin, ofloxacin, oxacillin, spectinomycin, streptomycin, streptomycin, sulfabenzamide, sulfacetamide, sulfadiazine, sulfadimidine, sulfametoxydiazine, sulfanilamide, trimethoprim, carbamazepine, ethosuximide, lamotrigine, primidone, cetirizine, chlorpheniramine, diphenhydramine, doxylamine, promethazine, sulfadimethoxine, benzothiazinone, butylated hydroxytoluene, tripelennamine, chlorpromazine, clozapine, haloperidol, olanzapine, paliperidone, quetiapine, ribavirin, meprobamate, acebutolol, atenolol, penbutolol, warfarin, salmeterol, aflatoxin B1, tetraxetan (DOTA), 4-[(6-methylpyrazin-2-yl) oxy]benzoate (MPOB), biotin, melamine, methotrexate, amphetamine, diethylpropion, dextromethorphan, pseudoephedrine, dihydrochlorothiazide, hydrochlorothiazide, clonazepam, diazepam, nitrazepam, rhodamine B, fluorescent brightener Ksn, zearalenone, Sudan Red1, acetominophen, acrylamide, benzoic acid, benzophenone, benzothiazine, mercaptobenzothiazole, erythrosine, Sudan, tartrazine, erythromycin, sirolimus, atropine, ethyl glucuronide, aflatoxin M1, methocarbamol, fentanyl, hydromorphone, morphine, remifentanil, tapentadol, tramadol, pregabalin, gabapentin, amitriptyline, desipramine, imipramine, nortriptyline, venlafaxine, dinitrophenyl, His-tag, PEG methoxy group, etodolac, ibuprofen, ketoprofen, meclofenamic acid, phenylbutazone, acetyl salicylic acid, acetamiprid, acetochlor, carbadazim, carbaryl, chlorothalonil, chlorpyrifos, fenpropathrin, imazalil, imidacloprid, parathion, abscisic acid, dibutyl phthalate, clonazepam, lorazepam, oxazepam, phenobarbital, secobarbital, zaleplon, zolpidem, trazodone, fluoxetine, fluvoxamine, cortisone, dexamethasone, dihydrotestosterone, fluocinolone, methylprednisolone, prednisolone, stanozolol, triamcinolone, mazindol, methamphetamine, methylphenidate, modafinil, chrysoidine, deoxynivalenol, fumonisin, microcystin Lr, ochratoxin, sterigmatocystin, T-2 toxin, sildenafil, tadalafil, scopolamine, florfenicol, pirlimycin, and sulfaquinoxaline.
180. The composition of any one of Clauses 170-179, wherein the activator is a small molecule activator.
181. The composition of Clause 180, wherein the small molecule activator is a fluorescein, a fluorescein derivative, or a tetraxetan (DOTA).
182. The composition of any one of Clauses 170-181, wherein the intracellular domain is derived from an endogenous cytokine receptor intracellular domain.
183. The composition of Clause 182, wherein the endogenous cytokine receptor intracellular domain is an IL2Rα intracellular domain, an IL2Rβ intracellular domain, an IL2Rγ intracellular domain, an IL4Rα intracellular domain, an IL7Rα intracellular domain, an IL9Rα intracellular domain, an IL15Rα intracellular domain, or an IL21Rα intracellular domain.
184. The composition of any one of Clauses 170-183, wherein the engineered immune cell is a T-cell, a B-cell, a natural killer cell (NK cell), a FcεRIγ deficient NK cell (g-NK cell), a neutrophil, an eosinophil, a macrophage, or a γδ T-cell.
185. The composition of any one of Clauses 170-184, wherein the cytokine receptor switch comprises a sequence of any one of SEQ ID NO: 1-SEQ ID NO: 7.
186. The composition of any one of Clauses 170-185, wherein the engineered immune cell further comprises a chimeric antigen receptor, or a polynucleotide encoding the chimeric antigen receptor.
187. The composition of Clause 186, wherein the chimeric antigen receptor is capable of binding CD19, CD20, CD22, CD123, CD33, CD3, CD4, CD8, BCMA, CD38, SLAMF7, GD2, GPRC5D, MUC16, HER2, EGFR, EGFRvIII, CLL-1, CD44v6, B7-H3, EphA2, GRP78, NKG2D, CD70, folate receptor-α, or mesothelin.
188. A pharmaceutical composition comprising the composition of any one of Clauses 170-187 and a pharmaceutically acceptable carrier.
189. A method of activating an engineered immune cell in a subject, the method comprising:
administering to the subject:
   an engineered immune cell expressing a cytokine receptor switch, wherein the
      cytokine receptor switch comprises:
      activator binding domain that binds to an activator,
      a signal peptide,
      a hinge domain,
      a transmembrane domain, and
      an intracellular domain comprising a cytokine receptor intracellular domain that is activated upon binding of the activator to the activator binding domain; and
   a bispecific agent comprising the activator linked to a targeting protein, wherein the targeting protein binds a lymphoid marker; and
delivering the engineered immune cell to a lymphoid organ, thereby activating the engineered immune cell.
190. The method of Clause 189, wherein the lymphoid organ is a lymph node, a spleen, a thymus, or bone marrow.
191. The method of Clause 189 or 190, further comprising binding the engineered immune cell to an antigen presenting cell, a T-cell, a B-cell, a lymphocyte, a lymphatic endothelial cell, a B cell, a macrophage, or a lymphoid organ stromal cell.
192. The method of Clause 191, wherein the antigen presenting cell is a dendritic cell.
193. The method of any one of Clauses 189-192, comprising promoting clonal expansion of the engineered immune cell.
194. The method of any one of Clauses 189-193, comprising promoting differentiation of the engineered immune cell.
195. The method of Clause 194, wherein the engineered immune cell differentiates into a memory T-cell.
196. The method of Clause 195, wherein the memory T-cell is a stem-cell memory T-cell, a central memory T-cell, an effector memory T-cell, or an effector memory re-expressing CD45RA T-cell.
197. The method of Clause 195 or 196, wherein the memory T-cell persists in the subject for longer than an effector T-cell.
198. The method of any one of Clauses 195-197, wherein the memory T-cell produces effector T-cells.
199. The method of any one of Clauses 189-198, comprising promoting expression of CCR7, CD45RA, CD95, or combinations thereof in the engineered immune cell.
200. The method of any one of Clauses 189-199, comprising binding the bispecific agent to the lymphoid marker via the targeting protein with an equilibrium dissociation constant (K_{D}) of no more than 1 μM, no more than 100 nM, no more than 10 nM, or no more than 1 nM.
201. The method of any one of Clauses 189-200, comprising retaining the engineered immune cell in a lymphoid organ for 6 to 96 hours, 12 to 72 hours, or 24 to 48 hours.
202. The method of any one of Clauses 189-201, wherein the engineered immune cell further expresses a chimeric antigen receptor, or a polynucleotide encoding the chimeric antigen receptor.
203. The method of Clause 202, wherein the chimeric antigen receptor binds a tumor antigen.
204. The method of Clause 203, wherein the tumor antigen is CD19, CD123, CD33, BCMA, CD38, CD3, CD4, CD8, SLAMF7, GPRC5D, MUC16, HER2, folate receptor-α, B7-H3, EphA2, GRP78, NKG2D, CD70, or mesothelin.
205. The method of Clause 203 or 204, further comprises binding the chimeric antigen receptor to a cancer cell expressing the tumor antigen.
206. A method of activating an engineered immune cell in a subject, the method comprising administering the composition of any one of Clauses 170-187 to the subject.
207. A method of treating a cancer in a subject, the method comprising:
administering to the subject:
   an engineered immune cell expressing a chimeric antigen receptor and a cytokine
      receptor switch, wherein the cytokine receptor switch comprises:
      activator binding domain that binds to an activator,
      a signal peptide,
      a hinge domain,
      a transmembrane domain, and
      an intracellular domain comprising a cytokine receptor intracellular domain that is activated upon binding of the activator to the activator binding domain; and
   a bispecific agent comprising the activator linked to a targeting protein, wherein the targeting protein binds a lymphoid marker; and
activating the engineered immune cell by delivering the engineered immune cell to a lymphoid organ, thereby treating the cancer.
208. The method of Clause 207, further comprising recruiting the immune cell to a cancer cell by binding the chimeric antigen receptor to a target antigen on the cancer cell.
209. The method of Clause 207 or 208, further comprising activating an immune response against the cancer cell.
210. The method of any one of Clauses 207-209, further comprising killing the cancer cell.
211. The method of any one of Clauses 207-210, wherein the chimeric antigen receptor binds a tumor antigen.
212. The method of Clause 211, wherein the tumor antigen is CD19, CD123, CD33, CD3, CD4, CD8, CD38, SLAMF7, BCMA, GPRC5D, MUC16, HER2, folate receptor-α, B7-H3, EphA2, GRP78, NKG2D, CD70, or mesothelin.
213. The method of any one of Clauses 207-212, wherein the cancer is acute myeloid leukemia, multiple myeloma, ovarian cancer, mesothelioma, non-Hodgkin lymphoma, acute lymphoblastic leukemia, mantle cell lymphoma, follicular lymphoma, glioma, pancreatic cancer, prostate cancer, or gastric cancer.
214. The method of any one of Clauses 207-213, comprising producing an anti-cancer effect in the subject that persists longer than an anti-cancer effect produced by an immune cell lacking the cytokine receptor switch.
215. A method of treating a cancer in a subject, the method comprising administering the composition of any one of Clauses 170-187 to the subject.

### CONCLUSION

Although many of the embodiments are described above with respect to compositions and methods for cancer immunotherapy, the technology is applicable to other applications and/or other approaches, such as immunotherapy for other types of diseases and conditions. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-29B.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. Additionally, the term “comprising” is used throughout to mean including at least the recited feature(s) such that any greater number of the same feature and/or additional types of other features are not precluded.

As used herein, the singular forms “a,” “an” and “the” include plural referents unless the context clearly dictates otherwise.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

As used herein, the term percent “identity,” in the context of two or more nucleic acid or polypeptide sequences, may refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity may be performed using the BLAST algorithm, which is described in Altschul et al. (J. Mol. Biol. 215:403-410 (1990)). Software for performing BLAST analyses is publicly available through the National Center for Biotechnology Information.

A composition can be administered alone or in combination with other treatments, either simultaneously or sequentially dependent upon the condition to be treated.

As used herein, the term “subject” broadly refers to any animal, including but not limited to, human and non-human animals (e.g., dogs, cats, cows, horses, sheep, pigs, poultry, fish, crustaceans, etc.).

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the term “therapeutically effective amount” is an amount that is effective to ameliorate a symptom of a disease. A therapeutically effective amount can be a “prophylactically effective amount” as prophylaxis can be considered therapy.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intraperitoneally, etc.) and the like.

As used herein, the term “treatment” means an approach to obtaining a beneficial or intended clinical result. The beneficial or intended clinical result can include alleviation of symptoms, a reduction in the severity of the disease, inhibiting an underlying cause of a disease or condition, steadying diseases in a non-advanced state, delaying the progress of a disease, and/or improvement or alleviation of disease conditions.

As used herein, the term “pharmaceutical composition” refers to the combination of an active ingredient with a carrier, inert or active, making the composition especially suitable for therapeutic or diagnostic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), glycerol, liquid polyethylene glycols, aprotic solvents such as dimethylsulfoxide, N-methylpyrrolidone and mixtures thereof, and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 21st Ed., Mack Publ. Co., Easton, Pa. (2005), incorporated herein by reference in its entirety.

As used herein, the term “transfection” refers to any method for introducing a nucleic acid into a cell, including both viral and non-viral methods, and encompasses both transient and stable modifications.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1.-215. (canceled)

216. A composition comprising:
an immune cell population, wherein the immune cell population comprises immune cells expressing a cytokine receptor switch comprising:
an activator binding domain,
a signal peptide,
a hinge domain,
a transmembrane domain, and
an intracellular domain, wherein the intracellular domain comprises a cytokine receptor intracellular domain,
wherein at least 20% of the immune cells in the immune cell population have a memory phenotype.

217. The composition of claim 216, wherein the intracellular domain comprises a sequence having at least 80% sequence identity to any one of SEQ ID NO: 29-SEQ ID NO: 34.

218. The composition of claim 216, wherein the intracellular domain comprises or is derived from an intracellular domain of any of the following: IL2Rα, IL2Rβ, IL2Rγ, IL4Rα, IL7Rα, IL15Rα, IL21Rα, IL1R, CD123, CD124, IL5Rα, IL5Rβ, CD126, CD132, CD129, IL11Rα, IL12Rβ1, IL12Rβ2, IL13Rα1, CD122, IL18R, IL23R, IL27Rα, CD130, or GM-CSF.

219. The composition of claim 216, wherein the intracellular domain comprises a single intracellular domain.

220. The composition of claim 216, wherein the intracellular domain comprises a plurality of intracellular domains in tandem.

221. The composition of claim 216, wherein the transmembrane domain comprises a sequence having at least 80% sequence identity to any one of SEQ ID NO: 23-SEQ ID NO: 28.

222. The composition of claim 216, wherein the transmembrane domain comprises or is derived from a transmembrane domain of any of the following: IL2Rα, IL2Rβ, IL2Rγ, IL4Rα, IL7Rα, IL15Rα, IL21Rα, IL1R, CD123, CD124, IL5Rα, IL5Rβ, CD126, CD132, CD129, IL11Rα, IL12Rβ1, IL12Rβ2, IL13Rα1, CD122, IL18R, IL23R, IL27Rα, CD130, an immunoglobulin, CD8, CD28, GM-CSF, or EpoR.

223. The composition of claim 216, wherein the hinge domain comprises a sequence having at least 80% sequence identity to SEQ ID NO: 22.

224. The composition of claim 216, wherein the hinge domain comprises or is derived from a hinge domain of any of the following: CD8, CD3, CD4, CD28, 4-1BB, CD28, OX40, ICOS, CD27, an immunoglobulin, or EpoR.

225. The composition of claim 216, wherein the signal peptide comprises a sequence having at least 80% sequence identity to any one of SEQ ID NO: 15-SEQ ID NO: 20.

226. The composition of claim 216, wherein the signal peptide comprises or is derived from a signal peptide of any of the following: IL2Rα, IL2Rβ, IL2Rγ, IL4Rα, IL7Rα, IL15Rα, IL21Rα, IL1R, CD123, CD124, IL5Rα, IL5Rβ, CD126, CD132, CD129, IL11Rα, IL12Rβ1, IL12Rβ2, IL13Rα1, CD122, IL18R, IL23R, IL27Rα, CD130, an immunoglobulin, CD8, CD28, or GM-CSF.

227. The composition of claim 216, wherein the activator binding domain comprises a single-chain variable fragment (scFv), a peptide, or a nanobody.

228. The composition of claim 216, wherein the activator binding domain comprises a sequence having at least 80% sequence identity to SEQ ID NO: 21.

229. The composition of claim 216, wherein the activator binding domain binds to an activator comprising fluorescein, a fluorescein derivative, or tetraxetan (DOTA).

230. The composition of claim 216, wherein the intracellular domain is in an active conformation when the activator binding domain is bound to an activator.

231. The composition of claim 230, wherein the active conformation of the intracellular domain is capable of activating a cytokine signaling pathway.

232. The composition of claim 231, wherein the activation of the cytokine signaling pathway causes conversion to a memory phenotype, upregulation of lymphoid homing markers, or a combination thereof.

233. The composition of claim 216, further comprising a bispecific agent comprising an activator and a targeting moiety, wherein the activator binding domain binds to the activator.

234. The composition of claim 233, wherein the targeting moiety binds to a tumor antigen.

235. The composition of claim 216, wherein the cytokine receptor switch comprises a sequence having at least 80% sequence identity to any one of SEQ ID NO: 1-SEQ ID NO: 7.
